(19) United States
(12) Patent Application Publication
(10) Pub. No.: US 2018/0305702 A1
(43) Pub. Date: Oct. 25, 2018

(54) Listeria-Based Immunogenic Compositions And Methods Of Use Thereof in Cancer Prevention And Treatment

(71) Applicant: Advaxis, Inc., Princeton, NJ (US)

(72) Inventors: Robert Petit, Newtown, PA (US); Michael F. Princiotta, Hightstown, NJ (US); Kyle Perry, Lawrenceville, NJ (US)

(73) Assignee: Advaxis, Inc., Princeton, NJ (US)

(21) Appl. No.: 15/563,447

(22) PCT Filed: Sep. 14, 2016
(86) PCT No.: PCT/US2016/051748

§ 371 Date: Sep. 29, 2017

Related U.S. Application Data
(60) Provisional application No. 62/218,896, filed on Sep. 15, 2015.

(51) Int. Cl. C12N 15/74; C07K 14/195; C12N 9/64; C07K 14/47; C12N 9/90; C12N 9/10

(52) U.S. Cl. CPC C12N 15/74; C07K 14/195; C12N 9/6424; C12Y 304/21077; C07K 14/4747; C07K 14/4748; C07K 2319/03; C12N 9/90; C12Y 501/01001; C12N 9/1096; C12Y 206/01021; C07K 2319/00; C12Y 304/21106

(57) ABSTRACT

Disclosed herein are recombinant Listeria strains comprising nucleotides encoding two or more heterologous antigens each fused to a truncated LLO, an N-terminal ActA or a PEST-sequence, methods of preparing same, and methods of inducing an immune response, and treating, inhibiting, or suppressing cancer or tumors comprising administering same.

### CROSS-REFERENCE TO RELATED APPLICATIONS

This application claims the benefit of U.S. Application No. 62/218,896, filed Sep. 15, 2015, herein incorporated by reference in its entirety for all purposes.

### REFERENCE TO A SEQUENCE LISTING SUBMITTED AS A TEXT FILE VIA EFS WEB

The Sequence Listing written in file 483704SEQLIST.txt is 556 kb, was created on Sep. 13, 2016, and is hereby incorporated by reference.

### BACKGROUND OF THE INVENTION

A great deal of pre-clinical evidence and early clinical trial data suggests that the anti-tumor capabilities of the immune system can be harnessed to treat patients with established cancers. The vaccine strategy takes advantage of tumor antigens associated with various types of cancers. Immunizing with live vaccines such as viral or bacterial vectors expressing a tumor-associated antigen is one strategy for eliciting strong CTL responses against tumors.

Listeria monocytogenes (Lm) is a gram positive, facultative intracellular bacterium that has direct access to the cytoplasm of antigen presenting cells, such as macrophages and dendritic cells, largely due to the pore-forming activity of listeriolysin-O (LLO). LLO is secreted by Lm following engulfment by the cells and perforates the phagolysosomal membrane, allowing the bacterium to escape the vacuole and enter the cytoplasm. LLO is very efficiently presented to the immune system via MHC class I molecules. Furthermore, Lm-derived peptides also have access to MHC class II presentation via the phagolysosome.

Cancer is a complex disease and combined therapeutic approaches are more likely to succeed. Not only tumor cells, but also the microenvironment that supports tumor growth, must be targeted to maximize the therapeutic efficacy. Most immunotherapies focus on single antigens to target tumor cells and therefore they have shown limited success against human cancers. A single therapeutic agent capable of targeting one or more targets, such as tumor cells and tumor microenvironment simultaneously would have an advantage over other immunotherapeutic approaches, especially if it results in a synergistic anti-tumor effect.

### SUMMARY OF THE INVENTION

In one aspect, the invention relates to a method of eliciting an anti-tumor or anti-cancer immune response in a subject, the method comprising the step of administering to said subject an effective amount of an immunogenic composition comprising a recombinant Listeria strain comprising a recombinant nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or an immunogenic fragment thereof, and wherein said Listeria expresses said fusion polypeptide, thereby eliciting an anti-tumor or anti-cancer immune response in said subject.

In one aspect, the invention relates to an immunogenic composition comprising a recombinant Listeria strain comprising a recombinant nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to an endoglin sequence or an immunogenic fragment thereof, wherein said Listeria strain comprises mutations in endogenous genes encoding a D-alanine racemase (dal) and a D-amino acid transferase (dat) gene, and in a virulence gene encoding an ActA (actA).

In another aspect, said recombinant nucleic acid molecule in said Listeria comprises a second open reading frame. In another aspect, said second open reading frame encodes a second fusion polypeptide, wherein said fusion polypeptide comprises a truncated listeriolysin O (LLO) protein, a truncated ActA protein, or a PEST amino acid sequence fused to a heterologous antigen or an immunogenic fragment thereof, and wherein said Listeria expresses said second fusion polypeptide.

In another aspect, said heterologous antigen is selected from prostate stem cell antigen (PSCA), prostate-specific antigen (PSA; KLK3), A Kinase Anchor Protein 4 (AKAP4), HPV E7, Hepsin (HPN/TMPRSS1), Prostate-specific G-protein-coupled receptor (PSGR/OR51E2), T-cell receptor γ-chain Alternate Reading-Frame Protein (TARP), survivin (Birc5), Mammalian Enabled Homolog (ENAH; hMENA), POTE paralogs, O-GlcNAc Transferase (OGT), KLK7, Secernin-1 (SCRN1), Fibroblast Activation Protein (FAP), Matrix Metallopeptidase 7 (MMP7), Milk Fat Globule-EGF Factor 8 Protein (MFGE8), Wilms Tumor 1 (WT1), Interferon-Stimulated Gene 15 Ubiquitin-Like Modifier (ISG15; G1P2), Acrosin Binding Protein (ACRBP; OY-TES-1), Kallikrein-Related Peptidase 4 (KLK4/prostase).

In one aspect, the invention further relates to a recombinant Listeria strain comprising a nucleic acid molecule comprising a first open reading frame encoding a fusion polypeptide, said fusion polypeptide comprising a prostate specific (PSA) antigen or a immunogenic fragment thereof fused to a truncated listeriolysin O (LLO), a truncated ActA or PEST amino acid sequence, and wherein said nucleic acid molecule further comprises a second open reading frame encoding a fusion polypeptide, said fusion polypeptide comprising a prostate-specific membrane antigen (PSMA) or an immunogenic fragment thereof fused to a truncated listeriolysin O (LLO), a truncated ActA or PEST amino acid sequence.

In another aspect, the invention further relates to a recombinant Listeria strain comprising a nucleic acid molecule comprising a first open reading frame encoding a fusion polypeptide, said fusion polypeptide comprising a prostate specific (PSA) antigen or an immunogenic fragment thereof fused to a truncated listeriolysin O (LLO), a truncated ActA or PEST amino acid sequence, and wherein said nucleic acid molecule further comprises a second open reading frame encoding a fusion polypeptide, said fusion polypeptide comprising a survivin antigen or an immunogenic fragment thereof fused to a truncated listeriolysin O (LLO), a truncated ActA or PEST amino acid sequence.

In one aspect, the invention relates to a method of inducing an anti-tumor immune response in a subject comprising administering to said subject the recombinant Listeria disclosed herein. In a related aspect, the immune response allows treating, suppressing, or inhibiting a cancer in a subject.

In one aspect, the invention relates to a recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof, a survivin antigen or an immunogenic fragment thereof, a prostate specific G-protein coupled receptor (PSGR) antigen or an immunogenic fragment thereof, and a hepsin antigen or an immunogenic fragment thereof. In a related aspect, the invention relates to an immunogenic composition comprising the recombinant Listeria strain. In a related aspect, the invention relates to a method of inducing an immune response against a tumor or cancer in a subject, comprising administering to the subject the recombinant Listeria strain or administering to the subject the immunogenic composition. In a related aspect, the invention relates to a method of preventing or treating a tumor or cancer in a subject, comprising administering to the subject the recombinant Listeria strain or the immunogenic composition.

In one aspect, the invention relates to a recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof and a survivin antigen or an immunogenic fragment thereof. In a related aspect, the invention relates to an immunogenic composition comprising the recombinant Listeria strain. In a related aspect, the invention relates to a method of inducing an immune response against a tumor or cancer in a subject, comprising administering to the subject the recombinant Listeria strain or administering to the subject the immunogenic composition. In a related aspect, the invention relates to a method of preventing or treating a tumor or cancer in a subject, comprising administering to the subject the recombinant Listeria strain or the immunogenic composition.

In one aspect, the invention relates to a recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof and a PSMA antigen or an immunogenic fragment thereof. In a related aspect, the invention relates to an immunogenic composition comprising the recombinant Listeria strain. In a related aspect, the invention relates to a method of inducing an immune response against a tumor or cancer in a subject, comprising administering to the subject the recombinant Listeria strain or administering to the subject the immunogenic composition. In a related aspect, the invention relates to a method of preventing or treating a tumor or cancer in a subject, comprising administering to the subject the recombinant Listeria strain or the immunogenic composition.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1. (A) Schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIG. 2. (A) Map of the pADV134 plasmid. (B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (C) Map of the pADV142 plasmid. (D) Western blot showed the expression of LLO-PSA fusion protein using anti-PSA and anti-LLO antibody.

FIG. 3. Schematic representation of monovalent and bivalent plasmids. Restriction sites that were used for cloning of antigen 1 (XhoI and SpeI) and antigen 2 (XbaI and SacI or BglII) are indicated. The black arrow represents the direction of transcription. p15 ori and RepR refers to E. coli and Listeria origin of replication. tLLO is truncated listeriolysin O protein. Bacillus-dal gene codes for D-alanine racemase which complements for the synthesis of D-alanine in Lm Δ dal dat strain.

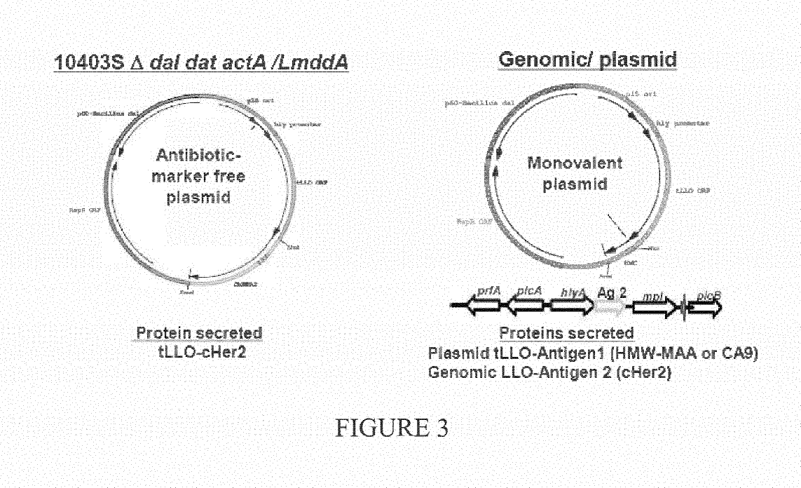

FIG. 4. (A) Plasmid stability in vitro of LmddA-LLO-PSA if cultured with and without selection pressure (D-alanine). Strain and culture conditions are listed first and plates used for CFU determination are listed after. (B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIG. 5. (A) In vivo clearance of the strain LmddA-LLO-PSA after administration of 10⁸ CFU in C57BL/6 mice. The number of CFU were determined by plating on BHI/str plates. The limit of detection of this method was 100 CFU. (B) Cell infection assay of J774 cells with 10403S, LmddA-LLO-PSA and XFL7 strains.

FIG. 6. (A) PSA tetramer-specific cells in the splenocytes of naïve and LmddA-LLO-PSA immunized mice on day 6 after the booster dose. (B) Intracellular cytokine staining for IFN-γ in the splenocytes of naïve and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naïve mice at different effector/target ratio using a caspase based assay (C) and a europium based assay (D). Number of IFNγ spots in naïve and immunized splenocytes obtained after stimulation for 24 h in the presence of PSA peptide or no peptide (E).

FIG. 7. Immunization with LmddA-142 induces regression of Tramp-C1-PSA (TPSA) tumors. Mice were left untreated (n=8) (A) or immunized i.p. with LmddA-142 (1×10⁸ CFU/mouse) (n=8) (B) or Lm-LLO-PSA (n=8) (C) on days 7, 14 and 21. Tumor sizes were measured for each individual tumor and the values expressed as the mean diameter in millimeters. Each line represents an individual mouse.

FIG. 8. (A) Analysis of PSA-tetramer⁺CD8⁺ T cells in the spleens and infiltrating T-PSA-23 tumors of untreated mice and mice immunized with either an Lm control strain or Lm-ddA-LLO-PSA (LmddA-142). (B) Analysis of CD4⁺ regulatory T cells, which were defined as CD25⁺FoxP3⁺, in the spleens and infiltrating T-PSA-23 tumors of untreated mice and mice immunized with either an Lm control strain or Lm-ddA-LLO-PSA.

FIG. 9. (A) Schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 760 bp corresponding to the klk3 gene.

FIG. 10. (A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (B) LLO produced by Lmdd-143 and LmddA-143 retains hemolytic activity. Sheep red blood cells were incubated with serial dilutions of bacterial culture supernatants and hemolytic activity measured by absorbance at 590 nm; (C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated time points. Lm 10403S was used as a control in these experiments.

FIG. 11. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIG. 12. Three Lm-based vaccines expressing distinct HMW-MAA fragments based on the position of previously mapped and predicted HLA-A2 epitopes were designed (A). The Lm-tLLO-HMW-MMA₂₁₆₀₋₂₂₅₈ (also referred as Lm-LLO-HMW-MAA-C) strain secretes a ˜62 kDa band corresponding to the tLLO-HMW-MAA₂₁₆₀₋₂₂₅₈ fusion protein (B). C57BL/6 mice (n=15) were inoculated s.c. with B16F10 cells and either immunized i.p. on days 3, 10 and 17 with Lm-tLLO-HMW-MAA₂₁₆₀₋₂₂₅₈ (n=8) or left untreated (n=7). BALB/c mice (n=16) were inoculated s.c. with RENCA cells and immunized i.p. on days 3, 10 and 17 with either Lm-HMW-MAA-C (n=8) or an equivalent dose of a control Lm vaccine. Mice immunized with the Lm-LLO-HMW-MAA-C impeded the growth of established tumors (C). FVB/N mice (n=13) were inoculated s.c. with NT-2 tumor cells and immunized i.p. on days 7, 14 and 21 with either Lm-HMW-MAA-C (n=5) or an equivalent dose of a control Lm vaccine (n=8). Immunization of mice with Lm-LLO-HMW-MAA-C significantly impaired the growth of tumors not engineered to express HMW-MAA, such as B16F10, RENCA and NT-2 (D). Tumor sizes were measured for each individual tumor and the values expressed as the mean diameter in millimeters ±SEM. *, P≤0.05, Mann-Whitney test.

FIG. 13. Immunization with Lm-HMW-MAA-C promotes tumor infiltration by CD8⁺ T cells and decreases the number of pericytes in blood vessels. (A) NT-2 tumors were removed and sectioned for immunofluorescence. Staining groups are numbered (1-3) and each stain is indicated on the right. Sequential tissues were either stained with the pan-vessel marker anti-CD31 or the anti-NG2 antibody for the HMW-MAA mouse homolog AN2, in conjunction with anti-CD8a for possible TILs. Group 3 shows isotype controls for the above antibodies and DAPI staining used as a nuclear marker. A total of 5 tumors were analyzed and a single representative image from each group is shown. CD8⁺ cells around blood vessels are indicated by arrows. (B) Sequential sections were stained for pericytes by using the anti-NG2 and anti-alpha-smooth-muscle-cell-actin (α-SMA) antibodies. Double staining/colocalization of these two antibodies (yellow in merge image) are indicative of pericyte staining (top). Pericyte colocalization was quantitated using Image Pro Software and the number of colocalized objects is shown in the graph (bottom). A total of 3 tumors were analyzed and a single representative image from each group is shown. *, P≤0.05, Mann-Whitney test. Graph shows mean±SEM.

FIG. 14. (A) LmddA244G/168. Listeria strain expressing chromosomal LLO-cHer2 was constructed by the method of double allelic homologous recombination between the chromosomal gene and the temperature sensitive Listeria shuttle plasmid to create LmddA-cHer2 (referred as LmddA244G). Further, to generate the bivalent strain, LmddA244G was transformed with the plasmid containing expression cassette for the fusion protein tLLO-HMC (pAdv168) (B) resulting in strain LmddA244G/168. LmddA strain was transformed with plasmid pAdv164, which contains expression cassette for tLLO-cHer2 fusion protein to create LmddA164 vaccine. LmddA backbone was transformed with plasmid pAv168, which contains expression cassette for tLLO-HMC (2160-2258 amino acid residues at the C-terminus of HMW-MAA or CSPG4) fusion protein to create LmddA168 vaccine. (C) Further, the expression and secretion of the two fusion proteins in LmddA244G/168, LLO-ChHer2 and tLLO-HMC was detected by western blot using anti-LLO and anti-FLAG antibodies respectively.

FIG. 15. Hemolytic activity of LmddA244G-168 was quantified using Sheep Red Blood cells. A 1.5 fold reduction in the hemolytic activity of bivalent immunotherapy LmddA244G-168 was observed when compared to 10403S. B. Intracellular growth of both bivalent and monovalent immunotherapies in J774 cell line. The intracellular growth of LmddA244G-168 was similar to monovalent immunotherapies LmddA164 and LmddA168.

FIG. 16. A. Established NT2 tumors were implanted with treated with mono therapies and bivalent therapy on days 6, 13 and 20. The naïve group is untreated mice. B. The percent tumor free mice in different treatment and untreated naïve group. C. The volume of established NT2 tumors after of LmddA244G-168 treatment.

FIG. 17. A. Generation of Her2 specific immune responses in mice after administration of monovalent (LmddA164) as well as bivalent immunotherapy (LmddA244G-168) expressing chimera Her2. The Her2 specific immune responses were evaluated in an ELIspot based assay using FvB IC1 peptide epitope-RLLQETELV (Seavey et al 2009, Clin Cancer Res. 2009 Feb. 1; 15(3):924-32. B. Generation of HMW-MAA-C specific immune responses in mice after administration of monovalent (LmddA168) as well as bivalent immunotherapy (LmddA244G-168) expressing HMW-MAA-C. The Her2 specific immune responses were evaluated in an ELISA based assay using affinity purified HMA-MAA-C protein fragment.

FIG. 18. Immunohistochemical (IHC) staining of tumors anti-CD3 antibody on day 27 of the tumor regression study. NT2 tumors were implanted on day 0 and were immunized on days 6, 13 and 20 with different immunotherapies (top left panel) untreated naïve group; (top right panel) mono immunotherapy (LmddA164); (bottom left panel) mono immunotherapy (LmddA168); and (bottom right panel) bivalent immunotherapy (LmddA244G-168).

FIG. 19. Immunohistochemical (IHC) staining of tumors anti-CD8 antibody on day 27 of the tumor regression study. NT2 tumors were implanted on day 0 and were immunized on days 6, 13 and 20 with different immunotherapies (top left panel) untreated naïve group; (top right panel) mono immunotherapy (LmddA164); (bottom left panel) mono immunotherapy (LmddA168); and (bottom right panel) bivalent immunotherapy (LmddA244G-168).

FIG. 20. Immunohistochemical (IHC) staining of tumors anti-CD4 antibody on day 27 of the tumor regression study. NT2 tumors were implanted on day 0 and were immunized on days 6, 13 and 20 with different immunotherapies (top left panel) untreated naïve group; (top right panel) mono immunotherapy (LmddA164); (bottom left panel) mono immunotherapy (LmddA168); and (bottom right panel) bivalent immunotherapy (LmddA244G-168).

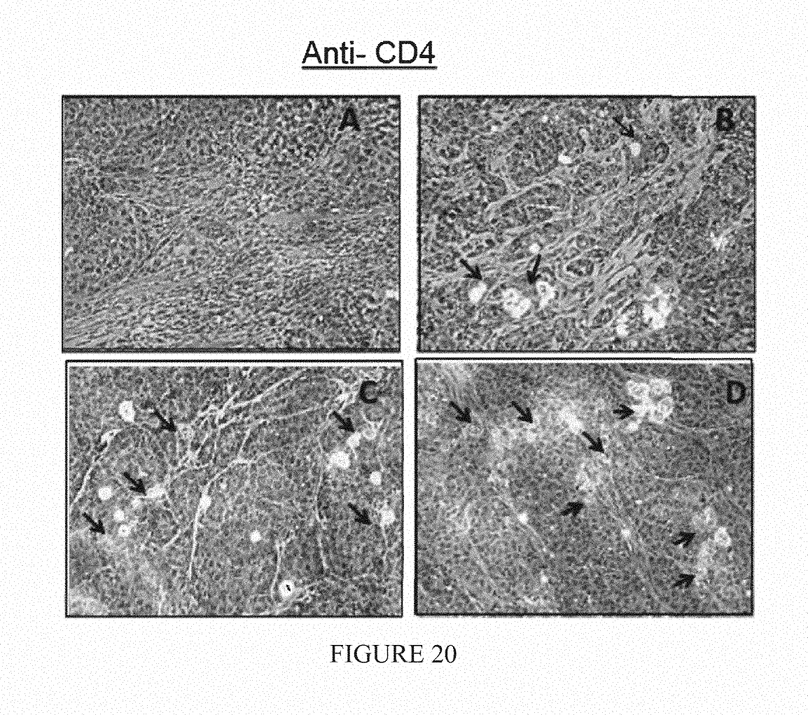

FIG. 21. Immunohistochemical (IHC) staining of tumors anti-CD31 antibody on day 27 of the tumor regression study. NT2 tumors were implanted on day 0 and were immunized on days 6, 13 and 20 with different immunotherapies (top left panel) untreated naïve group; (top right panel) mono immunotherapy (LmddA164); (bottom left panel) mono immunotherapy (LmddA168); and (bottom right panel) bivalent immunotherapy (LmddA244G-168).

FIG. 22. Graph showing the individual mice and the tumor sizes on the days of tumor measurement: days 11, 18, and 21 following administration of various Listeria-based constructs.

FIG. 23. Established 4T1 tumors were treated with mono therapies and bivalent therapy on days 3 and 10. The naïve group is untreated mice.

FIG. 24. Established 4T1 tumors were treated with mono therapies and bivalent therapy on days 1, 8, and 15. The naïve group is untreated mice.

FIG. 25. Established NT2 tumors were treated with mono therapies, bivalent therapy, or sequential mono therapies. The naïve group is untreated mice.

FIG. 26. Percentage of D^{b} PSA₆₅₋₇₃ dextramer positive CD8⁺ T cells after primary immunization with SIINFEKL minigene (LmddA324), PSA-Survivin-SIINFEKL-His and PSA-PSMA-SIINFEKL-His expressing Lm.

FIG. 27. Percentage of K^{b} OVA₂₅₇₋₂₆₄ dextramer positive CD8⁺ T cells after primary immunization with SIINFEKL minigene (LmddA324), PSA-Survivin-SIINFEKL-His and PSA-PSMA-SIINFEKL-His expressing Lm.

FIG. 28. Percentage of D^{b} PSA₆₅₋₇₃ dextramer positive CD8⁺ T cells after secondary (prime plus two boosts) immunization with SIINFEKL minigene (LmddA324), PSA-Survivin-SIINFEKL-His and PSA-PSMA-SIINFEKL-His expressing Lm.

FIG. 29. Percentage of K^{b} OVA₂₅₇₋₂₆₄ dextramer positive CD8⁺ T cells after secondary (prime plus two boosts) immunization with SIINFEKL minigene (LmddA324), PSA-Survivin-SIINFEKL-His and PSA-PSMA-SIINFEKL-His expressing Lm.

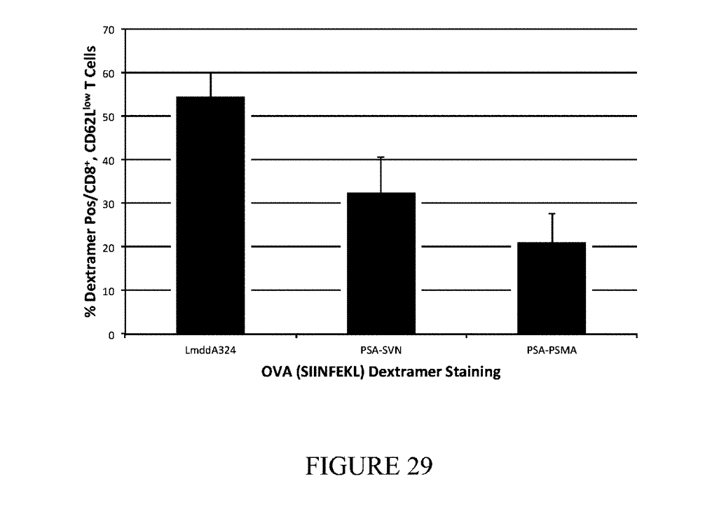

FIG. 30. Pseudocolor plots showing surface K^{b}—SIINFEKL expression levels for three PSA 2.0 Lm constructs and a SIINFEKL minigene expressing Lm as a positive control. Relative percentage of K^{b}-SIINFEKL positive cells is shown in the upper right corner of each plot. Pseudocolor plots are labeled with the specific Lm construct used for infection.

FIG. 31 is a schematic map of pAdv2142 with the tLLO-PSA-Survivin-PSGRΔTM-HepsinΔTM-SIINFEKL-6×HIS fusion protein expression cassette and other features labeled.

FIGS. 32A and 32B show colony PCR for presence of pAdv2142 in putative transformants. FIG. 32A shows colony PCR of putative MB2159+pAdv2142 transformants. All seven putative clones tested produced the expected ˜3 kb PCR product band corresponding to the presence of the PSA-Survivin-PSGRΔTMs-HepsinΔTM-SIINFEKL-6×HIS region of pAdv2142. Lanes: (1) Ladder; (2) putative clone #1; (3) putative clone #2; (4) putative clone #3; (5) putative clone #4; (6) putative clone #5; (7) putative clone #6; (8) putative clone #7. FIG. 32B shows colony PCR of putative LmddA+pAdv2142 transformants. All eight putative clones tested produced the expected ˜3 kb PCR product band corresponding to the presence of the PSA-Survivin-PSGRΔTMs-HepsinΔTM-SIINFEKL-6×HIS region of pAdv2142. Lanes: (1) Ladder; (2-5) unrelated PCR reactions; (6) putative clone #1; (7) putative clone #2; (8) putative clone #3; (9) putative clone #4; (10) putative clone #5; (11) putative clone #6; (12) putative clone #7; and (13) putative clone #13.

FIG. 33 shows in vitro SIINFEKL presentation of ADXS31-2142-infected DC2.4 cells. DC2.4 dendritic cells were infected with the indicated strains at an MOI of 20. At one hour post-infection, host cells were washed and the tissue culture medium replaced with fresh medium containing gentamycin to kill extracellular bacteria. At five hours post-infection, host cells were harvested and stained with Alexa647-conjugated 25D-1.16 antibody. The percentage of Alexa647⁺ DC2.4 cells was then assessed by flow cytometry. Whereas DC2.4 cells infected with a non-SIINFEKL-expressing Lm strain do not produce an appreciable Alexa647⁺ population (left panel, 0.37% of DC2.4), ADXS31-2142-infected DC2.4 cells produce a significant Alexa647⁺ population (right panel, 14.6% of DC2.4 cells).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details, as embodied herein. In other instances, well-known methods, procedures, and components have not been described in detail so as not to obscure the present invention.

In one aspect, disclosed herein is a recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to two or more heterologous antigens or immunogenic fragments thereof, such as two or more of a prostate specific antigen (PSA) antigen or an immunogenic fragment thereof, a survivin antigen or an immunogenic fragment thereof, a prostate specific G-protein coupled receptor (PSGR) antigen (e.g., PSGRΔTM) or an immunogenic fragment thereof, a hepsin antigen (e.g., hepsinΔTM) or an immunogenic fragment thereof, a prostate-specific membrane antigen (PSMA) antigen (e.g., PSMAΔTM) or an antigenic fragment thereof, and an AKAP4 antigen or an immunogenic fragment thereof. Alternatively, disclosed herein is a recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof and one or more additional heterologous antigens or immunogenic fragments thereof, such as one or more of a survivin antigen or an immunogenic fragment thereof, a prostate specific G-protein coupled receptor (PSGR) antigen (e.g., PSGRΔTM) or an immunogenic fragment thereof, a hepsin antigen (e.g., hepsinΔTM) or an immunogenic fragment thereof, a prostate-specific membrane antigen (PSMA) antigen (e.g., PSMAΔTM) or an antigenic fragment thereof, and an AKAP4 antigen or an immunogenic fragment thereof.

As an example, such a recombinant Listeria strain can comprise a nucleic acid molecule comprising a first open reading frame encoding a fusion polypeptide, the fusion polypeptide comprising a truncated LLO (tLLO), a truncated ActA, or a PEST amino acid sequence fused to a PSA antigen or an immunogenic fragment thereof, a survivin antigen or an immunogenic fragment thereof, a PSGR antigen (e.g., PSGRΔTM) or an immunogenic fragment thereof, and a hepsin antigen (e.g., hepsinΔTM) or an immunogenic fragment thereof (see, e.g., the nucleic acid sequence set forth in SEQ ID NO: 145 or the amino acid sequence set forth in SEQ ID NO: 183).

As another example, such a recombinant Listeria strain can comprise a nucleic acid molecule comprising a first open reading frame encoding a fusion polypeptide, the fusion polypeptide comprising a tLLO, a truncated ActA, or a PEST amino acid sequence fused to a PSA antigen or an immunogenic fragment thereof and a survivin antigen or an immunogenic fragment thereof (see, e.g., the nucleic acid sequence set forth in SEQ ID NO: 92 or 93 or the amino acid sequence set forth in SEQ ID NO: 117 or 118). As yet another example, such a recombinant Listeria strain can comprise a nucleic acid molecule comprising a first open reading frame encoding a fusion polypeptide, the fusion polypeptide comprising a tLLO, a truncated ActA, or a PEST amino acid sequence fused to a PSA antigen or an immunogenic fragment thereof and a PSMA antigen (e.g., PSMAΔTM) or an immunogenic fragment thereof (see, e.g., the nucleic acid sequence set forth in SEQ ID NO: 94 or the amino acid sequence set forth in SEQ ID NO: 119).

In other non-limiting examples, the fusion polypeptide comprises a tLLO, a truncated ActA, or a PEST amino acid sequence fused to a PSA antigen or an immunogenic fragment thereof and a PSGR antigen (e.g., PSGRΔTM) or an immunogenic fragment thereof (see, e.g., the nucleic acid sequence set forth in SEQ ID NO: 138 or 139 or the amino acid sequence set forth in SEQ ID NO: 176 or 177). In other non-limiting examples, the fusion polypeptide comprises a tLLO, a truncated ActA, or a PEST amino acid sequence fused to a PSA antigen or an immunogenic fragment thereof and a hepsin antigen (e.g., hepsinΔTM) or an immunogenic fragment thereof (see, e.g., the nucleic acid sequence set forth in SEQ ID NO: 140 or the amino acid sequence set forth in SEQ ID NO: 178). In other non-limiting examples, the fusion polypeptide comprises a tLLO, a truncated ActA, or a PEST amino acid sequence fused to a PSA antigen or an immunogenic fragment thereof and an AKAP4 antigen or an immunogenic fragment thereof (see, e.g., the nucleic acid sequence set forth in SEQ ID NO: 141 or the amino acid sequence set forth in SEQ ID NO: 179). In other non-limiting examples, the fusion polypeptide comprises a tLLO, a truncated ActA, or a PEST amino acid sequence fused to a PSA antigen or an immunogenic fragment thereof, a survivin antigen or an immunogenic fragment thereof, and a PSGR antigen (e.g., PSGRΔTM) or an immunogenic fragment thereof (see, e.g., the nucleic acid sequence set forth in SEQ ID NO: 142 or the amino acid sequence set forth in SEQ ID NO: 180). In other non-limiting examples, the fusion polypeptide comprises a tLLO, a truncated ActA, or a PEST amino acid sequence fused to a PSA antigen or an immunogenic fragment thereof, a survivin antigen or an immunogenic fragment thereof, and a hepsin antigen (e.g., hepsinΔTM) or an immunogenic fragment thereof (see, e.g., the nucleic acid sequence set forth in SEQ ID NO: 143 or the amino acid sequence set forth in SEQ ID NO: 181). In other non-limiting examples, the fusion polypeptide comprises a tLLO, a truncated ActA, or a PEST amino acid sequence fused to a PSA antigen or an immunogenic fragment thereof, a PSGR (e.g., PSGRΔTM) antigen or an immunogenic fragment thereof, and a hepsin antigen (e.g., hepsinΔTM) or an immunogenic fragment thereof (see, e.g., the nucleic acid sequence set forth in SEQ ID NO: 144 or the amino acid sequence set forth in SEQ ID NO: 182).

Examples of suitable LLO, truncated LLO, ActA, truncated ActA, and PEST amino acid sequences are disclosed elsewhere herein. Some examples of LLO proteins or truncated LLO proteins are set forth in SEQ ID NOS: 4, 7, 21-24, 107, and 158. Examples of a nucleic acid sequence encoding an LLO protein or truncated LLO protein are set forth in SEQ ID NOS: 82 and 120. Some examples of ActA proteins or truncated ActA proteins are set forth in SEQ ID NOS: 38, 40, and 41. Some examples of nucleic acids encoding ActA proteins or truncated ActA proteins are set forth in SEQ ID NOS: 39 and 43. Some examples of PEST sequences are set forth in SEQ ID NOS: 12-20. Other examples of LLO proteins or fragments, ActA proteins or fragments, and PEST sequences and nucleic acids encoding such LLO proteins or fragments, ActA proteins or fragments, and PEST sequences are disclosed elsewhere herein.

PSA is part of the subgroup of serine proteases and is expressed at moderate to high levels in prostate cancers. An exemplary PSA antigen or fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 108 or SEQ ID NO: 159. Such PSA antigens or fragments thereof can include heteroclitic mutations of specific T-cell epitopes of PSA and induced point mutations. Other examples of PSA antigens or fragments thereof include SEQ ID NOS: 45-47, 49, 51, 53, 55, 57, 59, 61-64, and 66. Examples of nucleic acids encoding PSA antigens or fragments thereof include SEQ ID NOS: 48, 50, 52, 54, 56, 58, 60, 65, 83, 121, and 193. Other examples of PSA antigens or fragments thereof and nucleic acids encoding such PSA antigens or fragments thereof are disclosed elsewhere herein.

Survivin is a member of the family of inhibitors of apoptosis involved in cell cycle progression. It is expressed in prostate cancers and is also expressed in breast cancer, colorectal cancer, bladder cancer, lung cancer, pancreatic cancer, renal cancer, lymphomas, and neuroblastomas. Overexpression in cancer tissue is associated with a poor prognosis. An exemplary survivin antigen or fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 109 or SEQ ID NO: 160. Such survivin antigens or fragments thereof can include heteroclitic mutations of specific T-cell epitopes of survivin and induced point mutations. Examples of nucleic acids encoding survivin antigens or fragments thereof include SEQ ID NOS: 84, 122, and 194. Other examples of survivin antigens or fragments thereof and nucleic acids encoding such survivin antigens or fragments thereof are disclosed elsewhere herein.

PSGR is a membrane protein including 7 transmembrane-spanning domains and is expressed in prostate cancer at a higher level than in normal prostate or benign prostatic hyperplasia. As one example, the PSGR antigen or immunogenic fragment thereof can be a PSGRAtransmembrane domain (ATM) antigen in which the transmembrane regions of PSGR have been removed. An exemplary PSGR antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 162 or SEQ ID NO: 161. Such PSGR antigens or fragments thereof can include heteroclitic mutations of specific T-cell epitopes of PSGR and induced point mutations. Examples of nucleic acids encoding survivin antigens or fragments thereof include SEQ ID NOS: 123, 124, 195, and 196. Other examples of PSGR antigens or fragments thereof and nucleic acids encoding such PSGR antigens or fragments thereof are disclosed elsewhere herein.

Hepsin is a type II transmembrane serine protease that is prominently overexpressed in prostate cancer but is also amplified in sarcomas, ovarian cancer, lung adenocarcinoma, lung squamous cell cancer, adenoid cystic cancer, breast cancer, uterine cancer, and colon cancer. It promotes prostate cancer metastasis, particularly to bone marrow. Levels of hepsin correlate with high Gleason score and are indicative of poor clinical outcome. As one example, the hepsin antigen or immunogenic fragment thereof can be a hepsinAtransmembrane domain (ATM) antigen in which the transmembrane regions of hepsin have been removed. An exemplary hepsin antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 164 or SEQ ID NO: 163. Such hepsin antigens or fragments thereof can include heteroclitic mutations of specific T-cell epitopes of hepsin and induced point mutations. Examples of nucleic acids encoding survivin antigens or fragments thereof include SEQ ID NOS: 125, 126, 197, and 198. Other examples of hepsin antigens or fragments thereof and nucleic acids encoding such hepsin antigens or fragments thereof are disclosed elsewhere herein.

PSMA is a type II transmembrane protein that is overexpressed in prostate cancers. As one example, the PSMA antigen or immunogenic fragment thereof can be a PSMAAtransmembrane domain (ATM) antigen in which the transmembrane regions of PSMA have been removed. An exemplary PSMA antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 110 or SEQ ID NO: 111. Such PSMA antigens or fragments thereof can include heteroclitic mutations of specific T-cell epitopes of PSMA and induced point mutations. Examples of nucleic acids encoding PSMA antigens or fragments thereof include SEQ ID NOS: 85 and 86. Other examples of PSMA antigens or fragments thereof and nucleic acids encoding such PSMA antigens or fragments thereof are disclosed elsewhere herein.

AKAP4 is a member of the cancer-testis antigen family and is expressed in prostate cancers, as well as in NSCLCs, ovarian cancers, cervical cancers, breast cancers, and multiple myelomas. An exemplary survivin antigen or fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 165. Such AKAP4 antigens or fragments thereof can include heteroclitic mutations of specific T-cell epitopes of AKAP4 and induced point mutations. Examples of nucleic acids encoding survivin antigens or fragments thereof include SEQ ID NO: 127. Other examples of AKAP4 antigens or fragments thereof and nucleic acids encoding such AKAP4 antigens or fragments thereof are disclosed elsewhere herein.

The PSA antigen or immunogenic fragment thereof, the survivin antigen or immunogenic fragment thereof and the two or more antigens or immunogenic fragments thereof can be in any order in the fusion protein. For example, in a fusion protein comprising a PSA antigen or immunogenic fragment thereof, a survivin antigen or immunogenic fragment thereof, a PSGR antigen or immunogenic fragment thereof, and a hepsin antigen or immunogenic fragment thereof, the PSA antigen or immunogenic fragment thereof, the survivin antigen or immunogenic fragment thereof, the PSGR antigen or immunogenic fragment thereof, and the hepsin antigen or immunogenic fragment thereof can be in any order in the fusion polypeptide. As one example, the PSA antigen or immunogenic fragment thereof, the survivin antigen or immunogenic fragment thereof, the PSGR antigen or immunogenic fragment thereof, and the hepsin antigen or immunogenic fragment thereof can be in the following order from N-terminal to C-terminal: PSA-survivin-PSGR (e.g., PSGRΔTM)-hepsin (e.g., hepsinΔTM). Similarly, the truncated LLO (tLLO), truncated ActA, or PEST amino acid sequence can be located anywhere within the fusion protein (e.g., N-terminal end, C-terminal-end, or internal) and can be fused to any one of the antigens or antigenic fragments. As one example, the fusion protein can comprise from N-terminal to C-terminal: tLLO-PSA-survivin-PSGR (e.g., PSGRΔTM)-hepsin (e.g., hepsinΔTM).

The tLLO, truncated ActA, or PEST amino acid sequence can be fused directly to the antigens or antigen fragments or can be fused to the antigens or antigen fragments via a linker. Likewise, the antigens or antigenic fragments can be fused directly to each other or can be fused indirectly via a linker. An exemplary linker is set forth in SEQ ID NO: 112 or SEQ ID NO: 166. Examples of nucleic acids encoding linkers are set forth in SEQ ID NOS: 87 and 128. For example, the PSA or immunogenic fragment thereof can be linked to the survivin or immunogenic fragment thereof by a first linker, the survivin or immunogenic fragment thereof can be linked to the PSGR (e.g., PSGRΔTM) or immunogenic fragment thereof via a second linker, and the PSGR (e.g., PSGRΔTM) or immunogenic fragment thereof can be linked to the hepsin (e.g., hepsinΔTM) or immunogenic fragment thereof via a third linker. An example of such a fusion protein comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with residues 1-973 of SEQ ID NO: 175 or residues 1-1414 of SEQ ID NO: 183. Other examples of fusion proteins comprise the sequence set forth in any one of SEQ ID NOS: 114-119 and 168-183. Examples of nucleic acids encoding fusion proteins include nucleic acids comprising the sequence set forth in any one of SEQ ID NOS: 89-94, 130-145, and 200. Other examples of fusion proteins and nucleic acids encoding such fusion proteins thereof are disclosed elsewhere herein.

Some fusion proteins further comprise a tag, such as a C-terminal tag or an N-terminal tag. Such tags can include, for example, polyhistidine (His) tags, SIINFEKL-S-6×His tags, FLAG tags, SIINFEKL-S-Flag tags, chitin binding protein (CBP), maltose binding protein (MBP), glutathione-S-transferase (GST), thioredoxin (TIRX), poly(NANP), or any other tag known in the art or as disclosed elsewhere herein.

In one example, the nucleic acid molecule is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% to the sequence set forth in SEQ ID NO: 202.

The nucleic acid molecule can be in any form. In one example, the nucleic acid molecule is operably integrated into the Listeria genome as described in further detail elsewhere herein. In another example, the nucleic acid molecule is in a plasmid as described in further detail elsewhere herein. For example, such a plasmid can be stably maintained in the recombinant Listeria strain in the absence of antibiotic selection. Thus, in some cases, the plasmid does not confer antibiotic resistance upon the recombinant Listeria strain.

The fusion polypeptide can be expressed from any promoter capable of driving expression in the Listeria strain. Examples of suitable promoters include an hly promoter, a prfA promoter, an actA promoter, or ap60 promoter. In one embodiment, the promoter is an hly promoter. Other suitable promoters are disclosed elsewhere herein.

Preferably, the Listeria strain is attenuated. Such an attenuated Listeria strain can comprise, for example, a mutation in one or more endogenous genes. Such mutations can comprise an inactivation, truncation, deletion, replacement, or disruption of the one or more endogenous genes or any other type of mutation. Different ways to attenuate Listeria strains are disclosed in further detail elsewhere herein. For example, the attenuated Listeria strain can comprise a mutation in an actA virulence gene, in an endogenous prfA gene, in endogenous D-alanine racemase (Dal) and D-amino acid transferase (Dat) genes, or a combination thereof. In one example, the attenuated Listeria strain comprises mutations in an actA virulence gene, in a D-alanine racemase (Dal) gene, and in a D-amino acid transferase (Dat) gene.

In some cases, the nucleic acid molecule can comprise a second open reading frame. As an example, the second open reading frame can encode a metabolic enzyme. Alternatively, the Listeria strain can comprise a second nucleic acid molecule comprising an open reading frame encoding a metabolic enzyme. Various types of nucleic acids encoding metabolic enzymes are disclosed in further detail elsewhere herein. For example, the Listeria strain can be an auxotrophic Listeria strain, and the metabolic enzyme can complement the auxotrophy of said auxotrophic Listeria strain. Examples of metabolic enzymes are disclosed in further detail elsewhere herein. For example, the metabolic enzyme can be an alanine racemase enzyme or a D-amino acid transferase enzyme. In one example, the Listeria strain is an attenuated Listeria strain comprising mutations in an actA virulence gene, in a D-alanine racemase (Dal) gene, and in a D-amino acid transferase (Dat) gene, and either the nucleic acid molecule comprises a second open reading frame encoding an alanine racemase enzyme or a D-amino acid transferase enzyme or the Listeria strain comprises a second nucleic acid molecule encoding an alanine racemase enzyme or a D-amino acid transferase enzyme. An example of a Dal gene is set forth in SEQ ID NO: 68, and an example of a Dat gene is set forth in SEQ ID NO: 70. An example of a Dal protein is set forth in SEQ ID NO: 69, and an example of a Dat protein is set forth in SEQ ID NO: 71.

The Listeria strain can be any type of Listeria strain, examples of which are disclosed elsewhere herein. Preferably, the Listeria strain is a recombinant Listeria monocytogenes strain. Likewise, preferably the Listeria strain is an auxotrophic Listeria strain. Optionally, the Listeria strain is capable of escaping a phagolysosome. Optionally, the Listeria strain has been passaged through an animal host.

Any of the Listeria strains disclosed herein can be used in an immunogenic composition. Such immunogenic compositions can further comprise an adjuvant. Examples of adjuvants include a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide. Other suitable adjuvants are disclosed elsewhere herein.

Any of the Listeria strains and any of the immunogenic compositions disclosed herein can be used in methods of inducing an immune response against a tumor or cancer in a subject, comprising administering to the subject the Listeria strain or the immunogenic composition. Likewise, any of the Listeria strains and any of the immunogenic compositions disclosed herein can be used in methods of preventing or treating a tumor or cancer in a subject, comprising administering to the subject the Listeria strain or the immunogenic composition. Examples of tumors or cancers include a PSA-expressing tumor or cancer, a survivin-expressing tumor or cancer, a PSGR-expressing tumor or cancer, or a hepsin-expressing tumor or cancer, such as a prostate tumor or cancer. Examples of doses and methods of administering are disclosed in further detail elsewhere herein.

In one embodiment, disclosed herein is a method of eliciting an anti-tumor or anti-cancer immune response in a subject, the method comprising the step of administering to said subject an effective amount of an immunogenic composition comprising a recombinant Listeria strain comprising a recombinant nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or an immunogenic fragment thereof, and wherein said Listeria expresses said fusion polypeptide, thereby eliciting an anti-tumor or anti-cancer immune response in said subject.

In one embodiment, disclosed herein is an immunogenic composition comprising a recombinant Listeria strain comprising a recombinant nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to an endoglin sequence or an immunogenic fragment thereof, wherein said Listeria strain comprises mutations in endogenous genes encoding a D-alanine racemase (dal) and a D-amino acid transferase (dat) gene, and in a virulence gene encoding an ActA (actA).

In another aspect, a recombinant nucleic acid molecule in a Listeria strain disclosed herein comprises a second open reading frame. In another aspect, said second open reading frame encodes a second fusion polypeptide, wherein said fusion polypeptide comprises a truncated listeriolysin O (LLO) protein, a truncated ActA protein, or a PEST amino acid sequence fused to a heterologous antigen or an immunogenic fragment thereof, and wherein said Listeria expresses said second fusion polypeptide.

In another embodiment, disclosed herein is a recombinant Listeria strain comprising a bivalent expression episome or plasmid comprising a first and a second nucleotide molecule encoding a first and a second fusion polypeptide, wherein said first and said second polypeptide each comprise a heterologous antigen fused to a truncated listeriolysin O (LLO) protein, a truncated ActA protein, or a PEST amino acid sequence. In another embodiment, each of said fusion polypeptide or each fusion partner in a fusion polypeptide is encoded in an open reading frame within said nucleic acid molecule.

In one embodiment, disclosed herein is a recombinant Listeria strain comprising a nucleic acid molecule comprising a first open reading frame encoding a fusion polypeptide, said fusion polypeptide comprising a prostate specific (PSA) antigen or a immunogenic fragment thereof fused to a truncated listeriolysin O (LLO), a truncated ActA or PEST amino acid sequence, and wherein said nucleic acid molecule further comprises a second open reading frame encoding a fusion polypeptide, said fusion polypeptide comprising a prostate-specific membrane antigen (PSMA) or an immunogenic fragment thereof fused to a truncated listeriolysin O (LLO), a truncated ActA or PEST amino acid sequence.

In another embodiment, disclosed herein is a recombinant Listeria strain comprising a nucleic acid molecule comprising a first open reading frame encoding a fusion polypeptide, said fusion polypeptide comprising a prostate specific (PSA) antigen or an immunogenic fragment thereof fused to a truncated listeriolysin O (LLO), a truncated ActA or PEST amino acid sequence, and wherein said nucleic acid molecule further comprises a second open reading frame encoding a fusion polypeptide, said fusion polypeptide comprising a survivin antigen or an immunogenic fragment thereof fused to a truncated listeriolysin O (LLO), a truncated ActA or PEST amino acid sequence.

In one embodiment, disclosed herein is a method of inducing an anti-tumor immune response in a subject comprising the step of administering to said subject the recombinant Listeria disclosed herein. In another embodiment, the immune response allows treating, suppressing, or inhibiting a cancer in a subject.

In one embodiment, disclosed herein is a recombinant Listeria strain comprising a first nucleotide molecule and a second nucleotide molecule encoding a first and a second polypeptide, wherein said first and said second polypeptide each comprise a heterologous antigen fused to a truncated listeriolysin O (LLO) protein, a truncated ActA protein, or a PEST amino acid sequence, wherein said first nucleotide molecule is in an extrachromosomal episome or extrachromosomal plasmid, and wherein said second nucleotide molecule is integrated into the Listeria genome.

In another embodiment, disclosed herein is a recombinant Listeria strain comprising a bivalent expression episome or plasmid comprising a nucleotide molecule encoding a first and a second fusion polypeptide, wherein said first and said second polypeptide each comprise a heterologous antigen fused to a truncated listeriolysin O (LLO) protein, a truncated ActA protein, or a PEST amino acid sequence.

In another embodiment, disclosed herein is a recombinant Listeria strain comprising a first and a second nucleic acid molecule, wherein said first nucleic acid molecule encodes a recombinant polypeptide comprising a carbonic anhydrase 9 (CA9) antigen or a functional fragment thereof fused to a truncated listeriolysin O (LLO), and wherein said second nucleic acid molecule encodes a chimeric HER (cHER2) protein fused to an endogenous LLO.

In another embodiment, a recombinant Listeria strain disclosed herein comprises a first nucleic acid molecule comprising a first open reading frame encoding a fusion polypeptide, said fusion polypeptide comprising a prostate specific (PSA) antigen or a functional fragment thereof fused to a truncated listeriolysin O (LLO), a truncated ActA or PEST amino acid sequence, and wherein said nucleic acid molecule further comprises a second open reading frame encoding a fusion polypeptide, said fusion polypeptide comprising a survivin antigen fused to a truncated listeriolysin O (LLO), a truncated ActA or PEST amino acid sequence.

In another embodiment, a recombinant Listeria strain disclosed herein comprises a first nucleic acid molecule comprising a first open reading frame encoding a fusion polypeptide, said fusion polypeptide comprising a prostate specific (PSA) antigen or a functional fragment thereof fused to a truncated listeriolysin O (LLO), a truncated ActA or PEST amino acid sequence, and wherein said nucleic acid molecule further comprises a second open reading frame encoding a fusion polypeptide, said fusion polypeptide comprising a prostate-specific membrane antigen (PSMA) fused to a truncated listeriolysin O (LLO), a truncated ActA or PEST amino acid sequence.

The invention discloses, in another embodiment, a method of increasing the efficacy of a Listeria-based immunotherapy, the method comprising sequentially or concomitantly administering two or more recombinant Listeria strains to a subject having a tumor. In a related aspect, the Listeria strains that are administered sequentially or concomitantly each comprise a nucleic acid molecule, said nucleic acid molecule encoding a recombinant polypeptide comprising a heterologous antigen fused to a PEST-containing polypeptide. In another related aspect, the heterologous antigen is chimeric HER (cHER2), CA9, PSA or HMW-MAA-C. In another embodiment, the method of increasing the efficacy of a Listeria-based immunotherapy enhances an antigen-specific immune response as a result of administering the same.

In one embodiment, disclosed herein is a method of producing a recombinant Listeria strain comprising a bivalent expression plasmid comprising a first and a second nucleotide molecule encoding a first and a second fusion polypeptide or comprising a nucleotide molecule encoding a first and a second fusion polypeptide, wherein said first and said second fusion polypeptide each comprise a heterologous antigen fused to a truncated listeriolysin O (LLO), a truncated ActA or PEST amino acid sequence, said method comprising the steps of:
a) obtaining a plasmid;
b) recombinantly fusing in said plasmid said first and second nucleotide molecule;
c) transforming into said recombinant Listeria said plasmid; and
d) expressing said fusion protein by culturing said Listeria under conditions conducive to protein expression;
   thereby producing a recombinant Listeria strain.

In another embodiment, disclosed herein is a method of producing the recombinant Listeria strain comprising a bivalent expression plasmid comprising a first and a second nucleotide molecule encoding a first and a second fusion polypeptide or comprising a nucleotide molecule encoding a first and a second fusion polypeptide, wherein said first and said second polypeptide each comprise a heterologous antigen fused to a truncated listeriolysin O (LLO), a truncated ActA or PEST amino acid sequence, said method comprising the steps of:
a) obtaining a plasmid;
b) recombinantly fusing in said plasmid said first and second nucleotide molecule; and
c) transforming into said recombinant Listeria said plasmid;
   thereby producing a recombinant Listeria strain.

In another embodiment, disclosed herein is a recombinant Listeria strain comprising a bivalent episomal expression vector, the vector comprising a first, and at least a second nucleic acid molecule encoding a heterologous antigenic polypeptide or a functional fragment thereof, wherein the first and the second nucleic acid molecules each encode the heterologous antigenic polypeptide or functional fragment thereof in an open reading frame with a truncated listeriolysin O (LLO), a truncated ActA or PEST amino acid sequence.

It will be appreciated that the term “bivalent” or “multivalent,” when in reference to a nucleotide molecule, plasmid, or vector may encompass a nucleotide molecule, nucleic acid, DNA sequence, plasmid, and the like, that expresses two (bivalent), three (multivalent), or more (multivalent) heterologous antigens each individually fused to a an N-terminal or truncated LLO, N-terminal ActA, or a PEST sequence or PEST peptide. In another embodiment, a bivalent plasmid encodes two heterologous antigens. In another embodiment, a bivalent plasmid encodes two different heterologous antigens. In another embodiment, the term “bivalent” is used interchangeably herein with “dual”. In another embodiment, a multivalent plasmid encodes three or more different heterologous antigens.

It will be appreciated that the term “bivalent” or “multivalent,” when in reference to a recombinant Listeria strain may encompass a Listeria strain that is capable of expressing two (bivalent) or more (multivalent) heterologous antigens. It will be appreciated that the term “bivalent” or “multivalent,” when in reference to a recombinant Listeria strain may encompass a Listeria strain that expresses two (bivalent) or more (multivalent) heterologous antigens. In one embodiment a bivalent Listeria strain comprises a bivalent plasmid that expresses two heterologous antigens from an extrachromosomal plasmid or episomal vector. In another embodiment, a bivalent Listeria strain expresses one heterologous antigen from the genome, (following integration of the heterologous antigen into the Listeria genome), and another heterologous antigen from a plasmid present in the cytoplasm of said Listeria strain. In another embodiment, a multivalent Listeria strain expresses three or more heterologous antigens from a plasmid. In another embodiment, a multivalent Listeria strain expresses three heterologous antigens, one from the genome (following integration of the heterologous antigen into the Listeria genome) and two from a plasmid present in the cytoplasm of the Listeria strain. In another embodiment, a multivalent Listeria strain expresses three heterologous antigens, two from the genome (following integration of the heterologous antigen into the Listeria genome) and one from a plasmid present in the cytoplasm of the Listeria strain. It will be well appreciated by a skilled artisan that a multivalent Listeria strain comprises the ability to express three or more heterologous antigens in total, where at least one is expressed from either the genome or from a plasmid (in any desired combination, i.e., 3 from the plasmid and 1 from the genome, 2 from the plasmid and 2 from the genome, 1 from the plasmid and 3 from the genome, etc.).

In another embodiment, a bivalent Listeria strain expresses one heterologous antigen from the genome in the context of a fusion protein with an endogenous LLO gene sequence, (following integration of the heterologous antigen into the frame of the LLO gene in the Listeria genome), and another heterologous antigen from a plasmid present in the cytoplasm of said Listeria strain, in the context of a fusion protein with a truncated listeriolysin O (LLO), a truncated ActA or PEST amino acid sequence. In another embodiment, a multivalent Listeria strain expresses three heterologous antigens from a plasmid, all in the context of a fusion protein with a PEST-containing polypeptide. In another embodiment, a multivalent Listeria strain expresses three heterologous antigens, one from the genome (following integration of the heterologous antigen into the endogenous LLO gene sequence in the Listeria genome) and two heterologous antigens from a plasmid present in the cytoplasm of the Listeria strain, in the context of a truncated listeriolysin O (LLO), a truncated ActA or PEST amino acid sequence. In another embodiment, a multivalent Listeria strain expresses three heterologous antigens, two from the genome (following integration of the heterologous antigen into the endogenous LLO gene sequence in the Listeria genome) and one from a plasmid present in the cytoplasm of the Listeria strain, all in the context of a fusion protein with a truncated listeriolysin O (LLO), a truncated ActA or PEST amino acid sequence. It will be well appreciated by a skilled artisan that a multivalent Listeria strain comprises the ability to express three or more heterologous antigens in total, where at least one is expressed from either the genome or from a plasmid (in any desired combination, i.e.—3 from the plasmid and 1 from the genome, 2 from the plasmid and 2 from the genome, 1 from the plasmid and 3 from the genome, etc.,) all in the context of a fusion protein with a truncated listeriolysin O (LLO), a truncated ActA or PEST amino acid sequence.

In one embodiment, disclosed herein is a recombinant Listeria strain comprising a minigene nucleic acid construct comprising an open reading frame encoding a chimeric protein, wherein said chimeric protein comprises a (i) bacterial secretion signal sequence; (ii) a ubiquitin (Ub) protein; (iii) a peptide; and, wherein said signal sequence, said ubiquitin and said peptide in i.-iii. are operatively linked in tandem order from the amino-terminus to the carboxy-terminus.

In one embodiment, disclosed herein is a recombinant attenuated Listeria strain comprising: (a) a nucleic acid molecule, the nucleic acid molecule comprising a first open reading frame encoding a fusion polypeptide, wherein the fusion polypeptide comprises an immunogenic polypeptide or fragment thereof fused to one or more heterologous antigens; or, (b) a minigene nucleic acid construct comprising one or more open reading frames encoding a chimeric protein, wherein the chimeric protein comprises: (i) a bacterial secretion signal sequence, (ii) a ubiquitin (Ub) protein, (iii) one or more antigenic peptides; and, wherein the signal sequence, the ubiquitin and one or more antigenic peptides in (i)-(iii) are operatively linked or arranged in tandem from the amino-terminus to the carboxy-terminus.

In one embodiment, the Listeria further comprises two or more open reading frames linked by a Shine-Dalgarno ribosome binding site nucleic acid sequence.

In another embodiment, the recombinant Listeria further comprises one to four open reading frames linked by a Shine-Dalgarno ribosome binding site nucleic acid sequence between each open reading frame. In another embodiment, each open reading frame comprises a different antigen peptide.

In another embodiment, disclosed herein is a method of eliciting an anti-tumor or anti-cancer response in a subject having a tumor or cancer, said method comprising the step of administering to said subject a recombinant Listeria comprising a minigene nucleic acid construct disclosed herein.

In one embodiment, disclosed herein is a method of treating a tumor or cancer in a subject, said method comprising the step of administering to said subject a recombinant Listeria comprising a minigene nucleic acid construct disclosed herein.

It will be appreciated by the skilled artisan that the term “nucleic acid” and grammatical equivalents thereof may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

It will be appreciated by the skilled artisan that the terms “cancer” and “tumor” may have all the same meanings and qualities.

In another embodiment, disclosed herein are compositions and methods for inducing an immune response against a tumor antigen. In another embodiment, the tumor antigen is a heterologous antigen. In another embodiment, the tumor antigen is a self-antigen. In another embodiment, provided herein are compositions and methods for inducing an immune response against an infectious disease antigen. In another embodiment, the infectious disease antigen is a heterologous antigen. In another embodiment, the compositions and methods of this invention are used for vaccinating against a tumor or a cancer.

In yet another embodiment, the compositions and methods of the present invention prevent the occurrence of escape mutations following treatment. In another embodiment, provided herein are compositions and methods for providing progression free survival to a subject suffering from a tumor or cancer. In another embodiment, disclosed herein are compositions and methods for immunizing a subject against a cancer or tumor. In another embodiment, disclosed herein are compositions and methods for immunizing a subject against a cancer or tumor. In another embodiment, the cancer is metastasis.

In one embodiment, disclosed herein is a recombinant attenuated Listeria strain comprising a nucleic acid construct encoding a chimeric protein. In another embodiment, the nucleic acid construct is a recombinant nucleic acid construct. In another embodiment, disclosed herein is a recombinant attenuated Listeria strain comprising a recombinant nucleic acid construct comprising an open reading frame encoding a bacterial secretion signal sequence (SS), a ubiquitin (Ub) protein, and a peptide sequence. In another embodiment, the nucleic acid construct encodes a chimeric protein comprising a bacterial secretion signal sequence, a ubiquitin protein, and a peptide sequence. In another embodiment, the bacteria secretion signal sequence is a Listeria signal sequence. In one embodiment, the chimeric protein is arranged in the following manner (SS-Ub-Peptide).

In one embodiment, the minigene nucleic acid construct disclosed herein comprises a codon that corresponds to the carboxy-terminus of the peptide moiety is followed by two stop codons to ensure termination of protein synthesis.

In one embodiment, provided herein is a recombinant attenuated Listeria strain comprising a nucleic acid construct encoding a chimeric protein. In another embodiment, the nucleic acid construct is a recombinant nucleic acid construct. In another embodiment, provided herein is a recombinant attenuated Listeria strain comprising a recombinant nucleic acid construct comprising an open reading frame encoding a bacterial secretion signal sequence (SS), a ubiquitin (Ub) protein, and a peptide sequence. In another embodiment, the nucleic acid construct encodes a chimeric protein comprising a bacterial secretion signal sequence, a ubiquitin protein, and a peptide sequence. In one embodiment, the chimeric protein is arranged in the following manner (SS-Ub-Peptide) wherein each component is operatively linked to each other starting with the signal sequence at the amino end and ending with the peptide sequence at the carboxy end.

In one embodiment, the minigene nucleic acid construct comprises a codon that corresponds to the carboxy-terminus of the peptide moiety is followed by two stop codons to ensure termination of protein synthesis.

In one embodiment, the chimeric proteins of the present invention are synthesized, in another embodiment, using recombinant DNA methodology. This involves, in one embodiment, creating a DNA sequence that encodes the chimeric protein, placing the DNA in an expression cassette, such as the plasmid of the present invention, under the control of a particular promoter/regulatory element, and expressing the protein. DNA encoding the chimeric protein (e.g. SS-Ub-peptide) of the present invention is prepared, in another embodiment, by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 15 1859-1862); and the solid support method of U.S. Pat. No. 4,458,066.

In another embodiment, DNA encoding the chimeric protein or the recombinant protein of the present invention is cloned using DNA amplification methods such as polymerase chain reaction (PCR). In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then ligated to produce the desired DNA sequence.

In one embodiment, nucleic acid sequences encoding chimeric proteins disclosed herein are transformed into a variety of host cells, including E. coli, other bacterial hosts, such as Listeria, yeast, and various higher eukaryotic cells such as the COS, CHO and HeLa cells lines and myeloma cell lines. Nucleic acid sequences encoding a chimeric protein provided herein are operably linked to appropriate expression control sequences for each host. Promoter/regulatory sequences are described in detail elsewhere herein. In another embodiment, the plasmid encoding a chimeric protein provided herein further comprises additional promoter regulatory elements, as well as a ribosome binding site and a transcription termination signal. For eukaryotic cells, the control sequences will include a promoter and an enhancer derived from e.g. immunoglobulin genes, SV40, cytomegalovirus, etc., and a polyadenylation sequence. In another embodiment, the sequences include splice donor and acceptor sequences.

In one embodiment, a minigene nucleic acid construct disclosed herein or a plasmid comprising the same comprises at least one ribosome binding site and at least one transcription termination signals that allow encoding of at least one chimeric protein as provided herein, each comprising a different peptide antigen. In one embodiment, the plasmid provided herein comprises 1 to 4 ribosome binding ribosome binding sites and 1 to 4 r transcription termination signals that allow encoding of 1 to 4 chimeric proteins as provided herein, each comprising a different peptide antigen. In one embodiment, the plasmid provided herein comprises 5 to 10 ribosome binding ribosome binding sites and 5 to 10 transcription termination signals that allow encoding of 5 to 10 chimeric proteins as provided herein, each comprising a different peptide antigen. In one embodiment, the plasmid provided herein comprises 11 to 20 ribosome binding ribosome binding sites and 11 to 20 transcription termination signals that allow encoding of 11 to 20 chimeric proteins as provided herein, each comprising a different peptide antigen. In one embodiment, the plasmid provided herein comprises 21 to 30 ribosome binding ribosome binding sites and 21 to 30 transcription termination signals that allow encoding of 21 to 30 chimeric proteins as provided herein, each comprising a different peptide antigen. In another embodiment, the ribosome binding sites are shine dalgarno ribosome binding sites.

In one embodiment, the term “operably linked” means that the transcriptional and translational regulatory nucleic acid, is positioned relative to any coding sequences in such a manner that transcription is initiated. Generally, this will mean that the promoter and transcriptional initiation or start sequences are positioned 5′ to the coding region. In another embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In another embodiment, the term “operably linked” refers to the joining of several open reading frames in a transcription unit each encoding a protein or peptide so as to result in expression of a chimeric protein or polypeptide that functions as intended.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, as an example, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. It will be understood by a skilled artisan that a “linker sequence” may encompass an amino acid sequence that joins two heterologous polypeptides, or fragments or domains thereof. In general, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

Recombinant or chimeric proteins, or fusion polypeptides disclosed herein may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid.

It is to be understood by a skilled artisan that the terms “polypeptide” and “protein” have all the same meanings and qualifications for the intended purpose of their use herein.

In one embodiment, a fusion polypeptide, or chimeric protein disclosed herein is expressed and secreted by a recombinant Listeria disclosed herein. In another embodiment, the fusion polypeptide, or chimeric protein disclosed herein comprises a C-terminal SIINFEKL-S-6×HIS tag. In another embodiment, the fusion polypeptide or chimeric protein disclosed herein comprises a FLAG tag or a SIINFEKL-S-FLAG tag (e.g., a C-terminal or an N-terminal FLAG tag or a C-terminal or an N-terminal SIINFEKL-S-FLAG tag). In another embodiment, the fusion polypeptide, or chimeric protein disclosed herein is expressed and secreted by a recombinant Listeria disclosed herein. In another embodiment, secretion of the antigen, or polypeptides (fusion or chimeric) disclosed herein is detected using a protein, molecule or antibody (or fragment thereof) that specifically binds to a polyhistidine (His) tag. In another embodiment, the fusion polypeptide, or chimeric protein disclosed herein is expressed and secreted by a recombinant Listeria disclosed herein. In another embodiment, secretion of the antigen, or polypeptides (fusion or chimeric) disclosed herein is detected using an antibody, protein or molecule that binds a SIINFEKL-S-6×HIS tag. In another embodiment, the fusion polypeptide of chimeric protein disclosed herein comprise any other tag know in the art, including, but not limited to chiti birnding protein (CBP), maltose binding protein (MBP), and glutathione-S-transferase (GST), thioredoxin (TRX) and poly(NANP).

The terms “antigen,” “antigen peptide.” “antigenic polypeptide,” “antigen fragment,” are used interchangeably herein and, as will be appreciated by a skilled artisan, may encompass polypeptides, or peptides (including recombinant peptides) that are loaded onto and presented on MHC class I and/or class II molecules on a host's cell's surface and can be recognized or detected by an immune cell of the host, thereby leading to the mounting of an immune response against the polypeptide, peptide or cell presenting the same. Similarly, the immune response may also extend to other cells within the host, including diseased cells such as tumor or cancer cells that express the same polypeptides or peptides.

In one embodiment, an antigen may be foreign, that is, heterologous to the host and is referred to as a “heterologous antigen” herein. In another embodiment, a heterologous antigen is heterologous to a Listeria strain disclosed herein that recombinantly expresses said antigen. In another embodiment, a heterologous antigen is heterologous to the host and a Listeria strain disclosed herein that recombinantly expresses said antigen. In another embodiment, the antigen is a self-antigen, which is an antigen that is present in the host but the host does not elicit an immune response against it because of immunologic tolerance. It will be appreciated by a skilled artisan that a heterologous antigen as well as a self-antigen may encompass a tumor antigen, a tumor-associated antigen or an angiogenic antigen. In addition, a heterologous antigen may encompass an infectious disease antigen.

In one embodiment, the terms “recombinant Listeria” and “live-attenuated Listeria” are used interchangeably herein and refer to a Listeria comprising at least one attenuating mutation, deletion or inactivation that expresses at least one fusion protein of an antigen fused to a truncated LLO, truncated ActA or PEST sequence embodied herein. In another embodiment, a recombinant Listeria disclosed herein is a recombinant Listeria monocytogenes.

It will also be appreciated by a skilled artisan that the terms “antigenic portion thereof,” “a fragment thereof” and “immunogenic portion thereof” in regard to a protein, peptide or polypeptide are used interchangeably herein and may encompass a protein, polypeptide, peptide, including recombinant forms thereof comprising a domain or segment that leads to the mounting of an immune response when present in, or, in some embodiments, detected by, a host, either alone, or in the context of a fusion protein, as described herein.

The terms “nucleic acid,” “nucleotide,” “nucleic acid molecule,” “oligonucleotide,” or “nucleotide molecule” are used interchangeably herein and may encompass a string of at least two base-sugar-phosphate combinations, as will be appreciated by a skilled artisan. The terms include, in one embodiment, DNA and RNA. It will also be appreciated by a skilled artisan that the terms may encompass the monomeric units of nucleic acid polymers. For example, RNA may be in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term may also encompass artificial nucleic acids that may contain other types of backbones but the same bases. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

The terms “amino acid” or “amino acids” are understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

It will be appreciated by a skilled artisan that the term “open reading frame” or “ORF” may encompass a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

It will be appreciated by a skilled artisan that the term “endogenous” may encompass an item that has developed or originated within the reference organism or arisen from causes within the reference organism. For example, endogenous refers to native.

It will also be appreciated by a skilled artisan that the term “fragment” may encompass a protein or polypeptide that is shorter or comprises fewer amino acids than the full length protein or polypeptide. In one embodiment, a fragment is an N-terminal fragment. In another embodiment, a fragment is a C-terminal fragment. In yet another embodiment, a fragment is an intrasequential section of the protein or peptide. It will be understood by a skilled artisan that a fragment as disclosed herein is a functional fragment, which may encompass an immunogenic fragment. In one embodiment, a fragment has more than 5 amino acids. In another embodiment, a fragment has 10-20 amino acids, 20-50 amino acids, 50-100 amino acids, 100-200 amino acids, 200-350 amino acids, or 350-500 amino acids.

In an alternate embodiment, the term “fragment” refers to a nucleic acid that is shorter or comprises fewer nucleotides than the full length nucleic acid. In one embodiment, a fragment is a 5′-terminal fragment. In another embodiment, a fragment is a 3′-terminal fragment. In yet another embodiment, a fragment encodes an intrasequential section of the protein. In one embodiment, a fragment has more than 5 nucleotides. In another embodiment, a fragment has 10-20 nucleotides, 20-50 nucleotides, 50-100 nucleotides, 100-200 nucleotides, 200-350 nucleotides, 350-500 or 500-1000 nucleotides. It will be appreciated by a skilled artisan that the term “functional” within the meaning of the invention, may encompass the innate ability of a protein, peptide, nucleic acid, fragment or a variant thereof to exhibit a biological activity. Such a biological activity may encompass having the potential to elicit an immune response when used as disclosed herein, an illustration of which may be to be used as part of a fusion protein). Such a biological function may encompass its binding property to an interaction partner, e.g., a membrane-associated receptor, or its trimerization property. In the case of functional fragments and the functional variants of the invention, these biological functions may in fact be changed, e.g., with respect to their specificity or selectivity, but with retention of the basic biological function.

It will be appreciated by a skilled artisan that the terms “fragment” or “functional fragment” may encompass an immunogenic fragment that is capable of eliciting an immune response when administered to a subject alone or as part of a pharmaceutical composition comprising a recombinant Listeria strain expressing said immunogenic fragment. In another embodiment, a functional fragment has biological activity as will be understood by a skilled artisan and as further disclosed herein.

In one embodiment, disclosed herein is a multivalent plasmid that delivers at least two antigens. In another embodiment, the plasmid is a dual or bivalent plasmid. In another embodiment, the dual, bivalent or multivalent plasmid is episomal in nature in that it remains extrachromosomal. In another embodiment, the dual or multivalent plasmid comprises sequences for integration into the Listeria chromosome.

In one embodiment, disclosed herein is a multivalent recombinant Listeria strain plasmid that expresses at least two antigens each fused to a truncated LLO, a truncated ActA or a PEST amino acid sequence. In another embodiment, the recombinant Listeria is a dual or bivalent Listeria.

In another embodiment, the recombinant nucleic acid backbone of a plasmid disclosed herein comprises SEQ ID NO: 1.

In one embodiment, a bivalent plasmid backbone comprises at least two nucleic acid sequences encoding at least two antigens. In another embodiment, the bivalent plasmid backbone comprises a nucleic acid sequences having at least two open reading frames encoding at least two antigens. In another embodiment, the bivalent plasmid backbone comprises a nucleic acid sequences having two open reading frames encoding two antigens. In another embodiment, the multivalent plasmid backbone comprises a nucleic acid sequences having at least three open reading frames encoding at least three antigens.

In another embodiment, the multivalent plasmid backbone comprises at least three nucleic acid sequences having at least three open reading frames encoding at least three antigens. In another embodiment, the multivalent plasmid backbone comprises a nucleic acid sequences having three open reading frames encoding three antigens. In another embodiment, the multivalent plasmid backbone comprises three nucleic acid sequences having three open reading frames encoding three antigens.

In one embodiment, antigens encoded by the bivalent Listeria strains disclosed herein include CA9, chimeric HER2 (cHER2), and HMW-MAA or a fragment thereof (see Examples 11-16 herein). In another embodiment, the HMW-MAA fragment is HMW-MAA-C (HMC).

In one embodiment, a Listeria strain LmddA244G disclosed herein comprises a nucleic acid sequence comprising an open reading frame encoding a cHER2 fused to an endogenous nucleic acid comprising an open reading frame encoding an LLO protein (see SEQ ID NO: 2), where the sequence at positions 1594-2850 represents the nucleic acid sequence encoding a cHER2, the sequence at positions 1-1587 represents the sequence encoding an endogenous LLO protein, and the “gtcgac” sequence at positions 1588-1593 represents the Sal I restriction site used to ligate the tumor antigen to the endogenous LLO. In one embodiment, the endogenous LLO-cHER2 fusion is a homolog of SEQ ID NO: 2. In another embodiment, the endogenous LLO-cHER2 fusion is a variant of SEQ ID NO: 2. In another embodiment, the endogenous LLO-cHER2 fusion is an isomer of SEQ ID NO: 2.

In one embodiment, the amino acid sequence of the fusion between a cHER2 and an endogenous LLO comprises SEQ ID NO: 3. In one embodiment, the endogenous LLO-cHER2 fusion is a homolog of SEQ ID NO: 3. In another embodiment, the endogenous LLO-cHER2 fusion is a variant of SEQ ID NO: 3. In another embodiment, the endogenous LLO-cHER2 fusion is an isomer of SEQ ID NO: 3.

In one embodiment, the amino acid sequence of endogenous LLO protein comprises SEQ ID NO: 4.

In one embodiment, LmddA164 comprises a nucleic acid sequence comprising an open reading frame encoding tLLO fused to cHER2, wherein said nucleic acid sequence comprises SEQ ID NO: 5, wherein the sequence at positions 1330 to 2586 encodes cHER2, the sequence at positions 1 to 1323 encodes tLLO, and the “ctcgag” sequence at positions 1324-1329 represents the Xho I restriction site used to ligate the tumor antigen to truncated LLO in the plasmid. In another embodiment, plasmid pAdv168 comprises SEQ ID NO: 5. In one embodiment, the truncated LLO-cHER2 fusion is a homolog of SEQ ID NO: 5. In another embodiment, the truncated LLO-cHER2 fusion is a variant of SEQ ID NO: 5. In another embodiment, the truncated LLO-cHER2 fusion is an isomer of SEQ ID NO: 5.

In one embodiment, an amino acid sequence of a tLLO fused to a cHER2 comprises SEQ ID NO: 6. In one embodiment, the truncated LLO-cHER2 fusion is a homolog of SEQ ID NO: 6. In another embodiment, the truncated LLO-cHER2 fusion is a variant of SEQ ID NO: 6. In another embodiment, the truncated LLO-cHER2 fusion is an isomer of SEQ ID NO: 6.

In one embodiment, an amino acid sequence of a truncated LLO (tLLO) comprises SEQ ID NO: 7.

In one embodiment, LmddA168 comprises a nucleic acid sequence comprising an open reading frame encoding tLLO fused to HMW-MAA-C(HMC) comprises SEQ ID NO: 8, wherein the sequence at positions 1330-1647 encodes HMC, the sequence at positions 1-1323 encodes tLLO, and the “ctcgag” sequence at positions 1324-1329 represents the Xho I restriction site used to ligate the tumor antigen to truncated LLO in the plasmid. In another embodiment, plasmid pAdv168 comprises SEQ ID NO: 8. In one embodiment, the truncated LLO-HMC fusion is a homolog of SEQ ID NO: 8. In another embodiment, the truncated LLO-HMC fusion is a variant of SEQ ID NO: 8. In another embodiment, the truncated LLO-HMC fusion is an isomer of SEQ ID NO: 8.

In one embodiment, an amino acid sequence of a tLLO fused to an HMC antigen comprises SEQ ID NO: 9. In one embodiment, the truncated LLO-HMC fusion is a homolog of SEQ ID NO: 9. In another embodiment, the truncated LLO-HMC fusion is a variant of SEQ ID NO: 9. In another embodiment, the truncated LLO-HMC fusion is an isomer of SEQ ID NO: 9.

In one embodiment, the sequence of HMC comprises SEQ ID NO: 10.

In one embodiment, the antigens are heterologous antigens to the bacteria host carrying the plasmid. In another embodiment, the antigens are heterologous antigens to the Listeria host carrying the plasmid.

In another embodiment, the recombinant episomal nucleic acid sequence encoding the plasmid backbone and at least two heterologous antigens comprises SEQ ID NO: 11. In another embodiment, the recombinant episomal nucleic acid sequence encoding the plasmid backbone and at least two heterologous antigens consists of SEQ ID NO: 11.

In one embodiment, disclosed herein is an immunotherapeutic composition comprising a recombinant Listeria strain, wherein said Listeria further comprises a bivalent or multivalent plasmid disclosed herein and an adjuvant, cytokine, chemokine, or a combination thereof. In one embodiment, disclosed herein is a vaccine comprising a recombinant Listeria strain, wherein said Listeria further comprises a bivalent or multivalent plasmid disclosed herein and an adjuvant, cytokine, chemokine, or a combination thereof. In another embodiment, disclosed herein is a pharmaceutical formulation comprising a recombinant Listeria strain, wherein said Listeria further comprises the bivalent or multivalent plasmid disclosed herein and an adjuvant, cytokine, chemokine, or a combination thereof.

In one embodiment of the present invention, disclosed herein is a recombinant Listeria strain comprising a first and second nucleic acid molecule, each said nucleic acid molecule encoding a heterologous antigenic polypeptide or fragment thereof, wherein the first nucleic acid molecule is integrated into the Listeria genome in an open reading frame with an endogenous LLO gene and wherein the second nucleic acid molecule is present in an episomal expression vector or plasmid within the recombinant Listeria strain.

In one embodiment, this invention provides a recombinant Listeria strain comprising a first and second nucleic acid molecule, each said nucleic acid molecule encoding a heterologous antigenic polypeptide fused to a truncated LLO, a truncated or N-terminal ActA protein or a PEST sequence.

In one embodiment, the first nucleic acid molecule is operably integrated into the Listeria genome as an open reading frame with an endogenous nucleic acid sequence encoding an LLO protein, an ActA protein or a PEST sequence. In one embodiment, the first nucleic acid molecule is operably integrated into the Listeria genome as an open reading frame with a nucleic acid sequence encoding LLO. In another embodiment, the first nucleic acid molecule is operably integrated into the Listeria genome as an open reading frame with a nucleic acid sequence encoding ActA. In one embodiment, the integration does not eliminate the functionality of LLO. In another embodiment, the integration does not eliminate the functionality of ActA. In one embodiment, the functionality of LLO or ActA is its native functionality.

In one embodiment, the LLO functionality is allowing the organism to escape from the phagolysosome, while in another embodiment, the LLO functionality is enhancing the immunogenicity of a polypeptide to which it is fused. In one embodiment, a recombinant Listeria disclosed herein retains LLO function, which in one embodiment, is hemolytic function and in another embodiment, is antigenic function. Other functions of LLO are known in the art, as are methods and assays for evaluating LLO functionality.

In one embodiment, a recombinant Listeria of the present invention has wild-type virulence, while in another embodiment, a recombinant Listeria of the present invention has attenuated virulence. In another embodiment, a recombinant Listeria disclosed herein is avirulent. In one embodiment, a recombinant Listeria of disclosed herein is sufficiently virulent to escape the phagolysosome and enter the cytosol. In one embodiment, a recombinant Listeria disclosed herein expresses a fused antigen-LLO protein. Thus, in one embodiment, the integration of the first nucleic acid molecule into the Listeria genome does not disrupt the structure nor, in another embodiment, the function of the endogenous LLO gene, ActA gene, or PEST-containing gene. In one embodiment, the integration of the first nucleic acid molecule into the Listeria genome does not disrupt the ability of said Listeria to escape the phagolysosome.

In another embodiment, either the second nucleic acid is integrated into the genome while the first is expressed from a plasmid. In another embodiment, the second nucleic acid molecule is operably integrated into the Listeria genome with said first nucleic acid molecule in an open reading frame with an endogenous polypeptide comprising a PEST sequence. Thus, in one embodiment, the first and second nucleic acid molecules are integrated in frame with a nucleic acid sequence encoding an LLO protein, while in another embodiment, they are integrated in frame with a nucleic acid sequence encoding an ActA protein. In another embodiment, the second nucleic acid molecule is operably integrated into the Listeria genome in an open reading frame with a nucleic acid sequence encoding a polypeptide comprising a PEST sequence in a site that is distinct from the integration site of the first nucleic acid molecule. In one embodiment, the first nucleic acid molecule is integrated in frame with a nucleic acid sequence encoding an LLO protein, while the second nucleic acid molecule is integrated in frame with a nucleic acid sequence encoding an ActA protein, while in another embodiment, the first nucleic acid molecule is integrated in frame with a nucleic acid sequence encoding an ActA protein, while the second nucleic acid molecule is integrated in frame with a nucleic acid sequence encoding a LLO protein.

In another embodiment, this invention provides a recombinant Listeria strain comprising a first nucleic acid molecule encoding a first heterologous antigenic polypeptide or fragment thereof and a second nucleic acid molecule encoding a second heterologous antigenic polypeptide or fragment thereof, wherein said first nucleic acid molecule is integrated into the Listeria genome such that the first heterologous antigenic polypeptide and an LLO, ActA or PEST sequence are expressed as a fusion protein. In one embodiment, the first heterologous antigenic polypeptide and the LLO, ActA or PEST sequence are translated in a single open reading frame, while in another embodiment, the first heterologous antigenic polypeptide and the LLO, ActA or PEST sequence are fused after being translated separately.

In one embodiment, the Listeria genome comprises a deletion of the endogenous ActA gene, which in one embodiment is a virulence factor. In one embodiment, such a deletion provides a more attenuated and thus safer Listeria strain for human use. According to this embodiment, the antigenic polypeptide is integrated in frame with LLO in the Listeria chromosome. In another embodiment, the integrated nucleic acid molecule is integrated into the ActA locus. In another embodiment, the chromosomal nucleic acid encoding ActA is replaced by a nucleic acid molecule encoding an antigen. In another embodiment, the Listeria strain comprises an inactivation of the endogenous actA gene. In another embodiment, the Listeria strain comprises a truncation of the endogenous actA gene. In another embodiment, the Listeria strain comprises a non-functional replacement of the endogenous actA gene. In another embodiment, the Listeria strain comprises a substitution of the endogenous actA gene. All of the above-mentioned modifications fall within the scope of what is considered to be a “mutation” of the endogenous actA gene.

In another embodiment, the Listeria strain disclosed herein comprises a mutation, deletion or an inactivation of the endogenous dal/dat and actA genes and such a Listeria strain is referred to herein as an “LmddA” strain.

In another embodiment, the Listeria strain disclosed herein comprises a mutation, deletion or an inactivation of the endogenous dal/dat/actA and prfA genes.

In one embodiment, the bivalent or multivalent plasmids disclosed herein comprise a replication control region. In one embodiment, a recombinant nucleic acid molecule encoding the bivalent or multivalent plasmid disclosed herein comprises a replication control region. In another embodiment, the plasmid control region regulates replication of the recombinant nucleic acid molecule.

In another embodiment, the plasmid control region comprises an open reading frame encoding a transcription repressor that represses heterologous antigen expression from the first or at least the second nucleic acid molecule. In another embodiment, the plasmid control region comprises an open reading frame encoding transcription inducer that induces heterologous antigen expression from the first or at least the second nucleic acid molecule. In another embodiment, the plasmid control region comprises an open reading frame encoding a transcription repressor that represses heterologous antigen expression from a first, second or third nucleic acid molecule. In another embodiment, the plasmid control region comprises an open reading frame encoding a transcription inducer that induces heterologous antigen expression from the first, second or third nucleic acid molecule.

In another embodiment, the plasmid replication regulation region enables the regulation of expression of exogenous heterologous antigen from each of the first or at least the second open reading frame of a recombinant nucleic acid molecule comprised by the Listeria or the plasmid disclosed herein. In another embodiment, the plasmid replication regulation region enables the regulation of expression of exogenous heterologous antigen from each of the first, second or third open reading frames.

In one embodiment, measuring metabolic burden in a bacteria such as a Listeria is accomplished by any means know in the art at the time of the invention which include but are not limited to, measuring growth rates of the vaccine strain, optical density readings, colony forming unit (CFU) plating, and the like. In another embodiment, the metabolic burden on the bacterial cell is determined by measuring the viability of the bacterial cell. Methods of measuring bacteria viability are readily known and available in the art, some of which include but are not limited to, bacteria plating for viability count, measuring ATP, and flow cytometry. In ATP staining, detection is based on using the luciferase reaction to measure the amount of ATP from viable cells, wherein the amount of ATP in cells correlates with cell viability. As to flow cytometry, this method can be used in various ways, also known in the art, for example after employing the use of viability dyes which are excluded by live bacterial cells and are absorbed or adsorbed by a dead bacterial cells. A skilled artisan would readily understand that these and any other methods known in the art for measuring bacterial viability can be used in the present invention. It is to be understood that a skilled artisan would be able to implement the knowledge available in the art at the time of the invention for measuring growth rates of the vaccine strain or expression of marker genes by the vaccine strain that enable determining the metabolic burden of the vaccine strain expressing multiple heterologous antigens or functional fragments thereof.

In another embodiment, the integrated nucleic acid molecule is integrated into the Listeria chromosome.

In one embodiment, said first nucleic acid molecule is a vector designed for site-specific homologous recombination into the Listeria genome. In another embodiment, the construct or heterologous gene is integrated into the Listerial chromosome using homologous recombination.

Techniques for homologous recombination are well known in the art, and are described, for example, in Frankel, F R, Hegde, S, Lieberman, J, and Y Paterson. Induction of a cell-mediated immune response to HIV gag using Listeria monocytogenes as a live vaccine vector. J. Immunol. 155: 4766-4774. 1995; Mata, M, Yao, Z, Zubair, A, Syres, K and Y Paterson, Evaluation of a recombinant Listeria monocytogenes expressing an HIV protein that protects mice against viral challenge. Vaccine 19:1435-45, 2001; Boyer, J D, Robinson, T M, Maciag, P C, Peng, X, Johnson, R S, Pavlakis, G, Lewis, M G, Shen, A, Siliciano, R, Brown, C R, Weiner, D, and Y Paterson. DNA prime Listeria boost induces a cellular immune response to SIV antigens in the Rhesus Macaque model that is capable of limited suppression of SIV239 viral replication. Virology. 333: 88-101, 2005. In another embodiment, homologous recombination is performed as described in U.S. Pat. No. 6,855,320. In another embodiment, a temperature sensitive plasmid is used to select the recombinants.

In another embodiment, the construct or heterologous gene is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in one embodiment, that a stable genomic insertion mutant can be formed. In another embodiment, the position in the genome where the foreign gene has been inserted by transposon mutagenesis is unknown.

In another embodiment, a construct or heterologous gene is integrated into the Listerial chromosome using phage integration sites (Lauer P, Chow M Y et al, Construction, characterization, and use of two LM site-specific phage integration vectors. J Bacteriol 2002; 184(15): 4177-86). In another embodiment, an integrase gene and attachment site of a bacteriophage (e.g. U153 or PSA listeriophage) is used to insert the heterologous gene into the corresponding attachment site, which can be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants.

In another embodiment, the first nucleic acid sequence of methods and compositions as disclosed herein is operably linked to a promoter/regulatory sequence. In another embodiment, the second nucleic acid sequence is operably linked to a promoter/regulatory sequence. In another embodiment, each of the nucleic acid sequences disclosed herein are operably linked to a promoter/regulatory sequence. In one embodiment, the promoter/regulatory sequence is present on an episomal plasmid comprising said nucleic acid sequence. In one embodiment, an endogenous Listeria promoter/regulatory sequence controls the expression of a nucleic acid sequence of the methods and compositions of the present invention.

In one embodiment, a fusion polypeptide disclosed herein is expressed from an hly promoter, a prfA promoter, an actA promoter, or a p60 promoter or any other suitable promoter known in the art. In another embodiment, a nucleic acid sequence disclosed herein is operably linked to a promoter, regulatory sequence, or a combination thereof that drives expression of the encoded peptide in the Listeria strain. Promoter, regulatory sequences, and combinations thereof useful for driving constitutive expression of a gene are well known in the art and include, but are not limited to, for example, the P_{hly}A, P_{act}A, hly, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide as disclosed herein is accomplished by placing the nucleic acid encoding the peptide under the control of an inducible or tissue-specific promoter/regulatory sequence. Examples of tissue-specific or inducible regulatory sequences, promoters, and combinations thereof which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter or regulatory sequence, which is either known or unknown, and which is capable of driving expression of the desired protein operably linked thereto. In one embodiment, a regulatory sequence is a promoter, while in another embodiment, a regulatory sequence is an enhancer, while in another embodiment, a regulatory sequence is a suppressor, while in another embodiment, a regulatory sequence is a repressor, while in another embodiment, a regulatory sequence is a silencer.

In one embodiment, the nucleic acid construct used for integration to the Listeria genome contains an integration site. In one embodiment, the site is a PhSA (phage from Scott A) attPP′ integration site. PhSA is, in another embodiment, the prophage of L. monocytogenes strain ScottA (Loessner, M. J., I. B. Krause, T. Henle, and S. Scherer. 1994. Structural proteins and DNA characteristics of 14 Listeria typing bacteriophages. J. Gen. Virol. 75:701-710, incorporated herein by reference), a serotype 4b strain that was isolated during an epidemic of human listeriosis. In another embodiment, the site is any another integration site known in the art.

In another embodiment, the nucleic acid construct contains an integrase gene. In another embodiment, the integrase gene is a PhSA integrase gene. In another embodiment, the integrase gene is any other integrase gene known in the art.

In one embodiment, the nucleic acid construct is a plasmid. In another embodiment, the nucleic acid construct is a shuttle plasmid. In another embodiment, the nucleic acid construct is an integration vector. In another embodiment, the nucleic acid construct is a site-specific integration vector. In another embodiment, the nucleic acid construct is any other type of nucleic acid construct known in the art.

The integration vector of methods and compositions disclosed herein is, in another embodiment, a phage vector. In another embodiment, the integration vector is a site-specific integration vector. In another embodiment, the vector further comprises an attPP′ site.

In another embodiment, the integration vector is a U153 vector. In another embodiment, the integration vector is an A118 vector. In another embodiment, the integration vector is a PhSA vector.

In another embodiment, the vector is an A511 vector (e.g. GenBank Accession No: X91069). In another embodiment, the vector is an A006 vector. In another embodiment, the vector is a B545 vector. In another embodiment, the vector is a B053 vector. In another embodiment, the vector is an A020 vector. In another embodiment, the vector is an A500 vector (e.g. GenBank Accession No: X85009). In another embodiment, the vector is a B051 vector. In another embodiment, the vector is a B052 vector. In another embodiment, the vector is a B054 vector. In another embodiment, the vector is a B055 vector. In another embodiment, the vector is a B056 vector. In another embodiment, the vector is a B101 vector. In another embodiment, the vector is a B110 vector. In another embodiment, the vector is a B111 vector. In another embodiment, the vector is an A153 vector. In another embodiment, the vector is a D441 vector. In another embodiment, the vector is an A538 vector. In another embodiment, the vector is a B653 vector. In another embodiment, the vector is an A513 vector. In another embodiment, the vector is an A507 vector. In another embodiment, the vector is an A502 vector. In another embodiment, the vector is an A505 vector. In another embodiment, the vector is an A519 vector. In another embodiment, the vector is a B604 vector. In another embodiment, the vector is a C703 vector. In another embodiment, the vector is a B025 vector. In another embodiment, the vector is an A528 vector. In another embodiment, the vector is a B024 vector. In another embodiment, the vector is a B012 vector. In another embodiment, the vector is a B035 vector. In another embodiment, the vector is a C707 vector.

In another embodiment, the vector is an A005 vector. In another embodiment, the vector is an A620 vector. In another embodiment, the vector is an A640 vector. In another embodiment, the vector is a B021 vector. In another embodiment, the vector is an HS047 vector. In another embodiment, the vector is an H10G vector. In another embodiment, the vector is an H8/73 vector. In another embodiment, the vector is an H19 vector. In another embodiment, the vector is an H21 vector. In another embodiment, the vector is an H43 vector. In another embodiment, the vector is an H46 vector. In another embodiment, the vector is an H107 vector. In another embodiment, the vector is an H108 vector. In another embodiment, the vector is an H110 vector. In another embodiment, the vector is an H163/84 vector. In another embodiment, the vector is an H312 vector. In another embodiment, the vector is an H340 vector. In another embodiment, the vector is an H387 vector. In another embodiment, the vector is an H391/73 vector. In another embodiment, the vector is an H684/74 vector. In another embodiment, the vector is an H924A vector. In another embodiment, the vector is an fMLUP5 vector. In another embodiment, the vector is a syn (=P35) vector. In another embodiment, the vector is a 00241 vector. In another embodiment, the vector is a 00611 vector. In another embodiment, the vector is a 02971A vector. In another embodiment, the vector is a 02971C vector. In another embodiment, the vector is a 5/476 vector. In another embodiment, the vector is a 5/911 vector. In another embodiment, the vector is a 5/939 vector. In another embodiment, the vector is a 5/11302 vector. In another embodiment, the vector is a 5/11605 vector. In another embodiment, the vector is a 5/11704 vector. In another embodiment, the vector is a 184 vector. In another embodiment, the vector is a 575 vector. In another embodiment, the vector is a 633 vector. In another embodiment, the vector is a 699/694 vector. In another embodiment, the vector is a 744 vector. In another embodiment, the vector is a 900 vector. In another embodiment, the vector is a 1090 vector. In another embodiment, the vector is a 1317 vector. In another embodiment, the vector is a 1444 vector. In another embodiment, the vector is a 1652 vector. In another embodiment, the vector is an 1806 vector. In another embodiment, the vector is an 1807 vector. In another embodiment, the vector is a 1921/959 vector. In another embodiment, the vector is a 1921/11367 vector. In another embodiment, the vector is a 1921/11500 vector. In another embodiment, the vector is a 1921/11566 vector. In another embodiment, the vector is a 1921/12460 vector. In another embodiment, the vector is a 1921/12582 vector. In another embodiment, the vector is a 1967 vector. In another embodiment, the vector is a 2389 vector. In another embodiment, the vector is a 2425 vector. In another embodiment, the vector is a 2671 vector. In another embodiment, the vector is a 2685 vector. In another embodiment, the vector is a 3274 vector. In another embodiment, the vector is a 3550 vector. In another embodiment, the vector is a 3551 vector. In another embodiment, the vector is a 3552 vector. In another embodiment, the vector is a 4276 vector. In another embodiment, the vector is a 4277 vector. In another embodiment, the vector is a 4292 vector. In another embodiment, the vector is a 4477 vector. In another embodiment, the vector is a 5337 vector. In another embodiment, the vector is a 5348/11363 vector. In another embodiment, the vector is a 5348/11646 vector. In another embodiment, the vector is a 5348/12430 vector. In another embodiment, the vector is a 5348/12434 vector. In another embodiment, the vector is a 10072 vector. In another embodiment, the vector is an 11355C vector. In another embodiment, the vector is an 11711A vector. In another embodiment, the vector is a 12029 vector. In another embodiment, the vector is a 12981 vector. In another embodiment, the vector is a 13441 vector. In another embodiment, the vector is a 90666 vector. In another embodiment, the vector is a 90816 vector. In another embodiment, the vector is a 93253 vector. In another embodiment, the vector is a 907515 vector. In another embodiment, the vector is a 910716 vector. In another embodiment, the vector is a NN-Listeria vector. In another embodiment, the vector is an 01761 vector. In another embodiment, the vector is a 4211 vector. In another embodiment, the vector is a 4286 vector. In another embodiment, the integration vector is any other site-specific integration vector known in the art that is capable of infecting Listeria.

In another embodiment, the integration vector or plasmid of methods and compositions as disclosed herein does not confer antibiotic resistance to the Listeria vaccine strain. In another embodiment, the integration vector or plasmid does not contain an antibiotic resistance gene.

In another embodiment, the present invention provides a recombinant nucleic acid encoding a recombinant polypeptide. In one embodiment, the nucleic acid comprises a sequence sharing at least 80% homology with a nucleic acid encoding a recombinant polypeptide disclosed herein. In another embodiment, the nucleic acid comprises a sequence sharing at least 85% homology with a nucleic acid encoding a recombinant polypeptide disclosed herein. In another embodiment, the nucleic acid comprises a sequence sharing at least 90% homology with a nucleic acid encoding a recombinant polypeptide disclosed herein. In another embodiment, the nucleic acid comprises a sequence sharing at least 95% homology with a nucleic acid encoding a recombinant polypeptide disclosed herein. In another embodiment, the nucleic acid comprises a sequence sharing at least 97% homology with a nucleic acid encoding a recombinant polypeptide disclosed herein. In another embodiment, the nucleic acid comprises a sequence sharing at least 99% homology with a nucleic acid encoding a recombinant polypeptide disclosed herein.

In one embodiment, disclosed herein is a method of producing a recombinant Listeria strain comprising a bivalent plasmid encoding two distinct heterologous antigens. In another embodiment, the plasmid is a multivalent plasmid that encodes 3 or more distinct heterologous antigens. In another embodiment, the plasmid is a multivalent plasmid that encodes 4 or more distinct heterologous antigens. In another embodiment, the plasmid is a multivalent plasmid that encodes 5 or more distinct heterologous antigens.

In one embodiment, the recombinant Listeria disclosed herein expresses at least one antigen encoded by the plasmids disclosed herein.

In one embodiment, disclosed is a method of producing a recombinant Listeria strain expressing two distinct heterologous antigens. In another embodiment, the recombinant Listeria expresses at least 3 or more distinct heterologous antigens. In another embodiment, the recombinant Listeria expresses 4 or more distinct heterologous antigens. In another embodiment, the recombinant Listeria expresses 5 or more distinct heterologous antigens.

In another embodiment, the method of producing a recombinant Listeria comprises transforming said recombinant Listeria with nucleic acid comprising a bivalent or multivalent plasmid. In one embodiment, the plasmid is an episomal plasmid that remains extrachromosomal. In another embodiment, the plasmid is an integrative plasmid. In yet another embodiment, the method disclosed herein comprises expressing the antigens and fusion proteins disclosed herein under conditions conducive to protein expression.

It will be appreciated by a skilled artisan that the nucleic acids disclosed herein comprise DNA vectors, RNA vectors, plasmids (extrachromosomal and/or integrative), etc., that may be used in the methods disclosed herein for generating any of the compositions disclosed herein.

In another embodiment, the recombinant Listeria strain may express more than two antigens, some of which are expressed from one or more nucleic acid molecules integrated into the Listeria chromosome and some of which are expressed via one or more episomal expression plasmids or vectors present in the recombinant Listeria strain. Thus, as disclosed hereinabove, in one embodiment, a recombinant Listeria strain as disclosed herein comprises two or more episomal expression plasmids, each of which expresses at least one distinct antigenic polypeptide. In one embodiment, one or more of the antigens are expressed as a fusion protein with LLO, which in one embodiment, is non-hemolytic LLO or truncated LLO. In one embodiment, a recombinant Listeria strain as disclosed herein targets tumors by eliciting immune responses to two separate antigens, which are expressed by two different cell types, which in one embodiment are a cell surface antigen and an anti-angiogenic polypeptide, while in another embodiment, a recombinant Listeria strain as disclosed herein targets tumors by eliciting an immune response to two different antigens expressed by the same cell type. In another embodiment, a recombinant Listeria strain as disclosed herein targets tumors by eliciting an immune response to two different antigens as disclosed herein or as are known in the art.

In one embodiment, a heterologous antigen disclosed herein is associated with the local tissue environment that is further associated with the development of or metastasis of cancer. In another embodiment, the heterologous antigen disclosed herein is associated with tumor immune evasion or resistance to cancer. In another embodiment, the heterologous antigen disclosed herein is an angiogenic antigen.

In one embodiment, a first antigen of the compositions and methods of disclosed herein is directed against a specific cell surface antigen or tumor target, and a second antigen is directed against an angiogenic antigen or tumor microenvironment. In another embodiment, the first and second antigens of the compositions and methods of the present invention are polypeptides expressed by tumor cells, or in another embodiment, polypeptides expressed in a tumor microenvironment. In another embodiment, the first antigen of the compositions and methods of the present invention is a polypeptide expressed by a tumor and the second antigen of the compositions and methods of the present invention is a receptor target, NO Synthetase, Arg-1, or other enzyme known in the art.

In one embodiment, disclosed herein is a method of producing a recombinant Listeria strain expressing two antigens, the method comprising, in one embodiment, genetically fusing a first nucleic acid encoding a first antigen and a second nucleic acid encoding a second antigen into the Listeria genome in an open reading frame with a native polypeptide comprising a PEST sequence. In another embodiment, the expressing said first and second antigens are produced under conditions conducive to antigenic expression in said recombinant Listeria strain.

In one embodiment, the recombinant Listeria strain of the composition and methods as disclosed herein comprises an episomal expression vector comprising the second nucleic acid molecule encoding a heterologous antigen. In another embodiment, the second nucleic acid molecule encoding a heterologous antigen is present in said episomal expression vector in an open reading frame with a truncated LLO, truncated ActA or a PEST amino acid sequence.

In another embodiment, an episomal expression vector of the methods and compositions as disclosed herein comprises an antigen fused in frame to a nucleic acid sequence encoding a PEST amino acid sequence. In one embodiment, the antigen is HMW-MAA, and in another embodiment, a HMW-MAA fragment. In another embodiment, the PEST-like AA sequence is KENSISSMAPPASPPASPKTPIEKKHADEIDK (SEQ ID NO: 12). In another embodiment, the PEST-like sequence is KENSISSMAPPASPPASPK (SEQ ID NO: 13). In another embodiment, fusion of an antigen to any LLO sequence that includes one of the PEST-like AA sequences enumerated herein can enhance cell mediated immunity against HMW-MAA.

In another embodiment, the PEST-like AA sequence is a PEST-like sequence from a Listeria ActA protein. In another embodiment, the PEST-like sequence is KTEEQPSEVNTGPR (SEQ ID NO: 14), KASVTDTSEGDLDSSMQSADESTPQPLK (SEQ ID NO: 15), KNEEVNASDFPPPPTDEELR (SEQ ID NO: 16), or RGGIPTSEEFSSLNSGDFTDDENSETTEEEIDR (SEQ ID NO: 17). In another embodiment, the PEST-like sequence is from Listeria seeligeri cytolysin, encoded by the lso gene. In another embodiment, the PEST-like sequence is RSEVTISPAETPESPPATP (SEQ ID NO: 18). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 19) at AA 35-51. In another embodiment, the PEST-like sequence is from Streptococcus equisimilis Streptolysin O, e.g. KQNTANTETTTTNEQPK (SEQ ID NO: 20) at AA 38-54. In another embodiment, the PEST-like sequence has a sequence selected from SEQ ID NO: 14-20. In another embodiment, the PEST-like sequence has a sequence selected from SEQ ID NO: 12-20. In another embodiment, the PEST-like sequence is another PEST-like AA sequence derived from a prokaryotic organism. In another embodiment, the PEST sequence is any other PEST sequence known in the art, including, but not limited to, those disclosed in United States Patent Publication No. 2014/0186387, which is incorporated by reference herein in its entirety.

Identification of PEST-like sequences is well known in the art, and is described, for example in Rogers S et al (Amino acid sequences common to rapidly degraded proteins: the PEST hypothesis. Science 1986; 234(4774):364-8, incorporated herein by reference) and Rechsteiner M et al (PEST sequences and regulation by proteolysis. Trends Biochem Sci 1996; 21(7):267-71, incorporated herein by reference). “PEST-like sequence” refers, in another embodiment, to a region rich in proline (P), glutamic acid (E), serine (S), and threonine (T) residues. In another embodiment, the PEST-like sequence is flanked by one or more clusters containing several positively charged amino acids. In another embodiment, the PEST-like sequence mediates rapid intracellular degradation of proteins containing it. In another embodiment, the PEST-like sequence fits an algorithm disclosed in Rogers et al. In another embodiment, the PEST-like sequence fits an algorithm disclosed in Rechsteiner et al. In another embodiment, the PEST-like sequence contains one or more internal phosphorylation sites, and phosphorylation at these sites precedes protein degradation. In one embodiment, a sequence referred to herein as a PEST-like sequence is a PEST sequence.

In one embodiment, PEST-like sequences of prokaryotic organisms are identified in accordance with methods such as described by, for example Rechsteiner and Rogers (1996, Trends Biochem. Sci. 21:267-271) for LM and in Rogers S et al (Science 1986; 234(4774):364-8). Alternatively, PEST-like AA sequences from other prokaryotic organisms can also be identified based on this method. Other prokaryotic organisms wherein PEST-like AA sequences would be expected to include, but are not limited to, other Listeria species. In one embodiment, the PEST-like sequence fits an algorithm disclosed in Rogers et al. In another embodiment, the PEST-like sequence fits an algorithm disclosed in Rechsteiner et al. In another embodiment, the PEST-like sequence is identified using the PEST-find program.

In another embodiment, identification of PEST motifs is achieved by an initial scan for positively charged amino acids R, H, and K within the specified protein sequence. All amino acids between the positively charged flanks are counted and only those motifs are considered further, which contain a number of amino acids equal to or higher than the window-size parameter. In another embodiment, a PEST-like sequence must contain at least 1 P, 1 D or E, and at least 1 S or T.

In another embodiment, the quality of a PEST motif is refined by means of a scoring parameter based on the local enrichment of critical amino acids as well as the motifs hydrophobicity. Enrichment of D, E, P, S and T is expressed in mass percent (w/w) and corrected for 1 equivalent of D or E, 1 of P and 1 of S or T. In another embodiment, calculation of hydrophobicity follows in principle the method of J. Kyte and R. F. Doolittle (Kyte, J and Dootlittle, R F. J. Mol. Biol. 157, 105 (1982), incorporated herein by reference. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine.

Hydropathy index=10*Kyte-Doolittle hydropathy index+45.

In another embodiment, a potential PEST motif's hydrophobicity is calculated as the sum over the products of mole percent and hydrophobicity index for each amino acid species. The desired PEST score is obtained as combination of local enrichment term and hydrophobicity term as expressed by the following equation:

PEST score=0.55*DEPST−0.5*hydrophobicity index.

In another embodiment, “PEST sequence,” “PEST-like sequence,” “PEST amino acid sequence” or “PEST-like sequence peptide” are used interchangeably here and refer to a peptide having a score of at least +5, using the above algorithm. In another embodiment, the term refers to a peptide having a score of at least 6. In another embodiment, the peptide has a score of at least 7. In another embodiment, the score is at least 8. In another embodiment, the score is at least 9. In another embodiment, the score is at least 10. In another embodiment, the score is at least 11. In another embodiment, the score is at least 12. In another embodiment, the score is at least 13. In another embodiment, the score is at least 14. In another embodiment, the score is at least 15. In another embodiment, the score is at least 16. In another embodiment, the score is at least 17. In another embodiment, the score is at least 18. In another embodiment, the score is at least 19. In another embodiment, the score is at least 20. In another embodiment, the score is at least 21. In another embodiment, the score is at least 22. In another embodiment, the score is at least 22. In another embodiment, the score is at least 24. In another embodiment, the score is at least 24. In another embodiment, the score is at least 25. In another embodiment, the score is at least 26. In another embodiment, the score is at least 27. In another embodiment, the score is at least 28. In another embodiment, the score is at least 29. In another embodiment, the score is at least 30. In another embodiment, the score is at least 32. In another embodiment, the score is at least 35. In another embodiment, the score is at least 38. In another embodiment, the score is at least 40. In another embodiment, the score is at least 45. Each possibility represents a separate embodiment of the methods and compositions as disclosed herein,

In another embodiment, the PEST sequence is identified using any other method or algorithm known in the art, e.g., the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each stretch of appropriate length (e.g. a 30-35 amino acid stretch) by assigning a value of 1 to the amino acids Ser, Thr, Pro, Glu, Asp, Asn, or Gln. The coefficient value (CV) for each of the PEST residue is 1 and for each of the other amino acids (non-PEST) is 0.

Each method for identifying a PEST sequence represents a separate embodiment as disclosed herein.

In another embodiment, the PEST sequence is any other PEST sequence known in the art.

In one embodiment, the present invention provides fusion proteins, which in one embodiment, are expressed by Listeria. In one embodiment, such fusion proteins comprise fusions to a tLLO, a truncated ActA or a PEST sequence. It will be understood by a skilled artisan that the term “PEST sequence” may encompass cases wherein a protein fragment comprises a PEST sequence having surrounding sequences other than the PEST sequence. In another embodiment, the protein fragment consists of the PEST sequence. Thus, in another embodiment, “fusion” refers to two peptides or protein fragments either linked together at their respective ends or embedded one within the other. It will be appreciated by a skilled artisan that the term “fused” may also encompass an operable linkage by covalent bonding. In one embodiment, the term encompasses recombinant fusion (of nucleic acid sequences or open reading frames thereof). In another embodiment, the term encompasses chemical conjugation.

In another embodiment, a recombinant Listeria strain of the compositions and methods as disclosed herein comprises a full length LLO polypeptide, which in one embodiment, is hemolytic.

In another embodiment, the recombinant Listeria strain comprises a non-hemolytic LLO polypeptide. In another embodiment, the polypeptide is an LLO fragment. In another embodiment, the polypeptide is a complete LLO protein. In another embodiment, the polypeptide is any LLO protein or fragment thereof known in the art.

In another embodiment, an LLO protein fragment is utilized in compositions and methods as disclosed herein. In one embodiment, a truncated LLO protein is encoded by the episomal expression vector as disclosed herein that expresses a polypeptide, that is, in one embodiment, an antigen, in another embodiment, an angiogenic factor, or, in another embodiment, both an antigen and angiogenic factor. In another embodiment, the LLO fragment is an N-terminal fragment.

In one embodiment, the terms “N-terminal LLO protein” and “truncated LLO (tLLO)” are used interchangeably herein.

In another embodiment, the N-terminal LLO fragment has the sequence set forth in SEQ ID NO: 21. In another embodiment, an LLO AA sequence of methods and compositions as disclosed herein comprises the sequence set forth in SEQ ID NO: 21. In another embodiment, the LLO AA sequence is a homologue of SEQ ID NO: 21. In another embodiment, the LLO AA sequence is a variant of SEQ ID NO: 21. In another embodiment, the LLO AA sequence is a fragment of SEQ ID NO: 21. In another embodiment, the LLO AA sequence is an isoform of SEQ ID NO: 21.

In another embodiment, the LLO fragment has the sequence set forth in SEQ ID NO: 22. In another embodiment, an LLO AA sequence of methods and compositions as disclosed herein comprises the sequence set forth in SEQ ID NO: 22. In another embodiment, the LLO AA sequence is a homologue of SEQ ID NO: 22. In another embodiment, the LLO AA sequence is a variant of SEQ ID NO: 22. In another embodiment, the LLO AA sequence is a fragment of SEQ ID NO: 22. In another embodiment, the LLO AA sequence is an isoform of SEQ ID NO: 22.

In one embodiment, the LLO protein used in the compositions and methods as disclosed herein comprises the sequence set forth in SEQ ID NO: 23 (GenBank Accession No. P13128; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine as disclosed herein. In another embodiment, an LLO AA sequence of methods and compositions as disclosed herein comprises the sequence set forth in SEQ ID NO: 23. In another embodiment, the LLO AA sequence is a homologue of SEQ ID NO: 23. In another embodiment, the LLO AA sequence is a variant of SEQ ID NO: 23. In another embodiment, the LLO AA sequence is a fragment of SEQ ID NO: 23. In another embodiment, the LLO AA sequence is an isoform of SEQ ID NO: 23. disclosed herein

The LLO protein used in the compositions and methods as disclosed herein has, in another embodiment, the sequence set forth in SEQ ID NO: 24. In another embodiment, an LLO AA sequence of methods and compositions as disclosed herein comprises the sequence set forth in SEQ ID NO: 24. In another embodiment, the LLO AA sequence is a homologue of SEQ ID NO: 24. In another embodiment, the LLO AA sequence is a variant of SEQ ID NO: 24. In another embodiment, the LLO AA sequence is a fragment of SEQ ID NO: 24. In another embodiment, the LLO AA sequence is an isoform of SEQ ID NO: 24. Each possibility represents a separate embodiment as disclosed herein.

In one embodiment, the amino acid sequence of the LLO polypeptide of the compositions and methods as disclosed herein is from the Listeria monocytogenes 10403S strain, as set forth in Genbank Accession No.: ZP_01942330, EBA21833, or is encoded by the nucleic acid sequence as set forth in Genbank Accession No.: NZ_AARZ01000015 or AARZ01000015.1. In another embodiment, the LLO sequence for use in the compositions and methods as disclosed herein is from Listeria monocytogenes, which in one embodiment, is the 4b F2365 strain (in one embodiment, Genbank accession number: YP_012823), the EGD-e strain (in one embodiment, Genbank accession number: NP_463733), or any other strain of Listeria monocytogenes known in the art.

In another embodiment, the LLO sequence for use in the compositions and methods as disclosed herein is from Flavobacteriales bacterium HTCC2170 (in one embodiment, Genbank accession number: ZP_01106747 or EAR01433; in one embodiment, encoded by Genbank accession number: NZ_AAOC01000003). In one embodiment, proteins that are homologous to LLO in other species, such as alveolysin, which in one embodiment, is found in Paenibacillus alvei (in one embodiment, Genbank accession number: P23564 or AAA22224; in one embodiment, encoded by Genbank accession number: M62709) may be used in the compositions and methods as disclosed herein. Other such homologous proteins are known in the art.

Each LLO protein and LLO fragment represents a separate embodiment of the methods and compositions as disclosed herein.

In another embodiment, homologues of LLO from other species, including known lysins, or fragments thereof may be used to create a fusion protein of LLO with an antigen of the compositions and methods as disclosed herein, which in one embodiment, is HMW-MAA, and in another embodiment is a fragment of HMW-MAA.

In another embodiment, the LLO fragment of methods and compositions as disclosed herein, is a PEST-like domain. In another embodiment, an LLO fragment that comprises a PEST sequence is utilized as part of a composition or in the methods as disclosed herein.

In another embodiment, the LLO fragment does not contain the activation domain at the carboxy terminus. In another embodiment, the LLO fragment does not include cysteine 484. In another embodiment, the LLO fragment is a non-hemolytic fragment. In another embodiment, the LLO fragment is rendered non-hemolytic by deletion or mutation of the activation domain. In another embodiment, the LLO fragment is rendered non-hemolytic by deletion or mutation of cysteine 484. In another embodiment, an LLO sequence is rendered non-hemolytic by deletion or mutation at another location.

In another embodiment, the LLO fragment consists of about the first 441 AA of the LLO protein. In another embodiment, the LLO fragment comprises about the first 400-441 AA of the 529 AA full length LLO protein. In another embodiment, the LLO fragment corresponds to AA 1-441 of an LLO protein disclosed herein. In another embodiment, the LLO fragment consists of about the first 420 AA of LLO. In another embodiment, the LLO fragment corresponds to AA 1-420 of an LLO protein disclosed herein. In another embodiment, the LLO fragment consists of about AA 20-442 of LLO. In another embodiment, the LLO fragment corresponds to AA 20-442 of an LLO protein disclosed herein. In another embodiment, any ALLO without the activation domain comprising cysteine 484, and in particular without cysteine 484, are suitable for methods and compositions as disclosed herein.

In another embodiment, the LLO fragment corresponds to the first 400 AA of an LLO protein. In another embodiment, the LLO fragment corresponds to the first 300 AA of an LLO protein. In another embodiment, the LLO fragment corresponds to the first 200 AA of an LLO protein. In another embodiment, the LLO fragment corresponds to the first 100 AA of an LLO protein. In another embodiment, the LLO fragment corresponds to the first 50 AA of an LLO protein, which in one embodiment, comprises one or more PEST-like sequences.

In another embodiment, the LLO fragment is a non-hemolytic LLO. In another embodiment, the non-hemolytic LLO comprises one or more PEST-like sequences.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein.

In another embodiment, a recombinant Listeria strain of the methods and compositions as disclosed herein comprise a nucleic acid molecule operably integrated into the Listeria genome as an open reading frame with an endogenous ActA sequence. In another embodiment, a recombinant Listeria strain of the methods and compositions as disclosed herein comprise an episomal expression vector comprising a nucleic acid molecule encoding fusion protein comprising an antigen fused to an ActA or a truncated ActA. In one embodiment, the expression and secretion of the antigen is under the control of an actA promoter and ActA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387. In one embodiment, the antigen is HMW-MAA, while in another embodiment, it's an immunogenic fragment of HMW-MAA.

In one embodiment, the present invention provides a recombinant polypeptide comprising an N-terminal fragment of an LLO protein fused to a heterologous antigen disclosed herein or fused to a fragment thereof. In another embodiment, a Her-2 chimeric protein of the methods and compositions of the present invention is a human Her-2 chimeric protein. In another embodiment, the Her-2 protein is a mouse Her-2 chimeric protein. In another embodiment, the Her-2 protein is a rat Her-2 chimeric protein. In another embodiment, the Her-2 protein is a primate Her-2 chimeric protein. In another embodiment, the Her-2 protein is a Her-2 chimeric protein of any other animal species or combinations thereof known in the art. Each possibility represents a separate embodiment of the present invention.

In another embodiment, a Her-2 protein is a protein referred to as “HER-2/neu,” “Erbb2,” “v-erb-b2,” “c-erb-b2,” “neu,” or “cNeu.” Each possibility represents a separate embodiment of the present invention.

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein, harbors 3 H2Dq and at least 17 of the mapped human MHC-class I epitopes of the Her2/neu antigen (fragments EC1, EC2, and IC1) as described in U.S. patent application Ser. No. 12/945,386, which is incorporated by reference herein in its entirety. In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the Her2-neu chimeric protein is fused to the first 441 amino acids of the Listeria-monocytogenes listeriolysin O (LLO) protein and is expressed from the chromosome of a recombinant Listeria disclosed herein, while an additional antigen is expressed from a plasmid present within the recombinant Listeria disclosed herein. In another embodiment, the Her2-neu chimeric protein is fused to the first 441 amino acids of the Listeria-monocytogenes listeriolysin O (LLO) protein and is expressed from a plasmid of a recombinant Listeria disclosed herein, while an additional antigen is expressed from the chromosome of the recombinant Listeria disclosed herein. In another embodiment, a recombinant Listeria disclosed herein is a Listeria monocytogenes attenuated auxotrophic strain LmddA.

In another embodiment, the HER-2 chimeric protein is encoded by a nucleic acid sequence comprising SEQ ID NO: 25.

In another embodiment, the Her-2 chimeric protein (cHER2) comprises SEQ ID NO: 26.

In one embodiment, the HER2 chimeric protein or fragment thereof of the methods and compositions disclosed herein does not include a signal sequence thereof. In another embodiment, omission of the signal sequence enables the HER2 fragment to be successfully expressed in Listeria, due the high hydrophobicity of the signal sequence.

In another embodiment, the fragment of a HER2 chimeric protein of methods and compositions of the present invention does not include a transmembrane domain (TM) thereof. In one embodiment, omission of the TM enables the HER2 fragment to be successfully expressed in Listeria, due the high hydrophobicity of the TM.

In one embodiment, the nucleic acid sequence of human-Her2/neu gene is the sequence set forth in SEQ ID NO: 27.

In another embodiment, the nucleic acid sequence encoding the human her2/neu EC1 fragment implemented into the chimera spans from 120-510 bp of the human EC1 region and is set forth in SEQ ID NO: 28.

In one embodiment, the complete EC1 human her2/neu fragment spans from (58-979 bp of the human her2/neu gene and is set forth in SEQ ID NO: 29.

In another embodiment, the nucleic acid sequence encoding the human her2/neu EC2 fragment implemented into the chimera spans from 1077-1554 bp of the human her2/neu EC2 fragment and includes a 50 bp extension, and is set forth in SEQ ID NO: 30.

In one embodiment, complete EC2 human her2/neu fragment spans from 907-1504 bp of the human her2/neu gene and is set forth in SEQ ID NO: 31.

In another embodiment, the nucleic acid sequence encoding the human her2/neu IC1 fragment implemented into the chimera is set forth in SEQ ID NO: 32.

In another embodiment, the nucleic acid sequence encoding the complete human her2/neu IC1 fragment spans from 2034-3243 of the human her2/neu gene and is set forth in SEQ ID NO: 33.

In one embodiment, the present invention provides a recombinant polypeptide comprising an N-terminal fragment of an LLO protein fused to a carbonic anhydrase 9 (or carbonic anhydrase IX) protein or fused to a fragment thereof. In one embodiment, the present invention provides a recombinant polypeptide consisting of an N-terminal fragment of an LLO protein fused to a carbonic anhydrase 9 or fused to a fragment thereof.

In another embodiment, the carbonic anhydrase 9 protein of the methods and compositions of the present invention is a human carbonic anhydrase 9 protein. In another embodiment, the carbonic anhydrase 9 protein is a mouse carbonic anhydrase 9 protein. In another embodiment, the carbonic anhydrase 9 protein is a rat carbonic anhydrase 9 protein. In another embodiment, the carbonic anhydrase 9 protein is a primate carbonic anhydrase 9 protein. In another embodiment, the carbonic anhydrase 9 protein is a carbonic anhydrase 9 protein of any other animal species or combinations thereof known in the art.

In one embodiment, the terms “carbonic anhydrase 9,” “carbonic anhydrase IX,” and “CA9” are used interchangeably herein.

In one embodiment, the nucleic acid sequence of the human-CA9 gene is the sequence set forth in SEQ ID NO: 34. In one embodiment, the CA9 nucleic acid sequence is a homolog of SEQ ID NO: 34. In another embodiment, the CA9 nucleic acid sequence is a variant of SEQ ID NO: 34. In another embodiment, the CA9 nucleic acid sequence is a fragment of SEQ ID NO: 34. In another embodiment the CA9 nucleic acid sequence is any sequence known in the art including, but not limited to, those set forth in GenBank Accession nos. NM_001216.2, XM_006716867.1, XM_006716868.1, and X66839.1.

In one embodiment, the amino acid sequence encoded by the human CA9 gene disclosed herein is the sequence set forth in SEQ ID NO: 35. In one embodiment, the CA9 amino acid sequence is a homolog of SEQ ID NO: 35. In another embodiment, the CA9 amino acid sequence is a variant of SEQ ID NO: 35. In another embodiment, the CA9 amino acid sequence is an isomer of SEQ ID NO: 35. In another embodiment, the CA9 amino acid sequence is a fragment of SEQ ID NO: 35. In another embodiment the CA9 amino acid sequence is any sequence known in the art including, but not limited to, those set forth in GenBank Accession nos. NP_001207.2, XP_006716930.1, XP_006716931.1, and CAA47315.1.

In another embodiment, the nucleic acid sequence encoding a truncated LLO-CA9 fusion comprises SEQ ID NO: 36, wherein the sequence at positions 1330-2487 encodes cHER2, the sequence at positions 1-1323 encodes tLLO, and the “ctcgag” sequence at positions 1324-1329 represents the Xho I restriction site used to ligate the tumor antigen to truncated LLO in the plasmid. In one embodiment, the truncated LLO-CA9 fusion is a homolog of SEQ ID NO: 36. In another embodiment, the truncated LLO-CA9 fusion is a variant of SEQ ID NO: 36. In another embodiment, the truncated LLO-CA9 fusion is an isomer of SEQ ID NO: 36.

In one embodiment, an amino acid sequence comprising a tLLO fused to CA9 comprises SEQ ID NO: 37. In one embodiment, the truncated LLO-CA9 fusion is a homolog of SEQ ID NO: 37. In another embodiment, the truncated LLO-CA9 fusion is a variant of SEQ ID NO: 37. In another embodiment, the truncated LLO-CA9 fusion is an isomer of SEQ ID NO: 37.

In another embodiment, the LmddA strain disclosed herein comprises a mutation.

In one embodiment, an antigen of the methods and compositions as disclosed herein is fused to an ActA protein, which in one embodiment, is an N-terminal fragment of an ActA protein, which in one embodiment, comprises or consists of the first 390 AA of ActA, in another embodiment, the first 418 AA of ActA, in another embodiment, the first 50 AA of ActA, in another embodiment, the first 100 AA of ActA, which in one embodiment, comprise a PEST sequence such as that provided in SEQ ID NO: 2. In another embodiment, an N-terminal fragment of an ActA protein utilized in methods and compositions as disclosed herein comprises or consists of the first 150 AA of ActA, in another embodiment, the first approximately 200 AA of ActA, which in one embodiment comprises 2 PEST sequences as described herein. In another embodiment, an N-terminal fragment of an ActA protein utilized in methods and compositions as disclosed herein comprises or consists of the first 250 AA of ActA, in another embodiment, the first 300 AA of ActA. In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly, as would be routine to a skilled artisan using sequence alignment tools such as NCBI BLAST that are well-known in the art.

In another embodiment, the N-terminal portion of the ActA protein comprises 1, 2, 3, or 4 PEST sequences, which in one embodiment are the PEST sequences specifically mentioned herein, or their homologs, as described herein or other PEST sequences as can be determined using the methods and algorithms described herein or by using alternative methods known in the art.

In one embodiment, the terms “N-terminal ActA” and “truncated ActA” are used interchangeably herein.

In one embodiment, an N-terminal fragment of an ActA protein utilized in methods and compositions as disclosed herein has, in another embodiment, the sequence set forth in SEQ ID NO: 38. In another embodiment, the ActA fragment comprises the sequence set forth in SEQ ID NO: 38. In another embodiment, the ActA fragment is any other ActA fragment known in the art. In another embodiment, the ActA protein is a homologue of SEQ ID NO: 38. In another embodiment, the ActA protein is a variant of SEQ ID NO: 38. In another embodiment, the ActA protein is an isoform of SEQ ID NO: 38. In another embodiment, the ActA protein is a fragment of SEQ ID NO: 38. In another embodiment, the ActA protein is a fragment of a homologue of SEQ ID NO: 38. In another embodiment, the ActA protein is a fragment of a variant of SEQ ID NO: 38. In another embodiment, the ActA protein is a fragment of an isoform of SEQ ID NO: 38.

In another embodiment, the recombinant nucleotide encoding a fragment of an ActA protein comprises the sequence set forth in SEQ ID NO: 39. In another embodiment, the recombinant nucleotide has the sequence set forth in SEQ ID NO: 39. In another embodiment, the recombinant nucleotide comprises any other sequence that encodes a fragment of an ActA protein.

An N-terminal fragment of an ActA protein utilized in methods and compositions as disclosed herein has, in another embodiment, the sequence set forth in SEQ ID NO: 40, which in one embodiment is the first 390 AA for ActA from Listeria monocytogenes, strain 10403S. In another embodiment, the ActA fragment comprises the sequence set forth in SEQ ID NO: 40. In another embodiment, the ActA fragment is any other ActA fragment known in the art. In another embodiment, the ActA protein is a homologue of SEQ ID NO: 40. In another embodiment, the ActA protein is a variant of SEQ ID NO: 40. In another embodiment, the ActA protein is an isoform of SEQ ID NO: 40. In another embodiment, the ActA protein is a fragment of SEQ ID NO: 40. In another embodiment, the ActA protein is a fragment of a homologue of SEQ ID NO: 40. In another embodiment, the ActA protein is a fragment of a variant of SEQ ID NO: 40. In another embodiment, the ActA protein is a fragment of an isoform of SEQ ID NO: 40.

In another embodiment, a truncated ActA protein comprises the sequence set forth in SEQ ID NO: 41.

In another embodiment, a truncated ActA sequence disclosed herein is further fused to an hly signal peptide at the N-terminus. In another embodiment, the truncated ActA fused to hly signal peptide comprises SEQ ID NO: 42. In another embodiment, a truncated ActA as set forth in SEQ ID NO: 42 is referred to as LA229.

In another embodiment, the recombinant nucleotide encoding a fragment of an ActA protein comprises the sequence set forth in SEQ ID NO: 43, which in one embodiment, is the first 1170 nucleotides encoding ActA in Listeria monocytogenes 10403S strain. In another embodiment, the recombinant nucleotide has the sequence set forth in SEQ ID NO: 43. In another embodiment, the recombinant nucleotide comprises any other sequence that encodes a fragment of an ActA protein.

In another embodiment, the ActA fragment is another ActA fragment known in the art, which in one embodiment, is any fragment comprising a PEST sequence. Thus, in one embodiment, the ActA fragment is amino acids 1-100 of the ActA sequence. In another embodiment, the ActA fragment is amino acids 1-200 of the ActA sequence. In another embodiment, the ActA fragment is amino acids 200-300 of the ActA sequence. In another embodiment, the ActA fragment is amino acids 300-400 of the ActA sequence. In another embodiment, the ActA fragment is amino acids 1-300 of the ActA sequence. In another embodiment, a recombinant nucleotide as disclosed herein comprises any other sequence that encodes a fragment of an ActA protein. In another embodiment, the recombinant nucleotide comprises any other sequence that encodes an entire ActA protein.

In one embodiment, the ActA sequence for use in the compositions and methods as disclosed herein is from Listeria monocytogenes, which in one embodiment, is the EGD strain, the 10403S strain (Genbank accession number: DQ054585) the NICPBP 54002 strain (Genbank accession number: EU394959), the S3 strain (Genbank accession number: EU394960), the NCTC 5348 strain (Genbank accession number: EU394961), the NICPBP 54006 strain (Genbank accession number: EU394962), the M7 strain (Genbank accession number: EU394963), the S19 strain (Genbank accession number: EU394964), or any other strain of Listeria monocytogenes which is known in the art.

In one embodiment, the sequence of the deleted actA region in the strain LmddAactA is as set forth in SEQ ID NO: 44. In one embodiment, the sequence at positions 583-753 contains an actA sequence element that is present in the LmddAactA strain. In one embodiment, the sequence gtcgac at positions 658-663 represent the site of junction of the N-T and C-T sequence.

In one embodiment, the recombinant Listeria strain of the compositions and methods as disclosed herein comprise a first or second nucleic acid molecule that encodes a High Molecular Weight-Melanoma Associated Antigen (HMW-MAA), or, in another embodiment, a fragment of HMW-MAA.

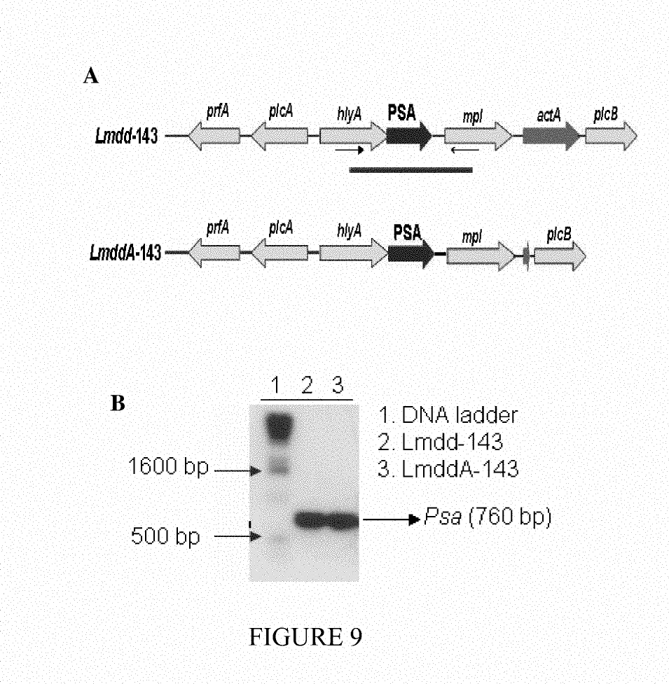

In one embodiment, HMW-MAA is also known as the melanoma chondroitin sulfate proteoglycan (MCSP), and in another embodiment, is a membrane-bound protein of 2322 residues. In one embodiment, HMW-MAA is expressed on over 90% of surgically removed benign nevi and melanoma lesions, and is also expressed in basal cell carcinoma, tumors of neural crest origin (e.g. astrocytomas, gliomas, neuroblastomas and sarcomas), childhood leukemias, and lobular breast carcinoma lesions. In another embodiment, HMW-MAA is highly expressed on both activated pericytes and pericytes in tumor angiogeneic vasculature which, in another embodiment is associated with neovascularization in vivo. In another embodiment, immunization of mice with the recombinant Listeria, as disclosed herein, that expresses a fragment of HMW-MAA (residues 2160 to 2258), impairs the growth of tumors not engineered to express HMW-MAA (FIG. 9D). In another embodiment, immunization of mice with the recombinant Listeria expressing a fragment of HMW-MAA (residues 2160 to 2258) decreases the number of pericytes in the tumor vasculature. In another embodiment, immunization of mice with the recombinant Listeria expressing a fragment of HMW-MAA (residues 2160 to 2258) causes infiltration of CD8⁺ T cells around blood vessels and into the tumor.

In one embodiment, a murine homolog of HMW-MAA, known as NG2 or AN2, has 80% homology to HMW-MAA, as well as similar expression pattern and function. In another embodiment, HMW-MAA is highly expressed on both activated pericytes and pericytes in tumor angiogenic vasculature. In one embodiment, activated pericytes are associated with neovascularization in vivo. In one embodiment, activated pericytes are involved in angiogenesis. In another embodiment, angiogenesis is important for survival of tumors. In another embodiment, pericytes in tumor angiogenic vasculature are associated with neovascularization in vivo. In another embodiment, activated pericytes are important cells in vascular development, stabilization, maturation and remodeling. Therefore, in one embodiment, besides its role as a tumor-associated antigen, HMW-MAA is also a potential universal target for anti-angiogenesis using an immunotherapeutic approach. As described herein (Example 8), results obtained using an Lm-based vaccine against this antigen has supported this possibility.

In another embodiment, one of the antigens of the methods and compositions disclosed herein is expressed in activated pericytes. In another embodiment, at least one of the antigens is expressed in activated pericytes.

The HMW-MAA protein from which HMW-MAA fragments as disclosed herein are derived is, in another embodiment, a human HMW-MAA protein. In another embodiment, the HMW-MAA protein is a mouse protein. In another embodiment, the HMW-MAA protein is a rat protein. In another embodiment, the HMW-MAA protein is a primate protein. In another embodiment, the HMW-MAA protein is from any other species known in the art. In another embodiment, the HMW-MAA protein is melanoma chondroitin sulfate proteoglycan (MCSP). In another embodiment, an AN2 protein is used in methods and compositions as disclosed herein. In another embodiment, an NG2 protein is used in methods and compositions as disclosed herein.

In another embodiment, the HMW-MAA protein of methods and compositions as disclosed herein has an AA sequence set forth in a GenBank entry having an Accession Numbers selected from NM_001897 and X96753. In another embodiment, the HMW-MAA protein is encoded by a nucleotide sequence set forth in one of the above GenBank entries. In another embodiment, the HMW-MAA protein comprises a sequence set forth in one of the above GenBank entries. In another embodiment, the HMW-MAA protein is a homologue of a sequence set forth in one of the above GenBank entries. In another embodiment, the HMW-MAA protein is a variant of a sequence set forth in one of the above GenBank entries. In another embodiment, the HMW-MAA protein is a fragment of a sequence set forth in one of the above GenBank entries. In another embodiment, the HMW-MAA protein is an isoform of a sequence set forth in one of the above GenBank entries disclosed herein.

The HMW-MAA fragment utilized in the present invention comprises, in another embodiment, AA 360-554. In another embodiment, the fragment consists essentially of AA 360-554. In another embodiment, the fragment consists of AA 360-554. In another embodiment, the fragment comprises AA 701-1130. In another embodiment, the fragment consists essentially of AA 701-1130. In another embodiment, the fragment consists of AA 701-1130. In another embodiment, the fragment comprises AA 2160-2258. In another embodiment, the fragment consists essentially of 2160-2258. In another embodiment, the fragment consists of 2160-2258.

In another embodiment, the recombinant Listeria of the compositions and methods as disclosed herein comprise a plasmid that encodes a recombinant polypeptide that is, in one embodiment, angiogenic, and in another embodiment, antigenic. In one embodiment, the polypeptide is HMW-MAA, and in another embodiment, the polypeptide is a HMW-MAA fragment. In another embodiment, the plasmid further encodes a non-HMW-MAA peptide. In one embodiment, the non-HMW-MAA peptide enhances the immunogenicity of the polypeptide. In one embodiment, the HMW-MAA fragment of methods and compositions as disclosed herein is fused to the non-HMW-MAA AA sequence. In another embodiment, the HMW-MAA fragment is embedded within the non-HMW-MAA AA sequence. In another embodiment, an HMW-MAA-derived peptide is incorporated into an LLO fragment, ActA protein or fragment, or PEST-like sequence disclosed herein.

The non-HMW-MAA peptide is, in one embodiment, a listeriolysin (LLO) polypeptide. In another embodiment, the non-HMW-MAA peptide is an ActA polypeptide. In another embodiment, the non-HMW-MAA peptide is a PEST-like polypeptide. In one embodiment, fusion to LLO, ActA, PEST-like sequences and fragments thereof enhances the cell-mediated immunogenicity of antigens. In one embodiment, fusion to LLO, ActA, PEST-like sequences and fragments thereof enhances the cell-mediated immunogenicity of antigens in a variety of expression systems. In another embodiment, the non-HMW-MAA peptide is any other immunogenic non-HMW-MAA peptide known in the art or disclosed herein.

In one embodiment, the recombinant Listeria strain of the compositions and methods as disclosed herein express a heterologous antigen that is expressed by a tumor cell. In one embodiment, the recombinant Listeria strain of the compositions and methods as disclosed herein comprise a first or second nucleic acid molecule that encodes a Prostate Specific Antigen (PSA), which in one embodiment, is a marker for prostate cancer that is highly expressed by prostate tumors, which in one embodiment is the most frequent type of cancer in American men and, in another embodiment, is the second cause of cancer related death in American men. In one embodiment, PSA is a kallikrein serine protease (KLK3) secreted by prostatic epithelial cells, which in one embodiment, is widely used as a marker for prostate cancer.

In one embodiment, the recombinant Listeria strain as disclosed herein comprises a nucleic acid molecule encoding KLK3 protein.

In another embodiment, the KLK3 protein has the sequence set forth in SEQ ID NO: 45 (GenBank Accession No. CAA32915). In another embodiment, the KLK3 protein is a homologue of SEQ ID NO: 45. In another embodiment, the KLK3 protein is a variant of SEQ ID NO: 45. In another embodiment, the KLK3 protein is an isomer of SEQ ID NO: 45. In another embodiment, the KLK3 protein is a fragment of SEQ ID NO: 45.

In another embodiment, the KLK3 protein has the sequence set forth in SEQ ID NO: 46. In another embodiment, the KLK3 protein is a homologue of SEQ ID NO: 46. In another embodiment, the KLK3 protein is a variant of SEQ ID NO: 46. In another embodiment, the KLK3 protein is an isomer of SEQ ID NO: 46. In another embodiment, the KLK3 protein is a fragment of SEQ ID NO: 46.

In another embodiment, the KLK3 protein has the sequence set forth in SEQ ID NO: 47 (GenBank Accession No. AAA59995.1). In another embodiment, the KLK3 protein is a homologue of SEQ ID NO: 47. In another embodiment, the KLK3 protein is a variant of SEQ ID NO: 47. In another embodiment, the KLK3 protein is an isomer of SEQ ID NO: 47. In another embodiment, the KLK3 protein is a fragment of SEQ ID NO: 47.

In another embodiment, the KLK3 protein is encoded by a nucleotide molecule having the sequence set forth in SEQ ID NO: 48 (GenBank Accession No. X14810). In another embodiment, the KLK3 protein is encoded by residues 401 . . . 446, 1688 . . . 1847, 3477 . . . 3763, 3907 . . . 4043, and 5413 . . . 5568 of SEQ ID NO: 48. In another embodiment, the KLK3 protein is encoded by a homologue of SEQ ID NO: 48. In another embodiment, the KLK3 protein is encoded by a variant of SEQ ID NO: 48. In another embodiment, the KLK3 protein is encoded by an isomer of SEQ ID NO: 48. In another embodiment, the KLK3 protein is encoded by a fragment of SEQ ID NO: 48.

In another embodiment, the KLK3 protein has the sequence set forth in SEQ ID NO: 49 (GenBank Accession No. NP_001025218). In another embodiment, the KLK3 protein is a homologue of SEQ ID NO: 49. In another embodiment, the KLK3 protein is a variant of SEQ ID NO: 49. In another embodiment, the KLK3 protein is an isomer of SEQ ID NO: 49. In another embodiment, the KLK3 protein is a fragment of SEQ ID NO: 49.

In another embodiment, the KLK3 protein is encoded by a nucleotide molecule having the sequence set forth in SEQ ID NO: 50 (GenBank Accession No. NM_001030047). In another embodiment, the KLK3 protein is encoded by residues 42-758 of SEQ ID NO: 50. In another embodiment, the KLK3 protein is encoded by a homologue of SEQ ID NO: 50. In another embodiment, the KLK3 protein is encoded by a variant of SEQ ID NO: 50. In another embodiment, the KLK3 protein is encoded by an isomer of SEQ ID NO: 50. In another embodiment, the KLK3 protein is encoded by a fragment of SEQ ID NO: 50.

In another embodiment, the KLK3 protein has the sequence set forth in SEQ ID NO: 51 (GenBank Accession No. NP_001025221). In another embodiment, the KLK3 protein is a homologue of SEQ ID NO: 51. In another embodiment, the KLK3 protein is a variant of SEQ ID NO: 51. In another embodiment, the sequence of the KLK3 protein comprises SEQ ID NO: 51. In another embodiment, the KLK3 protein is an isomer of SEQ ID NO: 51. In another embodiment, the KLK3 protein is a fragment of SEQ ID NO: 51.

In another embodiment, the KLK3 protein is encoded by a nucleotide molecule having the sequence set forth in SEQ ID NO: 52 (GenBank Accession No. NM_001030050). In another embodiment, the KLK3 protein is encoded by residues 42-758 of SEQ ID NO: 52. In another embodiment, the KLK3 protein is encoded by a homologue of SEQ ID NO: 52. In another embodiment, the KLK3 protein is encoded by a variant of SEQ ID NO: 52. In another embodiment, the KLK3 protein is encoded by an isomer of SEQ ID NO: 52. In another embodiment, the KLK3 protein is encoded by a fragment of SEQ ID NO: 52.

In another embodiment, the KLK3 protein that is the source of the KLK3 peptide has the sequence set forth in SEQ ID NO: 53 (GenBank Accession No. NP_001025220). In another embodiment, the KLK3 protein is a homologue of SEQ ID NO: 53. In another embodiment, the KLK3 protein is a variant of SEQ ID NO: 53. In another embodiment, the KLK3 protein is an isomer of SEQ ID NO: 53. In another embodiment, the KLK3 protein is a fragment of SEQ ID NO: 53.

In another embodiment, the KLK3 protein is encoded by a nucleotide molecule having the sequence set forth in SEQ ID NO: 54 (GenBank Accession No. NM_001030049). In another embodiment, the KLK3 protein is encoded by residues 42-758 of SEQ ID NO: 54. In another embodiment, the KLK3 protein is encoded by a homologue of SEQ ID NO: 54. In another embodiment, the KLK3 protein is encoded by a variant of SEQ ID NO: 54. In another embodiment, the KLK3 protein is encoded by an isomer of SEQ ID NO: 54. In another embodiment, the KLK3 protein is encoded by a fragment of SEQ ID NO: 54.

In another embodiment, the KLK3 protein has the sequence set forth in SEQ ID NO: 55 (GenBank Accession No. NP_001025219). In another embodiment, the KLK3 protein is a homologue of SEQ ID NO: 55. In another embodiment, the KLK3 protein is a variant of SEQ ID NO: 55. In another embodiment, the KLK3 protein is an isomer of SEQ ID NO: 55. In another embodiment, the KLK3 protein is a fragment of SEQ ID NO: 55.

In another embodiment, the KLK3 protein is encoded by a nucleotide molecule having the sequence set forth in SEQ ID NO: 56 (GenBank Accession No. NM_001030048). In another embodiment, the KLK3 protein is encoded by residues 42-758 of SEQ ID NO: 56. In another embodiment, the KLK3 protein is encoded by a homologue of SEQ ID NO: 56. In another embodiment, the KLK3 protein is encoded by a variant of SEQ ID NO: 56. In another embodiment, the KLK3 protein is encoded by an isomer of SEQ ID NO: 56. In another embodiment, the KLK3 protein is encoded by a fragment of SEQ ID NO: 56.

In another embodiment, the KLK3 protein has the sequence set forth in SEQ ID NO: 57 (GenBank Accession No. NP_001639). In another embodiment, the KLK3 protein is a homologue of SEQ ID NO: 57. In another embodiment, the KLK3 protein is a variant of SEQ ID NO: 57. In another embodiment, the KLK3 protein is an isomer of SEQ ID NO: 57. In another embodiment, the KLK3 protein is a fragment of SEQ ID NO: 57.

In another embodiment, the KLK3 protein is encoded by a nucleotide molecule having the sequence set forth in SEQ ID NO: 58 (GenBank Accession No. NM_001648). In another embodiment, the KLK3 protein is encoded by residues 42-827 of SEQ ID NO: 58. In another embodiment, the KLK3 protein is encoded by a homologue of SEQ ID NO: 58. In another embodiment, the KLK3 protein is encoded by a variant of SEQ ID NO: 58. In another embodiment, the KLK3 protein is encoded by an isomer of SEQ ID NO: 58. In another embodiment, the KLK3 protein is encoded by a fragment of SEQ ID NO: 58.

In another embodiment, the KLK3 protein has the sequence set forth in SEQ ID NO: 59 (GenBank Accession No. AAX29407.1). In another embodiment, the KLK3 protein is a homologue of SEQ ID NO: 59. In another embodiment, the KLK3 protein is a variant of SEQ ID NO: 59. In another embodiment, the KLK3 protein is an isomer of SEQ ID NO: 59. In another embodiment, the sequence of the KLK3 protein comprises SEQ ID NO: 59. In another embodiment, the KLK3 protein is a fragment of SEQ ID NO: 59.

In another embodiment, the KLK3 protein is encoded by a nucleotide molecule having the sequence set forth in SEQ ID NO: 60 (GenBank Accession No. BC056665). In another embodiment, the KLK3 protein is encoded by residues 47-832 of SEQ ID NO: 60. In another embodiment, the KLK3 protein is encoded by a homologue of SEQ ID NO: 60. In another embodiment, the KLK3 protein is encoded by a variant of SEQ ID NO: 60. In another embodiment, the KLK3 protein is encoded by an isomer of SEQ ID NO: 60. In another embodiment, the KLK3 protein is encoded by a fragment of SEQ ID NO: 60.

In another embodiment, the KLK3 protein has the sequence set forth in SEQ ID NO: 61 (GenBank Accession No. AJ459782). In another embodiment, the KLK3 protein is a homologue of SEQ ID NO: 61. In another embodiment, the KLK3 protein is a variant of SEQ ID NO: 61. In another embodiment, the KLK3 protein is an isomer of SEQ ID NO: 61. In another embodiment, the KLK3 protein is a fragment of SEQ ID NO: 61.

In another embodiment, the KLK3 protein has the sequence set forth in SEQ ID NO: 62 (GenBank Accession No. AJ512346). In another embodiment, the KLK3 protein is a homologue of SEQ ID NO: 62. In another embodiment, the KLK3 protein is a variant of SEQ ID NO: 62. In another embodiment, the KLK3 protein is an isomer of SEQ ID NO: 62. In another embodiment, the sequence of the KLK3 protein comprises SEQ ID NO: 62. In another embodiment, the KLK3 protein is a fragment of SEQ ID NO: 62.

In another embodiment, the KLK3 protein has the sequence set forth in SEQ ID NO: 63 (GenBank Accession No. AJ459784). In another embodiment, the KLK3 protein is a homologue of SEQ ID NO: 63. In another embodiment, the KLK3 protein is a variant of SEQ ID NO: 63. In another embodiment, the sequence of the KLK3 protein comprises SEQ ID NO: 63. In another embodiment, the KLK3 protein is an isomer of SEQ ID NO: 63. In another embodiment, the KLK3 protein is a fragment of SEQ ID NO: 63.

In another embodiment, the KLK3 protein has the sequence set forth in SEQ ID NO: 64 (GenBank Accession No. AJ459783). In another embodiment, the KLK3 protein is a homologue of SEQ ID NO: 64. In another embodiment, the KLK3 protein is a variant of SEQ ID NO: 64. In another embodiment, the KLK3 protein is an isomer of SEQ ID NO: 64. In another embodiment, the KLK3 protein is a fragment of SEQ ID NO: 64.

In another embodiment, the KLK3 protein is encoded by a nucleotide molecule having the sequence set forth in SEQ ID NO: 65 (GenBank Accession No. X07730). In another embodiment, the KLK3 protein is encoded by residues 67-1088 of SEQ ID NO: 65. In another embodiment, the KLK3 protein is encoded by a homologue of SEQ ID NO: 65. In another embodiment, the KLK3 protein is encoded by a variant of SEQ ID NO: 65. In another embodiment, the KLK3 protein is encoded by an isomer of SEQ ID NO: 65. In another embodiment, the KLK3 protein is encoded by a fragment of SEQ ID NO: 65.

In another embodiment, the KLK3 protein is encoded by a sequence set forth in one of the following GenBank Accession Numbers: BC005307, AJ310938, AJ310937, AF335478, AF335477, M27274, and M26663. In another embodiment, the KLK3 protein is encoded by a sequence set forth in one of the above GenBank Accession Numbers. Each possibility represents a separate embodiment of the methods and compositions as disclosed herein.

In another embodiment, the KLK3 protein is encoded by a sequence set forth in one of the following GenBank Accession Numbers: NM_001030050, NM_001030049, NM_001030048, NM_001030047, NM_001648, AJ459782, AJ512346, or AJ459784. Each possibility represents a separate embodiment of the methods and compositions as disclosed herein. In one embodiment, the KLK3 protein is encoded by a variation of any of the sequences described herein wherein the sequence lacks the sequence set forth in SEQ ID NO: 66.

In another embodiment, the KLK3 protein has the sequence that comprises a sequence set forth in one of the following GenBank Accession Numbers: X13943, X13942, X13940, X13941, and X13944.

In another embodiment, the KLK3 protein is any other KLK3 protein known in the art.

In another embodiment, the KLK3 peptide is any other KLK3 peptide known in the art. In another embodiment, the KLK3 peptide is a fragment of any other KLK3 peptide known in the art. Each type of KLK3 peptide represents a separate embodiment of the methods and compositions as disclosed herein.

“KLK3 peptide” refers, in another embodiment, to a full-length KLK3 protein. In another embodiment, the term refers to a fragment of a KLK3 protein. In another embodiment, the term refers to a fragment of a KLK3 protein that is lacking the KLK3 signal peptide. In another embodiment, the term refers to a KLK3 protein that contains the entire KLK3 sequence except the KLK3 signal peptide. “KLK3 signal sequence” refers, in another embodiment, to any signal sequence found in nature on a KLK3 protein. In another embodiment, a KLK3 protein of methods and compositions as disclosed herein does not contain any signal sequence.

In another embodiment, the kallikrein-related peptidase 3 (KLK3 protein) that is the source of a KLK3 peptide for use in the methods and compositions disclosed herein is a PSA protein. In another embodiment, the KLK3 protein is a P-30 antigen protein. In another embodiment, the KLK3 protein is a gamma-seminoprotein protein. In another embodiment, the KLK3 protein is a kallikrein 3 protein. In another embodiment, the KLK3 protein is a semenogelase protein. In another embodiment, the KLK3 protein is a seminin protein. In another embodiment, the KLK3 protein is any other type of KLK3 protein that is known in the art.

In another embodiment, the KLK3 protein is a splice variant 1 KLK3 protein. In another embodiment, the KLK3 protein is a splice variant 2 KLK3 protein. In another embodiment, the KLK3 protein is a splice variant 3 KLK3 protein. In another embodiment, the KLK3 protein is a transcript variant 1 KLK3 protein. In another embodiment, the KLK3 protein is a transcript variant 2 KLK3 protein. In another embodiment, the KLK3 protein is a transcript variant 3 KLK3 protein. In another embodiment, the KLK3 protein is a transcript variant 4 KLK3 protein. In another embodiment, the KLK3 protein is a transcript variant 5 KLK3 protein. In another embodiment, the KLK3 protein is a transcript variant 6 KLK3 protein. In another embodiment, the KLK3 protein is a splice variant RP5 KLK3 protein. In another embodiment, the KLK3 protein is any other splice variant KLK3 protein known in the art. In another embodiment, the KLK3 protein is any other transcript variant KLK3 protein known in the art.

In another embodiment, the KLK3 protein is a mature KLK3 protein. In another embodiment, the KLK3 protein is a pro-KLK3 protein. In another embodiment, the leader sequence has been removed from a mature KLK3 protein of methods and compositions disclosed herein.

In another embodiment, the KLK3 protein that is the source of a KLK3 peptide of methods and compositions as disclosed herein is a human KLK3 protein. In another embodiment, the KLK3 protein is a primate KLK3 protein. In another embodiment, the KLK3 protein is a KLK3 protein of any other species known in the art. In another embodiment, one of the above KLK3 proteins is referred to in the art as a “KLK3 protein.”

In another embodiment, KLK3-LLO fusions are provided in U.S. Pat. No. 9,012,141 which is incorporated by reference herein in its entirety. In another embodiment, the antigen of interest is HPV-E7. In another embodiment, the antigen is HPV-E6. In another embodiment, the antigen is Her-2/neu. In another embodiment, the antigen is NY-ESO-1. In another embodiment, the antigen is telomerase (TERT). In another embodiment, the antigen is stratum corneum chymotryptic enzyme (SCCE) and variants thereof. In another embodiment, the antigen is CEA. In another embodiment, the antigen is LMP-1. In another embodiment, the antigen is p53. In another embodiment, the antigen is carboxic anhydrase IX (CAIX). In another embodiment, the antigen is prostate-specific membrane antigen (PSMA). In another embodiment, the antigen is prostate stem cell antigen (PSCA). In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from human papilloma virus E7 (HPV-E7), HPV-E6, Her-2, NY-ESO-1, telomerase (TERT), Kallikrein-Related Peptidase 7 (SCCE; KLK7), HMW-MAA, WT-1, HIV-1 Gag, CEA, LMP-1, p53, PSMA, Prostate Stem Cell Antigen (PSCA), Proteinase 3, Tyrosinase related protein 2, Survivin (BIRC5), Mucl, prostate-specific antigen (PSA; KLK3), A Kinase Anchor Protein 4 (AKAP4), Hepsin (HPN/TMPRSS1), Prostate-specific G-protein-coupled receptor (PSGR/OR51E2), T-cell receptor γ-chain Alternate Reading-Frame Protein (TARP), Mammalian Enabled Homolog (ENAH; hMENA), POTE paralogs, O-GlcNAc Transferase (OGT), KLK7, Secernin-1 (SCRN1), Fibroblast Activation Protein (FAP), Matrix Metallopeptidase 7 (MMP7), Milk Fat Globule-EGF Factor 8 Protein (MFGE8), Wilms Tumor 1 (WT1), Interferon-Stimulated Gene 15 Ubiquitin-Like Modifier (ISG15; G1P2), Acrosin Binding Protein (ACRBP; OY-TES-1), Kallikrein-Related Peptidase 4 (KLK4/prostase) or a combination thereof.

In one embodiment, the E7 protein comprises SEQ ID NO: 67.

In another embodiment, the antigen is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; carbonic anhydrase 9 (CA9), a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, mesothelin, EGFRvIII, a MART1 antigen associated with melanoma, or the PSA antigen associated with prostate cancer. In another embodiment, the antigen for the compositions and methods disclosed herein are melanoma-associated antigens, which in one embodiment are TRP-2, MAGE-1, MAGE-3, gp-100, tyrosinase, HSP-70, beta-HCG, or a combination thereof.

In one embodiment, the recombinant nucleic acid disclosed herein may encode two separate antigens that serve as tumor targets, which in one embodiment are Prostate Specific Antigen (PSA) and Prostate Cancer Stem Cell (PSMA) antigen. In one embodiment, the recombinant nucleic acid molecule disclosed herein encodes two separate antigens that serve as tumor targets, which in one embodiment are PSA and survivin. In another embodiment, the recombinant nucleic acid molecule disclosed herein encodes two separate antigens that serve as tumor targets, which in one embodiment are cHer2 and CA9. In one embodiment, the each individual antigen of the two or more antigens expressed by a Listeria disclosed herein complement or synergize the immune response.

In another embodiment, the heterologous antigen disclosed herein is an angiogenic antigen that affects vascular growth. In one embodiment, the recombinant nucleic acid disclosed herein may encode two polypeptides each comprising an angiogenic antigen that affect vascular growth fused to a PEST-containing peptide disclosed herein. In one embodiment, the angiogenic antigen is any angiogenic antigen known in the art, including but not limited to EGFR-III and its related family members, VEGFR and its related family members, HMW-MAA. In one embodiment, the heterologous antigen disclosed herein may serve as both a tumor antigen an angiogenic factor. In one embodiment, the heterologous antigen is a tumor antigen. In another embodiment, the heterologous antigen is an inhibitor of the function or expression of ARG-1 or NOS or combination. In one embodiment, an inhibitor of NOS is NG-mono-methyl-L-arginine (L-NMMA), NG-nitro-L-arginine methyl ester (L-NAME), 7-NI, L-NIL, or L-NIO. In one embodiment, N-omega-nitro-L-arginine a nitric oxide synthase inhibitor and L-arginine competitive inhibitor may be encoded by the nucleic acid. In one embodiment, the second nucleic acid may encode an mRNA that inhibits function or expression of ARG-1 or NOS.

In one embodiment, a heterologous antigen expressed by the Listeria of the present invention may be a neuropeptide growth factor antagonist, which in one embodiment is [D-Arg1, D-Phe5, D-Trp7,9, Leu11] substance P, [Arg6, D-Trp7,9, NmePhe8] substance P(6-11). These and related embodiments are understood by one of skill in the art.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, or a combination thereof.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

The immune response induced by methods and compositions disclosed herein is, in another embodiment, a T cell response. In another embodiment, the immune response comprises a T cell response. In another embodiment, the response is a CD8+ T cell response. In another embodiment, the response comprises a CD8⁺ T cell response.

In one embodiment, a recombinant Listeria of the compositions and methods as disclosed herein comprise an angiogenic polypeptide. In another embodiment, anti-angiogenic approaches to cancer therapy are very promising, and in one embodiment, one type of such anti-angiogenic therapy targets pericytes. In another embodiment, molecular targets on vascular endothelial cells and pericytes are important targets for antitumor therapies. In another embodiment, the platelet-derived growth factor receptor (PDGF-B/PDGFR-3) signaling is important to recruit pericytes to newly formed blood vessels. Thus, in one embodiment, angiogenic polypeptides disclosed herein inhibit molecules involved in pericyte signaling, which in one embodiment, is PDGFR-3.

In one embodiment, the compositions of the present invention comprise an angiogenic factor, or an immunogenic fragment thereof, where in one embodiment, the immunogenic fragment comprises one or more epitopes recognized by the host immune system. In one embodiment, an angiogenic factor is a molecule involved in the formation of new blood vessels. In one embodiment, the angiogenic factor is VEGFR2. In another embodiment, an angiogenic factor of the present invention is Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods of the present invention is Fibroblast growth factors (FGF); VEGF; VEGFR and Neuropilin 1 (NRP-1); Angiopoietin 1 (Ang1) and Tie2; Platelet-derived growth factor (PDGF; BB-homodimer) and PDGFR; Transforming growth factor-beta (TGF-1), endoglin and TGF-β receptors; monocyte chemotactic protein-1 (MCP-1); Integrins αVβ3, αVβ5 and α5β1; VE-cadherin and CD31; ephrin; plasminogen activators; plasminogen activator inhibitor-1; Nitric oxide synthase (NOS) and COX-2; AC133; or Id1/Id3. In one embodiment, an angiogenic protein for use in the compositions and methods of the present invention is an angiopoietin, which in one embodiment, is Angiopoietin 1, Angiopoietin 3, Angiopoietin 4 or Angiopoietin 6. In one embodiment, endoglin is also known as CD105; EDG; HHT1; ORW; or ORW1. In one embodiment, endoglin is a TGF-beta co-receptor.

Examples of target antigens that may find use in the present invention include, but is not limited to: Wilm's tumor-1 associated protein (Wt-1), including Isoforms A, B, C, and D; MHC class I chain-related protein A (MICA); MHC class I chain-related protein B (MICB); gastrin and peptides thereof; gastrin/CCK-2 receptor (CCK-B); Glypican-3; Coactosin-like protein; Prostate acid phosphatase (PAP); Six-transmembrane epithelial antigen of prostate (STEAP); Prostate carcinoma antigen-1 (PCTA-1); Prostate tumor-inducing gene-1 (PTI-1); Prostate-specific gene with homology to G protein-coupled receptor; Prostase; Cancer-testis antigens; SCP-1; SSX-1, SSX-2, SSX-4; GAGE; CT7; CT8; CT10; LAGE-1; GAGE-3/6, GAGE-1, GAGE-2, GAGE-3, GAGE-4, GAGE-5, GAGE-6, GAGE-7 GAGE-8; BAGE; NT-SAR-35; CA-125; HIP1R; LMNA; KIAA1416; Seb4D; KNSL6; TRIP4; MDB2; HCAC5; DAM family of genes; RCAS1; RU2; CAMEL; Colon cancer-associated antigens, e.g., NY-CO-8, NY-CO-13, NY-CO-9, NY-CO-16, NY-CO-20, NY-CO-38, NY-CO-45, NY-CO-9/HDAC5; NY-CO-41/MBD2; NY-CO-42/TRIP4; NY-CO-95/KIAA1416; KNSL6; seb4D; N-Acetylglucosaminyl-transferase V (GnT-V); Elongation factor 2 mutated (ELF2M); HOM-MEL-40/SSX-2; BRDT; SAGE; HAGE; RAGE; Melanoma ubiquitous mutated (MUM-1); MUM-2 Arg-Gly mutation; MUM-3; LDLR/FUT fusion protein antigen of melanoma; NY-REN series of renal cancer antigens; NY-BR series of breast cancer antigens, e.g., NY-BR-62, NY-BR-75, NY-BR-85; BRCA-1, BRCA-2; DEK/CAN fusion protein; Ras, including with mutations in codon 12, 13, 59, or 61, e.g., mutations G12C, G12D, G12R, G12S, G12V, G13D, A59T, Q61H; K-RAS; H-RAS; N-RAS; BRAF; Melanoma antigens including HST-2; MDM-2; Methyl-CpG-binding proteins (MeCP2; MBD2); NA88-A; Histone deacetylases; Cyclophilin B (CYP-B); CA15-3; CA27.29; HsP70; GAGE/PAGE family; Kinesin-2; TATA element modulatory factor 1; tumor protein D53; NY alfa-fetoprotein (AFP); SART1; SART2; SART3; ART4; Preferentially expressed antigen of melanoma (PRAME); CAP1-6D enhancer agonist peptide; cdk4; cdk6; p16 (INK4); Rb protein; TEL; AML1; TEL/AML1; Telomerase (TERT); 707-AP; Annexin, e.g., Annexin II; CML-66; CLM-28; BLC2, BCL6; CD10 protein; CDC27; Sperm protein 17 (SP17); 14-3-3 zeta; MEMD; KIAA0471; TC21; Tyrosinase related proteins 1 and 2 (TRP-1, TRP-2); Gp-100/pmel-17; TARP; Nkx3.1; Melanocortin-1 receptor (MC1R); MUC-1, MUC-2; ETV6/AML1; E-cadherin;
cyclooxygenase-2 (COX-2); EphA2; and infectious disease related antigens all of which are listed in US Patent publication serial no. 2014/0186387, which is incorporated by reference herein.

In one embodiment, cancer vaccines as disclosed herein generate effector T cells that are able to infiltrate the tumor, destroy tumor cells and eradicate the disease. In one embodiment, naturally occurring tumor infiltrating lymphocytes (TILs) are associated with better prognosis in several tumors, such as colon, ovarian and melanoma. In colon cancer, tumors without signs of micrometastasis have an increased infiltration of immune cells and a Thl expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines as disclosed herein increase TILs, up-regulate adhesion molecules (in one embodiment, ICAM-1, V-CAM-1, VAP-1, E-selectin, or a combination thereof), up-regulate proinflammatory cytokines (in one embodiment, IFN-γ, TNF-α, IL-1, or a combination thereof), or a combination thereof.

In one embodiment, the compositions and methods as disclosed herein provide anti-angiogenesis therapy, which in one embodiment, may improve immunotherapy strategies. In one embodiment, the compositions and methods as disclosed herein circumvent endothelial cell anergy in vivo by up-regulating adhesion molecules in tumor vessels and enhancing leukocyte-vessel interactions, which increases the number of tumor infiltrating leukocytes, such as CD8⁺ T cells. Interestingly, enhanced anti-tumor protection correlates with an increased number of activated CD4⁺ and CD8⁺ tumor-infiltrating T cells and a pronounced decrease in the number of regulatory T cells in the tumor upon VEGF blockade.

In one embodiment, delivery of anti-angiogenic antigen simultaneously with a tumor-associated antigen to a host afflicted by a tumor as described herein, will have a synergistic effect in impacting tumor growth and a more potent therapeutic efficacy.

In another embodiment, targeting pericytes through vaccination will lead to cytotoxic T lymphocyte (CTL) infiltration, destruction of pericytes, blood vessel destabilization and vascular inflammation, which in another embodiment is associated with up-regulation of adhesion molecules in the endothelial cells that are important for lymphocyte adherence and transmigration, ultimately improving the ability of lymphocytes to infiltrate the tumor tissue. In another embodiment, concomitant delivery of a tumor-specific antigen generate lymphocytes able to invade the tumor site and kill tumor cells.

In one embodiment, the platelet-derived growth factor receptor (PDGF-B/PDGFR-3) signaling is important to recruit pericytes to newly formed blood vessels. In another embodiment, inhibition of VEGFR-2 and PDGFR-j3 concomitantly induces endothelial cell apoptosis and regression of tumor blood vessels, in one embodiment, approximately 40% of tumor blood vessels.

In another embodiment, said recombinant Listeria strain is an auxotrophic Listeria strain. In another embodiment, said auxotrophic Listeria strain is a dal/dat mutant. In another embodiment, the nucleic acid molecule is stably maintained in the recombinant bacterial strain in the absence of antibiotic selection.

In one embodiment, auxotrophic mutants useful as vaccine vectors may be generated in a number of ways. In another embodiment, D-alanine auxotrophic mutants can be generated, in one embodiment, via the disruption of both the dal gene and the dat gene to generate an attenuated auxotrophic strain of Listeria which requires exogenously added D-alanine for growth.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as disclosed herein, may be used as targets for mutagenesis of Listeria.

In one embodiment, said auxotrophic Listeria strain comprises an episomal expression vector comprising a metabolic enzyme that complements the auxotrophy of said auxotrophic Listeria strain. In another embodiment, the construct is contained in the Listeria strain in an episomal fashion. In another embodiment, the foreign antigen is expressed from a vector harbored by the recombinant Listeria strain. In another embodiment, said episomal expression vector lacks an antibiotic resistance marker. In one embodiment, an antigen of the methods and compositions as disclosed herein is genetically fused to a polypeptide comprising a PEST sequence. In another embodiment, said endogenous polypeptide comprising a PEST sequence is LLO. In another embodiment, said endogenous polypeptide comprising a PEST sequence is ActA.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, said metabolic enzyme is an amino acid metabolism enzyme. In another embodiment, said metabolic enzyme catalyzes a formation of an amino acid used for a cell wall synthesis in said recombinant Listeria strain. In another embodiment, said metabolic enzyme is an alanine racemase enzyme. In another embodiment, said metabolic enzyme is a D-amino acid transferase enzyme.

In another embodiment, the metabolic enzyme catalyzes the formation of an amino acid (AA) used in cell wall synthesis. In another embodiment, the metabolic enzyme catalyzes synthesis of an AA used in cell wall synthesis. In another embodiment, the metabolic enzyme is involved in synthesis of an AA used in cell wall synthesis. In another embodiment, the AA is used in cell wall biogenesis.

In another embodiment, the metabolic enzyme is a synthetic enzyme for D-glutamic acid, a cell wall component.

In another embodiment, the metabolic enzyme is encoded by an alanine racemase gene (dal) gene. In another embodiment, the dal gene encodes alanine racemase, which catalyzes the reaction L-alanine↔D-alanine.

The dal gene of methods and compositions of the methods and compositions as disclosed herein is encoded, in another embodiment, by the sequence set forth in SEQ ID NO: 68 (GenBank Accession No: AF038438). In another embodiment, the nucleotide encoding dal is homologous to SEQ ID NO: 68. In another embodiment, the nucleotide encoding dal is a variant of SEQ ID NO: 68. In another embodiment, the nucleotide encoding dal is a fragment of SEQ ID NO: 68. In another embodiment, the dal protein is encoded by any other dal gene known in the art.

In another embodiment, the dal protein has the sequence set forth in SEQ ID NO: 69 (GenBank Accession No: AF038428). In another embodiment, the dal protein is homologous to SEQ ID NO: 69. In another embodiment, the dal protein is a variant of SEQ ID NO: 69. In another embodiment, the dal protein is an isomer of SEQ ID NO: 69. In another embodiment, the dal protein is a fragment of SEQ ID NO: 69. In another embodiment, the dal protein is a fragment of a homologue of SEQ ID NO: 69. In another embodiment, the dal protein is a fragment of a variant of SEQ ID NO: 69. In another embodiment, the dal protein is a fragment of an isomer of SEQ ID NO: 69.

In another embodiment, the dal protein is any other Listeria dal protein known in the art. In another embodiment, the dal protein is any other gram-positive dal protein known in the art. In another embodiment, the dal protein is any other dal protein known in the art.

In another embodiment, the dal protein of methods and compositions as disclosed herein retains its enzymatic activity. In another embodiment, the dal protein retains 90% of wild-type activity. In another embodiment, the dal protein retains 80% of wild-type activity. In another embodiment, the dal protein retains 70% of wild-type activity. In another embodiment, the dal protein retains 60% of wild-type activity. In another embodiment, the dal protein retains 50% of wild-type activity. In another embodiment, the dal protein retains 40% of wild-type activity. In another embodiment, the dal protein retains 30% of wild-type activity. In another embodiment, the dal protein retains 20% of wild-type activity. In another embodiment, the dal protein retains 10% of wild-type activity. In another embodiment, the dal protein retains 5% of wild-type activity.

In another embodiment, the metabolic enzyme is encoded by a D-amino acid aminotransferase gene (dat). D-glutamic acid synthesis is controlled in part by the dat gene, which is involved in the conversion of D-glu+pyr to alpha-ketoglutarate+D-ala, and the reverse reaction.

In another embodiment, a dat gene utilized in the present invention has the sequence set forth in GenBank Accession Number AF038439. In another embodiment, the dat gene is any another dat gene known in the art.

The dat gene of methods and compositions of the methods and compositions as disclosed herein is encoded, in another embodiment, by the sequence set forth in SEQ ID NO: 70 (GenBank Accession No: AF038439). In another embodiment, the nucleotide encoding dat is homologous to SEQ ID NO: 70. In another embodiment, the nucleotide encoding dat is a variant of SEQ ID NO: 70. In another embodiment, the nucleotide encoding dat is a fragment of SEQ ID NO: 70. In another embodiment, the dat protein is encoded by any other dat gene known in the art.

In another embodiment, the dat protein has the sequence set forth in SEQ ID NO: 71 (GenBank Accession No: AF038439). In another embodiment, the dat protein is homologous to SEQ ID NO: 71. In another embodiment, the dat protein is a variant of SEQ ID NO: 71. In another embodiment, the dat protein is an isomer of SEQ ID NO: 71. In another embodiment, the dat protein is a fragment of SEQ ID NO: 71. In another embodiment, the dat protein is a fragment of a homologue of SEQ ID NO: 71. In another embodiment, the dat protein is a fragment of a variant of SEQ ID NO: 71. In another embodiment, the dat protein is a fragment of an isomer of SEQ ID NO: 71.

In another embodiment, the dat protein is any other Listeria dat protein known in the art. In another embodiment, the dat protein is any other gram-positive dat protein known in the art. In another embodiment, the dat protein is any other dat protein known in the art.

In another embodiment, the dat protein of methods and compositions of the methods and compositions as disclosed herein retains its enzymatic activity. In another embodiment, the dat protein retains 90% of wild-type activity. In another embodiment, the dat protein retains 80% of wild-type activity. In another embodiment, the dat protein retains 70% of wild-type activity. In another embodiment, the dat protein retains 60% of wild-type activity. In another embodiment, the dat protein retains 50% of wild-type activity. In another embodiment, the dat protein retains 40% of wild-type activity. In another embodiment, the dat protein retains 30% of wild-type activity. In another embodiment, the dat protein retains 20% of wild-type activity. In another embodiment, the dat protein retains 10% of wild-type activity. In another embodiment, the dat protein retains 5% of wild-type activity.

In another embodiment, the metabolic enzyme is encoded by dga. D-glutamic acid synthesis is also controlled in part by the dga gene, and an auxotrophic mutant for D-glutamic acid synthesis will not grow in the absence of D-glutamic acid (Pucci et al, 1995, J Bacteriol. 177: 336-342). In another embodiment, the recombinant Listeria is auxotrophic for D-glutamic acid. A further example includes a gene involved in the synthesis of diaminopimelic acid. Such synthesis genes encode beta-semialdehyde dehydrogenase, and when inactivated, renders a mutant auxotrophic for this synthesis pathway (Sizemore et al, 1995, Science 270: 299-302). In another embodiment, the dga protein is any other Listeria dga protein known in the art. In another embodiment, the dga protein is any other gram-positive dga protein known in the art.

In another embodiment, the metabolic enzyme is encoded by an air (alanine racemase) gene. In another embodiment, the metabolic enzyme is any other enzyme known in the art that is involved in alanine synthesis. In another embodiment, the metabolic enzyme is any other enzyme known in the art that is involved in L-alanine synthesis. In another embodiment, the metabolic enzyme is any other enzyme known in the art that is involved in D-alanine synthesis. In another embodiment, the recombinant Listeria is auxotrophic for D-alanine. Bacteria auxotrophic for alanine synthesis are well known in the art, and are described in, for example, E. coli (Strych et al, 2002, J. Bacteriol. 184:4321-4325), Corynebacterium glutamicum (Tauch et al, 2002, J. Biotechnol 99:79-91), and Listeria monocytogenes (Frankel et al, U.S. Pat. No. 6,099,848)), Lactococcus species, and Lactobacillus species, (Bron et al, 2002, Appl Environ Microbiol, 68: 5663-70). In another embodiment, any D-alanine synthesis gene known in the art is inactivated.

In another embodiment, the metabolic enzyme is an amino acid aminotransferase.

In another embodiment, the metabolic enzyme is encoded by serC, a phosphoserine aminotransferase. In another embodiment, the metabolic enzyme is encoded by asd (aspartate beta-semialdehyde dehydrogenase), involved in synthesis of the cell wall constituent diaminopimelic acid. In another embodiment, the metabolic enzyme is encoded by gsaB-glutamate-1-semialdehyde aminotransferase, which catalyzes the formation of 5-aminolevulinate from (S)-4-amino-5-oxopentanoate. In another embodiment, the metabolic enzyme is encoded by HemL, which catalyzes the formation of 5-aminolevulinate from (S)-4-amino-5-oxopentanoate. In another embodiment, the metabolic enzyme is encoded by aspB, an aspartate aminotransferase that catalyzes the formation of oxalozcetate and L-glutamate from L-aspartate and 2-oxoglutarate. In another embodiment, the metabolic enzyme is encoded by argF-1, involved in arginine biosynthesis. In another embodiment, the metabolic enzyme is encoded by aroE, involved in amino acid biosynthesis. In another embodiment, the metabolic enzyme is encoded by aroB, involved in 3-dehydroquinate biosynthesis. In another embodiment, the metabolic enzyme is encoded by aroD, involved in amino acid biosynthesis. In another embodiment, the metabolic enzyme is encoded by aroC, involved in amino acid biosynthesis. In another embodiment, the metabolic enzyme is encoded by hisB, involved in histidine biosynthesis. In another embodiment, the metabolic enzyme is encoded by hisD, involved in histidine biosynthesis. In another embodiment, the metabolic enzyme is encoded by hisG, involved in histidine biosynthesis. In another embodiment, the metabolic enzyme is encoded by metX, involved in methionine biosynthesis. In another embodiment, the metabolic enzyme is encoded by proB, involved in proline biosynthesis. In another embodiment, the metabolic enzyme is encoded by argR, involved in arginine biosynthesis. In another embodiment, the metabolic enzyme is encoded by argJ, involved in arginine biosynthesis. In another embodiment, the metabolic enzyme is encoded by thiI, involved in thiamine biosynthesis. In another embodiment, the metabolic enzyme is encoded by LMOf2365_1652, involved in tryptophan biosynthesis. In another embodiment, the metabolic enzyme is encoded by aroA, involved in tryptophan biosynthesis. In another embodiment, the metabolic enzyme is encoded by ilvD, involved in valine and isoleucine biosynthesis. In another embodiment, the metabolic enzyme is encoded by ilvC, involved in valine and isoleucine biosynthesis. In another embodiment, the metabolic enzyme is encoded by leuA, involved in leucine biosynthesis. In another embodiment, the metabolic enzyme is encoded by dapF, involved in lysine biosynthesis. In another embodiment, the metabolic enzyme is encoded by thrB, involved in threonine biosynthesis (all GenBank Accession No. NC_002973).

In another embodiment, the metabolic enzyme is a tRNA synthetase. In another embodiment, the metabolic enzyme is encoded by the trpS gene, encoding tryptophanyltRNA synthetase. In another embodiment, the metabolic enzyme is any other tRNA synthetase known in the art.

In another embodiment, the LmddA strain disclosed herein comprises a mutation, deletion or an inactivation of the dal/dat and actA chromosomal genes.

In another embodiment, a recombinant Listeria strain as disclosed herein has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the passaging attenuates the strain, or in another embodiment, makes the strain less virulent. Methods for passaging a recombinant Listeria strain through an animal host are well known in the art, and are described, for example, in U.S. patent application Ser. No. 10/541,614. Each possibility represents a separate embodiment of the methods and compositions as disclosed herein.

The recombinant Listeria strain of the methods and compositions as disclosed herein is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. In another embodiment, the sequences of Listeria proteins for use in the methods and compositions as disclosed herein are from any of the above-described strains.

In one embodiment, a Listeria monocytogenes strain as disclosed herein is the EGD strain, the 10403S strain, the NICPBP 54002 strain, the S3 strain, the NCTC 5348 strain, the NICPBP 54006 strain, the M7 strain, the S19 strain, or another strain of Listeria monocytogenes which is known in the art.

In another embodiment, the recombinant Listeria strain is a vaccine strain, which in one embodiment, is a bacterial vaccine strain.

In another embodiment, the recombinant Listeria strain utilized in methods of the present invention has been stored in a frozen cell bank. In another embodiment, the recombinant Listeria strain has been stored in a lyophilized cell bank. Each possibility represents a separate embodiment of the present invention.

In another embodiment, the cell bank of methods and compositions of the present invention is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art. Each possibility represents a separate embodiment of the present invention.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States. Each possibility represents a separate embodiment of the present invention.

In another embodiment, a recombinant Listeria strain utilized in methods of the present invention is from a batch of vaccine doses.

In another embodiment, a recombinant Listeria strain utilized in methods of the present invention is from a frozen stock produced by a method disclosed herein.

In another embodiment, a recombinant Listeria strain utilized in methods of the present invention is from a lyophilized stock produced by a method disclosed herein.

In another embodiment, a cell bank, frozen stock, or batch of vaccine doses of the present invention exhibits viability upon thawing of greater than 90%. In another embodiment, the thawing follows storage for cryopreservation or frozen storage for 24 hours. In another embodiment, the storage is for 2 days. In another embodiment, the storage is for 3 days. In another embodiment, the storage is for 4 days. In another embodiment, the storage is for 1 week. In another embodiment, the storage is for 2 weeks. In another embodiment, the storage is for 3 weeks. In another embodiment, the storage is for 1 month. In another embodiment, the storage is for 2 months. In another embodiment, the storage is for 3 months. In another embodiment, the storage is for 5 months. In another embodiment, the storage is for 6 months. In another embodiment, the storage is for 9 months. In another embodiment, the storage is for 1 year. Each possibility represents a separate embodiment of the present invention.

In another embodiment, a cell bank, frozen stock, or batch of vaccine doses of the present invention is cryopreserved by a method that comprises growing a culture of the Listeria strain in a nutrient media, freezing the culture in a solution comprising glycerol, and storing the Listeria strain at below −20 degrees Celsius. In another embodiment, the temperature is about −70 degrees Celsius. In another embodiment, the temperature is about ⁻70-⁻80 degrees Celsius.

In another embodiment of methods and compositions of the present invention, the culture (e.g. the culture of a Listeria vaccine strain that is used to produce a batch of Listeria vaccine doses) is inoculated from a cell bank. In another embodiment, the culture is inoculated from a frozen stock. In another embodiment, the culture is inoculated from a starter culture. In another embodiment, the culture is inoculated from a colony. In another embodiment, the culture is inoculated at mid-log growth phase. In another embodiment, the culture is inoculated at approximately mid-log growth phase. In another embodiment, the culture is inoculated at another growth phase.

In another embodiment of methods and compositions of the present invention, the solution used for freezing contains glycerol in an amount of 2-20%. In another embodiment, the amount is 2%. In another embodiment, the amount is 20%. In another embodiment, the amount is 1%. In another embodiment, the amount is 1.5%. In another embodiment, the amount is 3%. In another embodiment, the amount is 4%. In another embodiment, the amount is 5%. In another embodiment, the amount is 2%. In another embodiment, the amount is 2%. In another embodiment, the amount is 7%. In another embodiment, the amount is 9%. In another embodiment, the amount is 10%. In another embodiment, the amount is 12%. In another embodiment, the amount is 14%. In another embodiment, the amount is 16%. In another embodiment, the amount is 18%. In another embodiment, the amount is 222%. In another embodiment, the amount is 25%. In another embodiment, the amount is 30%. In another embodiment, the amount is 35%. In another embodiment, the amount is 40%. Each possibility represents a separate embodiment of the present invention.

In another embodiment, the solution used for freezing contains another colligative additive or additive with anti-freeze properties, in place of glycerol. In another embodiment, the solution used for freezing contains another colligative additive or additive with anti-freeze properties, in addition to glycerol. In another embodiment, the additive is mannitol. In another embodiment, the additive is DMSO. In another embodiment, the additive is sucrose. In another embodiment, the additive is any other colligative additive or additive with anti-freeze properties that is known in the art.

In one embodiment, a vaccine is a composition which elicits an immune response to an antigen or polypeptide in the composition as a result of exposure to the composition. In another embodiment, the vaccine additionally comprises an adjuvant, cytokine, chemokine, or combination thereof. In another embodiment, the vaccine or composition additionally comprises antigen presenting cells (APCs), which in one embodiment are autologous, while in another embodiment, they are allogeneic to the subject.

In one embodiment, a “vaccine” is a composition which elicits an immune response in a host to an antigen or polypeptide in the composition as a result of exposure to the composition. In one embodiment, the immune response is to a particular antigen or to a particular epitope on the antigen. In one embodiment, the vaccine may be a peptide vaccine, in another embodiment, a DNA vaccine. In another embodiment, the vaccine may be contained within and, in another embodiment, delivered by, a cell, which in one embodiment is a bacterial cell, which in one embodiment, is a Listeria. In one embodiment, a vaccine may prevent a subject from contracting or developing a disease or condition, wherein in another embodiment, a vaccine may be therapeutic to a subject having a disease or condition. In one embodiment, a vaccine of the present invention comprises a composition of the present invention and an adjuvant, cytokine, chemokine, or combination thereof.

In another embodiment, the present invention provides an immunogenic composition comprising a recombinant Listeria of the present invention. In another embodiment, the immunogenic composition of methods and compositions of the present invention comprises a recombinant vaccine vector of the present invention. In another embodiment, the immunogenic composition comprises a plasmid of the present invention. In another embodiment, the immunogenic composition comprises an adjuvant. In one embodiment, a vector of the present invention may be administered as part of a vaccine composition. Each possibility represents a separate embodiment of the present invention.

In another embodiment, a vaccine of the present invention is delivered with an adjuvant. In one embodiment, the adjuvant favors a predominantly Thl-mediated immune response. In another embodiment, the adjuvant favors a Thl-type immune response. In another embodiment, the adjuvant favors a Thl-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, the adjuvant is MPL. In another embodiment, the adjuvant is QS21. In another embodiment, the adjuvant comprises a granulocyte/macrophage colony-stimulating factor (GM-CSF) protein or a nucleotide molecule encoding a GM-CSF protein. In another embodiment, the adjuvant is a TLR agonist. In another embodiment, the adjuvant is a TLR4 agonist. In another embodiment, the adjuvant is monophosphoryl lipid A. In another embodiment, the adjuvant is a TLR9 agonist. In another embodiment, the adjuvant is Resiquimod®. In another embodiment, the adjuvant is imiquimod. In another embodiment, the adjuvant is a CpG oligonucleotide. In another embodiment, the adjuvant is a cytokine or a nucleic acid encoding same. In another embodiment, the adjuvant is a chemokine or a nucleic acid encoding same. In another embodiment, the adjuvant is IL-12 or a nucleic acid encoding same. In another embodiment, the adjuvant is IL-6 or a nucleic acid encoding same. In another embodiment, the adjuvant is a lipopolysaccharide. In another embodiment, the adjuvant is as described in Fundamental Immunology, 5th ed (August 2003): William E. Paul (Editor); Lippincott Williams & Wilkins Publishers; Chapter 43: Vaccines, GJV Nossal, which is hereby incorporated by reference. In another embodiment, the adjuvant is any other adjuvant known in the art. Each possibility represents a separate embodiment of the methods and compositions as disclosed herein.

In one embodiment, disclosed herein is a method of inducing an immune response to an antigen in a subject comprising administering a recombinant Listeria strain to said subject. In one embodiment, disclosed herein is a method of inducing an anti-angiogenic immune response to an antigen in a subject comprising administering a recombinant Listeria strain to said subject. In another embodiment, said recombinant Listeria strain comprises a first and second nucleic acid molecule. In another embodiment, each said nucleic acid molecule encodes a heterologous antigen. In yet another embodiment, said first nucleic acid molecule is operably integrated into the Listeria genome as an open reading frame with an endogenous polypeptide comprising a PEST sequence.

In one embodiment, disclosed herein is a method of treating, suppressing, or inhibiting at least one cancer in a subject comprising administering a recombinant Listeria strain to said subject. In another embodiment, said recombinant Listeria strain comprises a first and second nucleic acid molecule. In another embodiment, each said nucleic acid molecule encoding a heterologous antigen. In yet another embodiment, said first nucleic acid molecule is operably integrated into the Listeria genome as an open reading frame with a nucleic acid sequence encoding an endogenous polypeptide comprising a PEST sequence. In another embodiment, at least one of said antigens is expressed by at least one cell of said cancer cells.

In one embodiment, disclosed herein is a method of delaying the onset to a cancer in a subject comprising administering a recombinant Listeria strain to said subject. In another embodiment, disclosed herein is a method of delaying the progression to a cancer in a subject comprising administering a recombinant Listeria strain to said subject. In another embodiment, disclosed herein is a method of extending the remission to a cancer in a subject comprising administering a recombinant Listeria strain to said subject. In another embodiment, disclosed herein is a method of decreasing the size of an existing tumor in a subject comprising administering a recombinant Listeria strain to said subject. In another embodiment, disclosed herein is a method of preventing the growth of an existing tumor in a subject comprising administering a recombinant Listeria strain to said subject. In another embodiment, disclosed herein is a method of preventing the growth of new or additional tumors in a subject comprising administering a recombinant Listeria strain to said subject.

In one embodiment, cancer or tumors may be prevented in specific populations known to be susceptible to a particular cancer or tumor. In one embodiment, such susceptibility may be due to environmental factors, such as smoking, which in one embodiment, may cause a population to be subject to lung cancer, while in another embodiment, such susceptibility may be due to genetic factors, for example a population with BRCA1/2 mutations may be susceptible, in one embodiment, to breast cancer, and in another embodiment, to ovarian cancer. In another embodiment, one or more mutations on chromosome 8q24, chromosome 17q12, and chromosome 17q24.3 may increase susceptibility to prostate cancer, as is known in the art. Other genetic and environmental factors contributing to cancer susceptibility are known in the art.

In one embodiment, the recombinant Listeria strain is administered to the subject at a dose of 1×10⁶-1×10⁷ CFU. In another embodiment, the recombinant Listeria strain is administered to the subject at a dose of 1×10⁷-1×10⁸ CFU. In another embodiment, the recombinant Listeria strain is administered to the subject at a dose of 1×10⁸-3.31×10¹⁰ CFU. In another embodiment, the recombinant Listeria strain is administered to the subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-15×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁷ organisms. In another embodiment, the dose is 1.5×10⁷ organisms. In another embodiment, the dose is 2×10⁸ organisms. In another embodiment, the dose is 3×10⁷ organisms. In another embodiment, the dose is 4×10⁷ organisms. In another embodiment, the dose is 5×10⁷ organisms. In another embodiment, the dose is 6×10⁷ organisms. In another embodiment, the dose is 7×10⁷ organisms. In another embodiment, the dose is 8×10⁷ organisms. In another embodiment, the dose is 10×10⁷ organisms. In another embodiment, the dose is 1.5×10⁸ organisms. In another embodiment, the dose is 2×10⁸ organisms. In another embodiment, the dose is 2.5×10⁸ organisms. In another embodiment, the dose is 3×10⁸ organisms. In another embodiment, the dose is 3.3×10⁸ organisms. In another embodiment, the dose is 4×10⁸ organisms. In another embodiment, the dose is 5×10⁸ organisms. Each dose and range of doses represents a separate embodiment of the present invention.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms. Each dose and range of doses represents a separate embodiment of the present invention.

It will be appreciated by the skilled artisan that the term “Boosting” may encompass administering an additional vaccine or immunogenic composition or recombinant Listeria strain dose to a subject. In another embodiment of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In another embodiment, a method of present invention further comprises the step of boosting the human subject with a recombinant Listeria strain as disclosed herein. In another embodiment, the recombinant Listeria strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster. In another embodiment, the methods of the present invention further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost vaccine is experimentally determined by the skilled artisan. In another embodiment, the period between a prime and a boost vaccine is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost vaccine is administered 8-10 weeks after the prime vaccine.

In another embodiment, a method of the present invention further comprises boosting the human subject with a recombinant Listeria strain disclosed herein. In another embodiment, a method of the present invention comprises the step of administering a booster dose of an immunogenic composition comprising the recombinant Listeria strain disclosed herein. In another embodiment, the booster dose is an alternate form of said immunogenic composition. In another embodiment, the methods of the present invention further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the recombinant Listeria disclosed herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Vaccine 20:1226-31 (2002); Tanghe, A., Infect. Immun. 69:3041-7 (2001). Providing antigen in different forms in the prime and the boost injections appears to maximize the immune response to the antigen. DNA vaccine priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Vaccine 20:1039-45 (2002); Billaut-Mulot, O. et al., Vaccine 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, the first or second nucleic acid molecule encodes a prostate specific antigen (PSA) and the method is for treating, inhibiting or suppressing prostate cancer. In another embodiment, the first or second nucleic acid molecule encodes PSA and the method is for treating, inhibiting or suppressing ovarian cancer. In another embodiment, the first or second nucleic acid molecule encodes PSA and the method is treating, inhibiting, or suppressing metastasis of prostate cancer, which in one embodiment, comprises metastasis to bone, and in another embodiment, comprises metastasis to other organs. In another embodiment, the first or second nucleic acid molecule encodes PSA and the method is for treating, inhibiting or suppressing metastasis of prostate cancer to bones. In yet another embodiment the method is for treating, inhibiting, or suppressing metastasis of prostate cancer to other organs. In another embodiment, the first or second nucleic acid molecule encodes PSA and the method is for treating, inhibiting or suppressing breast cancer. In another embodiment, the first or second nucleic acid molecule encodes PSA and the method is for treating, inhibiting or suppressing both ovarian and breast cancer.

The cancer that is the target of methods and compositions as disclosed herein is, in another embodiment, a melanoma. In another embodiment, the cancer is a sarcoma. In another embodiment, the cancer is a carcinoma. In another embodiment, the cancer is a mesothelioma (e.g. malignant mesothelioma). In another embodiment, the cancer is a glioma. In another embodiment, the cancer is a germ cell tumor. In another embodiment, the cancer is a choriocarcinoma.

In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma.

In another embodiment, the cancer is a non-small cell lung cancer (NSCLC). In another embodiment, the cancer is a colon cancer. In another embodiment, the cancer is a lung cancer. In another embodiment, the cancer is an ovarian cancer. In another embodiment, the cancer is a uterine cancer. In another embodiment, the cancer is a thyroid cancer. In another embodiment, the cancer is a hepatocellular carcinoma. In another embodiment, the cancer is a thyroid cancer. In another embodiment, the cancer is a liver cancer. In another embodiment, the cancer is a renal cancer. In another embodiment, the cancer is a kaposis. In another embodiment, the cancer is a sarcoma. In another embodiment, the cancer is another carcinoma or sarcoma.

In one embodiment, the compositions and methods as disclosed herein can be used to treat solid tumors related to or resulting from any of the cancers as described hereinabove. In another embodiment, the tumor is a Wilms' tumor. In another embodiment, the tumor is a desmoplastic small round cell tumor.

In another embodiment, the present invention provides a method of impeding angiogenesis of a solid tumor in a subject, comprising administering to the subject a composition comprising a recombinant Listeria encoding a heterologous antigen. In another embodiment, the antigen is HMW-MAA. In another embodiment, the antigen is fibroblast growth factor (FGF). In another embodiment, the antigen is vascular endothelial growth factor (VEGF). In another embodiment, the antigen is any other antigen known in the art to be involved in angiogenesis. In another embodiment, the methods and compositions of impeding angiogenesis of a solid tumor in a subject, as disclosed herein, comprise administering to the subject a composition comprising a recombinant Listeria encoding two heterologous antigens. In another embodiment, the methods and compositions of impeding angiogenesis of a solid tumor in a subject, as disclosed herein, comprise administering to the subject a composition comprising a mixture of two recombinant Listeria strains wherein each strain encodes a different heterologous antigens. In yet another embodiment, the methods and compositions of impeding angiogenesis of a solid tumor in a subject, as disclosed herein, comprise administering to the subject a composition comprising a recombinant Listeria strains encoding a first heterologous antigen, followed by administering to the subject a composition comprising a recombinant Listeria strains encoding a second heterologous antigen. In another embodiment, one of the two heterologous antigens is HMW-MAA. In another embodiment, the antigen is any other antigen known in the art to be involved in angiogenesis.

Methods for assessing efficacy of prostate cancer vaccines are well known in the art, and are described, for example, in Dzojic H et al (Adenovirus-mediated CD40 ligand therapy induces tumor cell apoptosis and systemic immunity in the TRAMP-C2 mouse prostate cancer model. Prostate. 2006 Jun. 1; 66(8):831-8), Naruishi K et al (Adenoviral vector-mediated RTVP-1 gene-modified tumor cell-based vaccine suppresses the development of experimental prostate cancer. Cancer Gene Ther. 2006 July; 13(7):658-63), Sehgal I et al (Cancer Cell Int. 2006 Aug. 23; 6:21), and Heinrich J E et al (Vaccination against prostate cancer using a live tissue factor deficient cell line in Lobund-Wistar rats. Cancer Immunol Immunother 2007; 56(5):725-30).

In another embodiment, the prostate cancer model used to test methods and compositions as disclosed herein is the TPSA23 (derived from TRAMP-C1 cell line stably expressing PSA) mouse model. In another embodiment, the prostate cancer model is a 178-2 BMA cell model. In another embodiment, the prostate cancer model is a PAIII adenocarcinoma cells model. In another embodiment, the prostate cancer model is a PC-3M model. In another embodiment, the prostate cancer model is any other prostate cancer model known in the art.

In another embodiment, the vaccine is tested in human subjects, and efficacy is monitored using methods well known in the art, e.g. directly measuring CD4⁺ and CD8⁺ T cell responses, or measuring disease progression, e.g. by determining the number or size of tumor metastases, or monitoring disease symptoms (cough, chest pain, weight loss, etc.). Methods for assessing the efficacy of a prostate cancer vaccine in human subjects are well known in the art, and are described, for example, in Uenaka A et al (T cell immunomonitoring and tumor responses in patients immunized with a complex of cholesterol-bearing hydrophobized pullulan (CHP) and NY-ESO-1 protein. Cancer Immun. 2007 Apr. 19; 7:9) and Thomas-Kaskel A K et al (Vaccination of advanced prostate cancer patients with PSCA and PSA peptide-loaded dendritic cells induces DTH responses that correlate with superior overall survival. Int J Cancer. 2006 Nov. 15; 119(10):2428-34).

In another embodiment, the present invention provides a method of treating benign prostate hyperplasia (BPH) in a subject. In another embodiment, the present invention provides a method of treating Prostatic Intraepithelial Neoplasia (PIN) in a subject.

In one embodiment, disclosed herein is a recombinant Listeria strain comprising a nucleic acid molecule operably integrated into the Listeria genome. In another embodiment said nucleic acid molecule encodes (a) an endogenous polypeptide comprising a PEST sequence and (b) a polypeptide comprising an antigen in an open reading frame.

In one embodiment, disclosed herein is a method of treating, suppressing, or inhibiting at least one tumor in a subject, comprising administering a recombinant Listeria strain to said subject. In another embodiment, said recombinant Listeria strain comprises a first and second nucleic acid molecule. In another embodiment, each said nucleic acid molecule encodes a heterologous antigen. In another embodiment, said first nucleic acid molecule is operably integrated into the Listeria genome as an open reading frame with a native polypeptide comprising a PEST sequence and wherein said antigen is expressed by at least one cell of said tumor.

In one embodiment, “antigen” is used herein to refer to a substance that when placed in contact with an organism, results in a detectable immune response from the organism. An antigen may be a lipid, peptide, protein, carbohydrate, nucleic acid, or combinations and variations thereof.

In one embodiment, “variant” refers to an amino acid or nucleic acid sequence (or in other embodiments, an organism or tissue) that is different from the majority of the population but is still sufficiently similar to the common mode to be considered to be one of them, for example splice variants.

In one embodiment, “isoform” refers to a version of a molecule, for example, a protein, with only slight differences compared to another isoform, or version, of the same protein. In one embodiment, isoforms may be produced from different but related genes, or in another embodiment, may arise from the same gene by alternative splicing. In another embodiment, isoforms are caused by single nucleotide polymorphisms.

In one embodiment, “fragment” refers to a protein or polypeptide that is shorter or comprises fewer amino acids than the full length protein or polypeptide. In another embodiment, fragment refers to a nucleic acid that is shorter or comprises fewer nucleotides than the full length nucleic acid. In another embodiment, the fragment is an N-terminal fragment. In another embodiment, the fragment is a C-terminal fragment. In one embodiment, the fragment is an intrasequential section of the protein, peptide, or nucleic acid. In one embodiment, the fragment is a functional fragment. In another embodiment, the fragment is an immunogenic fragment. In one embodiment, a fragment has 10-20 nucleic or amino acids, while in another embodiment, a fragment has more than 5 nucleic or amino acids, while in another embodiment, a fragment has 100-200 nucleic or amino acids, while in another embodiment, a fragment has 100-500 nucleic or amino acids, while in another embodiment, a fragment has 50-200 nucleic or amino acids, while in another embodiment, a fragment has 10-250 nucleic or amino acids.

In one embodiment, “immunogenicity” or “immunogenic” is used herein to refer to the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by, in one embodiment, a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

In one embodiment, a “homologue” refers to a nucleic acid or amino acid sequence which shares a certain percentage of sequence identity with a particular nucleic acid or amino acid sequence. In one embodiment, a sequence useful in the composition and methods as disclosed herein may be a homologue of a particular LLO sequence or N-terminal fragment thereof, ActA sequence or N-terminal fragment thereof, or PEST-like sequence described herein or known in the art. In another embodiment, a sequence useful in the composition and methods as disclosed herein may be a homologue of an antigenic polypeptide, which in one embodiment, is CA9, cHER2 or HMW-MAA or a functional fragment thereof. In one embodiment, a homolog of a polypeptide and, in one embodiment, the nucleic acid encoding such a homolog, of the present invention maintains the functional characteristics of the parent polypeptide. For example, in one embodiment, a homolog of an antigenic polypeptide of the present invention maintains the antigenic characteristic of the parent polypeptide. In another embodiment, a sequence useful in the composition and methods as disclosed herein may be a homologue of any sequence described herein. In one embodiment, a homologue shares at least 70% identity with a particular sequence. In another embodiment, a homologue shares at least 72% identity with a particular sequence. In another embodiment, a homologue shares at least 75% identity with a particular sequence. In another embodiment, a homologue shares at least 78% identity with a particular sequence. In another embodiment, a homologue shares at least 80% identity with a particular sequence. In another embodiment, a homologue shares at least 82% identity with a particular sequence. In another embodiment, a homologue shares at least 83% identity with a particular sequence. In another embodiment, a homologue shares at least 85% identity with a particular sequence. In another embodiment, a homologue shares at least 87% identity with a particular sequence. In another embodiment, a homologue shares at least 88% identity with a particular sequence. In another embodiment, a homologue shares at least 90% identity with a particular sequence. In another embodiment, a homologue shares at least 92% identity with a particular sequence. In another embodiment, a homologue shares at least 93% identity with a particular sequence. In another embodiment, a homologue shares at least 95% identity with a particular sequence. In another embodiment, a homologue shares at least 96% identity with a particular sequence. In another embodiment, a homologue shares at least 97% identity with a particular sequence. In another embodiment, a homologue shares at least 98% identity with a particular sequence. In another embodiment, a homologue shares at least 99% identity with a particular sequence. In another embodiment, a homologue shares 100% identity with a particular sequence.

In one embodiment, it is to be understood that a homolog of any of the sequences as disclosed herein and/or as described herein is considered to be a part of the invention.

In one embodiment, “functional” within the meaning of the invention, is used herein to refer to the innate ability of a protein, peptide, nucleic acid, fragment or a variant thereof to exhibit a biological activity or function. In one embodiment, such a biological function is its binding property to an interaction partner, e.g., a membrane-associated receptor, and in another embodiment, its trimerization property. In the case of functional fragments and the functional variants of the invention, these biological functions may in fact be changed, e.g., with respect to their specificity or selectivity, but with retention of the basic biological function.

In one embodiment, “treating” refers to both therapeutic treatment and prophylactic or preventative measures, wherein the object is to prevent or lessen the targeted pathologic condition or disorder as described herein. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” or “impeding” refers, inter alia, to delaying the onset of symptoms, preventing relapse to a disease, decreasing the number or frequency of relapse episodes, increasing latency between symptomatic episodes, or a combination thereof. In one embodiment, “suppressing,” or “inhibiting” refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of a particular disease or disorder, while in one embodiment, “secondary” refers to a symptom that is derived from or consequent to a primary cause. In one embodiment, the compounds for use in the present invention treat primary or secondary symptoms or secondary complications. In another embodiment, “symptoms” may be any manifestation of a disease or pathological condition.

In some embodiments, the term “comprising” refers to the inclusion of other recombinant polypeptides, amino acid sequences, or nucleic acid sequences, as well as inclusion of other polypeptides, amino acid sequences, or nucleic acid sequences, that may be known in the art, which in one embodiment may comprise antigens or Listeria polypeptides, amino acid sequences, or nucleic acid sequences. In some embodiments, the term “consisting essentially of” refers to a composition for use in the methods as disclosed herein, which has the specific recombinant polypeptide, amino acid sequence, or nucleic acid sequence, or fragment thereof. However, other polypeptides, amino acid sequences, or nucleic acid sequences may be included that are not involved directly in the utility of the recombinant polypeptide(s). In some embodiments, the term “consisting” refers to a composition for use in the methods as disclosed herein having a particular recombinant polypeptide, amino acid sequence, or nucleic acid sequence, or fragment or combination of recombinant polypeptides, amino acid sequences, or nucleic acid sequences or fragments as disclosed herein, in any form or embodiment as described herein.

In one embodiment, the compositions for use in the methods as disclosed herein are administered intravenously. In another embodiment, the vaccine is administered orally, whereas in another embodiment, the vaccine is administered parenterally (e.g., subcutaneously, intramuscularly, and the like).

Further, in another embodiment, the compositions or vaccines are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

In one embodiment, the route of administration may be parenteral. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. For intranasal administration or application by inhalation, solutions or suspensions of the compounds mixed and aerosolized or nebulized in the presence of the appropriate carrier suitable. Such an aerosol may comprise any agent described herein. In one embodiment, the compositions as set forth herein may be in a form suitable for intracranial administration, which in one embodiment, is intrathecal and intracerebroventricular administration. In one embodiment, the regimen of administration will be determined by skilled clinicians, based on factors such as exact nature of the condition being treated, the severity of the condition, the age and general physical condition of the patient, body weight, and response of the individual patient, etc.

In one embodiment, parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

In one embodiment, sustained or directed release compositions can be formulated, e.g., liposomes or those wherein the active compound is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

In one embodiment, for liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, compositions of this invention are pharmaceutically acceptable. In one embodiment, the term “pharmaceutically acceptable” refers to any formulation which is safe, and provides the appropriate delivery for the desired route of administration of an effective amount of at least one compound for use in the present invention. This term refers to the use of buffered formulations as well, wherein the pH is maintained at a particular desired value, ranging from pH 4.0 to pH 9.0, in accordance with the stability of the compounds and route of administration.

In one embodiment, a composition of or used in the methods of this invention may be administered alone or within a composition. In another embodiment, compositions of this invention admixture with conventional excipients, i.e., pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, the compositions for use of the methods and compositions as disclosed herein may be administered with a carrier/diluent. Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In one embodiment, the compositions of the methods and compositions as disclosed herein may comprise the composition of this invention and one or more additional compounds effective in preventing or treating cancer. In some embodiments, the additional compound may comprise a compound useful in chemotherapy, which in one embodiment, is Cisplatin. In another embodiment, Ifosfamide, Fluorouracilor5-FU, Irinotecan, Paclitaxel (Taxol), Docetaxel, Gemcitabine, Topotecan or a combination thereof, may be administered with a composition as disclosed herein for use in the methods as disclosed herein. In another embodiment, Amsacrine, Bleomycin, Busulfan, Capecitabine, Carboplatin, Carmustine, Chlorambucil, Cisplatin, Cladribine, Clofarabine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Gliadelimplants, Hydroxycarbamide, Idarubicin, Ifosfamide, Irinotecan, Leucovorin, Liposomaldoxorubicin, Liposomaldaunorubicin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Satraplatin, Streptozocin, Tegafur-uracil, Temozolomide, Teniposide, Thiotepa, Tioguanine, Topotecan, Treosulfan, Vinblastine, Vincristine, Vindesine, Vinorelbine, or a combination thereof, may be administered with a composition as disclosed herein for use in the methods as disclosed herein.

In another embodiment, fusion proteins as disclosed herein are prepared by a process comprising subcloning of appropriate sequences, followed by expression of the resulting nucleotide. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid. In another embodiment, a similar strategy is used to produce a protein wherein an HMW-MAA fragment is embedded within a heterologous peptide.

In one embodiment, the present invention also provides a recombinant Listeria comprising a nucleic acid molecule encoding a polypeptide comprising a heterologous antigen or fragment thereof fused to a PEST-containing sequence, wherein said nucleic acid molecule is episomal in said Listeria.

In one embodiment, disclosed herein is a recombinant Listeria capable of expressing and secreting two distinct heterologous antigens. In another embodiment, the first and second antigen are distinct. In another embodiment, said first and second antigens are concomitantly expressed. In another embodiment, said first or second antigen are expressed at the same level. In another embodiment, said first or second antigen are differentially expressed.

In another embodiment, gene or protein expression is determined by methods that are well known in the art which in another embodiment comprise real-time PCR, northern blotting, immunoblotting, etc. In another embodiment, said first or second antigen's expression is controlled by an inducible system, while in another embodiment, said first or second antigen's expression is controlled by a constitutive promoter. In another embodiment, inducible expression systems are well known in the art.

In one embodiment, disclosed herein is a method of preparing a recombinant Listeria capable of expressing and secreting two distinct heterologous antigens that target tumor cells and angiogenesis concomitantly. In another embodiment, said method of preparing said recombinant Listeria comprises the steps of genetically fusing a first antigen into the genome that is operably linked to an open reading frame encoding a first polypeptide or fragment thereof comprising a PEST sequence and transforming said recombinant Listeria with an episomal expression vector encoding a second antigen that is operably linked to an open reading frame encoding a second polypeptide or fragment thereof comprising a PEST sequence. In another embodiment, said method of preparing said recombinant Listeria comprises the steps of genetically fusing a first antigen into the genome that is operably linked to an open reading frame encoding a first polypeptide or fragment thereof comprising a PEST sequence and genetically fusing a second antigen that is operably linked to an open reading frame encoding a second polypeptide or fragment thereof comprising a PEST sequence.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.) In another embodiment, the Listeria vaccine strain as disclosed herein is transformed by electroporation.

In one embodiment, disclosed herein is a method of inducing an immune response to an antigen in a subject comprising administering a recombinant Listeria strain to said subject, wherein said recombinant Listeria strain comprises a first and second nucleic acid molecule, each said nucleic acid molecule encoding a heterologous antigenic polypeptide or fragment thereof, wherein said first nucleic acid molecule is operably integrated into the Listeria genome as an open reading frame with a nucleic acid encoding an endogenous polypeptide comprising a PEST sequence.

In another embodiment, disclosed herein is a method of inhibiting the onset of cancer, said method comprising the step of administering a recombinant Listeria composition that expresses two distinct heterologous antigens specifically expressed in said cancer.

In one embodiment, disclosed herein is a method of treating a subject having a tumor or cancer, said method comprising the step of administering a pharmaceutical composition or formulation comprising a recombinant Listeria disclosed herein that expresses two or more distinct heterologous antigens specifically expressed on said tumor.

In another embodiment, the recombinant Listeria expressing two or more heterologous antigens fused to a PEST-containing sequence (such as N-terminal LLO, N-terminal ActA, or a PEST sequence or peptide), targets two or more different tumors or cancers, or metastases in a subject having said tumors or cancers or metastases.

In another embodiment, disclosed herein is a method of ameliorating symptoms that are associated with a cancer in a subject, said method comprising the step of administering a recombinant Listeria composition that expresses two or more distinct heterologous antigens specifically expressed in said cancer.

In one embodiment, disclosed herein is a method of protecting a subject from cancer, said method comprising the step of administering a recombinant Listeria composition that expresses two distinct heterologous antigens specifically expressed in said cancer.

In another embodiment, disclosed herein is a method of delaying onset of cancer, said method comprising the step of administering a recombinant Listeria composition that expresses two or more distinct heterologous antigens specifically expressed in said cancer. In another embodiment, disclosed herein is a method of treating metastatic cancer, said method comprising the step of administering a recombinant Listeria composition that expresses two or more distinct heterologous antigens specifically expressed in said cancer. In another embodiment, disclosed herein is a method of preventing metastatic cancer or micrometastatis, said method comprising the step of administering a recombinant Listeria composition that expresses two or more distinct heterologous antigens specifically expressed in said cancer. In another embodiment, the recombinant Listeria composition is administered orally or parenterally.

In another embodiment, a pharmaceutical composition comprising the recombinant Listeria disclosed herein is administered intravenously, subcutaneously, intranasally, intramuscularly, or injected into a tumor site or into a tumor.

In another embodiment of the methods and compositions as disclosed herein, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment as disclosed herein.

The terms “polypeptide,” “peptide” and “recombinant peptide” refer, in another embodiment, to a peptide or polypeptide of any length. In another embodiment, a peptide or recombinant peptide as disclosed herein has one of the lengths enumerated above for an HMW-MAA fragment. Each possibility represents a separate embodiment of the methods and compositions as disclosed herein. In one embodiment, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are disclosed hereinunder.

In one embodiment, “antigenic polypeptide” is used herein to refer to a polypeptide, peptide or recombinant peptide as described hereinabove that is foreign to a host and leads to the mounting of an immune response when present in, or, in another embodiment, detected by, the host.

In one embodiment, the term “oligonucleotide” is interchangeable with the term “nucleic acid” and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than 500 generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

The term “nucleic acid” or “nucleic acid sequence” refers to a deoxyribonucleotide or ribonucleotide oligonucleotide in either single- or double-stranded form. The term encompasses nucleic acids, i.e., oligonucleotides, containing known analogues of natural nucleotides which have similar or improved binding properties, for the purposes desired, as the reference nucleic acid. The term also includes nucleic acids which are metabolized in a manner similar to naturally occurring nucleotides or at rates that are improved thereover for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see, e.g., Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Mulligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., in WO 97/03211; WO 96/39154; Mata (1997) Toxicol. Appi. Pharmacol. 144:189-197. Other synthetic backbones encompasses by the term include methyl-phosphonate linkages or alternating methyiphosphonate and phosphodiester linkages (Strauss-Soukup (1997) Biochemistry 36:8692-8698), and benzylphosphonate linkages (Samstag (1996) Antisense Nucleic Acid Drug Dev. 6:153-156). The term nucleic acid is used interchangeably with gene, cDNA, mRNA, oligonucleotide primer, probe and amplification product.

In one embodiment of the methods and compositions as disclosed herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions as disclosed herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

It will be appreciated by a skilled artisan that the term “operably linked” may mean that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

It will be understood by a skilled artisan that the term “open reading frame” or “ORF” may encompass a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, “endogenous” disclosed herein describes an item that has developed or originated within the reference organism or arisen from causes within the reference organism. In another embodiment, endogenous refers to native.

In another embodiment, a method of the present invention further comprises boosting the subject with a recombinant Listeria strain disclosed herein. In another embodiment, a method of the present invention comprises the step of administering a booster dose of vaccine comprising the recombinant Listeria strain disclosed herein.

In one embodiment, “fused” refers to operable linkage by covalent bonding. In one embodiment, the term includes recombinant fusion (of nucleic acid sequences or open reading frames thereof). In another embodiment, the term includes chemical conjugation.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the methods and compositions as disclosed herein.

In another embodiment, conjugation is used to introduce genetic material and/or plasmids into bacteria. Methods for conjugation are well known in the art, and are described, for example, in Nikodinovic J et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9).

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient.

It will be appreciated by a skilled artisan that the term “attenuation,” may encompass a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD.sub.50) is preferably increased above the LD.sub.50 of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present invention are therefore environmentally safe in that they are incapable of uncontrolled replication.

In one embodiment, the Listeria disclosed herein expresses a heterologous polypeptide, as described herein, in another embodiment, the Listeria as disclosed herein secretes a heterologous polypeptide, as described herein, and in another embodiment, the Listeria as disclosed herein expresses and secretes a heterologous polypeptide, as described herein. In another embodiment, the Listeria as disclosed herein comprises a heterologous polypeptide, and in another embodiment, comprises a nucleic acid that encodes a heterologous polypeptide.

In one embodiment, Listeria strains disclosed herein may be used in the preparation of vaccines or immunotherapies described herein. In one embodiment, Listeria strains as disclosed herein may be used in the preparation of peptide vaccines. Methods for preparing peptide vaccines are well known in the art and are described, for example, in EP1408048, United States Patent Application Number 20070154953, and OGASAWARA et al (Proc. Natl. Acad. Sci. USA Vol. 89, pp. 8995-8999, October 1992). In one embodiment, peptide evolution techniques are used to create an antigen with higher immunogenicity. Techniques for peptide evolution are well known in the art and are described, for example in U.S. Pat. No. 6,773,900.

In one embodiment, the vaccines of the methods and compositions disclosed herein may be administered to a host vertebrate animal, preferably a mammal, and more preferably a human, either alone or in combination with a pharmaceutically acceptable carrier. In another embodiment, the vaccine is administered in an amount effective to induce an immune response to the Listeria strain itself or to a heterologous antigen which the Listeria species has been modified to express. In another embodiment, the amount of vaccine to be administered may be routinely determined by one of skill in the art when in possession of the present disclosure. In another embodiment, a pharmaceutically acceptable carrier may include, but is not limited to, sterile distilled water, saline, phosphate buffered solutions or bicarbonate buffered solutions. In another embodiment, the pharmaceutically acceptable carrier selected and the amount of carrier to be used will depend upon several factors including the mode of administration, the strain of Listeria and the age and disease state of the vaccinee. In another embodiment, administration of the vaccine may be by an oral route, or it may be parenteral, intranasal, intramuscular, intravascular, intrarectal, intraperitoneal, or any one of a variety of well-known routes of administration. In another embodiment, the route of administration may be selected in accordance with the type of infectious agent or tumor to be treated.

In one embodiment, the present invention provides a recombinant Listeria strain comprising a nucleic acid molecule encoding a heterologous antigenic polypeptide or fragment thereof, wherein said nucleic acid molecule is operably integrated into the Listeria genome in an open reading frame with an endogenous PEST-containing gene.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. In one embodiment, the term “subject” does not exclude an individual that is healthy in all respects and does not have or show signs of disease or disorder.

In one embodiment, disclosed herein are kits comprising the pharmaceutical compositions or formulations comprising the recombinant Listeria disclosed herein.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

| SEQ | | |
|---|---|---|
| ID | | |
| NO | Type | Description |
| 1 | DNA | Recombinant Nucleic Acid Backbone |
| 2 | DNA | cHER2 Fused to an Endogenous Nucleic |
| | | Acid Comprising an Open Reading Frame |
| | | Encoding an LLO Protein |
| 3 | Protein | Fusion of cHER2 to Endogenous LLO |
| 4 | Protein | LLO |
| 5 | DNA | tLLO fused to cHer2 |
| 6 | Protein | tLLO Fused to cHer2 |
| 7 | Protein | tLLO |
| 8 | DNA | tLLO Fused to HMW-MAA-C (HMC) |
| 9 | Protein | tLLO Fused to HMW-MAA-C (HMC) |
| 10 | Protein | HMC |
| 11 | DNA | Recombinant Episomal Nucleic Acid |
| | | Sequence Encoding the Plasmid Backbone |
| | | and at Least Two Heterologous Antigens |
| 12 | Protein | PEST-Like Sequence |
| 13 | Protein | PEST-Like Sequence |
| 14 | Protein | PEST-Like Sequence |
| 15 | Protein | PEST-Like Sequence |
| 16 | Protein | PEST-Like Sequence |
| 17 | Protein | PEST-Like Sequence |
| 18 | Protein | PEST-Like Sequence |
| 19 | Protein | PEST-Like Sequence |
| 20 | Protein | PEST-Like Sequence |
| 21 | Protein | N-Terminal LLO Fragment |
| 22 | Protein | LLO Fragment |
| 23 | Protein | LLO Protein |
| 24 | Protein | LLO Protein |
| 25 | DNA | Nucleic Acid Encoding Her-2 Chimeric Protein |
| 26 | Protein | Her-2 Chimeric Protein (cHER2) |
| 27 | DNA | Nucleic Acid Sequence of Human Her2/Neu Gene |
| 28 | DNA | Nucleic Acid Encoding Human |
| | | Her2/Neu EC1 Fragment |
| 29 | DNA | Nucleic Acid Encoding EC1 Human |
| | | Her2/Neu Fragment |
| 30 | DNA | Nucleic Acid Encoding Human |
| | | Her2/Neu EC2 Fragment |
| 31 | DNA | Nucleic Acid Encoding EC2 Human |
| | | Her2/Neu Fragment |
| 32 | DNA | Nucleic Acid Encoding Human |
| | | Her2/Neu IC1 Fragment |
| 33 | DNA | Nucleic Acid Encoding Human |
| | | Her2/Neu IC1 Fragment |
| 34 | DNA | Human CA9 Gene |
| 35 | Protein | Amino Acid Sequence Encoded by Human CA9 Gene |
| 36 | DNA | Nucleic acid sequence encoding a |
| | | truncated LLO-CA9 fusion |
| 37 | Protein | tLLO Fused to CA9 |
| 38 | Protein | N-Terminal Fragment of ActA |
| 39 | DNA | Nucleic Acid Encoding ActA Fragment |
| 40 | Protein | N-Terminal Fragment of ActA |
| 41 | Protein | Truncated ActA Protein |
| 42 | Protein | Truncated ActA Fused to Hly Signal Peptide |
| 43 | DNA | Nucleic Acid Encoding ActA Fragment |
| 44 | DNA | Deleted ActA Region |
| 45 | Protein | KLK3 Protein |
| 46 | Protein | KLK3 Protein |
| 47 | Protein | KLK3 Protein |
| 48 | DNA | Nucleic Acid Encoding KLK3 Protein |
| 49 | Protein | KLK3 Protein |
| 50 | DNA | Nucleic Acid Encoding KLK3 Protein |
| 51 | Protein | KLK3 Protein |
| 52 | DNA | Nucleic Acid Encoding KLK3 Protein |
| 53 | Protein | KLK3 Protein |
| 54 | DNA | Nucleic Acid Encoding KLK3 Protein |
| 55 | Protein | KLK3 Protein |
| 56 | DNA | Nucleic Acid Encoding KLK3 Protein |
| 57 | Protein | KLK3 Protein |
| 58 | DNA | Nucleic Acid Encoding KLK3 Protein |
| 59 | Protein | KLK3 Protein |
| 60 | DNA | Nucleic Acid Encoding KLK3 Protein |
| 61 | Protein | KLK3 Protein |
| 62 | Protein | KLK3 Protein |
| 63 | Protein | KLK3 Protein |
| 64 | Protein | KLK3 Protein |
| 65 | DNA | Nucleic Acid Encoding KLK3 Protein |
| 66 | Protein | KLK3 Protein Fragment |
| 67 | Protein | E7 Protein |
| 68 | DNA | Dal Gene |
| 69 | Protein | Dal Protein |
| 70 | DNA | Dat Gene |
| 71 | Protein | Dat Protein |
| 72 | DNA | pAdv142 Plasmid |
| 73 | DNA | Adv271-actAF1 |
| 74 | DNA | Adv272-actAR1 |
| 75 | DNA | Adv273-actAF2 |
| 76 | DNA | Adv274-actAR2 |
| 77 | DNA | Adv 305 |
| 78 | DNA | Adv304 |
| 79 | Protein | Peptide Epitope for IFN-Gamma ELISpot |
| 80 | Protein | H-2 D^{b} PSA₆₅₋₇₃ |
| 81 | Protein | H-2 K^{b} OVA₂₅₇₋₂₆₄ |
| 82 | DNA | tLLO |
| 83 | DNA | PSA |
| 84 | DNA | Survivin |
| 85 | DNA | PSMA |
| 86 | DNA | PSMAΔTM |
| 87 | DNA | Linker |
| 88 | DNA | SIINFEKL-6xHis Tag |
| 89 | DNA | PSA-Survivin |
| 90 | DNA | PSA-Survivin-Tags |
| 91 | DNA | PSA-PSMA-Tags |
| 92 | DNA | tLLO-PSA-Survivin |
| 93 | DNA | tLLO-PSA-Survivin-Tags |
| 94 | DNA | tLLO-PSA-PSMA-Tags |
| 95 | DNA | pAdv134 |
| 96 | DNA | pAdv134-PSA-Survivin |
| 97 | DNA | pAdv134-PSA-Survivin-Tags |
| 98 | DNA | pAdv134-PSA-PSMA-Tags |
| 99 | DNA | Adv16 |
| 100 | DNA | Adv295 |
| 101 | DNA | Adv786 PSA Forward |
| 102 | DNA | Adv774 Survivin Forward |
| 103 | DNA | Adv775: PSMA Forward1 |
| 104 | DNA | Adv776: PSMA Forward2 |
| 105 | DNA | Adv777 PSMA Forward3 |
| 106 | DNA | Adv778 PSMA Forward4 |
| 107 | Protein | tLLO |
| 108 | Protein | PSA |
| 109 | Protein | Survivin |
| 110 | Protein | PSMA |
| 111 | Protein | PSMAΔTM |
| 112 | Protein | Linker |
| 113 | Protein | SIINFEKL-6xHis Tag |
| 114 | Protein | PSA-Survivin |
| 115 | Protein | PSA-Survivin-Tags |
| 116 | Protein | PSA-PSMA-Tags |
| 117 | Protein | tLLO-PSA-Survivin |
| 118 | Protein | tLLO-PSA-Survivin Tags |
| 119 | Protein | tLLO-PSA-PSMA Tags |
| 120 | DNA | tLLO |
| 121 | DNA | PSA |
| 122 | DNA | Survivin |
| 123 | DNA | PSGR |
| 124 | DNA | PSGRΔTM |
| 125 | DNA | Hepsin |
| 126 | DNA | HepsinΔTM |
| 127 | DNA | AKAP4 |
| 128 | DNA | Linker |
| 129 | DNA | SIINFEKL-6xHis Tag |
| 130 | DNA | PSA-PSGR-Tags |
| 131 | DNA | PSA-PSGRΔTransmembrane Domains-Tags |
| 132 | DNA | PSA-HepsinΔTM-Tags |
| 133 | DNA | PSA-AKAP4-Tags |
| 134 | DNA | PSA-Survivin-PSGRΔTM-Tags |
| 135 | DNA | PSA-Survivin-HepsinΔTM-Tags |
| 136 | DNA | PSA-PSGRΔTM-HepsinΔTM-Tags |
| 137 | DNA | PSA-Survivin-PSGRΔTM-HepsinΔTM-Tags |
| 138 | DNA | tLLO-PSA-PSGR-Tags |
| 139 | DNA | tLLO-PSA-PSGRΔTM-Tags |
| 140 | DNA | tLLO-PSA-HepsinΔTM-Tags |
| 141 | DNA | tLLO-PSA-AKAP4-Tags |
| 142 | DNA | tLLO-PSA-Survivin-PSGRΔTM-Tags |
| 143 | DNA | tLLO-PSA-Survivin-HepsinΔTM-Tags |
| 144 | DNA | tLLO-PSA-PSGRΔTM-HepsinΔTM-Tags |
| 145 | DNA | tLLO-PSA-Survivin-PSGRΔTM-HepsinΔTM-Tags |
| 146 | DNA | Adv16 |
| 147 | DNA | Adv295 |
| 148 | DNA | Adv786 PSA Forward |
| 149 | DNA | Adv774 Survivin Forward |
| 150 | DNA | PSGR Forward |
| 151 | DNA | Hepsin Forward |
| 152 | DNA | AKAP4 Forward1 |
| 153 | DNA | AKAP4 Forward2 |
| 154 | DNA | AKAP4 Forward3 |
| 155 | DNA | AKAP4 Forward4 |
| 156 | DNA | tLLO-PSA Clontech Forward |
| 157 | DNA | Tags-pAdv134 Clontech Reverse |
| 158 | Protein | tLLO |
| 159 | Protein | PSA |
| 160 | Protein | Survivin |
| 161 | Protein | PSGR |
| 162 | Protein | PSGRΔTM |
| 163 | Protein | Hepsin |
| 164 | Protein | HepsinΔTM |
| 165 | Protein | AKAP4 |
| 166 | Protein | Linker |
| 167 | Protein | SIINFEKL-6xHis Tag |
| 168 | Protein | PSA-PSGR-Tags |
| 169 | Protein | PSA-PSGRΔTM-Tags |
| 170 | Protein | PSA-HepsinΔTM-Tags |
| 171 | Protein | PSA-AKAP4-Tags |
| 172 | Protein | PSA-Survivin-PSGRΔTM-Tags |
| 173 | Protein | PSA-Survivin-HepsinΔTM-Tags |
| 174 | Protein | PSA-PSGRΔTM-HepsinΔTM-Tags |
| 175 | Protein | PSA-Survivin-PSGRΔTM-HepsinΔTM-Tags |
| 176 | Protein | tLLO-PSA-PSGR-Tags |
| 177 | Protein | tLLO-PSA-PSGRΔTM-Tags |
| 178 | Protein | tLLO-PSA-HepsinΔTM-Tags |
| 179 | Protein | tLLO-PSA-AKAP4-Tags |
| 180 | Protein | tLLO-PSA-Survivin-PSGRΔTM-Tags |
| 181 | Protein | tLLO-PSA-Survivin-HepsinΔTM-Tags |
| 182 | Protein | tLLO-PSA-PSGRΔTM-HepsinΔTM-Tags |
| 183 | Protein | tLLO-PSA-Survivin-PSGRΔTM-HepsinΔTM-Tags |
| 184 | DNA | ADV710 |
| 185 | DNA | Adv711 |
| 186 | DNA | Adv16 |
| 187 | DNA | Adv295 |
| 188 | DNA | Adv774 |
| 189 | DNA | Adv786 |
| 190 | DNA | Adv827 |
| 191 | DNA | Adv828 |
| 192 | DNA | pAdv134-MCS DNA Sequence |
| 193 | DNA | Human PSA Target DNA (Sequence ID: BC005307.1). |
| 194 | DNA | Human Survivin Target DNA |
| 195 | DNA | Human PSGR DNA (Sequence ID: CCDS7751.1). |
| 196 | DNA | Human PSGRΔTM Target DNA |
| 197 | DNA | Human Hepsin DNA (Sequence ID: CCDS32993.1). |
| 198 | DNA | Human HepsinΔTM Target DNA |
| 199 | DNA | SIINFEKL-6xHIS Epitope Tag DNA |
| 200 | DNA | PSA-Survivin-PSGRΔTM-HepsinΔTM- |
| | | SIINFEKL-6xHIS Target Insert DNA |
| 201 | DNA | pUC57kan-Insert DNA Sequence |
| 202 | DNA | pAdv2142 DNA Sequence |

### LISTING OF EMBODIMENTS

The subject matter disclosed herein includes, but is not limited to, the following embodiments:

1. A recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof, a survivin antigen or an immunogenic fragment thereof, a prostate specific G-protein coupled receptor (PSGR) antigen or an immunogenic fragment thereof, and a hepsin antigen or an immunogenic fragment thereof.

2. The recombinant Listeria strain of embodiment 1, wherein the PSGR antigen or immunogenic fragment thereof is a PSGRΔtransmembrane domain (ATM) antigen, and the hepsin antigen or immunogenic fragment thereof is a hepsinΔTM antigen.

3. The recombinant Listeria strain of embodiment 2, wherein the PSA antigen or immunogenic fragment thereof, the survivin antigen or immunogenic fragment thereof, the PSGRΔTM antigen or immunogenic fragment thereof, and the hepsinΔTM antigen or immunogenic fragment thereof are in the following order from N-terminal to C-terminal: PSA-survivin-PSGRΔTM-hepsinΔTM.

4. The recombinant Listeria strain of embodiment 3, wherein the truncated LLO (tLLO), the truncated ActA, or the PEST amino acid sequence is fused to the PSA antigen or immunogenic fragment thereof.

5. The recombinant Listeria strain embodiment 4, wherein the fusion polypeptide comprises from N-terminal to C-terminal: tLLO-PSA-survivin-PSGRΔTM-hepsinΔTM.

6. The recombinant Listeria strain of any one of embodiments 3-5, wherein the PSA or immunogenic fragment thereof is linked to the survivin or immunogenic fragment thereof by a first linker, the survivin or immunogenic fragment thereof is linked to the PSGRΔTM or immunogenic fragment thereof via a second linker, and the PSGRΔTM or immunogenic fragment thereof is linked to the hepsinΔTM or immunogenic fragment thereof via a third linker.

7. The recombinant Listeria strain of any one of embodiments 1-6, wherein the PSA antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 108.

8. The recombinant Listeria strain of any one of embodiments 1-7, wherein the survivin antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 109.

9. The recombinant Listeria strain of any one of embodiments 1-8, wherein the PSGR antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 162.

10. The recombinant Listeria strain of any one of embodiments 1-9, wherein the hepsin antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 164.

11. The recombinant Listeria strain of any one of embodiments 1-10, wherein the fusion polypeptide comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with residues 1-973 of SEQ ID NO: 175 or residues 1-1414 of SEQ ID NO: 183.

12. The recombinant Listeria strain of any one of embodiments 1-11, wherein the nucleic acid molecule is operably integrated into the Listeria genome.

13. The recombinant Listeria strain of any one of embodiments 1-11, wherein the nucleic acid molecule is in a plasmid.

14. The recombinant Listeria strain of embodiment 13, wherein the plasmid is stably maintained in the recombinant Listeria strain in the absence of antibiotic selection.

15. The recombinant Listeria strain of embodiment 13 or 14, wherein the plasmid does not confer antibiotic resistance upon the recombinant Listeria strain.

16. The recombinant Listeria strain of any one of embodiments 1-15, wherein the recombinant Listeria strain is attenuated.

17. The recombinant Listeria strain of embodiment 16, wherein the attenuated Listeria strain comprises a mutation in one or more endogenous genes.

18. The recombinant Listeria strain of embodiment 17, wherein the one or more endogenous genes comprise an actA virulence gene.

19. The recombinant Listeria strain of embodiment 17, wherein the one or more endogenous genes comprise an endogenous prfA gene.

20. The recombinant Listeria strain of embodiment 17 or 18, wherein the one or more endogenous genes comprise D-alanine racemase (Dal) and D-amino acid transferase (Dat) genes.

21. The recombinant Listeria strain of any one of embodiments 17-20, wherein the mutation comprises an inactivation, truncation, deletion, replacement or disruption of the one or more endogenous genes.

22. The recombinant Listeria strain of any one of embodiments 1-21, wherein the nucleic acid molecule comprises a second open reading frame.

23. The recombinant Listeria strain of embodiment 22, wherein the second open reading frame encodes a metabolic enzyme.

24. The recombinant Listeria strain of embodiment 23, wherein the metabolic enzyme is an alanine racemase enzyme or a D-amino acid transferase enzyme.

25. The recombinant Listeria strain of any one of embodiments 1-24, wherein the fusion polypeptide is expressed from an hly promoter, aprfA promoter, an actA promoter, or a p60 promoter, preferably an hly promoter, or wherein the nucleic acid molecule is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the sequence set forth in SEQ ID NO: 202.

26. The recombinant Listeria strain of any one of embodiments 1-25, wherein the recombinant Listeria strain is a recombinant Listeria monocytogenes strain.

27. The recombinant Listeria strain of any one of embodiments 1-26, wherein the recombinant Listeria strain has been passaged through an animal host.

28. The recombinant Listeria strain of any one of embodiments 1-27, wherein the recombinant Listeria strain is an auxotrophic Listeria strain.

29. The recombinant Listeria strain of any one of embodiments 1-28, wherein the recombinant Listeria strain is capable of escaping a phagolysosome.

30. An immunogenic composition comprising the recombinant Listeria strain of any one of embodiments 1-29.

31. The immunogenic composition of embodiment 30, wherein the immunogenic composition further comprises an adjuvant.

32. The immunogenic composition of embodiment 31, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

33. A method of inducing an immune response against a tumor or cancer in a subject, comprising administering to the subject the recombinant Listeria strain of any one of embodiments 1-29 or the immunogenic composition of any one of embodiments 30-32.

34. A method of preventing or treating a tumor or cancer in a subject, comprising administering to the subject the recombinant Listeria strain of any one of embodiments 1-29 or the immunogenic composition of any one of embodiments 30-32.

35. The method of embodiment 33 or 34, wherein the tumor or cancer is a PSA-expressing tumor or cancer, a survivin-expressing tumor or cancer, a PSGR-expressing tumor or cancer, or a hepsin-expressing tumor or cancer.

36. The method of embodiment 35, wherein the tumor or cancer is a PSA-expressing tumor or cancer, a survivin-expressing tumor or cancer, a PSGR-expressing tumor or cancer, and a hepsin-expressing tumor or cancer.

37. The method of any one of embodiments 33-36, wherein the tumor or cancer is a prostate tumor or cancer.

38. A recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof and a survivin antigen or an immunogenic fragment thereof.

39. The recombinant Listeria strain of embodiment 38, wherein the PSA antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 108.

40. The recombinant Listeria strain of embodiment 38 or 39, wherein the survivin antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 109.

41. The recombinant Listeria strain of any one of embodiments 38-40, wherein the fusion polypeptide comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with residues 1-382 of SEQ ID NO: 115 or residues 1-825 of SEQ ID NO: 117.

42. A recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) antigen or an immunogenic fragment thereof and a prostate-specific membrane antigen (PSMA) antigen or an immunogenic fragment thereof.

43. The recombinant Listeria strain of embodiment 42, wherein the PSA antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 108.

44. The recombinant Listeria strain of embodiment 42 or 43, wherein the PSMA antigen or immunogenic fragment thereof comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with SEQ ID NO: 111.

45. The recombinant Listeria strain of any one of embodiments 42-44, wherein the fusion polypeptide comprises, consists essentially of, or consists of an amino acid sequence having at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity with residues 1-967 of SEQ ID NO: 116 or residues 1-1410 of SEQ ID NO: 119.

46. The recombinant Listeria strain of any one of embodiments 38-45, wherein the nucleic acid molecule is operably integrated into the Listeria genome.

47. The recombinant Listeria strain of any one of embodiments 38-45, wherein the nucleic acid molecule is in a plasmid.

48. The recombinant Listeria strain of embodiment 47, wherein the plasmid is stably maintained in the recombinant Listeria strain in the absence of antibiotic selection.

49. The recombinant Listeria strain of embodiment 47 or 48, wherein the plasmid does not confer antibiotic resistance upon the recombinant Listeria strain.

50. The recombinant Listeria strain of any one of embodiments 38-49, wherein the recombinant Listeria strain is attenuated.

51. The recombinant Listeria strain of embodiment 50, wherein the attenuated Listeria strain comprises a mutation in one or more endogenous genes.

52. The recombinant Listeria strain of embodiment 51, wherein the one or more endogenous genes comprise an actA virulence gene.

53. The recombinant Listeria strain of embodiment 51, wherein the one or more endogenous genes comprise an endogenous prfA gene.

54. The recombinant Listeria strain of embodiment 51 or 52, wherein the one or more endogenous genes comprise D-alanine racemase (Dal) and D-amino acid transferase (Dat) genes.

55. The recombinant Listeria strain of any one of embodiments 51-54, wherein the mutation comprises an inactivation, truncation, deletion, replacement or disruption of the one or more endogenous genes.

56. The recombinant Listeria strain of any one of embodiments 38-55, wherein the nucleic acid molecule comprises a second open reading frame.

57. The recombinant Listeria strain of embodiment 56, wherein the second open reading frame encodes a metabolic enzyme.

58. The recombinant Listeria strain of embodiment 57, wherein the metabolic enzyme is an alanine racemase enzyme or a D-amino acid transferase enzyme.

59. The recombinant Listeria strain of any one of embodiments 38-58, wherein the fusion polypeptide is expressed from an hly promoter, aprfA promoter, an actA promoter, or a p60 promoter.

60. The recombinant Listeria strain of any one of embodiments 38-59, wherein the recombinant Listeria strain is a recombinant Listeria monocytogenes strain.

61. The recombinant Listeria strain of any one of embodiments 38-60, wherein the recombinant Listeria strain has been passaged through an animal host.

62. The recombinant Listeria strain of any one of embodiments 38-61, wherein the recombinant Listeria strain is an auxotrophic Listeria strain.

63. The recombinant Listeria strain of any one of embodiments 38-62, wherein the recombinant Listeria strain is capable of escaping a phagolysosome.

64. An immunogenic composition comprising the recombinant Listeria strain of any one of embodiments 38-63.

65. The immunogenic composition of embodiment 64, wherein the immunogenic composition further comprises an adjuvant.

66. The immunogenic composition of embodiment 65, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

67. A method of inducing an immune response against a tumor or cancer in a subject, comprising administering to the subject the recombinant Listeria strain of any one of embodiments 38-63 or the immunogenic composition of any one of embodiments 64-66.

68. A method of preventing or treating a tumor or cancer in a subject, comprising administering to the subject the recombinant Listeria strain of any one of embodiments 38-63 or the immunogenic composition of any one of embodiments 64-66.

69. The method of embodiment 67 or 68, wherein the tumor or cancer is a PSA-expressing tumor or cancer, a survivin-expressing tumor or cancer, a PSGR-expressing tumor or cancer, or a hepsin-expressing tumor or cancer.

70. The method of embodiment 69, wherein the tumor or cancer is a PSA-expressing tumor or cancer, a survivin-expressing tumor or cancer, a PSGR-expressing tumor or cancer, and a hepsin-expressing tumor or cancer.

71. The method of any one of embodiments 67-70, wherein the tumor or cancer is a prostate tumor or cancer.

72. A method of eliciting an anti-tumor or anti-cancer immune response in a subject, comprising administering to the subject an effective amount of an immunogenic composition comprising a recombinant Listeria strain comprising a recombinant nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a first heterologous antigen or an immunogenic fragment thereof, and wherein the recombinant Listeria strain expresses the fusion polypeptide, thereby eliciting an anti-tumor or anti-cancer immune response in the subject.

73. The method of embodiment 72, wherein the recombinant nucleic acid molecule in the recombinant Listeria strain comprises a second open reading frame.

74. The method of embodiment 73, wherein the second open reading frame encodes a second fusion polypeptide comprising a truncated listeriolysin O (LLO) protein, a truncated ActA protein, or a PEST amino acid sequence fused to a second heterologous antigen or an immunogenic fragment thereof, and wherein the Listeria expresses the second fusion polypeptide.

75. The method of any one of embodiments 72-74, wherein the first heterologous antigen or the second heterologous antigen is selected from prostate stem cell antigen (PSCA), prostate-specific antigen (PSA; KLK3), prostate-specific membrane antigen (PSMA), PAP, Nkx3.1, Ssx2, A Kinase Anchor Protein 4 (AKAP4), HPV E7, Hepsin (HPN/TMPRSS1), Prostate-specific G-protein-coupled receptor (PSGR/OR51E2), T-cell receptor γ-chain Alternate Reading-Frame Protein (TARP), survivin (Birc5), Mammalian Enabled Homolog (ENAH; hMENA), POTE paralogs, O-GlcNAc Transferase (OGT), KLK7, Secernin-1 (SCRN1), Fibroblast Activation Protein (FAP), Matrix Metallopeptidase 7 (MMP7), Milk Fat Globule-EGF Factor 8 Protein (MFGE8), Wilms Tumor 1 (WT1), Interferon-Stimulated Gene 15 Ubiquitin-Like Modifier (ISG15; G1P2), Acrosin Binding Protein (ACRBP; OY-TES-1), and Kallikrein-Related Peptidase 4 (KLK4/prostase).

76. The method of embodiment 75, wherein the HPV E7 comprises, consists essentially of, or consists of SEQ ID NO: 67.

77. The method of any one of embodiments 72-76, wherein the recombinant nucleic acid molecule is in a plasmid in the recombinant Listeria strain.

78. The method of embodiment 77, wherein the plasmid is an integrative plasmid.

79. The method of embodiment 77, wherein the plasmid is an episomal plasmid.

80. The method of embodiment 79, wherein the plasmid is stably maintained in the recombinant Listeria strain in the absence of antibiotic selection.

81. The method of any one of embodiments 77-80, wherein the plasmid does not confer antibiotic resistance upon the recombinant Listeria strain.

82. The method of any one of embodiments 72-81, wherein the recombinant Listeria strain is attenuated.

83. The method of embodiment 82, wherein the attenuated recombinant Listeria strain comprises a mutation in one or more endogenous genes.

84. The method of embodiment 83, wherein the one or more endogenous genes comprise an actA virulence gene.

85. The method of embodiment 83, wherein the one or more endogenous genes comprise an endogenous prfA gene.

86. The method of embodiment 83 or 84, wherein the one or more endogenous genes comprise D-alanine racemase (Dal) and D-amino acid transferase (Dat) genes.

87. The method of any one of embodiments 83-86, wherein the mutation comprises an inactivation, truncation, deletion, replacement, or disruption of the one or more endogenous genes.

88. The method of any one of embodiments 73 and 77-87, wherein the second open reading frame encodes a metabolic enzyme.

89. The method of embodiment 88, wherein the metabolic enzyme encoded by the second open reading frame is an alanine racemase enzyme or a D-amino acid transferase enzyme.

90. The method of any one of embodiments 72-87, wherein the recombinant nucleic acid molecule further comprises a third open reading frame.

91. The method of embodiment 90, wherein the third open reading frame encodes a metabolic enzyme.

92. The method of embodiment 91, wherein the metabolic enzyme encoded by the third open reading frame is an alanine racemase enzyme or a D-amino acid transferase enzyme.

93. The method of any one of embodiments 72-92, wherein the first fusion polypeptide is expressed from an hly promoter, aprfA promoter, an actA promoter, or ap60 promoter.

94. The method of any one of embodiments 72-93, wherein the recombinant Listeria strain is a recombinant Listeria monocytogenes strain.

95. The method of any one of embodiments 72-94, wherein the recombinant Listeria strain has been passaged through an animal host.

96. The method of any one of embodiments 72-95, wherein the administering induces epitope spreading to additional tumor associated antigens.

97. The method of any one of embodiments 72-96, wherein the tumor or the cancer comprises a breast tumor or cancer, a gastric tumor or cancer, an ovarian tumor or cancer, a brain tumor or cancer, a cervical tumor or cancer, an endometrial tumor or cancer, a glioblastoma, a lung cancer, a bladder tumor or cancer, a pancreatic tumor or cancer, melanoma, a colorectal tumor or cancer, or any combination thereof.

98. The method of any one of embodiments 72-97, wherein the tumor or the cancer is a metastasis.

99. The method of any one of embodiments 72-98, wherein the method allows preventing the recurrence of a tumor or a cancer in the subject, or inhibiting metastasis of a tumor or a cancer in the subject, or any combination thereof.

100. The method of any one of embodiments 72-99 wherein the method allows treating a subject having a tumor or suffering from a cancer.

101. The method according to any of embodiments 72-100, wherein the immunogenic composition further comprises an adjuvant.

102. The method of embodiment 101, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

103. The method of any one of embodiments 100-102, wherein the treating reduces or halts the growth of the tumor or the cancer.

104. The method of any one of embodiments 100-102, wherein the treating reduces or halts metastasis of the tumor or the cancer.

105. The method of any one of embodiments 100-102, wherein the treating elicits and maintains an anti-tumor or anti-cancer immune response in the subject.

106. The method of any one of embodiments 100-102, wherein the treating extends the survival time of the subject.

107. An immunogenic composition comprising a recombinant Listeria strain comprising a recombinant nucleic acid molecule, the nucleic acid molecule comprising a first open reading frame encoding a first fusion polypeptide, wherein the first fusion polypeptide comprises a truncated listeriolysin O (LLO) protein, a truncated ActA protein, or a PEST amino acid sequence fused to an endoglin sequence or an immunogenic fragment thereof, wherein the Listeria strain comprises mutations in endogenous D-alanine racemase (dal), D-amino acid transferase (dat), and ActA (actA) genes.

108. The immunogenic composition of embodiment 107, wherein the recombinant nucleic acid molecule in the Listeria comprises a second open reading frame.

109. The immunogenic composition of embodiment 108, wherein the second open reading frame encodes a second fusion polypeptide, wherein the second fusion polypeptide comprises a truncated listeriolysin O (LLO) protein, a truncated ActA protein, or a PEST amino acid sequence fused to a heterologous antigen or an immunogenic fragment thereof, and wherein the recombinant Listeria strain expresses the second fusion polypeptide.

110. The immunogenic composition of embodiment 109, wherein the heterologous antigen is selected from prostate stem cell antigen (PSCA), prostate-specific antigen (PSA; KLK3), prostate-specific membrane antigen (PSMA), PAP, Nkx3.1, Ssx2, A Kinase Anchor Protein 4 (AKAP4), HPV E7, Hepsin (HPN/TMPRSS1), Prostate-specific G-protein-coupled receptor (PSGR/OR51E2), T-cell receptor γ-chain Alternate Reading-Frame Protein (TARP), survivin (Birc5), Mammalian Enabled Homolog (ENAH; hMENA), POTE paralogs, O-GlcNAc Transferase (OGT), KLK7, Secernin-1 (SCRN1), Fibroblast Activation Protein (FAP), Matrix Metallopeptidase 7 (MMP7), Milk Fat Globule-EGF Factor 8 Protein (MFGE8), Wilms Tumor 1 (WT1), Interferon-Stimulated Gene 15 Ubiquitin-Like Modifier (ISG15; G1P2), Acrosin Binding Protein (ACRBP; OY-TES-1), and Kallikrein-Related Peptidase 4 (KLK4/prostase).

111. The immunogenic composition of embodiment 110, wherein the HPV E7 comprises, consists essentially of, or consists of SEQ ID NO: 67.

112. The immunogenic composition of any one of embodiments 107-111, wherein the recombinant nucleic acid molecule is in a plasmid in the recombinant Listeria strain.

113. The immunogenic composition of embodiment 112, wherein the plasmid is an integrative plasmid.

114. The immunogenic composition of embodiment 113, wherein the plasmid is an episomal plasmid.

115. The immunogenic composition of any one of embodiments 112-114, wherein the plasmid is stably maintained in the recombinant Listeria strain in the absence of antibiotic selection.

116. The immunogenic composition of any one of embodiments 112-115, wherein the plasmid does not confer antibiotic resistance upon the recombinant Listeria strain.

117. The immunogenic composition of any one of embodiments 107-116, wherein the recombinant Listeria strain is attenuated.

118. The immunogenic composition of embodiment 117, wherein the attenuated Listeria strain comprises a mutation in one or more endogenous genes.

119. The immunogenic composition of embodiment 118, wherein the one or more endogenous genes comprise an actA virulence gene.

120. The immunogenic composition of embodiment 118, wherein the one or more endogenous genes comprise an endogenous prfA gene.

121. The immunogenic composition of embodiment 118 or 119, wherein the one or more endogenous gene comprise D-alanine racemase (Dal) and D-amino acid transferase (Dat) genes.

122. The immunogenic composition of any one of embodiments 118-121, wherein the mutation comprises an inactivation, truncation, deletion, replacement or disruption of the one or more endogenous genes.

123. The immunogenic composition of any one of embodiments 108 and 112-121, wherein the second open reading frame encodes a metabolic enzyme.

124. The immunogenic composition of embodiment 123, wherein the metabolic enzyme is an alanine racemase enzyme or a D-amino acid transferase enzyme.

125. The immunogenic composition of any one of embodiments 107-122, wherein the recombinant nucleic acid molecule further comprises a third open reading frame.

126. The immunogenic composition of embodiment 125, wherein the third open reading frame encodes a metabolic enzyme.

127. The immunogenic composition of embodiment 126, wherein the metabolic enzyme is an alanine racemase enzyme or a D-amino acid transferase enzyme.

128. The immunogenic composition of any one of embodiments 107-127, wherein the first fusion polypeptide is expressed from an hly promoter, aprfA promoter, an actA promoter, or a p60 promoter.

129. The immunogenic composition of any one of embodiments 107-128, wherein the recombinant Listeria strain is a recombinant Listeria monocytogenes strain.

130. The immunogenic composition of any one of embodiments 107-129, wherein the recombinant Listeria strain has been passaged through an animal host.

131. The immunogenic composition according to any of embodiments 107-130, wherein the composition further comprises an adjuvant.

132. The immunogenic composition of embodiment 131, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

133. A recombinant Listeria strain comprising a nucleic acid molecule comprising a first open reading frame encoding a first fusion polypeptide, the first fusion polypeptide comprising a prostate specific (PSA) antigen or an immunogenic fragment thereof fused to a truncated listeriolysin O (LLO), a truncated ActA, or a PEST amino acid sequence, and wherein the nucleic acid molecule further comprises a second open reading frame encoding a second fusion polypeptide, the second fusion polypeptide comprising a survivin antigen or an immunogenic fragment thereof fused to a truncated listeriolysin O (LLO), a truncated ActA, or a PEST amino acid sequence.

134. A recombinant Listeria strain comprising a nucleic acid molecule comprising a first open reading frame encoding a first fusion polypeptide, the first fusion polypeptide comprising a prostate specific (PSA) antigen or a immunogenic fragment thereof fused to a truncated listeriolysin O (LLO), a truncated ActA, or a PEST amino acid sequence, and wherein the nucleic acid molecule further comprises a second open reading frame encoding a second fusion polypeptide, the second fusion polypeptide comprising a prostate-specific membrane antigen (PSMA) or an immunogenic fragment thereof fused to a truncated listeriolysin O (LLO), a truncated ActA, or a PEST amino acid sequence.

135. The recombinant Listeria strain of embodiment 133 or 134, wherein the nucleic acid molecule is operably integrated into the Listeria genome.

136. The recombinant Listeria strain of embodiment 133 or 134, wherein the nucleic acid molecule is in a plasmid.

137. The recombinant Listeria strain of embodiment 136, wherein the plasmid is stably maintained in the recombinant Listeria strain in the absence of antibiotic selection.

138. The recombinant Listeria strain of embodiment 136 or 137, wherein the plasmid does not confer antibiotic resistance upon the recombinant Listeria.

139. The recombinant Listeria strain of any one of embodiments 133-138, wherein the recombinant Listeria strain is attenuated.

140. The recombinant Listeria strain of embodiment 139, wherein the attenuated Listeria strain comprises a mutation in one or more endogenous genes.

141. The recombinant Listeria strain of embodiment 140, wherein the one or more endogenous genes comprise an actA virulence gene.

142. The recombinant Listeria strain of embodiment 140, wherein the one or more endogenous genes comprise an endogenous prfA gene.

143. The recombinant Listeria strain of embodiment 140 or 141, wherein the one or more endogenous genes comprise D-alanine racemase (Dal) and D-amino acid transferase (Dat) genes.

144. The recombinant Listeria strain of any one of embodiments 140-143, wherein the mutation comprises an inactivation, truncation, deletion, replacement or disruption of the one or more endogenous genes.

145. The recombinant Listeria strain of any one of embodiments 133-144, wherein the nucleic acid further comprises a third open reading frame.

146. The recombinant Listeria strain of embodiment 145, wherein the third open reading frame encodes a metabolic enzyme.

147. The recombinant Listeria strain of embodiment 146, wherein the metabolic enzyme is an alanine racemase enzyme or a D-amino acid transferase enzyme.

148. The recombinant Listeria strain of any one of embodiments 133-147, wherein the first fusion polypeptide and/or the second fusion polypeptide is expressed from an hly promoter, aprfA promoter, an actA promoter, or ap60 promoter.

149. The recombinant Listeria strain of any one of embodiments 133-148, wherein the recombinant Listeria strain is a recombinant Listeria monocytogenes strain.

150. The recombinant Listeria strain of any one of embodiments 133-149, wherein the recombinant Listeria strain has been passaged through an animal host.

151. The recombinant Listeria strain of any one of embodiments 133-150, wherein the recombinant Listeria strain is an auxotrophic Listeria strain.

152. The recombinant Listeria strain of any one of embodiments 133-151, wherein the recombinant Listeria strain is capable of escaping a phagolysosome.

153. An immunogenic composition comprising the recombinant Listeria strain of any one of embodiments 133-152.

154. The immunogenic composition of embodiment 153, wherein the immunogenic composition further comprises an adjuvant.

155. The immunogenic composition of embodiment 154, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

156. A method of inducing an immune response against a tumor or cancer in a subject, the method comprising administering to the subject the recombinant Listeria strain of any one of embodiments 133-152 or the immunogenic composition of any one of embodiments 153-155.

157. The method of embodiment 156, wherein the tumor or cancer is a PSA-expressing and/or a survivin-expressing tumor or cancer.

158. The method of embodiment 156, wherein the tumor or cancer is a PSA-expressing and/or a PSMA-expressing tumor or cancer.

159. A method of preventing or treating a tumor or cancer in a subject, the method comprising the steps of administering to the subject the recombinant Listeria strain of any one of embodiments 133-152 or the immunogenic composition of any one of embodiments 153-155.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

The following examples are presented in order to more fully illustrate the preferred embodiments of the invention. They should in no way be construed, however, as limiting the broad scope of the invention.

### EXAMPLES

A recombinant Lm that secretes PSA fused to tLLO (Lm-LLO-PSA) was developed. This strain elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector. We recently developed a new strain for the PSA vaccine based on the pADV142 plasmid, which has no antibiotic resistance markers, and referred as LmddA-142 (Table 1). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains

| Plasmids | Features |
|---|---|
| pGG55 | pAM401/pGB354 shuttle plasmid with gram (−) and gram (+) cm |
| | resistance, LLO-E7 expression cassette and a copy of Lm prfA gene |
| pTV3 | Derived from pGG55 by deleting cm genes and inserting |
| | the Lm dal gene |
| pADV119 | Derived from pTV3 by deleting the prfA gene |
| pADV134 | Derived from pADV119 by replacing the Lm dal gene |
| | by the Bacillus dal gene |
| pADV142 | Derived from pADV134 by replacing HPV16 e7 with klk3 |
| pADV168 | Derived from pADV134 by replacing HPV16 e7 with |
| | hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly gene in |
| | the chromosome |
| LmddA-143 | 10403S dal⁽⁻⁾ dat⁽⁻⁾ actA⁽⁻⁾ with klk3 fused to the hly gene in the |
| | chromosome |
| LmddA-168 | 10403S dal⁽⁻⁾ dat⁽⁻⁾ actA⁽⁻⁾ pADV168 |
| Lmdd-143/134 | Lmdd-143 pADV134 |
| LmddA-143/134 | LmddA-143 pADV134 |
| Lmdd-143/168 | Lmdd-143 pADV168 |
| LmddA-143/168 | LmddA-143 pADV168 |

The sequence of the plasmid pAdv142 (6523 bp) was the sequence set forth in SEQ ID NO: 72. This plasmid was sequenced at Genewiz facility from the E. coli strain on 2-20-08.

### Example 1: Construction of Attenuated Listeria Strain-LmddAactA and Insertion of the Human Klk3 Gene in Frame to the Hly Gene in the Lmdd and Lmdda Strains

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat AactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligos Adv 271/272) and downstream (625 bp-oligos Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in ΔactA/pKSV7 (pAdv120).

TABLE 2
Sequence of primers that were used for the amplification of DNA
sequences upstream and downstream of actA.

| | | SEQ ID |
|---|---|---|
| Primer | Sequence | NO: |
| Adv271-actAF1 | cg GAATTCGGATCCgcgccaaatcattggttgattg | 73 |
| Adv272-actAR1 | gcgaGTCGACgtcggggttaatcgtaatgcaattggc | 74 |
| Adv273-actAF2 | gcgaGTCGACccatacgacgttaattcttgcaatg | 75 |
| Adv274-actAR2 | gataCTGCAGGGATCCttcccttctcggtaatcagtcac | 76 |

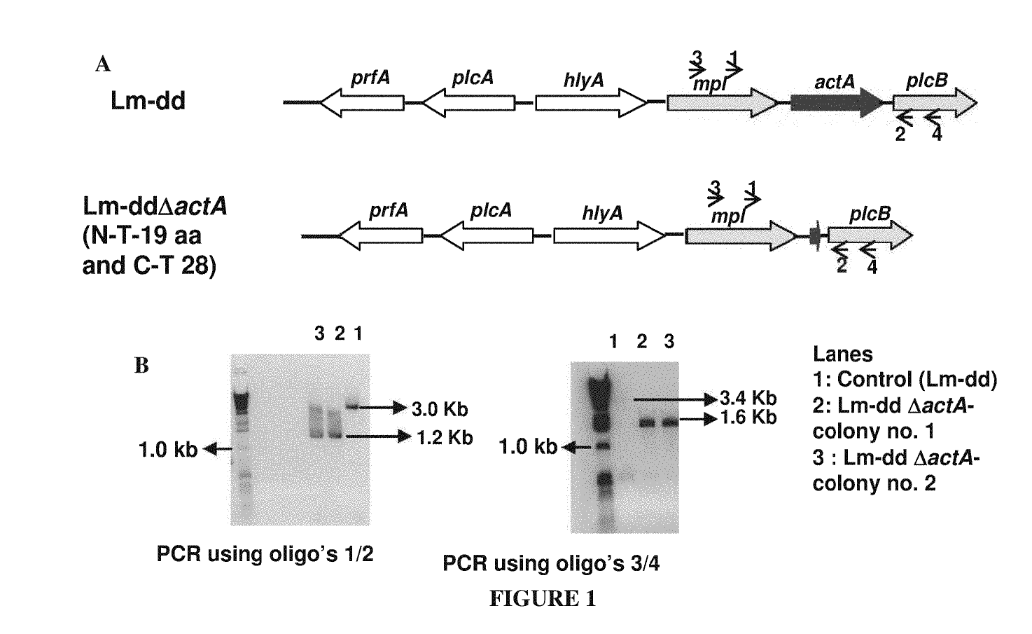

The deletion of the gene from its chromosomal location was verified using primers that bind externally to the actA deletion region, which are shown in FIG. 1 as primer 3 (Adv 305-tgggatggccaagaaattc, SEQ ID NO: 77) and primer 4 (Adv304-ctaccatgtcttccgttgcttg; SEQ ID NO: 78). The PCR analysis was performed on the chromosomal DNA isolated from Lmdd and LmddAactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddAactA was 1.2 Kb and 1.6 Kb. Thus, PCR analysis in FIG. 1 confirms that the 1.8 kb region of actA was deleted in the LmddAactA strain. DNA sequencing was also performed on PCR products to confirm the deletion of actA containing region in the strain, LmddAactA.

### Example 2: Construction of the Antibiotic-Independent Episomal Expression System for Antigen Delivery by Lm Vectors

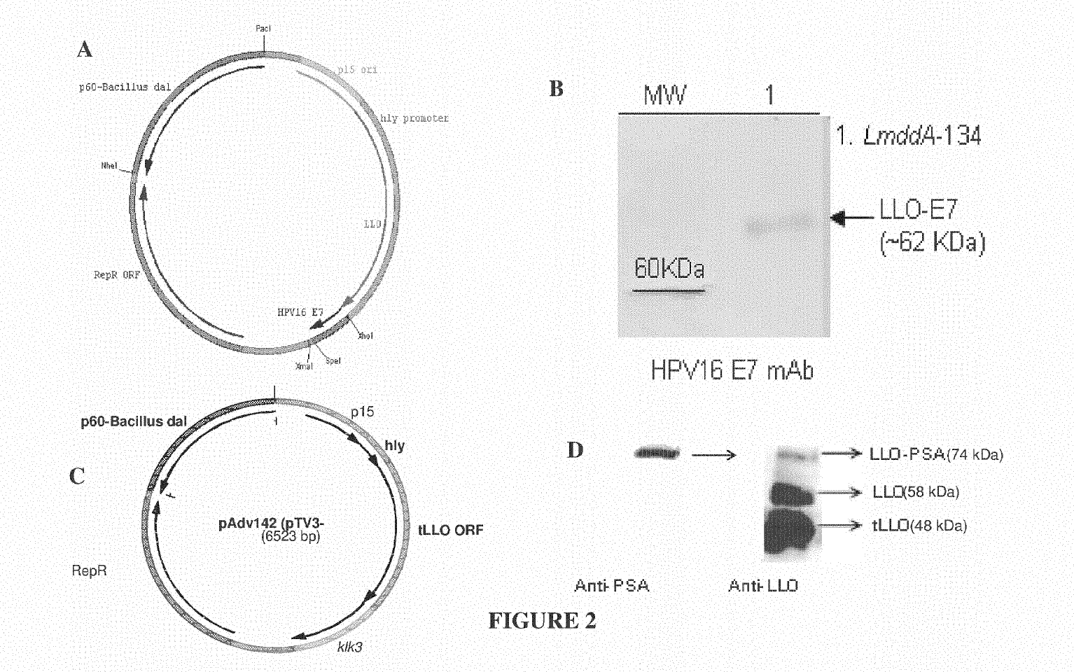

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3: In Vitro and In Vivo Stability of the Strain LmddA-LLO-PSA

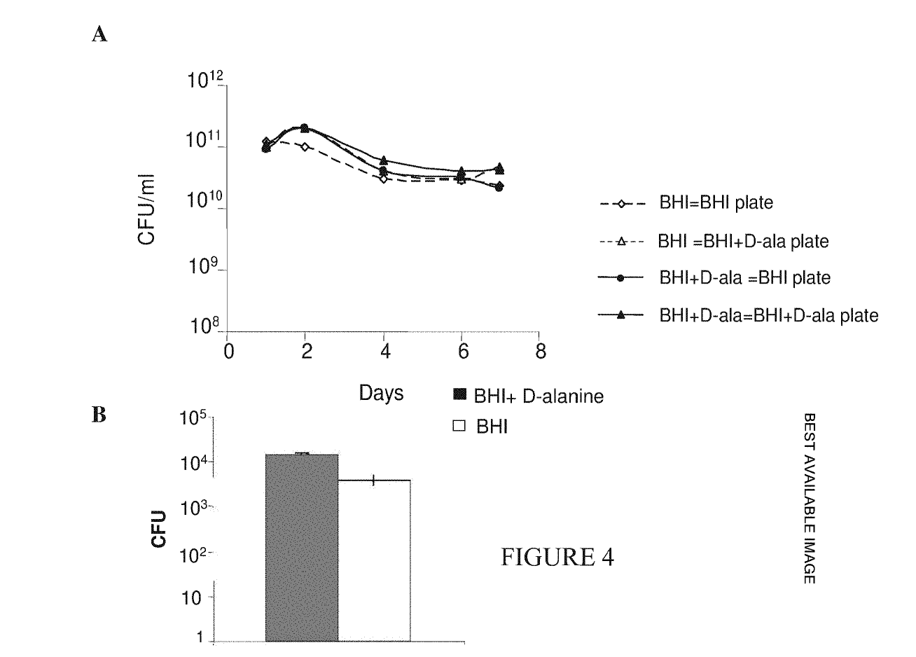

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 4A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 gg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 4B).

### Example 4: In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

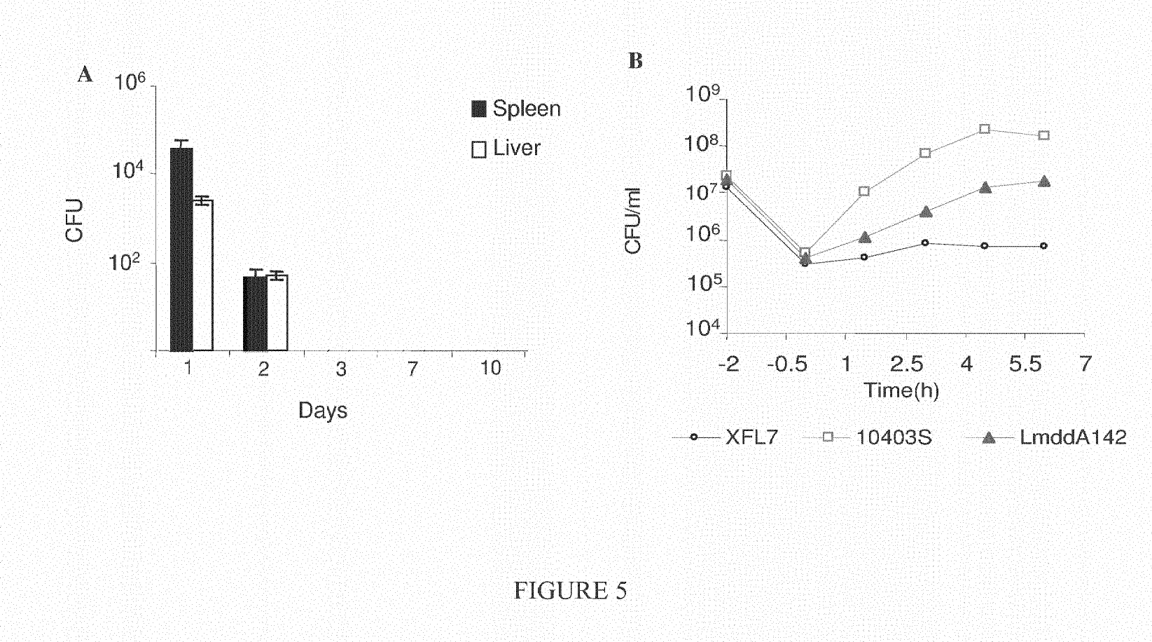

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 5A).

To determine if the attenuation of LmddA-LLO-PSA attenuated the ability of the strain LmddA-LLO-PSA to infect macrophages and grow intracellularly, we performed a cell infection assay. Mouse macrophage-like cell line such as J774A.1 were infected in vitro with Listeria constructs and intracellular growth was quantified. The positive control strain, wild type Listeria strain 10403S grows intracellularly, and the negative control XFL7, aprfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 5B). The results indicate that LmddA-LLO-PSA has the ability to infect macrophages and grow intracytoplasmically.

### Example 5: Immunogenicity of the Strain-LmddA-LLO-PSA in C57BL/6 Mice

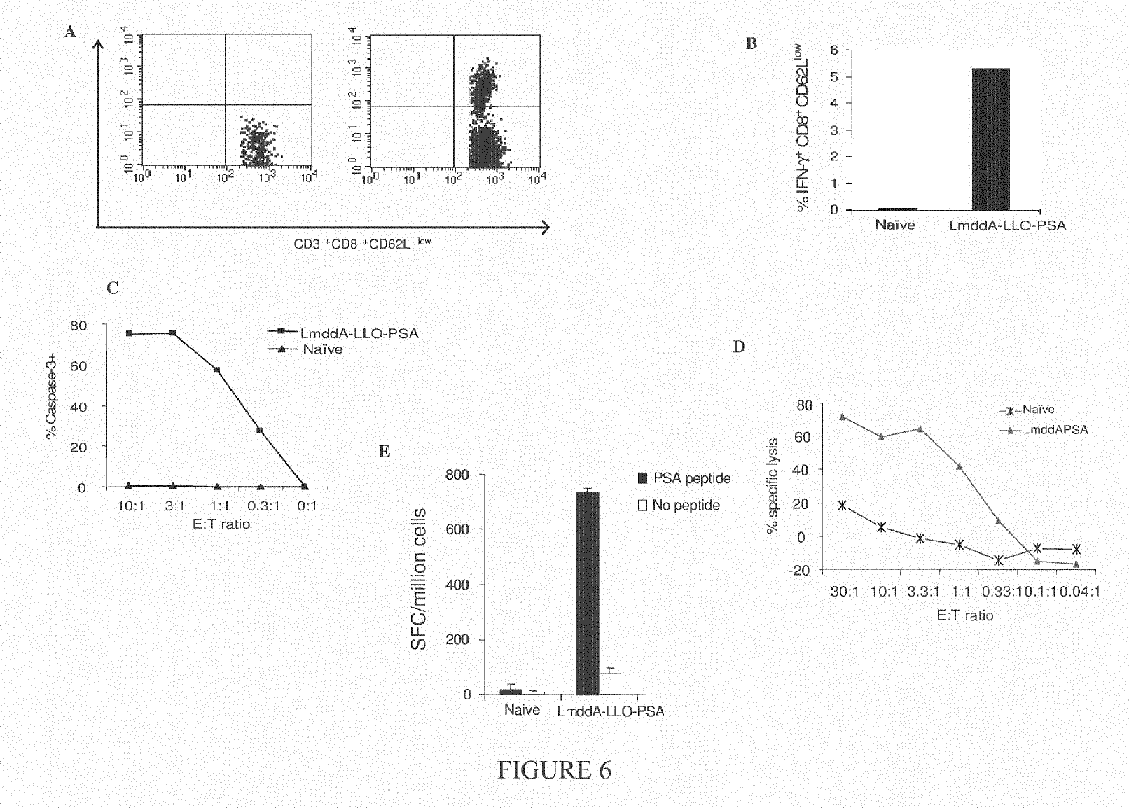

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 6A).

The functional ability of the PSA-specific T cells to secrete IFN-γ after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naïve mice (FIG. 6B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 6C) and Europium release (FIG. 6D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, we observed there was a 20-fold increase in the number of spots for IFN-γ in splenocytes from mice immunized with LmddA-LLO-PSA stimulated with specific peptide when compared to the splenocytes of the naïve mice (FIG. 6E).

### Example 6: Immunization with the LmddA-142 Strains Induces Regression of a Tumor Expressing PSA and Infiltration of the Tumor by PSA-Specific CTLs

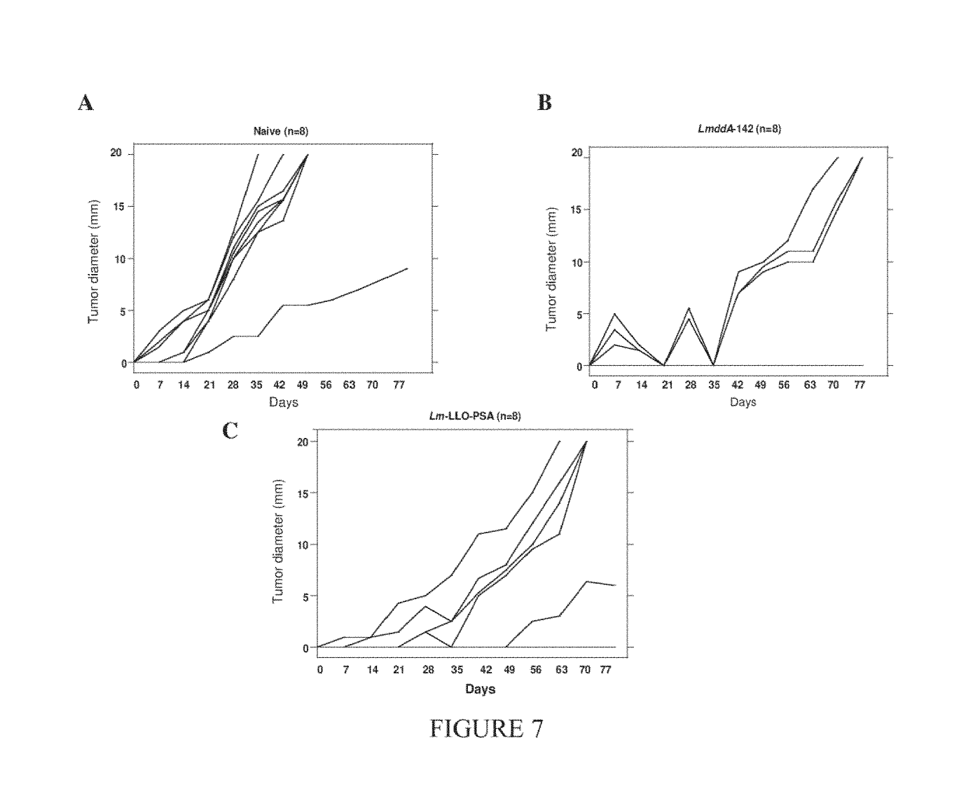

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 7A). The mice immunized with LmddA-142 were all tumor-free until day 35 and gradually 3 out of 8 mice developed tumors, which grew at a much slower rate as compared to the naïve mice (FIG. 7B). Five out of eight mice remained tumor free through day 70. As expected, Lm-LLO-PSA-vaccinated mice had fewer tumors than naïve controls and tumors developed more slowly than in controls (FIG. 7C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C1 tumors that were engineered to express PSA in more than 60% of the experimental animals (FIG. 7B), compared to none in the untreated group (FIG. 7A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naïve or control (Lm-LLO-E7) Listeria, or with LmddA-LLO-PSA. Tumors were excised on day 21 and were analyzed for the population of CD8⁺CD62L^{low} PSA^{tetramer+} and CD4⁺CD25⁺FoxP3⁺ regulatory T cells infiltrating in the tumors.

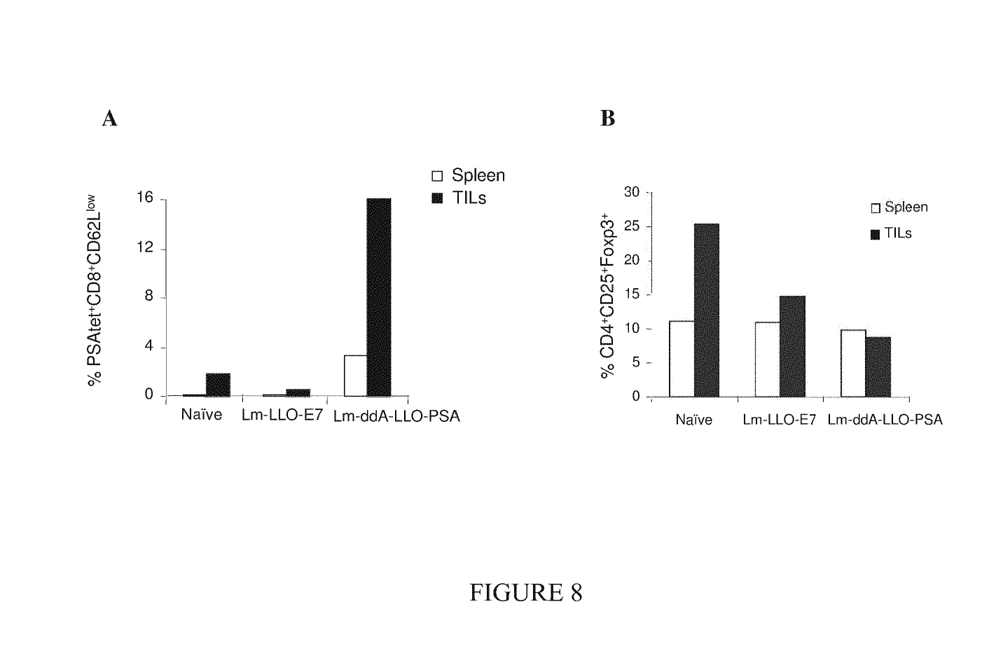

A very low number of CD8⁺CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8⁺CD62L^{low} PSA^{tetramer+} cells in spleen was 7.5 fold less than in tumor (FIG. 8A).

In addition, the presence of CD4⁺/CD25⁺/Foxp3⁺ T regulatory cells (regs) in the tumors of untreated mice and Listeria immunized mice was determined. Interestingly, immunization with Listeria resulted in a considerable decrease in the number of CD4⁺CD25⁺FoxP3⁺ T-regs in tumor but not in spleen (FIG. 8B). However, the construct LmddA-LLO-PSA had a stronger impact in decreasing the frequency of CD4⁺CD25⁺FoxP3⁺ T-regs in tumors when compared to the naïve and Lm-LLO-E7 immunized group (FIG. 7B).

Thus, the LmddA-142 vaccine can induce PSA-specific CD8⁺ T cells that are able to infiltrate the tumor site (FIG. 8A). Interestingly, Immunization with LmddA-142 was associated with a decreased number of regulatory T cells in the tumor (FIG. 7B), probably creating a more favorable environment for an efficient anti-tumor CTL activity.

### Example 7: Lmdd-143 and LmddA-143 Secretes a Functional LLO Despite the PSA Fusion

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie. 1992; 74 (7-8) p 705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 9A). The insertion of klk3 in frame with hly into the chromosome was verified by PCR (FIG. 9B) and sequencing (data not shown) in both constructs.

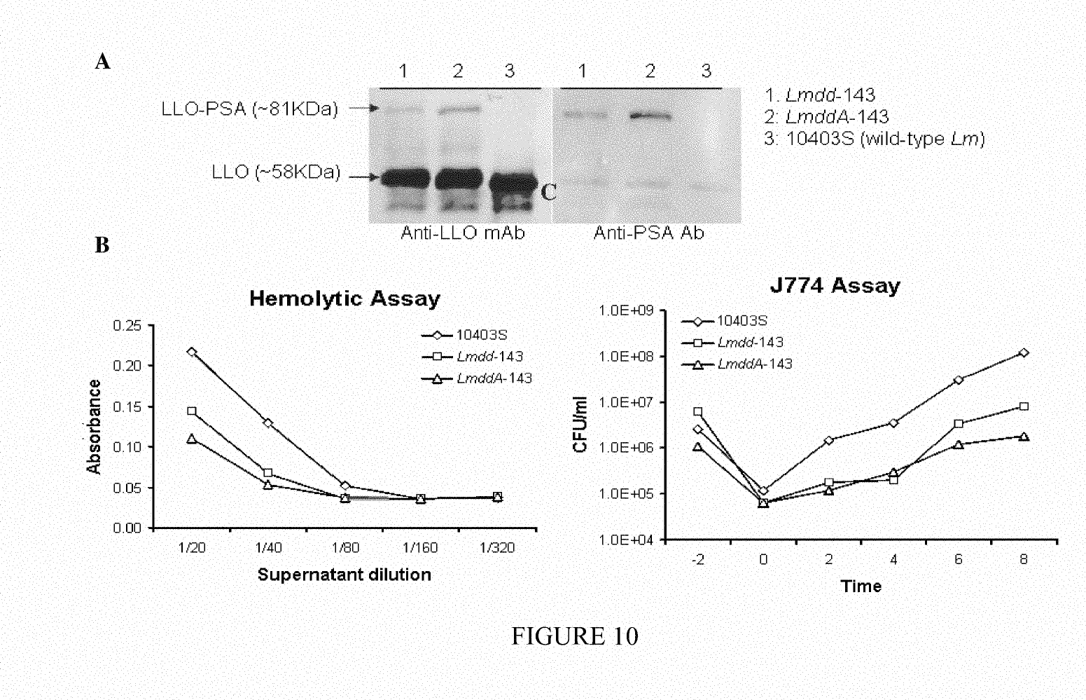

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 10A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 10B). In agreement with these results, both Lmdd-143 and LmddA-143 were able to replicate intracellularly in the macrophage-like J774 cell line (FIG. 10C).

### Example 8: Both Lmdd-143 and LmddA-143 Elicit Cell-Mediated Immune Responses Against the PSA Antigen

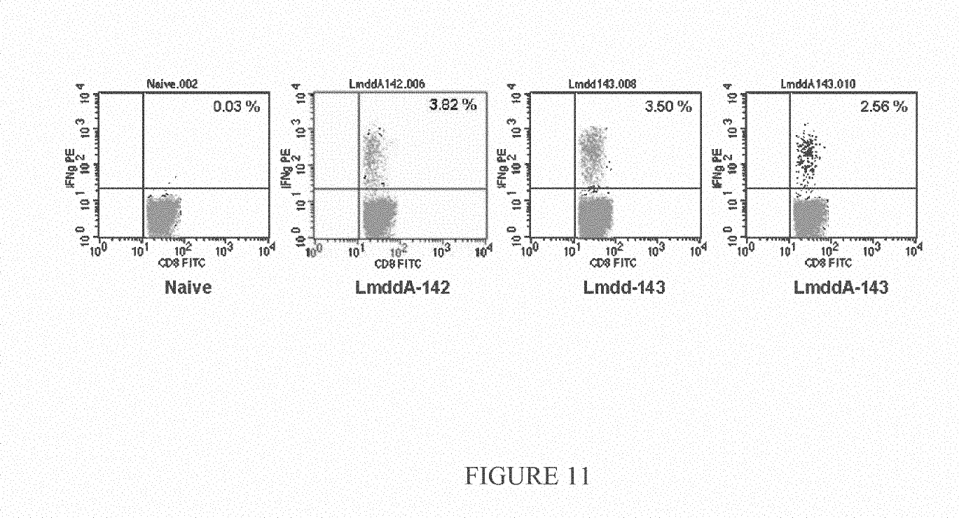

After showing that both Lmdd-143 and LmddA-143 are able to secrete PSA fused to LLO, we investigated if these strains could elicit PSA-specific immune responses in vivo. C57Bl/6 mice were either left untreated or immunized twice with the Lmdd-143, LmddA-143 or LmddA-142. PSA-specific CD8⁺ T cell responses were measured by stimulating splenocytes with the PSA₆₅₋₇₄ peptide and intracellular staining for IFN-γ. As shown in FIG. 11, the immune response induced by the chromosomal and the plasmid-based vectors is similar.

### Example 9: A Recombinant Lm Strain Secreting a LLO-HMW-MAA Fusion Protein Results in a Broad Antitumor Response

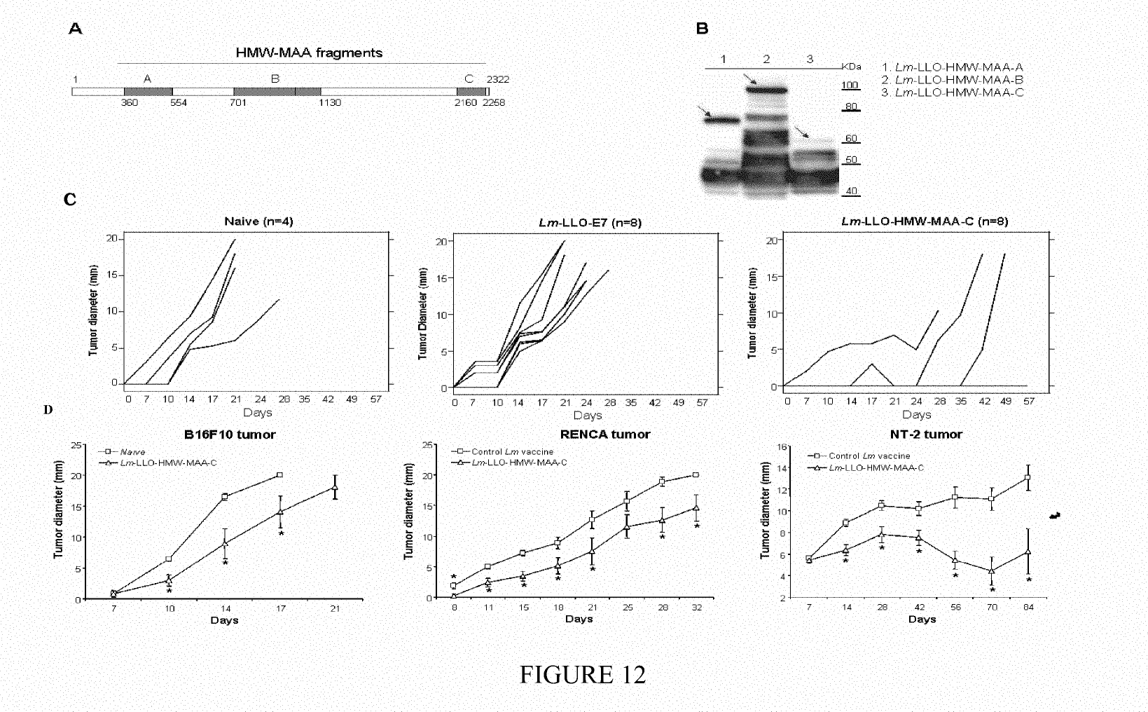

Three Lm-based vaccines expressing distinct HMW-MAA fragments based on the position of previously mapped and predicted HLA-A2 epitopes were designed (FIG. 12A). The Lm-tLLO-HMW-MMA₂₁₆₀₋₂₂₅₈ (also referred as Lm-LLO-HMW-MAA-C) is based on the avirulent Lm XFL-7 strain and a pGG55-based plasmid. This strain secretes a ˜62 kDa band corresponding to the tLLO-HMW-MAA₂₁₆₀₋₂₂₅₈ fusion protein (FIG. 12B). The secretion of tLLO-HMW-MAA₂₁₆₀₋₂₂₅₈ is relatively weak likely due to the high hydrophobicity of this fragment, which corresponds to the HMW-MAA transmembrane domain. Using B16F10 melanoma cells transfected with the full-length HMW-MAA gene, we observed that up to 62.5% of the mice immunized with the Lm-LLO-HMW-MAA-C could impede the growth of established tumors (FIG. 12C). This result shows that HMW-MAA can be used as a target antigen in vaccination strategies. Interestingly, we also observed that immunization of mice with Lm-LLO-HMW-MAA-C significantly impaired the growth of tumors not engineered to express HMW-MAA, such as B16F10, RENCA and NT-2 (FIG. 12D), which were derived from distinct mouse strains. In the NT-2 tumor model, which is a mammary carcinoma cell line expressing the rat HER-2/neu protein and is derived from the FVB/N transgenic mice, immunization with Lm-LLO-HMW-MAA-C 7 days after tumor inoculation not only impaired tumor growth but also induced regression of the tumor in 1 out of 5 mice (FIG. 12D).

### Example 10: Immunization of Mice with Lm-LLO-HMW-MAA-C Induces Infiltration of the Tumor Stroma by CD8⁺ T Cells and a Significant Reduction in the Pericyte Coverage in the Tumor Vasculature

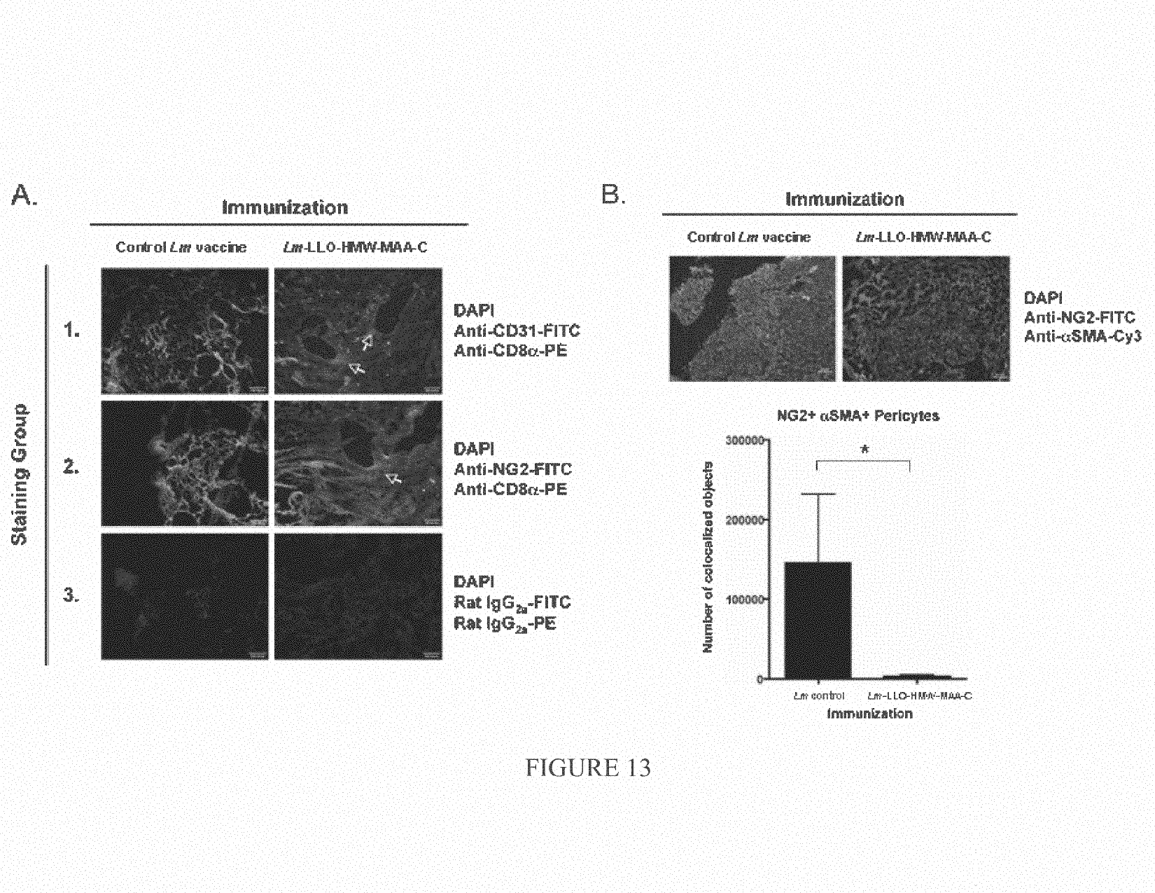

Although NT-2 cells do not express the HMW-MAA homolog NG2, immunization of FVB/N mice with Lm-LLO-HMW-MAA-C significantly impaired the growth of NT-2 tumors and eventually led to tumor regression (FIG. 12D). This tumor model was used to evaluate CD8⁺ T cells and pericytes in the tumor site by immunofluorescence. Staining of NT-2 tumor sections for CD8 showed infiltration of CD8⁺ T cells into the tumors and around blood vessels in mice immunized with the Lm-LLO-HMW-MAA-C vaccine, but not in mice immunized with the control vaccine (FIG. 13A). Pericytes in NT-2 tumors were also analyzed by double staining with uSMA and NG2 (murine homolog of HMW-MAA) antibodies. Data analysis from three independent NT-2 tumors showed a significant decrease in the number of pericytes in mice immunized with Lm-LLO-HMW-MAA-C, as compared to control (P≤0.05) (FIG. 13B). Similar results were obtained when the analysis was restricted to cells stained for aSMA, which is not targeted by the vaccine (data not shown). Thus, Lm-LLO-HMW-MAA-C vaccination impacts blood vessel formation in the tumor site by targeting pericytes.

### Example 11: Development of a Recombinant L. monocytogenes Vector with Enhanced Anti-Tumor Activity by Concomitant Expression and Secretion of LLO-PSA and tLLO-HMW-MAA21₆₀₂₂₅₈ Fusion Proteins, Eliciting Immune Responses to Both Heterologous Antigens

### Materials and Methods:

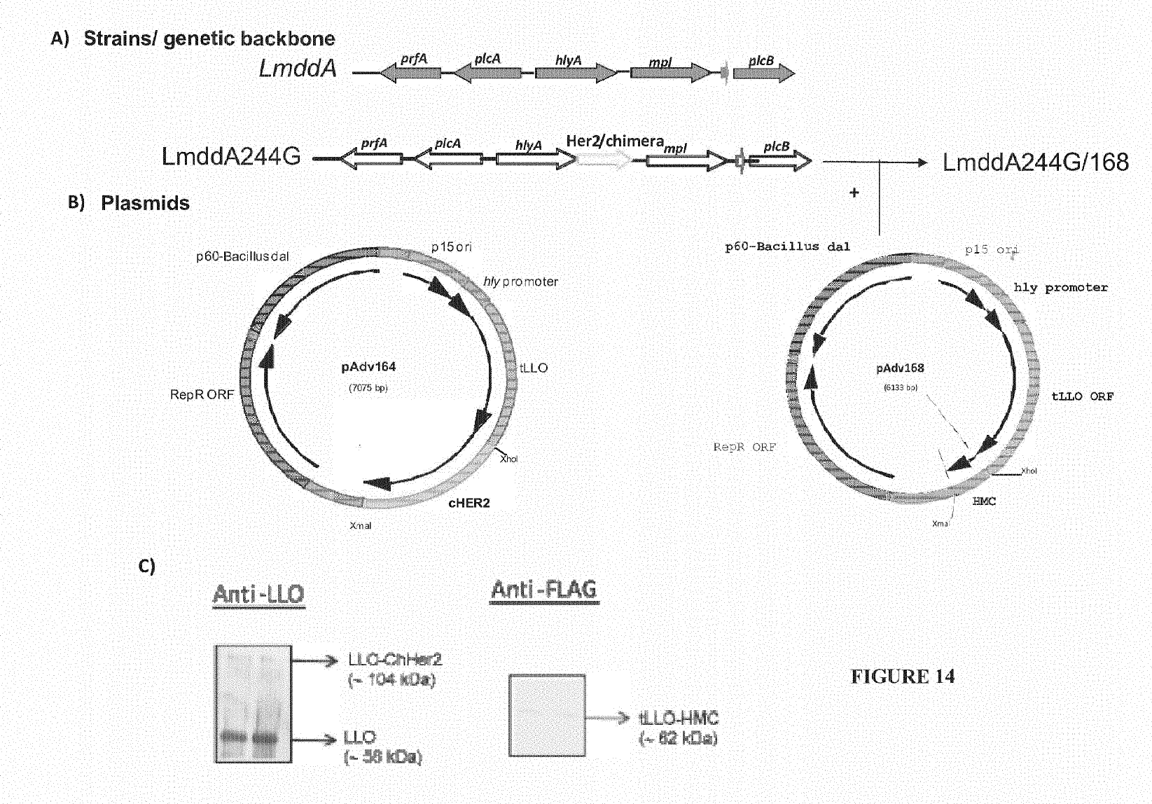

Construction of the pADV168 plasmid. The HMW-MAA-C fragment is excised from a pCR2.1-HMW-MAA₂₁₆₀₋₂₂₅₈ plasmid by double digestion with XhoI and XmaI restriction endonucleases. This fragment is cloned in the pADV134 plasmid already digested with XhoI and XmaI to excise the E7 gene. The pADV168 plasmid (FIG. 14B) is electroporated into electrocompetent the dal⁽⁻⁾ data⁽⁻⁾ E. coli strain MB2159 and positive clones screened for RFLP and sequence analysis.

Construction of Lmdd-143/168, LmddA-143/168 and the Control Strains LmddA-168, Lmdd-143/134 and LmddA-143/134.

Lmdd, Lmdd-143 and LmddA-143 is transformed with either pADV168 (FIG. 14B) or pADV134 plasmid. Transformants are selected on Brain-Heart Infusion-agar plates supplemented with streptomycin (250 μg/ml) and without D-alanine (BHIs medium). Individual clones are screened for LLO-PSA, tLLO-HMW-MAA₂₁₆₀₋₂₂₅₈ and tLLO-E7 secretion in bacterial culture supernatants by Western-blot using an anti-LLO, anti-PSA or anti-E7 antibody. A selected clone from each strain will be evaluated for in vitro and in vivo virulence. Each strain is passaged twice in vivo to select the most stable recombinant clones. Briefly, a selected clone from each construct is grown and injected i.p to a group of 4 mice at 1×10⁸ CFU/mouse. Spleens are harvested on days 1 and 3, homogenized and plated on BHIs-agar plates. After the first passage, one colony from each strain is selected and passaged in vivo for a second time. To prevent further attenuation of the vector, to a level impairing its viability, constructs in two vectors with distinct attenuation levels (Lmdd-143/168, LmddA-143/168) are generated.

Construction of Listeria Strain Engineered to Express and Secrete Two Antigens as Fusion Proteins, LmddA244G.

The antigen Her2 chimera was genetically fused to the genomic listeriolysin O (FIG. 14A) and the second antigen HMW-MAA-C(HMC) was fused to a truncated listeriolysin O in the plasmid. The secretion of fusion proteins LLO-ChHer2 and tLLO-HMC were detected by western blot using anti-LLO and anti-FLAG antibodies respectively (see FIG. 14C).

Hemolytic Assay.

To determine the ability of genomic LLO to cause phagolysosomal escape a hemolytic assay was performed using secreted supernatant of control wild type 10403S and LmddA244G-168 and sheep red blood cells as target cells.

In Vitro Intracellular Replication in J774 Cells.

An in vitro intracellular growth assay was performed using a murine macrophage-like J774 cell line. Briefly, J774 cells were infected for 1 hour in medium without antibiotics at MOI of 1:1 with either one of the mono vaccines (LmddA164 and LmddA168—each generated by transforming an individual Listeria strain with pADV164 and another with pADV168, to arrive at LmddA164 and LmddA168, respectively—see FIG. 14B) or bivalent immunotherapy. At 1 h post-infection, cells were treated with 10 μg/ml of gentamicin to kill extracellular bacteria. Samples were harvested at regular time intervals and cells lysed with water to quantify the number of intracellular CFU. Ten-fold serial dilutions of the lysates are plated in duplicates on BHI plates and colony-forming units (CFU) were counted in each sample.

In Vivo Virulence Studies.

Groups of four C57BL/6 mice (7 weeks old) are injected i.p. with two different doses (1×10⁸ and 1×10⁹ CFUs/dose) of Lmdd-143/168, LmddA-143/168, LmddA-168, Lmdd-143/134 or LmddA-143/134 strains. Mice are followed-up for 2 weeks for survival and LD₅₀ estimation. An LD₅₀ of >1×10⁸ constitutes an acceptable value based on previous experience with other Lm-based vaccines.

### Results

Once the pADV168 plasmid is successfully constructed, it is sequenced for the presence of the correct HMW-MAA sequence. This plasmid in these new strains express and secrete the LLO fusion proteins specific for each construct. These strains are highly attenuated, with an LD50 of at least 1×10⁸ CFU and likely higher than 1×10⁹ CFU for the actA-deficient (LmddA) strains, which lack the actA gene and consequently the ability of cell-to-cell spread.

A recombinant Lm (LmddA-cHer2/HMC) was generated. This Lm strain expresses and secretes a chimeric Her2 (cHer2) protein chromosomally as fusion to genomic listeriolysin O (LLO) and a fragment of HMW-MAA₂₁₆₀₋₂₂₅₈ (also named HMW-MAA C or HMC) using a plasmid as fusion to truncated LLO (tLLO), to target tumor cells and tumor vasculature concomitantly referred as LmddA244G-168. The expression and secretion of both the fusion proteins tLLO-HMC and LLO-cHer2 from LmddA244G-168 was detected by western blot using anti-FLAG and anti-LLO specific antibodies (FIG. 14B). Furthermore, the vaccine LmddA244G-168 was passaged twice in vivo in mice to stabilize the virulence of LmddA-244G and to confirm that it retained the expression of recombinant fusion proteins (FIG. 14B). The vaccine LmddA244G-168 retained its ability to express and secrete both the fusion proteins, tLLO-HMC and LLO-cHer2 after two in vivo mice passages.

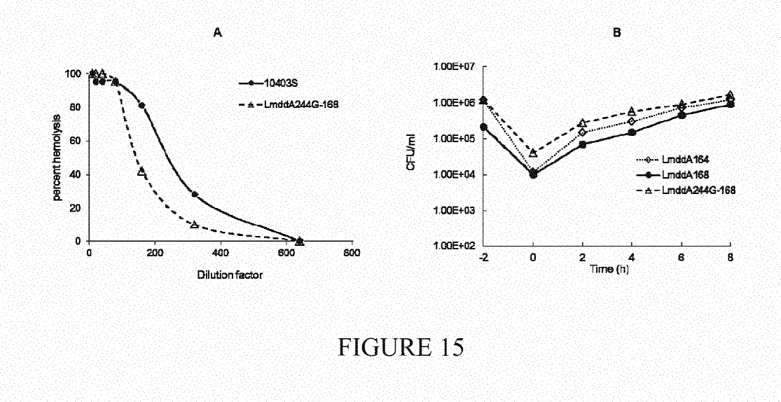

The strain LmddA244G-168, expresses chromosomal LLO as fusion protein LLO-cHer2 which may impact the functional ability of LLO to cause phagolysosomal escape. To determine this hemolytic assay was performed using secreted supernatant of control wild type 10403S and LmddA244G-168 and sheep red blood cells as target cells. As indicated in FIG. 15A, there was a 1.5 fold reduction in the hemolytic ability of LmddA244G-168 when compared to wild type highly virulent Lm strain 10403S.

Additionally, to examine if the expression of fusion protein LLO-cHer2 did not cause any deleterious effect on the ability of LmddA-cHer2/HMC to infect macrophages and its intracellular growth, a cell infection assay was performed using mouse macrophage like cells J774. The results as specified in FIG. 15B showed that intracellular growth behavior of different Listeria-based immunotherapies expressing either single or dual antigens were similar suggesting that the co-expression of two antigens did not cause any change in the ability of LmddA244G-168 to present target intracellular proteins for immunological responses.

### Example 12: Detection of Immune Responses and Anti-Tumor Effects Elicited Upon Immunization with Lmdd-244G/168

Immune responses to cHer2 and HMW-MAA are studied in mice upon immunization with Lmdd-244G-168 strain using standard methods, such as detection of IFN-γ production against these antigens. The therapeutic efficacy of dual-expression vectors are tested in the NT2 breast tumor model.

IFN-γ ELISpot.

We evaluated the ability of bivalent immunotherapy to generate immune responses specific for the two antigens Her2 and HMW-MAA in FvB mice. Mice (3/group) were immunized with different immunotherapies such as LmddA134 (Lm-control), LmddA164 and LmddA244G/168 on day 0 and boosted on day 14. Her2/neu specific immune responses were detected in the spleens harvested on day 21. The IFN-γ ELispot assay was done according to the kit instructions and spleen cells were stimulated with peptide epitope specific for the intracellular region (RLLQETELV) (SEQ ID NO: 79).

IFN-γ ELISA.

The generation of HMW-MAA-C specific immune responses in the splenocytes of immunized mice was determined by stimulating cells with HMA-MAA-C protein for 2 days. The IFN-γ release was detected by ELISA performed using mouse interferon-gamma ELISA kit.

Anti-Tumor Efficacy.

The antitumor efficacy was examined using mouse NT2 breast tumor model. FvB mice were implanted with 1×10⁶ NT2 cells on day 0 and established tumors on right flank were treated starting day 6 with three immunizations at weekly intervals with different immunotherapies. Tumors were monitored twice a week until the end of the study. Mice were euthanized if the tumor diameter was greater than 1.5 cm.

### Results

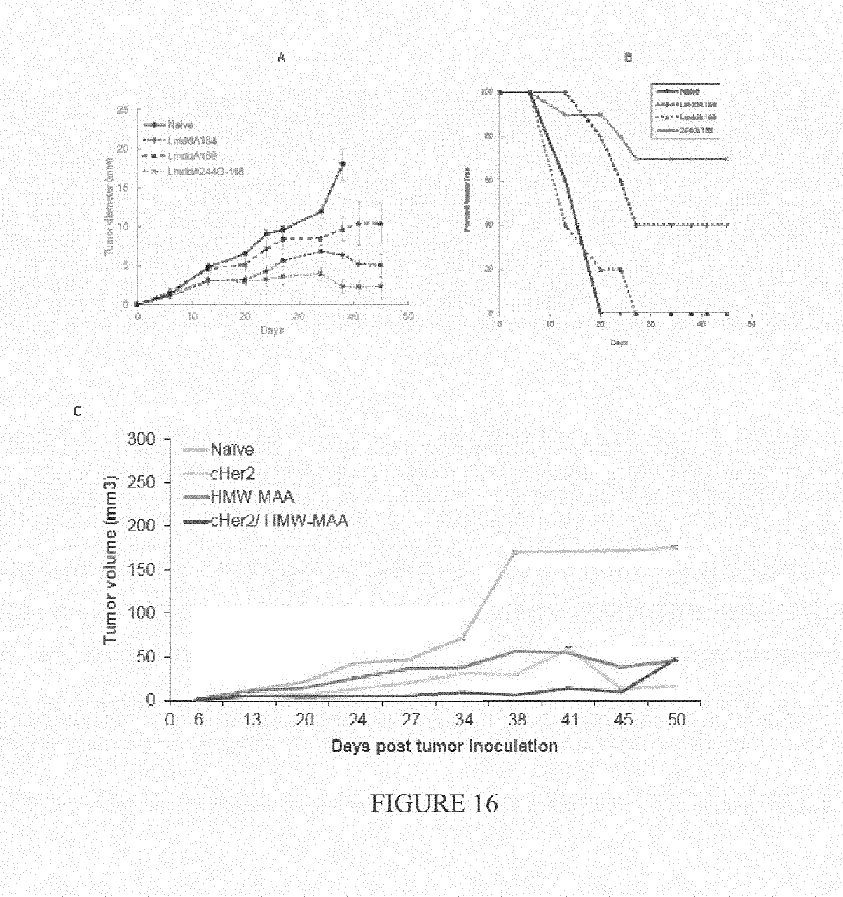

Next, the anti-tumor therapeutic efficacy of LmddA244G was examined using mouse NT2 breast tumor model. The FvB mice bearing established NT2 tumors on right flank were treated with three immunizations at one week interval with different immunotherapies expressing either mono antigens LmddA164 (ChHer2), LmddA168 (HMC) or bivalent immunotherapy LmddA244G-168. Treatment with both mono- and bivalent-immunotherapy caused a reduction of NT2 tumor as indicated in FIGS. 16A and 16C. However, a stronger impact on the control of NT2 tumor growth was observed after treatment with bivalent-immunotherapy. Additional analysis on the percent tumor free mice in each group confirmed that treatment with bivalent immunotherapy generated maximum tumor-free mice (70%) when compared to mono-immunotherapy (less than 40%) treated groups. These observations support that targeting two antigens concurrently using Listeria monocytogenes as vector for therapy resulted in enhanced anti-tumor efficacy.

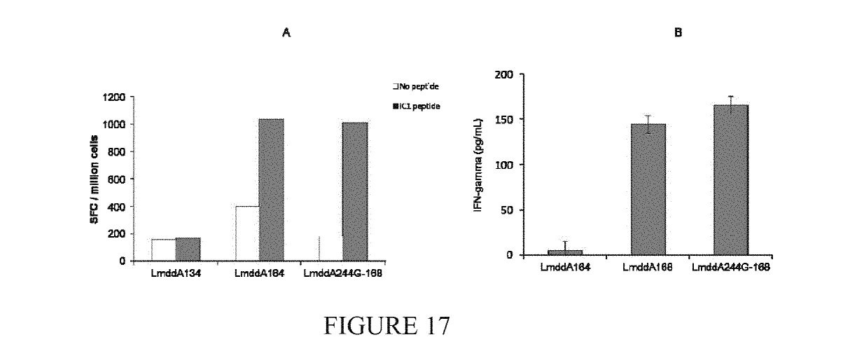

The ability of bivalent immunotherapy was evaluated to generate immune responses specific for the two antigens Her2 and HMW-MAA in FvB mice. Mice were immunized with different immunotherapies such as LmddA134 (irrelevant control), LmddA164 and LmddA244G/168 on day 0 and boosted on day 14. Her2/neu specific immune responses were detected using an ELISpot based assay using peptide epitope specific for intracellular region. Both mono and bivalent-immunotherapy expressing Her2 generated comparable levels of immune responses detected using ELISpot-based assay (see FIG. 17).

The generation and for HMW-MAA-C specific immune responses in the splenocytes of immunized mice was detected using ELISA. The expression of tumor antigen from Lm using either single copy (mono immunotherapy) or multicopy (bivalent immunotherapy) based expression generates comparable level of antigen-specific immune responses (see FIG. 17).

### Example 13: Immunization with Either Lmdd-143/168 or LmddA-143/168 Results in Pericyte Destruction, Up-Regulation of Adhesion Molecules in Endothelial Cells and Enhanced Infiltration of TILs Specific for PSA

Characterization of tumor infiltrating lymphocytes and endothelial cell-adhesion molecules induced upon immunization with Lmdd-143/168 or LmddA-143/168. The tumors from mice immunized with either Lmdd-143/168 or LmddA-143/168 are analyzed by immunofluorescence to study expression of adhesion molecules by endothelial cells, blood vessel density and pericyte coverage in the tumor vasculature, as well as infiltration of the tumor by immune cells, including CD8 and CD4 T cells. TILs specific for the PSA antigen are characterized by tetramer analysis and functional tests.

Analysis of Tumor Infiltrating Lymphocytes (TILs).

TPSA23 cells embedded in matrigel are inoculated s.c in mice (n=3 per group), which are immunized on days 7 and 14 with either Lmdd-143/168 or LmddA-143/168, depending on which one is the more effective according to results obtained in anti-tumor studies. For comparison, mice are immunized with LmddA-142, LmddA-168, a control Lm vaccine or left untreated. On day 21, the tumors are surgically excised, washed in ice-cold PBS and minced with a scalpel. The tumors are treated with dispase to solubilize the Matrigel and release single cells for analysis. PSA-specific CD8⁺ T cells are stained with a PSA65-74 H-2Db tetramer-PE and anti-mouse CD8-FITC, CD3-PerCP-Cy5.5 and CD62L-APC antibodies. To analyze regulatory T cell in the tumor, TILs are stained with CD4-FITC, CD3-PerCP-Cy5.5 and CD25-APC and subsequently permeabilized for FoxP3 staining (anti-FoxP3-PE, Milteny Biotec). Cells are analyzed by a FACS Calibur cytometer and CellQuestPro software (BD Biosciences).

Immunofluorescence.

On day 21 post tumor inoculation, the TPSA23 tumors embedded in matrigel are surgically excised and a fragment immediately cryopreserved in OCT freezing medium. The tumor fragments are cryosectioned for 8-10 μm thick sections. For immunofluorescence, samples are thawed and fixed using 4% formalin. After blocking, sections are stained with antibodies in blocking solution in a humidified chamber at 37° C. for 1 hour. DAPI (Invitrogen) staining are performed according to manufacturer instructions. For intracellular stains (aSMA), incubation is performed in PBS/0.1% Tween/1% BSA solution. Slides are cover-slipped using a mounting solution (Biomeda) with anti-fading agents, set for 24 hours and kept at 4° C. until imaging using Spot Image Software (2006) and BX51 series Olympus fluorescent microscope. CD8, CD4, FoxP3, aSMA, NG2, CD31, ICAM-1, VCAM-1 and VAP-1 are evaluated by immunofluorescence.

Statistical Analysis:

Non-parametric Mann-Whitney and Kruskal-Wallis tests are applied to compare tumor sizes among different treatment groups. Tumor sizes are compared at the latest time-point with the highest number of mice in each group (8 mice). A p-value of less than 0.05 is considered statistically significant in these analyses.

### Results

Immunization of TPSA23-bearing mice with the Lmdd-143/168 and LmddA-143/168 results in higher numbers of effector TILs specific to PSA and also decreases pericyte coverage of the tumor vasculature. Further, cell-adhesion markers are significantly up-regulated in immunized mice.

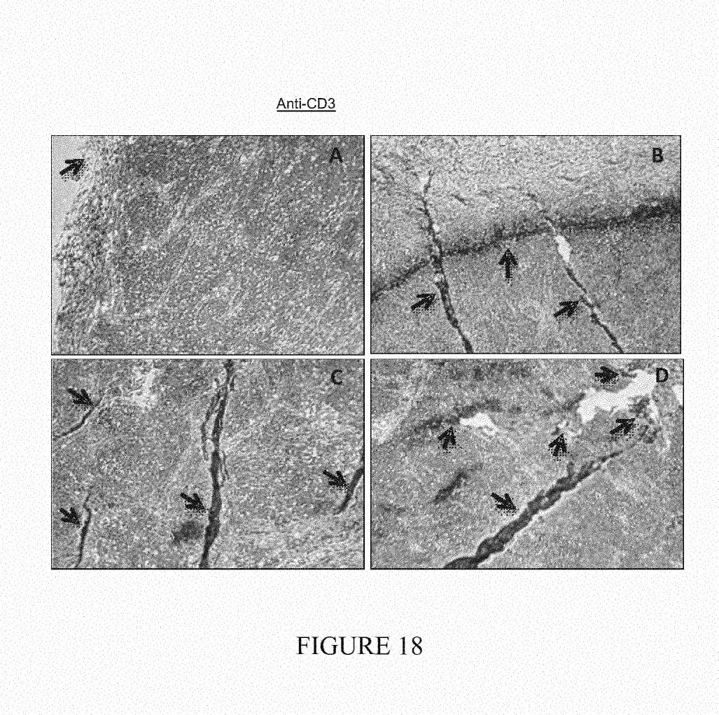

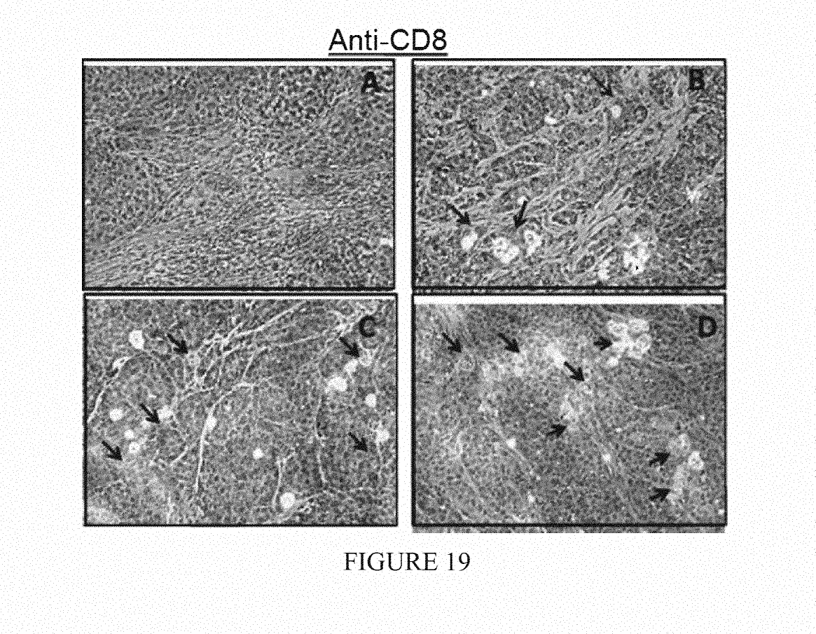

An increased infiltration of T cells in the tumors treated with Bivalent-immunotherapy was observed using anti-CD3 and anti-CD8 staining as compared to the monovalent treated groups (FIGS. 18-19).

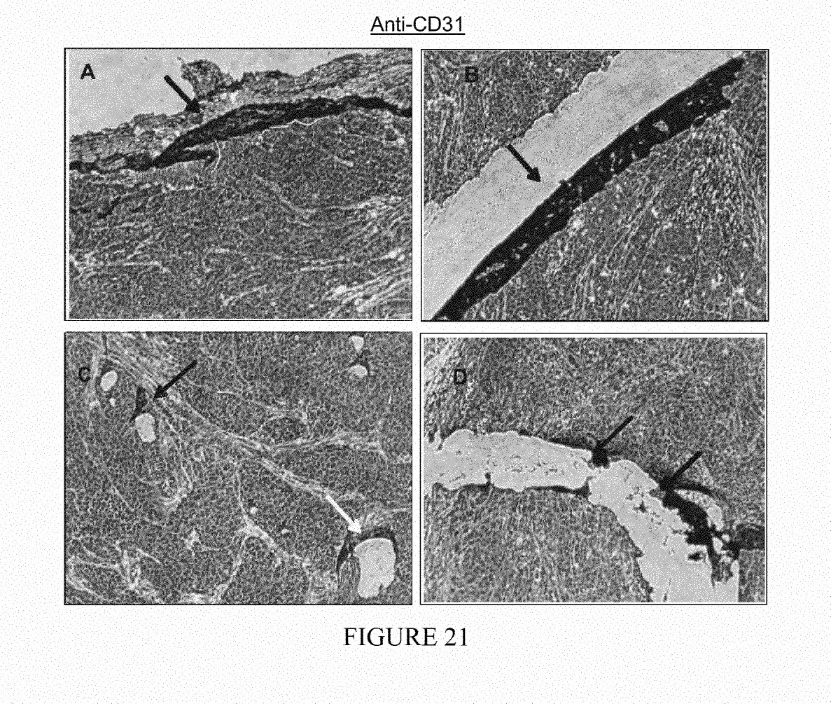

In addition there was an increase in the infiltration of CD4⁺ T cells in the tumors of both LMddA168 and LmddA244-168 treated groups. Further, a reduction in the staining of blood vessels by anti-CD31 (FIG. 21) was observed in the LmddA168 and LmddA244G-168 treatment groups.

### Example 14: Anti-Tumor Efficacy of a Dual cHER2-CA9 Listeria Vaccine on the Growth of 4T1 Tumors Implanted in the Mammary Glands of Balb/c Mice

Experimental Details:

A recombinant Lm (LmddA-cHer2/CA9) was generated. This Lm strain expresses and secretes a chimeric Her2 (cHer2) protein chromosomally as fusion to genomic listeriolysin O (LLO) and a fragment of human Carbonic Anhydrase 9 (CA9) using a plasmid as fusion to truncated LLO (tLLO), to multiply target tumor cells.

TABLE 3

| | 4T1 Tumor | Vaccine | Vaccine | Measure- |
|---|---|---|---|---|
| | Implantation | Dose 1 | Boost | ment |
| Group | (7 × 10³) | (1 × 10⁸ CFU) | (1 × 10⁸ CFU) | Dates |
| Naïve-PBS | Day 0 | Day 3 | Day 10 | 1X/week |
| LmddA-PSA | Day 0 | Day 3 | Day 10 | 1X/week |
| LmddA-cHER2 | Day 0 | Day 3 | Day 10 | 1X/week |
| LmddA-CA9 | Day 0 | Day 3 | Day 10 | 1X/week |
| LmddA- | Day 0 | Day 3 | Day 10 | 1X/week |
| cHER2-CA9 | | | | |

Vaccine titers:
LmddA-PSA-6.5 × 10⁸
LmddA-CA9-1.4 × 10¹⁰
LmddA-cHER2-1.05 × 10¹⁰
Dual cHer2-CA9 (LmddA)-1.5 × 10⁹

Experimental Protocols:

4T1 cells were grown in RPMI containing 10% FBS, 2 mM L-Glu, 1.5 g/L sodium bicarbonate, 4.5 g/L glucose, ImM sodium pyruvate, and 10 mM HEPES. On the day of injection, cells were trypsinized then washed 2× in PBS. Cells were counted and resuspended at 7×10³ cells/50 μl.

Tumors were implanted in the mammary glands of each of the mice. There are 16 mice per group. The mice were vaccinated 3 days later. On day 4, 4 mice in each group were euthanized and examined for tumor growth. Mice were given the boost of each vaccine on day 10. On day 11, 4 mice in each group were euthanized and tumors were measured. On day 18, 4-5 mice in each group were euthanized and tumors were measured. On day 21, the remaining mice in each group were euthanized and the tumors were measured.

### Results

On day 4, the tumors are barely palpable, so no measurements were made.

TABLE 4

| | | | | | | | | | |
|---|---|---|---|---|---|---|---|---|---|
| PBS | PBS Average | PSA | PSA Average | CA9 | CA9 Average | HER2 | Her2 Average | Dual | Dual Average |

Jan. 20, 2012—Day 11

| | | | | | | | | | |
|---|---|---|---|---|---|---|---|---|---|
| 3.7 × 4.13 | 3.915 | 3.99 × 2.73 | 3.36 | 1.3 × 2.1 | 1.7 | 2.3 × 3.2 | 2.75 | 0 | 0 |
| 2.1 × 1.4 | 1.75 | 3.58 × 4.91 | 4.245 | 3.3 × 4.1 | 3.7 | 1.3 × 3.2 | 2.25 | 0 | 0 |
| 3.2 × 2.4 | 2.8 | 1.93 × 2.3 | 2.115 | 2.2 × 3.1 | 2.65 | 2.1 × 2.2 | 2.15 | 1.1 × 1.3 | 1.2 |
| 1.2 × 3.1 | 2.15 | 2.2 × 3.1 | 2.65 | 2.2 × 1.4 | 1.8 | 1.2 × 3.1 | 2.15 | 2.1 × 3.2 | 2.65 |
| Average | 2.65 | | 3.09 | | 2.46 | | 2.33 | | 0.96 |

Jan. 27, 2012—Day 18

| | | | | | | | | | |
|---|---|---|---|---|---|---|---|---|---|
| 3.87 × 7.02, | 9.465 | 5.8 × 11.12 | 8.46 | 4.18 × 3.49, | 6.88 | 4.74 × 6.34 | 5.54 | 5.24 × 4.59 | 4.915 |
| 6.1 × 1.94 | | | | 2.75 × 3.34 | | | | | |
| 3.28 × 11.26 | 7.27 | 6.02 × 7.5, | 11.9 | 5.72 × 7.23 | 6.475 | 3.73 × 7.34 | 5.535 | 4.92 × 4.87 | 4.895 |
| | | 3.54 × 6.74 | | | | | | | |
| 6.97 × 7.86, | 11.335 | 5.06 × 7.18, | 9.7 | 4.08 × 7.64 | 5.86 | 2.97 × 5.34 | 4.155 | 3 × 5.55 | 4.275 |
| 2.63 × 5.21 | | 3.72 × 3.44 | | | | | | | |
| 4.47 × 8.82 | 6.645 | 9.17 × 10.49 | 9.83 | 4.08 × 3.54 | 3.81 | 7.41 × 5.05 | 6.23 | 2.89 × 6.73, | 8.43 |
| | | | | | | | | 2.87 × 4.37 | |
| 8.63 × 4.52, | 10.375 | 1 found dead | | 1 found dead | | 5.7 × 5.95 | 5.825 | 2.82 × 5.27 | 4.045 |
| 5 × 2.6 | | | | | | | | | |
| Average | 9.018 | | 9.9725 | | 5.76 | | 5.42 | | 5.312 |

Jan. 30, 2012—Day 21

| PBS | PBS Average | PSA | PSA Average | CA9 | CA9 Average | Her2 | Her2 Average | Dual | Dual Average |
|---|---|---|---|---|---|---|---|---|---|
| 5.7 × 8.82, | 11.615 | 7.53 × 10.63 | 9.08 | 4.86 × 9.68 | 7.24 | 8.72 × 10.78, | 11.605 | 4.12 × 6.18 | 5.15 |
| 2.4 × 6.31 | | | | | | 1.3 × 2.41 | | | |
| 10.27 × 7.62 | 8.945 | 8.38 × 11.61 | 9.995 | 5.03 × 8.38 | 6.705 | 6.8 × 5.91 | 6.355 | 4.76 × 6.36 | 5.56 |
| 1 found dead | | 8.66 × 9.41 | 9.035 | 1 found dead | | 1 found dead | | 1 found dead | |
| Average | 10.28 | | 9.37 | | 6.97 | | 8.98 | | 5.355 |

The numbers in Table 4 show that the dual vaccine (recombinant Listeria expressing two heterologous antigens) initially (day 11) has a large impact on the tumor mass (FIG. 21). Two of the mice euthanized had no tumors and the others were smaller than the control and around the size of the mono-CA9 and cHER2 vaccinated mice. By day 18, multiple tumors can be measured in some of the mice in several of the groups. The PBS and PSA control mice have much larger tumors than the mono-CA9 and cHER2 or the dual vaccine groups. The dual vaccine group has one outlier with a large tumor burden, otherwise the average for that group would have been the smallest. The experiment was terminated early as the mice in several groups were looking very sick and had been dying. However, at the last measurement, the mice in the dual vaccine group had the smallest tumors (FIG. 21). This may be due to the level of control on tumor growth that was seen early on.

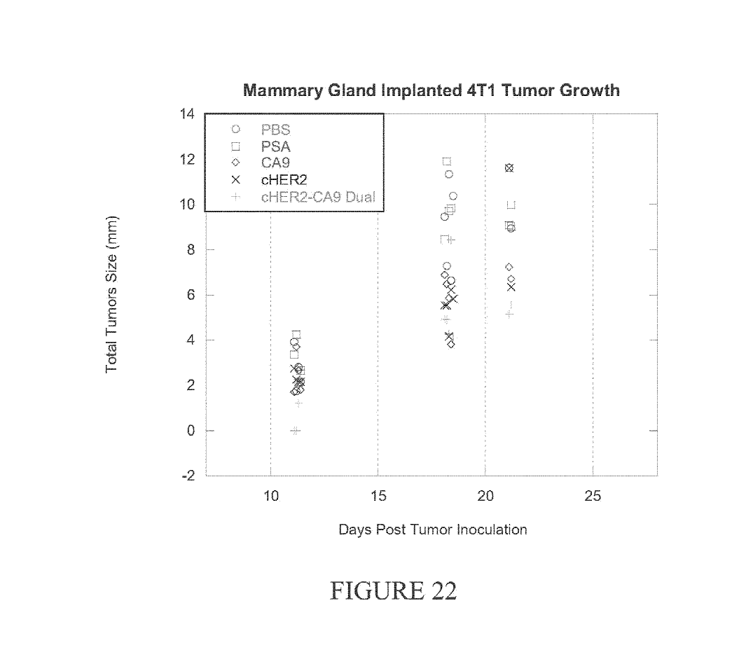

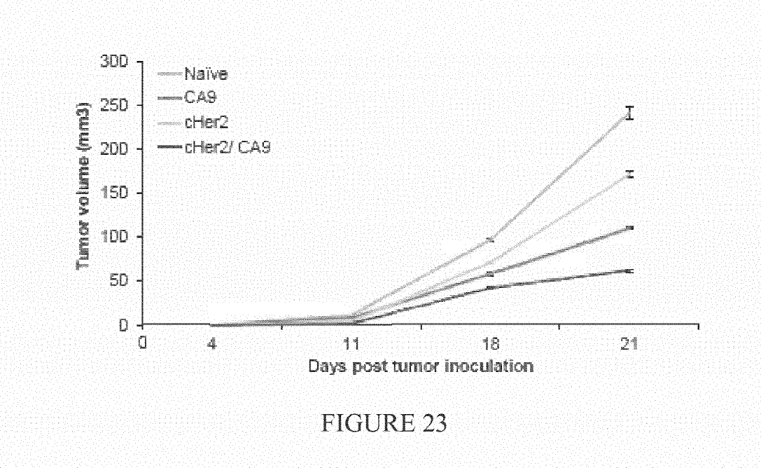

In conclusion, the dual vaccine shows an initial level of tumor control in the 4T1 model that is higher than levels achieved with the mono-vaccines or the control mice as the dual vaccine groups have the smallest tumor burden at the end of the experiment (see FIGS. 22 and 23).

### Example 15: Anti-Tumor Efficacy of a Dual cHER2-HMW-MAA Listeria Vaccine on the Growth of 4T1 Tumors Implanted in the Mammary Glands of Balb/c Mice

Experimental Details:

A recombinant Lm (LmddA-cHer2/HMW-MAA) was generated. This Lm strain expresses and secretes a chimeric Her2 (cHer2) protein chromosomally as fusion to genomic listeriolysin O (LLO) and high molecular weight melanoma associated antigen (HMW-MAA) using a plasmid as fusion to truncated LLO (tLLO), to multiply target tumor cells.

TABLE 5

| | 4T1-HMW- | Immunotherapy | | | |
|---|---|---|---|---|---|
| | MAA Tumor | Dose 1 | Dose 2 | Dose 3 | Measure- |
| | Implantation | (1 × 10⁸ | (1 × 10⁸ | (1 × 10⁸ | ment |
| Groups | (1 × 10⁴) | CFU) | CFU) | CFU) | Dates |
| Naïve-PBS | Day 0 | Day 1 | Day 8 | Day 15 | 1X/Week |
| cHer2 | Day 0 | Day 1 | Day 8 | Day 15 | 1X/Week |
| HMW-MAA | Day 0 | Day 1 | Day 8 | Day 15 | 1X/Week |
| cHer2/HMW-MAA | Day 0 | Day 1 | Day 8 | Day 15 | 1X/Week |

Vaccine titers:
LmddA-PSA-6.5 × 10⁸
LmddA-HMW-MMA-1.4 × 10¹⁰
LmddA-cHER2-1.05 × 10¹⁰
Dual cHer2-HMW-MMA (LmddA)-1.5 × 10⁹

Experimental Protocols:

4T1 cells were grown in RPMI containing 10% FBS, 2 mM L-Glu, 1.5 g/L sodium bicarbonate, 4.5 g/L glucose, 1 mM sodium pyruvate, and 10 mM HEPES. On the day of injection, cells were trypsinized then washed 2× in PBS. Cells were counted and resuspended at 7×10³ cells/50l.

Tumors were implanted in the mammary glands of each of the mice. There are 16 mice per group. The mice were vaccinated 3 days later. On day 8, 4 mice in each group were euthanized and examined for tumor growth. Mice were given the boost of each vaccine on day 8. On day 15, 4 mice in each group were euthanized and tumors were measured. Mice were given another boost of each vaccine on day 15. On day 15, 21, 28 and 35, 4-5 mice in each group were euthanized and tumors were measured. On days 42, the remaining mice in each group were euthanized and the tumors were measured.

### Results

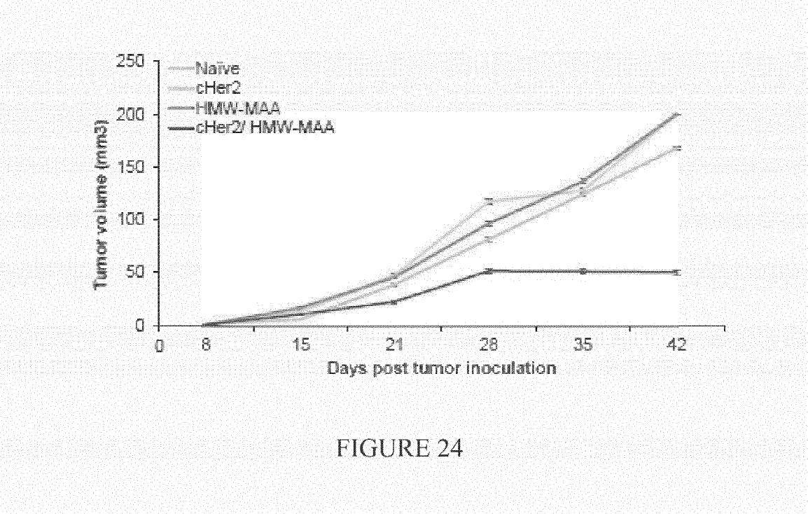

The results are summarized in FIG. 24. The graphs show that the dual vaccine (recombinant Listeria expressing two heterologous antigens) has a large impact on the tumor volume (FIG. 24). The volumes of tumors in mice receiving bivalent therapy were smaller than both the control and the mono-HMW-MMA and cHER2 vaccinated mice. The PBS and PSA control mice have tumors that are comparable in volume to the mono-HMW-MMA and cHER2 groups.

In conclusion, the dual vaccine shows an initial level of tumor control in the 4T1 model that is higher than levels achieved with the mono-vaccines or the control mice as the dual vaccine groups have the smallest tumor burden at the end of the experiment (see FIG. 24).

### Example 16: Comparative Study of Anti-Tumor Efficacy of a Dual and Sequential cHER2-HMW-MAA Listeria Vaccine on the Growth of NT2 Breast Tumor Model

Experimental Details:

The antitumor efficacy was examined using mouse NT2 breast tumor model. FvB mice were implanted with 1×10⁶ NT2 cells on day 0 and established tumors on right flank were treated starting day 6 with three immunizations at weekly intervals with different immunotherapies. Tumors were monitored twice a week until the end of the study. Mice were euthanized if the tumor diameter was greater than 1.5 cm.

TABLE 6

| | NT2 Tumor | | |
|---|---|---|---|
| | Implantation | Immunotherapy | Measurement |
| Groups | (1 × 10⁶) | Doses (1 × 10⁸ CFU) starting on Day 7 | Dates |
| Naïve-PBS | Day 0 | PBS; 5 doses; one week apart | 2X/Week |
| cHer2 | Day 0 | 5 doses; one week apart | 2X/Week |
| HMW-MAA | Day 0 | 5 doses; one week apart | 2X/Week |
| cHer2 + HMW-MAA | Day 0 | 5 doses; one week apart | 2X/Week |
| cHer2 followed by | Day 0 | Doses one week apart; 3 doses of cHer2 | 2X/Week |
| HMW-MAA | | followed by 3 doses of HMW-MAA | |

### Results

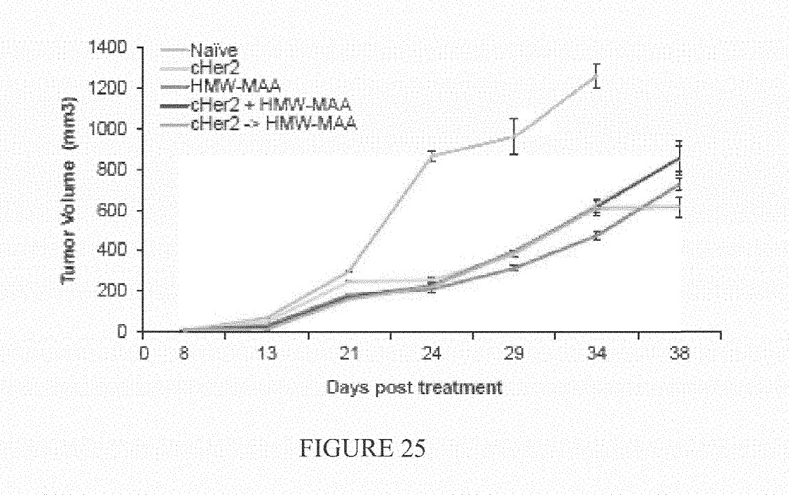

The anti-tumor therapeutic efficacy of different listeria vaccine regiments was examined using mouse NT2 breast tumor model. The FvB mice bearing established NT2 tumors on right flank were treated with five immunizations of 1×10⁸ at one week intervals with different immunotherapies expressing either mono antigens LmddA164 (ChHer2), LmddA168 (HMC), or combination of therapies expressing both antigens administered simultaneously (bivalent therapy) (see Table 6). In addition, a combination vs sequential therapy was carried out with different immunotherapies expressing either mono antigens LmddA164 (ChHer2), LmddA168 (HMC), a combination of therapies expressing both antigens administered simultaneously (bivalent therapy), or a combination of sequential administration of each mono antigen (cHer2 followed by HMW-MAA). In the latter, 3 weekly doses of LmddA164 (ChHer2) were administered and were followed by 3 weekly doses of LmddA168 (HMC) (see Table 6). The results are summarized in FIG. 25. All the regiments caused approximately equivalent reduction of NT2 tumor volume as indicated in FIG. 25. These observations show that simultaneous or sequential administration of two monovalent constructs was at least comparable to bivalent constructs in controlling tumor growth (FIG. 25).

### Example 17: Generation of PSA and SIINFEKL-Specific Responses in C57BL/6 Mice after Immunization with PSA-SVN, PSA-PSMA and SIINFEKL Minigene

### Methods

### Strain Construction:

The DNA sequences XhoI site-PSA-Survivin-XmaI site, XhoI site-PSA-Survivin-tags-XmaI site, and XhoI site-PSA-PSMA-tags-XmaI site were synthesized by Genewiz, Inc. (South Plainfield, N.J.) and shipped to Advaxis in carrier plasmid pUC57-Kan. The target sequence for PSA is set forth in SEQ ID NO: 83 (amino acid sequence in SEQ ID NO: 108), the target sequence for survivin in set forth in SEQ ID NO: 84 (amino acid sequence in SEQ ID NO: 109), and the target sequence for PSMA is set forth in SEQ ID NO: 85 (amino acid sequence in SEQ ID NO: 110) or SEQ ID NO: 86 if the transmembrane domain sequence is deleted (amino acid sequence in SEQ ID NO: 111). Examples of DNA sequences encoding PSA-antigen (e.g., survivin or PSMA) fusion proteins are set forth in SEQ ID NOS: 89-91 (amino acid sequences set forth in SEQ ID NOS: 114-116). An exemplary linker used to link the PSA-encoding and the survivin-encoding or PSMA-encoding sequence is set forth in SEQ ID NO: 87 (amino acid sequence in SEQ ID NO: 112), and an exemplary SIINFEKL-6×His tag is set forth in SEQ ID NO: 88 (amino acid sequence in SEQ ID NO: 113). Each carrier plasmid was transformed into chemically competent Top10 E. coli (Invitrogen) for storage and to allow mass plasmid preparation. Each carrier plasmid was purified using a QIAGEN Plasmid Midi Kit (Qiagen) per the manufacturer's instructions. In order to isolate XhoI site-PSA-Survivin-XmaI site, XhoI site-PSA-Survivin-tags-XmaI site, and XhoI site-PSA-PSMA-tags-XmaI inserts, each carrier plasmid each restriction enzyme digested overnight with XhoI and XmaI (New England Biolabs) and recovered by agarose gel electrophoresis and gel extraction using a GENECLEAN Kit (MPBio) as per the manufacturer's instructions. XhoI site-PSA-Survivin-XmaI site, XhoI site-PSA-Survivin-tags-XmaI site, and XhoI site-PSA-PSMA-tags-XmaI site inserts were ligated into similarly cut pAdv134 using T4 DNA ligase (New England Biolabs) to generate pAdv134-PSA-Survivin, pAdv134-PSA-Survivin-tags, and pAdv134-PSA-PSMA-tags, respectively. Plasmid insert sequences were then confirmed by DNA sequencing. The pAdv134 sequence is set forth in SEQ ID NO: 95. Exemplary pAdv134 sequences with DNA sequences encoding PSA-antigen fusion proteins are set forth in SEQ ID NOS: 96-98). Primer sequences used in this example are set forth in SEQ ID NOS: 99-106.

pAdv134-PSA-Survivin, pAdv134-PSA-Survivin-tags, and pAdv134-PSA-PSMA-tags were then electroporated into LmddA to generate strains LmddA-PSA-Survivin, LmddA-PSA-Survivin-tags, and LmddA-PSA-PSMA-tags, respectively. SIINFEKL-tagged tLLO-fusion protein expression from strains LmddA-PSA-Survivin-tags and LmddA-PSA-PSMA-tags was then confirmed by DC4 SIINFEKL presentation assay. Exemplary DNA sequences encoding tLLO-PSA-antigen fusion proteins are set forth in SEQ ID NOS: 92-94 (amino acid sequences in SEQ ID NOS: 117-119). The tLLO-encoding sequence is set forth in SEQ ID NO: 82 (amino acid sequence set forth in SEQ ID NO: 107). LmddA-PSA-Survivin, LmddA-PSA-Survivin-tags, and LmddA-PSA-PSMA-tags were then mouse in vivo passaged twice and SIINFEKL-tagged tLLO-fusion protein expression from strains LmddA-PSA-Survivin-tags and LmddA-PSA-PSMA-tags was then reconfirmed.

### PSA Immunogenicity Study

This assay examines the generation of PSA and SIINFEKL-specific immunity in mice immunized with two different PSA-SIINFEKL tagged constructs. SIINFEKL minigene is included as a positive control. The PSA and SIINFEKL-specific immune response is detected by dextramer (Immudex) staining using the known T cell epitopes for C57BL/6 mice, H-2 D^{b} PSA₆₅₋₇₃ (HCIRNKSVI) and H-2 K^{b} OVA₂₅₇₋₂₆₄ (SIINFEKL). The details of the immunization schedule and strains are given in Tables 7 and 8.

TABLE 7
Immunization schedule

| Construct | Titer | Mice/Group | Dose 1 | Dose 2 | Dose 3 | Spleen harvest |
|---|---|---|---|---|---|---|
| Control listeria | 1.8 × 10⁹ | 5 | Aug. 5, 2015 | None | None | Aug. 13, 2015 |
| LmddA324 | | | | | | |
| Control listeria | 1.8 × 10⁹ | 5 | Aug. 5, 2015 | Aug. 19, 2015 | Sep. 1, 2015 | Sep. 9, 2015 |
| LmddA324 | | | | | | |
| PSA-SVN | 1.7 × 10⁹ | 5 | Aug. 5, 2015 | None | None | Aug. 13, 2015 |
| (P2 g6-1 #1) | | | | | | |
| PSA-SVN | 1.7 × 10⁹ | 5 | Aug. 5, 2015 | Aug. 19, 2015 | Sep. 1, 2015 | Sep. 9, 2015 |
| (P2 g6-1 #1) | | | | | | |
| PSA-PSMA | 1.9 × 10⁹ | 5 | Aug. 5, 2015 | None | None | Aug. 13, 2015 |
| (P2 12-1-1 #1) | | | | | | |
| PSA-PSMA | 1.9 × 10⁹ | 5 | Aug. 5, 2015 | Aug. 19, 2015 | Sep. 1, 2015 | Sep. 9, 2015 |
| (P2 12-1-1 #1) | | | | | | |

TABLE 8
Dose preparations

| | Titre | Dose | |
|---|---|---|---|
| Construct | (CFU/mL) | (CFU) | Dose preparation |
| LmddA 324 | 1.8 × 10⁹ | 1 × 10⁸ | Thaw and dilute |
| | | | 1 mL of dose with |
| | | | 0.8 mL of PBS |
| PSA-SVN-TAG | 1.7 × 10⁹ | 1 × 10⁸ | Thaw and dilute |
| (P2 g6-1 #1) | | | 1 mL of dose with |
| | | | 0.7 mL of PBS |
| PSA-PSMA-TAG | 1.9 × 10⁹ | 1 × 10⁸ | Thaw and dilute |
| (P2 12-1-1 #1) | | | 1 mL of dose with |
| | | | 0.9 mL of PBS |

Results after Primary Immunization

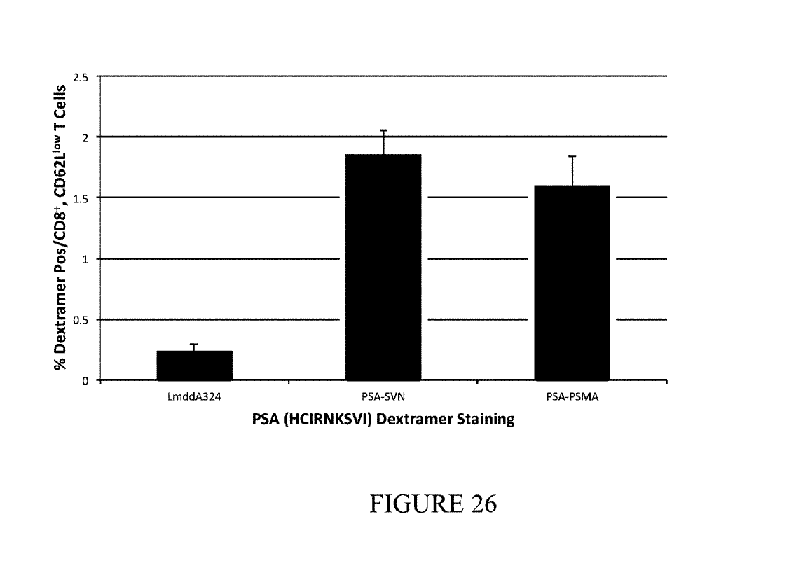

Three groups of 5 C57BL/6 mice/group were immunized iv with LmddA expressing SIINFEKL minigene, PSA-Survivin-SIINFEKL-His or PSA-PSMA-SIINFEKL-His. Splenocytes were harvested at day 8 following immunization. Splenocytes were screened for responses to H-2 D^{b} PSA₆₅₋₇₃ (HCIRNKSVI) (SEQ ID NO: 80) and H-2 K^{b} OVA257-264 (SIINFEKL) (SEQ ID NO: 81) by dextramer staining. The CD8⁺ T cell responses following primary immunization to the PSA and SIINFEKL peptides are shown in FIGS. 26 & 27, respectively.

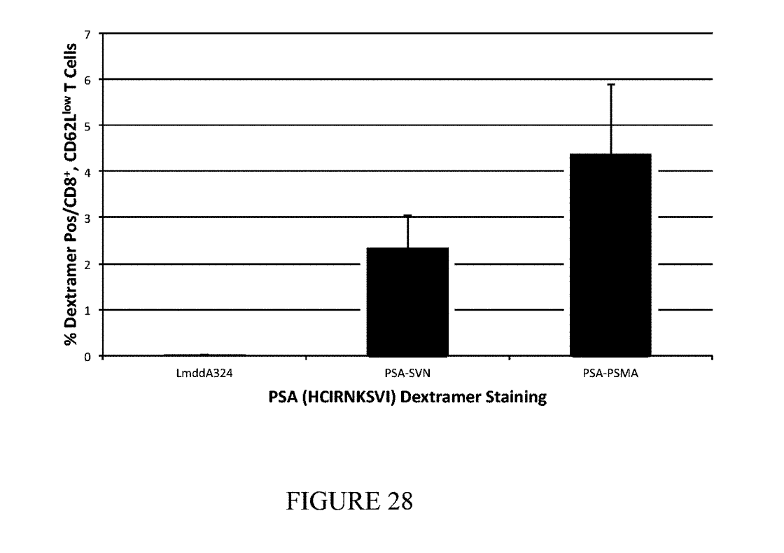

An additional three groups of 5 C57BL/6 mice/group were immunized iv with LmddA expressing SIINFEKL minigene, PSA-Survivin-SIINFEKL-His or PSA-PSMA-SIINFEKL-His, followed by two booster immunizations at two week intervals following primary immunization. Splenocytes were harvested at day 7 following the second, final booster immunization. Splenocytes were screened for responses to H-2 D^{b} PSA₆₅₋₇₃ (HCIRNKSVI) and H-2 K^{b} OVA₂₅₇₋₂₆₄ (SIINFEKL) by dextramer staining. The CD8⁺ T cell responses following primary immunization to the PSA and SIINFEKL peptides are shown in FIGS. 28 & 29, respectively.

A measurable CD8⁺ T cell response to the D^{b} restricted PSA₆₅₋₇₃ peptide was generated in mice following a single primary immunization with both the PSA-Survivin and PSA-PSMA constructs (FIG. 26). Booster immunization resulted in an increased percentage of CD8⁺ T cells in the spleens of immunized mice, with a greater relative increase observed in the PSA-PSMA immunized mice compared to those immunized with the shorter PSA-Survivin Lm (FIG. 28).

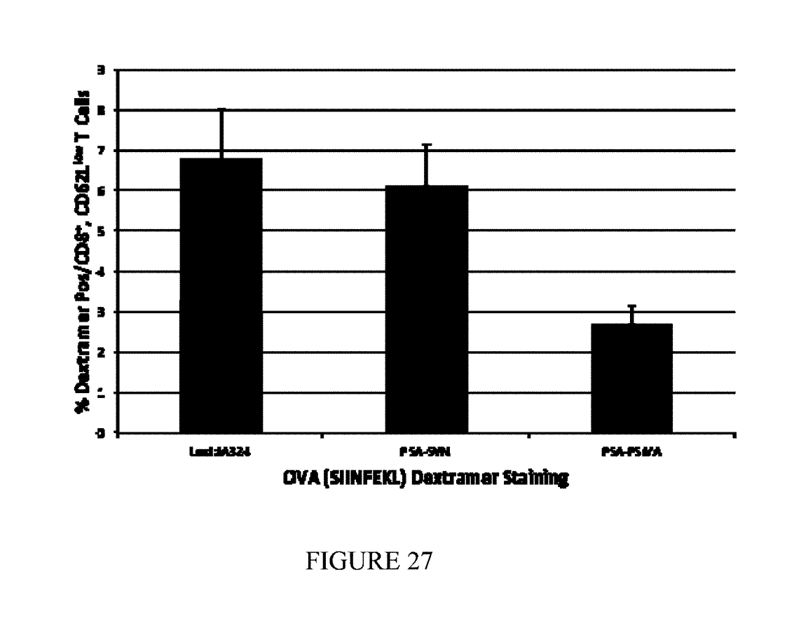

The T cell response to the K^{b} OVA₂₅₇₋₂₆₄ peptide was greater than the response to the PSA₆₅₋₇₃ peptide in mice after primary immunization with either PSA containing Lm vector (FIGS. 26 & 27). Secondary responses to the K^{b} OVA₂₅₇₋₂₆₄ peptide increased by a significantly greater degree compared to the increase seen to the PSA₆₅₋₇₃ peptide following secondary immunization with either PSA containing Lm strain (FIGS. 27 & 29). Groups of mice immunized with the OVA₂₅₇₋₂₆₄ minigene construct were included as a positive control for the response to the K^{b} restricted OVA₂₅₇₋₂₆₄ peptide.

The sequences associated with this example are set forth in SEQ ID NOS: 80-119.

### Example 18: Measuring Expression, Processing and Presentation of OVA₂₅₇₋₂₆₄ (SIINFEKL) Containing Listeria Strain Constructs Expressing PSA and an Additional Tumor-Associated Antigen (PSA 2.0 Constructs) Using an In Vitro Cell Based Assay

### Strain Construction

The DNA sequences encoding the 8 designed XhoI site-PSA (SEQ ID NO: 121; encoded amino acid sequence: SEQ ID NO: 159) plus antigen (e.g. survivin (SEQ ID NO: 122; encoded amino acid sequence: SEQ ID NO: 160) or AKAP4 (SEQ ID NO: 127; encoded amino acid sequence: SEQ ID NO: 165) or Hepsin (SEQ ID NOS: 125, 126; encoded amino acid sequences: SEQ ID NOS: 163, 164) or PSGR (SEQ ID NOS: 123, 124; encoded amino acid sequences SEQ ID NOS: 161, 162)) -XmaI site were synthesized by Genewiz, Inc. (South Plainfield, N.J.) and shipped to Advaxis in carrier plasmid pUC57-Kan. Examples of DNA sequences encoding PSA-antigen fusions are set forth in SEQ ID NOS: 130-137 (amino acid sequence set forth in SEQ ID NOS: 168-175). An exemplary linker sequence linking PSA to another antigen or linking two antigens is set forth in SEQ ID NO: 128 (amino acid sequence set forth in SEQ ID NO: 166). In exemplary SIINFEKL-6×His tag-encoding sequence is set forth in SEQ ID NO: 129 (amino acid sequence set forth in SEQ ID NO: 167). Each carrier plasmid was transformed into chemically competent Top 10 E. coli (Invitrogen) for storage and to allow mass plasmid preparation. The synthesized inserts were cloned into pAdv134 via homologous recombination using the In-Fusion Cloning kit (Clontech) to generate the 8 distinct pAdv134-PSA-antigen target plasmids. These plasmids were transfected into LmddA to generate the 8 distinct LmddA constructs and were then evaluated for tLLO-PSA-antigen target-tags fusion protein expression by SIINFEKL presentation assay. Examples of DNA sequences encoding tLLO-PSA-antigen fusion proteins are set forth in SEQ ID NOS: 138-145 (amino acid sequence set forth in SEQ ID NOS: 176-183). An exemplary tLLO-encoding sequence is set forth in SEQ ID NO: 120 (amino acid sequence set forth in SEQ ID NO: 158). Strains that express the constructs were passaged in vivo through mice twice and protein expression reverified to create the final immunotherapy strains. Primer sequences used in this example are set forth in SEQ ID NOS: 146-157.

### SIINFEKL Presentation Assay

This assay was developed to allow for the rapid in vitro determination of antigen secretion by Lm and MHC class I presentation of antigen by the infected cells. Briefly, a murine dendritic-like cell line (DC2.4) was infected with the appropriate Lm strain at an MOI of 20. Gentamicin was added after 1 h to kill any extracellular bacteria not taken up by the DC2.4 cells. Cells were incubated at 37° C. for an additional 4-5 h. Cells were then stained with Alexa Fluor 647 conjugated 25D-1.16 antibody. The 25D-1.16 antibody binds only to cell surface MHC class I K^{b} molecules presenting the OVA₂₅₇₋₂₆₄ SIINFEKL peptide. In this assay, only those cells infected with Lm secreting proteins containing the SIINFEKL peptide motif are positive for staining with the 25D-1.16 antibody. Surface staining for K^{b-}SIINFEKL is linear and, therefore, is used as a semi-quantitative measurement of antigen expression in Lm infected cells.

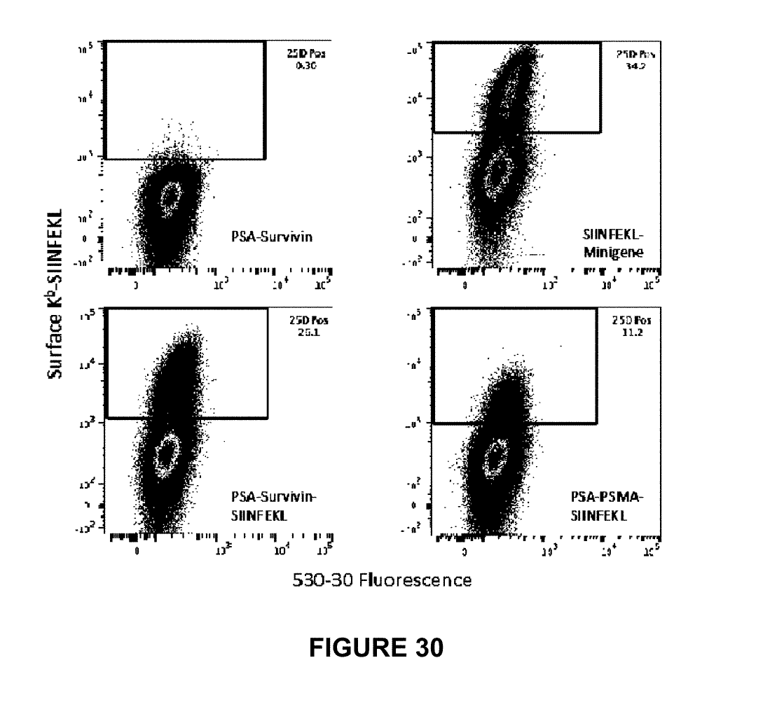

In this assay, DC2.4 were infected with four PSA-expressing Lm vectors. One construct has a PSA-Survivin without a SIINFEKL moiety at the carboxy-terminus. This construct was included as a negative control. Two Lm constructs expressing either PSA-Survivin-SIINFEKL or PSA-PSMA-SIINFEKL were used to evaluate antigen expression using the 25D-1.16 antibody system. An Lm strain expressing only the minimal SIINFEKL peptide, without a PSA 2.0 related moiety was included as a positive control. The results for these four constructs are shown in FIG. 30. Staining for 25D-1.16 is shown on the Y-axis. The relative percentage of cells expressing surface K^{b}—SIINFEKL is shown in the upper right corner of the pseudocolor plots. The mean fluorescence intensity (MFI) values for the K^{b-}SIINFEKL positive populations of the three SIINFEKL containing constructs are as follows:

| | Mean fluorescence |
|---|---|
| Construct | intensity (MFI) |
| PSA-PSMA-SIINFEKL | 2045 |
| PSA-Survivin-SIINFEKL | 4783 |
| PSA-SIINFEKL minigene | 14852 |

The average MFI value for K^{b}-SIINFEKL negative populations is 300.

The sequences associated with this example are set forth in SEQ ID NOS: 120-183.

### Example 19: Construction of ADXS31-2142 (PSA 2.0)

### 1. Introduction.

The ADXS31-2142 immunotherapy is based on Lm Δ dal dat actA (LmddA), which expresses four human antigens (Prostate Specific Antigen (PSA), Survivin, Prostate specific G-protein coupled receptor (PSGR) ATM (transmembrane domain), and Hepsin ATM), fused to a truncated fragment of the listerial listeriolysin O (tLLO) and a C-terminal SIINFEKL-6×HIS epitope tag for downstream characterization. A description of the backbone strain LmddA is provided in the previous examples.

### 2. Methods.

2.1. Construction of pAdv2142 Plasmid.

Plasmid pAdv2142 was constructed by modification of the plasmid pAdv134 described in the previous examples. Wallecha, A., Construction of an attenuated Listeria monocytogenes-based vaccine expressing human prostate specific antigen (PSA) ADXS31-142., in RPT-RD-0012011. p. 18. Primer pair Adv710/Adv711 were annealed and purified to produce the multi-cloning site (MCS) insert (Table 9). pAdv134 and MCS insert were then XhoI/XmaI restriction digested, the resultant ˜5.6 kb pAdv134 fragment and MCS restriction products were purified and ligated to generate plasmid pAdv134-MCS (SEQ ID NO: 192; XbaI restriction site at residues 2418-2423, and XmaI restriction site at residues 2448-2453). Insertion of the MCS insert into pAdv134-MCS was then confirmed by DNA sequencing of the tLLO-MCS junction using primer Adv16.

An insert encoding a fusion protein containing the four antigenic targets PSA, Survivin, PSGRΔTMs (i.e., PSGR with transmembrane domains removed), and HepsinΔTM (i.e., hepsin with transmembrane domains removed) with a C-terminal SIINFEKL-6×HIS tag was chemically synthesized and provided in carrier plasmid pUC57kan. The nucleic acid sequence for the insert is set forth in SEQ ID NO: 200 (XbaI site at residues 1-6, XmaI site at residues 2983-2988). The nucleic acid sequence for the insert in the carrier plasmid pUC57kan is set forth in SEQ ID NO: 201 (XbaI site at residues 419-424, XmaI site at residues 3401-3406, and insert at residues 419-3406. See also SEQ ID NOS: 192-202 for antigenic target and fusion insert DNA sequences. Plasmids pAdv134-MCS and pUC57 kan-insert were XbaI/XmaI restriction digested, the resultant ˜5.6 kb pAdv134-MCS fragment and ˜2.9 kb pUC57 kan-insert restriction products were then purified and ligated to generated plasmid pAdv2142 (SEQ ID NO: 202). Generation of the desired tLLO-PSA/Survivin/PSGRΔTMs/HepsinΔTM-SIINFEKL-6×HIS antigenic fusion protein expression cassette in pAdv2142 (residues 1077-5399 of SEQ ID NO: 202) was confirmed by DNA sequencing using construct/insert junction-specific and insert-specific primers (Table 10).

TABLE 9
Sequences of Primers Used in Generation of
pAdv134-MCS.

| | | SEQ ID |
|---|---|---|
| Oligo | DNA sequence (5′-3′) | NO. |
| Adv710 | GATCCTCGAGGAGCTCCTGCAGTCTAGAGTCGA | 184 |
| | CACTAGTGGATCCAGATCTCCCGGGGATC | |
| Adv711 | GATCCCCGGGAGATCTGGATCCACTAGTGTCGA | 185 |
| | CTCTAGACTGCAGGAGCTCCTCGAGGATC | |

TABLE 10
Sequences of Primers Used for pAdv134-MCS and pAdv2142
Sequencing.

| Oligo | DNA Sequence (5′-3′) | Target Region | SEQ ID NO. |
|---|---|---|---|
| Adv16 | CATCGATCACTCTGGA | pAdv134-MCS | 186 |
| Adv295 | CTAACTCCAATGTTACTTG | pAdv134-MCS | 187 |
| Adv774 | CCTGGCAGCCCTTTCTCAAG | Survivin | 188 |
| Adv786 | GCAGCATTGAACCAGAGGAG | PSA | 189 |
| Adv827 | CGAGAGATTAGCTTTGAGGCCTG | PSGR | 190 |
| Adv828 | GAGGCCGTTTCTTGGCCG | Hepsin | 191 |

2.2. Construction of ADXS31-2142 Immunotherapy.

In order to generate a PSA/Survivin/PSGR/Hepsin-targeting L. monocytogenes immunotherapy strain, plasmid pAdv2142 was transformed into Listeria monocytogenes strain LmddA via electroporation and transformants were selected on BHI-streptomycin agar plates to generate strain ADXS31-2142. Successful maintenance of pAdv2142 by ADXS31-2142 was then confirmed by colony PCR with primer pair Adv16/Adv295, resulting in the expected ˜3 kb band. The sequence of the tLLO-PSA/Survivin/PSGRΔTMs/HepsinΔTM-SIINFEKL-6×HIS antigenic fusion protein region of the plasmid in strain ADXS31-2142 was additionally confirmed.

2.3. In Vitro Evaluation of ADXS31-2142 Immunogenicity.

In order to evaluate the immunogenicity, 2×10⁶ DC2.4 murine dendritic cells were infected with ADXS31-2142 at a multiplicity of infection of 20. At one hour post-infection, host cells were washed and tissue culture medium containing gentamycin was added to kill all extracellular bacteria. At five hours post-infection host cells were harvested, stained with Alexa 647-conjugated 25D1-1.16 antibody to determine the population of cells presenting SIINFEKL epitope complexed with class I MHC, the percentage of Alexa647-positive host cells was assessed by flow cytometry versus DC2.4 cells infected with control bacterial strains.

### 3. Results.

3.1. Construction of the Antibiotic-Free Plasmid pAdv2142.

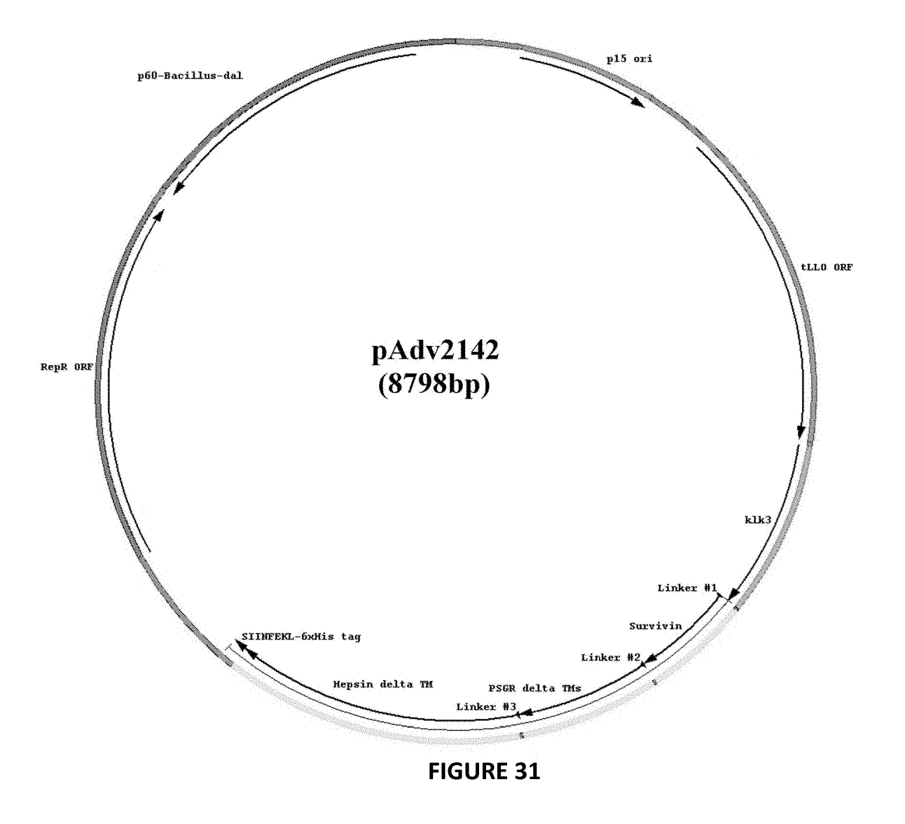

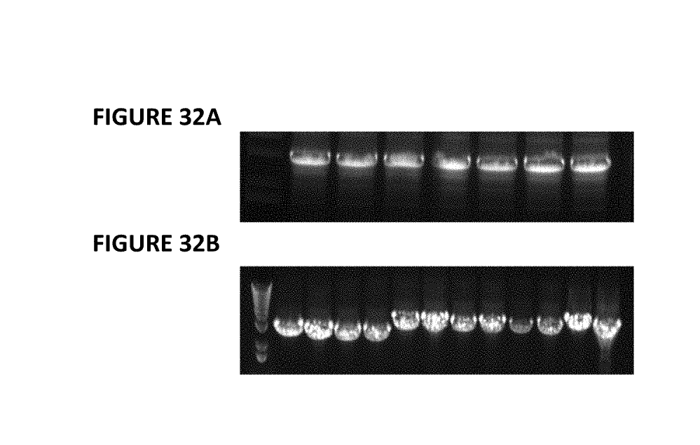

In order to generate an improved prostate cancer-targeting Lm immunotherapy strain, a tLLO-antigen fusion protein expression plasmid encoding antigenic targets in addition to PSA was desired for incorporation in Lm strain LmddA. Survivin, PGSR, and Hepsin were selected as additional antigenic target proteins as like PSA they are also differentially expressed in prostate cancer versus normal tissue. A DNA sequence encoding a PSA-Survivin-PSGRΔTMs-HepsinΔTM-SIINFEKL-6×HIS protein insert flanked by unique XbaI and XmaI restriction sites was chemically synthesized and ligated into these restriction sites in pAdv134-MCS to generate pAdv2142 (FIG. 31). Proper insertion of the insert sequence in pAdv2142 was verified by colony PCR of putative MB2159+pAdv2142 transformants (FIG. 32A) using primer pair Adv16/Adv295, with positive transformant colonies producing PCR products with the expected size of ˜3 kb corresponding to the PSA-Survivin-PSGRΔTM-HepsinΔTM-SIINFEKL-6×HIS insert. The correct tLLO-PSA-Survivin-PSGRΔTM-HepsinΔTM-SIINFEKL-6×HIS ORF was then confirmed by DNA sequencing. In pAdv2142, expression of the tLLO-PSA-Survivin-PSGRΔTM-HepsinΔTM-SIINFEKL-6×HIS fusion protein is under the control of the Lm hly promotor. The inclusion of the C-terminal SIINFEKL-6×HISepitope tag allows the assessment of tLLO-PSA-Survivin-PSGRΔTM-HepsinΔTM-SIINFEKL-6×HIS fusion protein expression by either measurement of SIINFEKL epitope presentation during in vitro DC2.4 dendritic cell infection or by anti-6×HIS western blot of TCA-precipitated bacterial culture supernatants. Additional components of the pAdv2142 plasmid include the constitutive dalbs expression cassette for plasmid selection in D-alanine auxotrophic E. coli strain MB2159 and Lm strain LmddA, Gram-negative origin of replication (p15 ori) and Gram-positive origin of replication (RepR) for maintenance of plasmid in E. coli and Lm, respectively.

To generate the PSA/Survivin/PSGR/Hepsin-targeting immunotherapy strain, pAdv2142 was the transformed into electrocompetent LmddA. Positive LmddA+pAdv2142 transformants were then identified by colony PCR using primer pair Adv16/Adv295 (FIG. 32B) and a positive clone was selected and named ADXS31-2142. Resequencing of the PSA-Survivin-PSGRΔTM-HepsinΔTM-SIINFEKL-6×HIS insert sequence from the pAdv2142 plasmid repurified from ADXS31-2142 confirmed the presence of the correct sequence, demonstrating that the tLLO-PSA-Survivin-PSGRΔTM-HepsinΔTM-SIINFEKL-6×HIS fusion protein expression cassette is genetically stable in this strain.

3.2. In vitro Expression/Secretion of tLLO-PSA-Survivin-PSGRΔTM-HepsinΔTM-SIINFEKL-6×HIS Fusion Protein by ADXS31-2142.

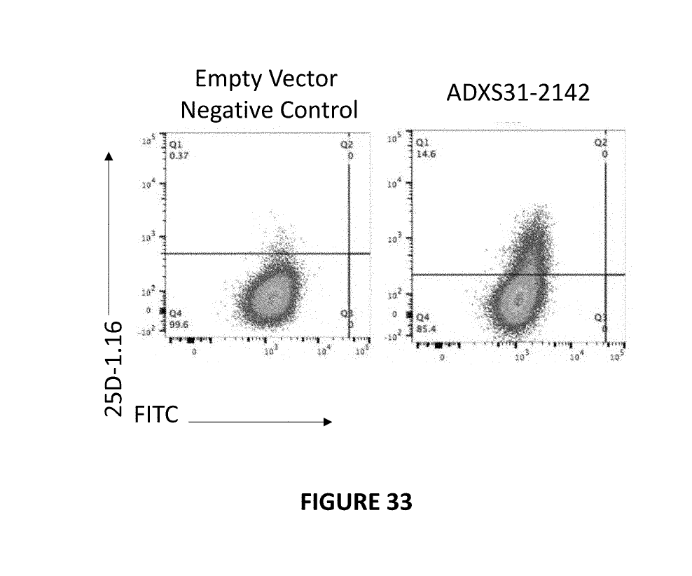

The expression and secretion of tLLO-PSA-Survivin-PSGRΔTMs-HepsinΔTM-SIINFEKL-6×HIS fusion protein by ADXS31-2142 was confirmed by an in vitro infection/flow cytometry assay. The inclusion of the eight amino acid SIINFEKL moiety in the antigenic protein allows the use of this epitope as a surrogate for the expression, secretion, proteosomal processing, and presentation of the entire protein. The commercially available 25D-1.16 antibody specifically detects the SIINFEKL peptide bound to murine H-2K^{B}. Porgador, A., et al., Localization, quantitation, and in situ detection of specific peptide-MHC class I complexes using a monoclonal antibody. Immunity, 1997. 6(6): p. 715-26. Placement of the SIINFEKL moiety at the C-terminus of the antigenic protein ensures that the entire protein must be expressed and secreted by the bacterium and available for host cell antigen presentation to enable 25D-1.16 antibody binding to ADXS31-2142-infected host cells. By infecting the H-2K^{b}-expressing dendritic cell line in vitro with ADXS31-2142 and staining infected host cells with Alexa647-conjugated 25D-1.16 antibody, tLLO-PSA-Survivin-PSGRΔTMs-HepsinΔTM-SIINFEKL-6×HIS fusion protein expression and secretion can then be measured by flow cytometry. We observed a positive 25D-1.16 signal in the DC2.4 cells infected with the ADXS31-2142, but not in the DC2.4 cells infected with a non-SIINFEKL-expressing control strain (FIG. 33). This indicates that ADXS31-2142 successfully expresses and secretes the tLLO-PSA-Survivin-PSGRΔTMs-HepsinΔTM-SIINFEKL-6×HIS fusion protein into the host cell cytosol and immunogenically competent.

In addition to the ability to test tLLO-PSA-Survivin-PSGRΔTMs-HepsinΔTM-SIINFEKL-6×HIS protein expression/secretion by the above in vitro SIINFEKL presentation assay, antigenic fusion protein expression and secretion may also be assessed by Western blot analysis of TCA-precipitated ADXS31-2142 culture supernatants using antibodies against several of the protein regions. While untested, a band of ˜157 kDa would be expected following Western blot of ADXS31-2142 culture supernatants probed with anti-LLO, anti-PSA, anti-Survivin, and anti-6×HIS antibodies.

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A recombinant Listeria strain comprising a nucleic acid molecule comprising a first open reading frame encoding a fusion polypeptide, the fusion polypeptide comprising a truncated listeriolysin O (LLO), a truncated ActA, or a PEST amino acid sequence fused to a prostate specific antigen (PSA) or an immunogenic fragment thereof, a survivin antigen or an immunogenic fragment thereof, a prostate specific G-protein coupled receptor (PSGR) antigen or an immunogenic fragment thereof, and a hepsin antigen or an immunogenic fragment thereof.

2. The recombinant Listeria strain of claim 1, wherein the PSGR antigen or immunogenic fragment thereof is a PSGRΔtransmembrane domain (ΔTM) antigen, and the hepsin antigen or immunogenic fragment thereof is a hepsinΔTM antigen.

3. The recombinant Listeria strain of claim 2, wherein the PSA or immunogenic fragment thereof, the survivin antigen or immunogenic fragment thereof, the PSGRΔTM antigen or immunogenic fragment thereof, and the hepsinΔTM antigen or immunogenic fragment thereof are in the following order from N-terminal to C-terminal: PSA-survivin-PSGRΔTM-hepsinΔTM.

4. The recombinant Listeria strain of claim 3, wherein the truncated LLO (tLLO), the truncated ActA, or the PEST amino acid sequence is fused to the PSA or immunogenic fragment thereof.

5. The recombinant Listeria strain claim 4, wherein the fusion polypeptide comprises from N-terminal to C-terminal: tLLO-PSA-survivin-PSGRΔTM-hepsinΔTM.

6. The recombinant Listeria strain of claim 5, wherein the PSA or immunogenic fragment thereof is linked to the survivin or immunogenic fragment thereof by a first linker, the survivin or immunogenic fragment thereof is linked to the PSGRΔTM or immunogenic fragment thereof via a second linker, and the PSGRΔTM or immunogenic fragment thereof is linked to the hepsinΔTM or immunogenic fragment thereof via a third linker.

7. The recombinant Listeria strain of claim 1, wherein the PSA or immunogenic fragment thereof comprises an amino acid sequence having at least 90% sequence identity with SEQ ID NO: 108.

8. The recombinant Listeria strain of claim 1, wherein the survivin antigen or immunogenic fragment thereof comprises an amino acid sequence having at least 90% sequence identity with SEQ ID NO: 109.

9. The recombinant Listeria strain of claim 1, wherein the PSGR antigen or immunogenic fragment thereof comprises an amino acid sequence having at least 90% sequence identity with SEQ ID NO: 162.

10. The recombinant Listeria strain of claim 1, wherein the hepsin antigen or immunogenic fragment thereof comprises an amino acid sequence having at least 90% sequence identity with SEQ ID NO: 164.

11. The recombinant Listeria strain of claim 1, wherein the fusion polypeptide comprises an amino acid sequence having at least 90% sequence identity with residues 1-973 of SEQ ID NO: 175 or residues 1-1414 of SEQ ID NO: 183.

12. The recombinant Listeria strain of claim 1, wherein the nucleic acid molecule is operably integrated into the Listeria genome.

13. The recombinant Listeria strain of claim 1, wherein the nucleic acid molecule is in a plasmid.

14. The recombinant Listeria strain of claim 13, wherein the plasmid does not confer antibiotic resistance upon the recombinant Listeria strain and is stably maintained in the recombinant Listeria strain in the absence of antibiotic selection.

15. (canceled)

16. The recombinant Listeria strain of claim 1, wherein the recombinant Listeria strain is attenuated.

17. The recombinant Listeria strain of claim 16, wherein the attenuated Listeria strain comprises a mutation comprising an inactivation, truncation, deletion, replacement or disruption in one or more endogenous genes.

18. The recombinant Listeria strain of claim 17, wherein the one or more endogenous genes comprise an actA virulence gene.

19. The recombinant Listeria strain of claim 17, wherein the one or more endogenous genes comprise an endogenous prfA gene.

20. The recombinant Listeria strain of claim 18, wherein the one or more endogenous genes comprise a dal gene and a dat gene.

21. (canceled)

22. The recombinant Listeria strain of claim 20, wherein the nucleic acid molecule comprises a second open reading frame encoding a metabolic enzyme.

23. (canceled)

24. The recombinant Listeria strain of claim 22, wherein the metabolic enzyme is an alanine racemase enzyme or a D-amino acid transferase enzyme.

25. The recombinant Listeria strain of claim 1, wherein the fusion polypeptide is expressed from an hly promoter, a prfA promoter, an actA promoter, or a p60 promoter.

26. The recombinant Listeria strain of claim 25, wherein the fusion polypeptide is expressed from an hly promoter.

27. The recombinant Listeria strain of claim 1, wherein the nucleic acid molecule is at least 90% identical to the sequence set forth in SEQ ID NO: 202.

28. The recombinant Listeria strain of claim 1, wherein the recombinant Listeria strain is a recombinant Listeria monocytogenes strain.

29. The recombinant Listeria strain of claim 1, wherein the recombinant Listeria strain has been passaged through an animal host.

30. The recombinant Listeria strain of claim 1, wherein the recombinant Listeria strain is an auxotrophic Listeria strain.

31. The recombinant Listeria strain of claim 5, wherein the recombinant Listeria strain is an attenuated and auxotrophic Listeria monocytogenes strain comprising an inactivation, truncation, deletion, replacement or disruption in an endogenous actA gene, an endogenous dal gene, and an endogenous dat gene, and
wherein the nucleic acid molecule is in a plasmid that does not confer antibiotic resistance upon the recombinant Listeria strain and is stably maintained in the recombinant Listeria strain in the absence of antibiotic selection, and
wherein the nucleic acid molecule comprises a second open reading frame encoding a D-amino acid transferase enzyme.

32-40. (canceled)

41. The recombinant Listeria strain of claim 31, wherein the PSA or immunogenic fragment thereof comprises an amino acid sequence having at least 90% sequence identity with SEQ ID NO: 108,
the survivin antigen or immunogenic fragment thereof comprises an amino acid sequence having at least 90% sequence identity with SEQ ID NO: 109,
the PSGR antigen or immunogenic fragment thereof comprises an amino acid sequence having at least 90% sequence identity with SEQ ID NO: 162, and
the hepsin antigen or immunogenic fragment thereof comprises an amino acid sequence having at least 90% sequence identity with SEQ ID NO: 164.

42-80. (canceled)


SEQUENCE LISTING (202 sequences)

SEQ ID NO: 1 - DNA, 6733 bp, Artificial Sequence
  Synthetic
ggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagactagttctagatttatcacgtacccatttccccgcatcttttatttttttaaatactttagggaaaaatggtttttgatttgcttttaaaggttgtggtgtagactcgtctgctgactgcatgctagaatctaagtcactttcagaagcatccacaactgactctttcgccacttttctcttatttgcttttgttggtttatctggataagtaaggctttcaagctcactatccgacgacgctatggcttttcttctttttttaatttccgctgcgctatccgatgacagacctggatgacgacgctccacttgcagagttggtcggtcgactcctgaagcctcttcatttatagccacatttcctgtttgctcaccgttgttattattgttattcggacctttctctgcttttgctttcaacattgctattaggtctgctttgttcgtatttttcactttattcgatttttctagttcctcaatatcacgtgaacttacttcacgtgcagtttcgtatcttggtcccgtatttacctcgcttggctgctcttctgttttttcttcttcccattcatctgtgtttagactggaatcttcgctatctgtcgctgcaaatattatgtcggggttaatcgtaatgcagttggcagtaatgaaaactaccatcatcgcacgcataaatctgtttaatcccacttatactccctcctcgtgatacgctaatacaacctttttagaacaaggaaaattcggccttcattttcactaatttgttccgttaaaaattggattagcagttagttatcttcttaattagctaatataagaaaaaatattcatgaattattttaagaatatcacttggagaattaatttttctctaacatttgttaatcagttaaccccaactgcttcccaagcttcacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaat

SEQ ID NO: 2 - DNA, 2850 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccgtggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttattcaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgcctggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaagtcgacacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 3 - protein, 950 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIEVDTHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 4 - protein, 529 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 5 - DNA, 2586 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 6 - protein, 862 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 7 - protein, 441 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 8 - DNA, 1647 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgaggccactgagccttacaatgctgcccggccctacagcgtggccctgctcagtgtccccgaggccgcccggacggaagcagggaagccagagagcagcacccccacaggcgagccaggccccatggcatccagccctgagcccgctgtggccaagggaggcttcctgagcttccttgaggccaacatgttcagcgtcatcatccccatgtgcctggtacttctgctcctggcgctcatcctgcccctgctcttctacctccgaaaacgcaacaagacgggcaagcatgacgtccaggattacaaggatgacgacgataag

SEQ ID NO: 9 - protein, 549 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEATEPYNAARPYSVALLSVPEAARTEAGKPESSTPTGEPGPMASSPEPAVAKGGFLSFLEANMFSVIIPMCLVLLLLALILPLLFYLRKRNKTGKHDVQDYKDDDDK

SEQ ID NO: 10 - protein, 106 aa, Artificial Sequence
  Synthetic
ATEPYNAARPYSVALLSVPEAARTEAGKPESSTPTGEPGPMASSPEPAVAKGGFLSFLEANMFSVIIPMCLVLLLLALILPLLFYLRKRNKTGKHDVQDYKDDDDK

SEQ ID NO: 11 - DNA, 8317 bp, Artificial Sequence
  Synthetic
ggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaactagtctagtggtgatggtgatgatggagctcagatctgtctaagaggcagccatagggcataagctgtgtcaccagctgcaccgtggatgtcaggcagatgcccagaaggcgggagacatatggggagcccacaccagccatcacgtatgcttcgtctaagatttctttgttggctttgggggatgtgttttccctcaacactttgatggccactggaattttcacattctccccatcagggatccagatgcccttgtagactgtgccaaaagcgccagatccaagcaccttcaccttcctcagctccgtctctttcaggatccgcatctgcgcctggttgggcatcgctccgctaggtgtcagcggctccaccagctccgtttcctgcagcagtctccgcatcgtgtacttccggatcttctgctgccctcgggcgcacagctggtggcaggccaggccctcgcccacacactcgtcctctggccggttggcagtgtggagcagagcttggtgcgggttccgaaagagctggtcccagggcaccgtgtgcacgaagcagaggtgggtgttatggtggatgagggccagtccactgcccagttccctcagtgagcgcagccccagccagctgatgcccagcccttgcagggtcagcgagtaggcgccattgtgcagaattcgtccccggattacttgcaggttctggaagacgctgaggtcaggcaggctgtccggccatgctgagatgtataggtaacctgtgatctcttccagagtctcaaacacttggagctgctctggctggagcggggcagtgttggaggctgggtccccatcaaagctctccggcagaaatgccaggctcccaaagatcttcttgcagccagcaaactcctggatattcttccacaaaatcgtgtcctggtagcagagctgggggttccgctggatcaagacccctcctttcaagatctctgtgaggcttcgaagctgcagctcccgcaggcctcctggggaggcccctgtgacaggggtggtattgttcagcgggtctccattgtctagcacggccagggcatagttgtcctcaaagagctgggtgcctcgcacaatccgcagcctctgcagtgggacctgcctcacttggttgtgagcgatgagcacgtagccctgcacctcctggatatcctgcaggaaggacaggctggcattggtgggcaggtaggtgagttccaggtttccctgcaccacctggcagccctggtagaggtggcggagcatgtccaggtgggttctagatttatcacgtacccatttccccgcatcttttatttttttaaatactttagggaaaaatggtttttgatttgcttttaaaggttgtggtgtagactcgtctgctgactgcatgctagaatctaagtcactttcagaagcatccacaactgactctttcgccacttttctcttatttgcttttgttggtttatctggataagtaaggctttcaagctcactatccgacgacgctatggcttttcttctttttttaatttccgctgcgctatccgatgacagacctggatgacgacgctccacttgcagagttggtcggtcgactcctgaagcctcttcatttatagccacatttcctgtttgctcaccgttgttattattgttattcggacctttctctgcttttgctttcaacattgctattaggtctgctttgttcgtatttttcactttattcgatttttctagttcctcaatatcacgtgaacttacttcacgtgcagtttcgtatcttggtcccgtatttacctcgcttggctgctcttctgttttttcttcttcccattcatctgtgtttagactggaatcttcgctatctgtcgctgcaaatattatgtcggggttaatcgtaatgcagttggcagtaatgaaaactaccatcatcgcacgcataaatctgtttaatcccacttatactccctcctcgtgatacgctaatacaacctttttagaacaaggaaaattcggccttcattttcactaatttgttccgttaaaaattggattagcagttagttatcttcttaattagctaatataagaaaaaatattcatgaattattttaagaatatcacttggagaattaatttttctctaacatttgttaatcagttaaccccaactgcttcccaagcttcacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaat

SEQ ID NO: 12 - protein, 32 aa, Artificial Sequence
  Synthetic
KENSISSMAPPASPPASPKTPIEKKHADEIDK

SEQ ID NO: 13 - protein, 19 aa, Artificial Sequence
  Synthetic
KENSISSMAPPASPPASPK

SEQ ID NO: 14 - protein, 14 aa, Artificial Sequence
  Synthetic
KTEEQPSEVNTGPR

SEQ ID NO: 15 - protein, 28 aa, Artificial Sequence
  Synthetic
KASVTDTSEGDLDSSMQSADESTPQPLK

SEQ ID NO: 16 - protein, 20 aa, Artificial Sequence
  Synthetic
KNEEVNASDFPPPPTDEELR

SEQ ID NO: 17 - protein, 33 aa, Artificial Sequence
  Synthetic
RGGIPTSEEFSSLNSGDFTDDENSETTEEEIDR

SEQ ID NO: 18 - protein, 19 aa, Artificial Sequence
  Synthetic
RSEVTISPAETPESPPATP

SEQ ID NO: 19 - protein, 17 aa, Artificial Sequence
  Synthetic
KQNTASTETTTTNEQPK

SEQ ID NO: 20 - protein, 17 aa, Artificial Sequence
  Synthetic
KQNTANTETTTTNEQPK

SEQ ID NO: 21 - protein, 441 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 22 - protein, 416 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 23 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 24 - protein, 416 aa, Artificial Sequence; identical to SEQ ID NO: 22
  Synthetic

SEQ ID NO: 25 - DNA, 1260 bp, Artificial Sequence
  Synthetic
acccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaa

SEQ ID NO: 26 - protein, 419 aa, Artificial Sequence
  Synthetic
THLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 27 - DNA, 3798 bp, Artificial Sequence
  Synthetic
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctga

SEQ ID NO: 28 - DNA, 393 bp, Artificial Sequence
  Synthetic
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaag

SEQ ID NO: 29 - DNA, 921 bp, Artificial Sequence
  Synthetic
gccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggat

SEQ ID NO: 30 - DNA, 477 bp, Artificial Sequence
  Synthetic
aatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgaggg

SEQ ID NO: 31 - DNA, 597 bp, Artificial Sequence
  Synthetic
tacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagag

SEQ ID NO: 32 - DNA, 391 bp, Artificial Sequence
  Synthetic
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagact

SEQ ID NO: 33 - DNA, 1209 bp, Artificial Sequence
  Synthetic
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggct

SEQ ID NO: 34 - DNA, 1158 bp, Artificial Sequence
  Synthetic
cagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctt

SEQ ID NO: 35 - protein, 386 aa, Artificial Sequence
  Synthetic
QRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLL

SEQ ID NO: 36 - DNA, 2487 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagcagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctt

SEQ ID NO: 37 - protein, 829 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLL

SEQ ID NO: 38 - protein, 390 aa, Artificial Sequence
  Synthetic
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 39 - DNA, 1170 bp, Artificial Sequence
  Synthetic
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 40 - protein, 390 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 41 - protein, 200 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 42 - protein, 226 aa, Artificial Sequence
  Synthetic
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 43 - DNA, 1170 bp, Artificial Sequence
  Synthetic
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 44 - DNA, 1256 bp, Artificial Sequence
  Synthetic
gcgccaaatcattggttgattggtgaggatgtctgtgtgcgtgggtcgcgagatgggcgaataagaagcattaaagatcctgacaaatataatcaagcggctcatatgaaagattacgaatcgcttccactcacagaggaaggcgactggggcggagttcattataatagtggtatcccgaataaagcagcctataatactatcactaaacttggaaaagaaaaaacagaacagctttattttcgcgccttaaagtactatttaacgaaaaaatcccagtttaccgatgcgaaaaaagcgcttcaacaagcagcgaaagatttatatggtgaagatgcttctaaaaaagttgctgaagcttgggaagcagttggggttaactgattaacaaatgttagagaaaaattaattctccaagtgatattcttaaaataattcatgaatattttttcttatattagctaattaagaagataactaactgctaatccaatttttaacggaacaaattagtgaaaatgaaggccgaattttccttgttctaaaaaggttgtattagcgtatcacgaggagggagtataagtgggattaaacagatttatgcgtgcgatgatggtggttttcattactgccaattgcattacgattaaccccgacgtcgacccatacgacgttaattcttgcaatgttagctattggcgtgttctctttaggggcgtttatcaaaattattcaattaagaaaaaataattaaaaacacagaacgaaagaaaaagtgaggtgaatgatatgaaattcaaaaaggtggttctaggtatgtgcttgatcgcaagtgttctagtctttccggtaacgataaaagcaaatgcctgttgtgatgaatacttacaaacacccgcagctccgcatgatattgacagcaaattaccacataaacttagttggtccgcggataacccgacaaatactgacgtaaatacgcactattggctttttaaacaagcggaaaaaatactagctaaagatgtaaatcatatgcgagctaatttaatgaatgaacttaaaaaattcgataaacaaatagctcaaggaatatatgatgcggatcataaaaatccatattatgatactagtacatttttatctcatttttataatcctgatagagataatacttatttgccgggttttgctaatgcgaaaataacaggagcaaagtatttcaatcaatcggtgactgattaccgagaagggaa

SEQ ID NO: 45 - protein, 261 aa, Artificial Sequence
  Synthetic
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 46 - protein, 237 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANP

SEQ ID NO: 47 - protein, 237 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 48 - DNA, 5873 bp, Artificial Sequence
  Synthetic
ggtgtcttaggcacactggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacaagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccacttcaagcccatacccccagtcccctccatattgcaacagtcctcactcccacaccaggtccccgctccctcccacttaccccagaactttcttcccatttgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttctttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgagtccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttccccctgtcaaccctgattcccctgatctagcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgaacacacgcacgggatgggcctgggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggagctgagggaggagaattgcattgaacctggaggttgaggttgcagtgagccgagaccgtgccactgcactccagcctgggtgacagagtgagactccgcctcaaaaaaaaaaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggcaggctacctacccacttggaaacccacgccaaagccgcatctacagctgagccactctgaggcctcccctccccggcggtccccactcagctccaaagtctctctcccttttctctcccacactttatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgcccttttaccctcttccttttcccttggttctctcagttctgtatctgcccttcaccctctcacactgctgtttcccaactcgttgtctgtattttggcctgaactgtgtcttcccaaccctgtgttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgagggaaaggtgagtgggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaacacctgcgcaggggaggggagggcctgcggcacctgggggagcagagggaacagcatctggccaggcctgggaggaggggcctagagggcgtcaggagcagagaggaggttgcctggctggagtgaaggatcggggcagggtgcgagagggaacaaaggacccctcctgcagggcctcacctgggccacaggaggacactgcttttcctctgaggagtcaggaactgtggatggtgctggacagaagcaggacagggcctggctcaggtgtccagaggctgcgctggcctcctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttcctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaagtccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagttaggatggggtgtctgtgttatttgtgggatacagagatgaaagaggggtgggatcc

SEQ ID NO: 49 - protein, 238 aa, Artificial Sequence
  Synthetic
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 50 - DNA, 1906 bp, Artificial Sequence
  Synthetic
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 51 - protein, 69 aa, Artificial Sequence
  Synthetic
MWVPVVFLTLSVTWIGAAPLILSRIVGGWECEKHSQPWQVLVASRGRAVCGGVLVHPQWVLTAAHCIRK

SEQ ID NO: 52 - DNA, 554 bp, Artificial Sequence
  Synthetic
agccccaagcttaccacctgcacccggagagctgtgtcaccatgtgggtcccggttgtcttcctcacccttccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgttcctctatgtctctttgtgtcgctttcattatgtctcttggtaactggcttcggttgtgtctctccgtgtgactattttgttctctctctccctctcttctctgtcttcagt

SEQ ID NO: 53 - protein, 220 aa, Artificial Sequence
  Synthetic
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 54 - DNA, 1341 bp, Artificial Sequence
  Synthetic
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 55 - protein, 218 aa, Artificial Sequence
  Synthetic
MWVPVVFLTLSVTWIGAAPLILSRIVGGWECEKHSQPWQVLVASRGRAVCGGVLVHPQWVLTAAHCIRKPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 56 - DNA, 1325 bp, Artificial Sequence
  Synthetic
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattacccaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 57 - protein, 261 aa, Artificial Sequence; identical to SEQ ID NO: 45
  Synthetic

SEQ ID NO: 58 - DNA, 1464 bp, Artificial Sequence
  Synthetic
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 59 - protein, 261 aa, Artificial Sequence; identical to SEQ ID NO: 45
  Synthetic

SEQ ID NO: 60 - DNA, 1495 bp, Artificial Sequence
  Synthetic
gggggagccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtagggagagggactggaccccctggaagctgattcactatggggggaggtgtattgaagtcctccagacaaccctcagatttgatgatttcctagtagaactcacagaaataaagagctgttatactgcgaaaaaaaaaaaaaaaaaaaaaaaaaa

SEQ ID NO: 61 - protein, 218 aa, Artificial Sequence
  Synthetic
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVA

SEQ ID NO: 62 - protein, 227 aa, Artificial Sequence
  Synthetic
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSVSHPYSQDLEGKGEWGP

SEQ ID NO: 63 - protein, 104 aa, Artificial Sequence
  Synthetic
MWVPVVFLTLSVTWIGERGHGWGDAGEGASPDCQAEALSPPTQHPSPDRELGSFLSLPAPLQAHTPSPSILQQSSLPHQVPAPSHLPQNFLPIAQPAPCSQLLY

SEQ ID NO: 64 - protein, 261 aa, Artificial Sequence; identical to SEQ ID NO: 45
  Synthetic

SEQ ID NO: 65 - DNA, 1729 bp, Artificial Sequence
  Synthetic
aagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtagcctctcgtggcagggcagtctgcggcggtgttctggtgcacccccagtgggtcctcacagctacccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgaaccaaggagggagggtcttcctttggcatgggatggggatgaagtaaggagagggactgaccccctggaagctgattcactatggggggaggtgtattgaagtcctccagacaaccctcagatttgatgatttcctagtagaactcacagaaataaagagctgttatactgtgaa

SEQ ID NO: 66 - protein, 24 aa, Artificial Sequence
  Synthetic
MWVPVVFLTLSVTWIGAAPLILSR

SEQ ID NO: 67 - protein, 97 aa, Artificial Sequence
  Synthetic
HGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 68 - DNA, 1107 bp, Artificial Sequence
  Synthetic
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 69 - protein, 368 aa, Artificial Sequence
  Synthetic
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 70 - DNA, 870 bp, Artificial Sequence
  Synthetic
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 71 - protein, 289 aa, Artificial Sequence
  Synthetic
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 72 - DNA, 6523 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 73 - DNA, 36 bp, Artificial Sequence
  Synthetic
cggaattcggatccgcgccaaatcattggttgattg

SEQ ID NO: 74 - DNA, 37 bp, Artificial Sequence
  Synthetic
gcgagtcgacgtcggggttaatcgtaatgcaattggc

SEQ ID NO: 75 - DNA, 35 bp, Artificial Sequence
  Synthetic
gcgagtcgacccatacgacgttaattcttgcaatg

SEQ ID NO: 76 - DNA, 39 bp, Artificial Sequence
  Synthetic
gatactgcagggatccttcccttctcggtaatcagtcac

SEQ ID NO: 77 - DNA, 19 bp, Artificial Sequence
  Synthetic
tgggatggccaagaaattc

SEQ ID NO: 78 - DNA, 22 bp, Artificial Sequence
  Synthetic
ctaccatgtcttccgttgcttg

SEQ ID NO: 79 - protein, 9 aa, Artificial Sequence
  Synthetic
RLLQETELV

SEQ ID NO: 80 - protein, 9 aa, Artificial Sequence
  Synthetic
HCIRNKSVI

SEQ ID NO: 81 - protein, 8 aa, Artificial Sequence
  Synthetic
SIINFEKL

SEQ ID NO: 82 - DNA, 1323 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 83 - DNA, 711 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 84 - DNA, 423 bp, Artificial Sequence
  Synthetic
ggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggat

SEQ ID NO: 85 - DNA, 2250 bp, Artificial Sequence
  Synthetic
atgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttacataattttacacagataccacatttagcaggaacagaacaaaactttcagcttgcaaagcaaattcaatcccagtggaaagaatttggcctggattctgttgagctagctcattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacgagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 86 - DNA, 2178 bp, Artificial Sequence
  Synthetic
atgtggaatctccttcacgaaaccgactcggctgtggccaccgcgcgccgcccgcgcaaatcctccaatgaagctactaacattactccaaagcataatatgaaagcatttttggatgaattgaaagctgagaacatcaagaagttcttacataattttacacagataccacatttagcaggaacagaacaaaactttcagcttgcaaagcaaattcaatcccagtggaaagaatttggcctggattctgttgagctagctcattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacgagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 87 - DNA, 12 bp, Artificial Sequence
  Synthetic
ggtggtggaggt

SEQ ID NO: 88 - DNA, 57 bp, Artificial Sequence
  Synthetic
gcacgtagtataatcaactttgaaaaactgagtcatcatcatcatcatcattaataa

SEQ ID NO: 89 - DNA, 1152 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattaataa

SEQ ID NO: 90 - DNA, 1203 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatgcacgtagtataatcaactttgaaaaactgagtcatcatcatcatcatcattaataa

SEQ ID NO: 91 - DNA, 2958 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgtggaatctccttcacgaaaccgactcggctgtggccaccgcgcgccgcccgcgcaaatcctccaatgaagctactaacattactccaaagcataatatgaaagcatttttggatgaattgaaagctgagaacatcaagaagttcttacataattttacacagataccacatttagcaggaacagaacaaaactttcagcttgcaaagcaaattcaatcccagtggaaagaatttggcctggattctgttgagctagctcattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacgagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagccgcacgtagtataatcaactttgaaaaactgagtcatcatcatcatcatcattaataa

SEQ ID NO: 92 - DNA, 2481 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattaataa

SEQ ID NO: 93 - DNA, 2532 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatgcacgtagtataatcaactttgaaaaactgagtcatcatcatcatcatcattaataa

SEQ ID NO: 94 - DNA, 4287 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgtggaatctccttcacgaaaccgactcggctgtggccaccgcgcgccgcccgcgcaaatcctccaatgaagctactaacattactccaaagcataatatgaaagcatttttggatgaattgaaagctgagaacatcaagaagttcttacataattttacacagataccacatttagcaggaacagaacaaaactttcagcttgcaaagcaaattcaatcccagtggaaagaatttggcctggattctgttgagctagctcattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacgagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagccgcacgtagtataatcaactttgaaaaactgagtcatcatcatcatcatcattaataa

SEQ ID NO: 95 - DNA, 6138 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaactagtgactacaaggacgatgacgacaagtgatacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 96 - DNA, 6961 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattaataacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 97 - DNA, 7012 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatgcacgtagtataatcaactttgaaaaactgagtcatcatcatcatcatcattaataacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 98 - DNA, 8767 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgtggaatctccttcacgaaaccgactcggctgtggccaccgcgcgccgcccgcgcaaatcctccaatgaagctactaacattactccaaagcataatatgaaagcatttttggatgaattgaaagctgagaacatcaagaagttcttacataattttacacagataccacatttagcaggaacagaacaaaactttcagcttgcaaagcaaattcaatcccagtggaaagaatttggcctggattctgttgagctagctcattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacgagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagccgcacgtagtataatcaactttgaaaaactgagtcatcatcatcatcatcattaataacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 99 - DNA, 16 bp, Artificial Sequence
  Synthetic
catcgatcactctgga

SEQ ID NO: 100 - DNA, 19 bp, Artificial Sequence
  Synthetic
ctaactccaatgttacttg

SEQ ID NO: 101 - DNA, 20 bp, Artificial Sequence
  Synthetic
gcagcattgaaccagaggag

SEQ ID NO: 102 - DNA, 20 bp, Artificial Sequence
  Synthetic
cctggcagccctttctcaag

SEQ ID NO: 103 - DNA, 22 bp, Artificial Sequence
  Synthetic
gtggaatctccttcacgaaacc

SEQ ID NO: 104 - DNA, 21 bp, Artificial Sequence
  Synthetic
ggttaaaaatgcccagctggc

SEQ ID NO: 105 - DNA, 24 bp, Artificial Sequence
  Synthetic
caattttgtttgcaagctgggatg

SEQ ID NO: 106 - DNA, 26 bp, Artificial Sequence
  Synthetic
ctcccttttgattgtcgagattatgc

SEQ ID NO: 107 - protein, 441 aa, Artificial Sequence; identical to SEQ ID NO: 7
  Synthetic

SEQ ID NO: 108 - protein, 237 aa, Artificial Sequence; identical to SEQ ID NO: 46
  Synthetic

SEQ ID NO: 109 - protein, 141 aa, Artificial Sequence
  Synthetic
GAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 110 - protein, 750 aa, Artificial Sequence [sequence omitted]

SEQ ID NO: 111 - protein, 726 aa, Artificial Sequence
  Synthetic
MWNLLHETDSAVATARRPRKSSNEATNITPKHNMKAFLDELKAENIKKFLHNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 112 - protein, 4 aa, Artificial Sequence
  Synthetic
GGGG

SEQ ID NO: 113 - protein, 17 aa, Artificial Sequence
  Synthetic
ARSIINFEKLSHHHHHH

SEQ ID NO: 114 - protein, 382 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 115 - protein, 399 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDARSIINFEKLSHHHHHH

SEQ ID NO: 116 - protein, 984 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMWNLLHETDSAVATARRPRKSSNEATNITPKHNMKAFLDELKAENIKKFLHNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVAARSIINFEKLSHHHHHH

SEQ ID NO: 117 - protein, 825 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 118 - protein, 842 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDARSIINFEKLSHHHHHH

SEQ ID NO: 119 - protein, 1427 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMWNLLHETDSAVATARRPRKSSNEATNITPKHNMKAFLDELKAENIKKFLHNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVAARSIINFEKLSHHHHHH

SEQ ID NO: 120 - DNA, 1323 bp, Artificial Sequence; identical to SEQ ID NO: 82
  Synthetic

SEQ ID NO: 121 - DNA, 711 bp, Artificial Sequence; identical to SEQ ID NO: 83
  Synthetic

SEQ ID NO: 122 - DNA, 423 bp, Artificial Sequence; identical to SEQ ID NO: 84
  Synthetic

SEQ ID NO: 123 - DNA, 960 bp, Artificial Sequence
  Synthetic
atgagttcctgcaacttcacacatgccacctttgtgcttattggtatcccaggattagagaaagcccatttctgggttggcttccctctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagttgtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaag

SEQ ID NO: 124 - DNA, 555 bp, Artificial Sequence
  Synthetic
atgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaag

SEQ ID NO: 125 - DNA, 1251 bp, Artificial Sequence
  Synthetic
atggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctc

SEQ ID NO: 126 - DNA, 1194 bp, Artificial Sequence
  Synthetic
atggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctc

SEQ ID NO: 127 - DNA, 2562 bp, Artificial Sequence
  Synthetic
atgatggcgtactctgatactacaatgatgtctgatgatattgactggttacgcagccacaggggtgtgtgcaaggtagatctctacaacccagaaggacagcaagatcaggaccggaaagtgatatgctttgtcgatgtgtccaccctgaatgtagaagataaagattacaaggatgctgctagttccagctcagaaggcaacttaaacctgggaagtctggaagaaaaagagattatcgtgatcaaggacactgagaagaaagaccagtctaagacagagggatctgtatgccttttcaaacaagctccctctgatcctgtaagtgtcctcaactggcttctcagtgatctccagaagtatgccttgggtttccaacatgcactgagcccctcaacctctacctgtaaacataaagtaggagacacagagggcgaatatcacagagcatcctctgagaactgctacagtgtctatgccgatcaagtgaacatagattatttgatgaacagacctcaaaacctacgtctagaaatgacagcagctaaaaacaccaacaataatcaaagtccttcagctcctccagccaaacctcctagcactcagagagcagtcatttcccctgatggagaatgttctatagatgacctttccttctacgtcaaccgactatcttctctggtaatccagatggcccataaggaaatcaaggagaagttggaaggtaaaagcaaatgccttcatcattcaatctgtccatcccctgggaacaaagagagaatcagtccccgaactcctgcgagcaagattgcttctgaaatggcctatgaagctgtggaactgacagctgcagaaatgcgtggcactggagaggagtccagggaaggtggccagaaaagctttctatatagcgaattatccaacaagagcaaaagtggagacaaacagatgtcccagagagagagcaaagaatttgcagattccatcagcaaggggctcatggtttatgcaaatcaggtggcatctgacatgatggtctctctcatgaagaccttgaaagtgcacagctctgggaagccaattccagcatctgtggtcctgaagagggtgttgctaaggcacaccaaggagattgtgtccgatttgattgattcttgcatgaagaacctgcataatattactggggtcctgatgactgactcagactttgtctcagctgtcaagagaaatctgttcaaccagtggaaacaaaatgctacagacatcatggaggccatgctgaagcgcttggtcagtgcccttataggtgaggagaaggagactaagtctcagagtctgtcatatgcatctttaaaagctgggtcccatgatcccaaatgcaggaatcagagtcttgaattctccaccatgaaagctgaaatgaaagagagggacaaaggcaaaatgaaatcagacccatgcaagtcactgactagtgctgagaaagtcggtgaacacattctcaaagagggcctaaccatctggaaccaaaagcaaggaaactcatgcaaggtggctaccaaagcatgcagcaataaagatgagaaaggagaaaagatcaatgcttccacagattcactggccaaggacctgattgtctctgcccttaagctgatccagtaccatctgacccagcagactaagggcaaagatacatgtgaagaagactgtcctggttccaccatgggctatatggctcagagtactcaatatgaaaagtgtggaggtggccaaagtgccaaagcactttcagtgaaacaactagaatctcacagagcccctggaccatccacctgtcaaaaggagaaccaacacctggactcccagaaaatggatatgtcaaacatcgttctaatgctgattcagaaactgcttaatgagaaccccttcaaatgtgaggatccatgcgaaggtgagaacaagtgttctgagcccagggcaagcaaagcagcttccatgtccaacagatctgacaaagcggaagaacaatgccaggagcatcaagaacttgactgtaccagtgggatgaagcaagcgaacgggcaatttatagataaactagtagaatctgtgatgaagctctgccttatcatggctaagtatagcaacgatggggcagcccttgctgagttggaagaacaagcagcctcggcaaataagcccaatttcaggggcaccagatgcattcacagtggtgcaatgccacagaactatcaagactctcttggacatgaagtaattgtcaataatcagtgctctacaaatagcttgcagaagcagctccaggctgtcctgcagtggattgcagcctcccagtttaacgtgcccatgctctacttcatgggagataaggatggacaactggaaaagcttcctcaggtttcagctaaagcagcagagaaggggtacagtgtaggaggtcttcttcaagaggtcatgaagtttgccaaggaacggcaaccagatgaagctgtgggaaaggtggccaggaaacagttgctggactggctgctcgctaacctg

SEQ ID NO: 128 - DNA, 12 bp, Artificial Sequence
  Synthetic
ggtggtggaggt

SEQ ID NO: 129 - DNA, 57 bp, Artificial Sequence; identical to SEQ ID NO: 88
  Synthetic

SEQ ID NO: 130 - DNA, 1740 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgagttcctgcaacttcacacatgccacctttgtgcttattggtatcccaggattagagaaagcccatttctgggttggcttccctctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagttgtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaaggcacgtagtataatcaactttgaaaaactgagtcatcatcatcatcatcattaataa

SEQ ID NO: 131 - DNA, 1335 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaaggcacgtagtataatcaactttgaaaaactgagtcatcatcatcatcatcattaataa

SEQ ID NO: 132 - DNA, 1974 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 133 - DNA, 3342 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgatggcgtactctgatactacaatgatgtctgatgatattgactggttacgcagccacaggggtgtgtgcaaggtagatctctacaacccagaaggacagcaagatcaggaccggaaagtgatatgctttgtcgatgtgtccaccctgaatgtagaagataaagattacaaggatgctgctagttccagctcagaaggcaacttaaacctgggaagtctggaagaaaaagagattatcgtgatcaaggacactgagaagaaagaccagtctaagacagagggatctgtatgccttttcaaacaagctccctctgatcctgtaagtgtcctcaactggcttctcagtgatctccagaagtatgccttgggtttccaacatgcactgagcccctcaacctctacctgtaaacataaagtaggagacacagagggcgaatatcacagagcatcctctgagaactgctacagtgtctatgccgatcaagtgaacatagattatttgatgaacagacctcaaaacctacgtctagaaatgacagcagctaaaaacaccaacaataatcaaagtccttcagctcctccagccaaacctcctagcactcagagagcagtcatttcccctgatggagaatgttctatagatgacctttccttctacgtcaaccgactatcttctctggtaatccagatggcccataaggaaatcaaggagaagttggaaggtaaaagcaaatgccttcatcattcaatctgtccatcccctgggaacaaagagagaatcagtccccgaactcctgcgagcaagattgcttctgaaatggcctatgaagctgtggaactgacagctgcagaaatgcgtggcactggagaggagtccagggaaggtggccagaaaagctttctatatagcgaattatccaacaagagcaaaagtggagacaaacagatgtcccagagagagagcaaagaatttgcagattccatcagcaaggggctcatggtttatgcaaatcaggtggcatctgacatgatggtctctctcatgaagaccttgaaagtgcacagctctgggaagccaattccagcatctgtggtcctgaagagggtgttgctaaggcacaccaaggagattgtgtccgatttgattgattcttgcatgaagaacctgcataatattactggggtcctgatgactgactcagactttgtctcagctgtcaagagaaatctgttcaaccagtggaaacaaaatgctacagacatcatggaggccatgctgaagcgcttggtcagtgcccttataggtgaggagaaggagactaagtctcagagtctgtcatatgcatctttaaaagctgggtcccatgatcccaaatgcaggaatcagagtcttgaattctccaccatgaaagctgaaatgaaagagagggacaaaggcaaaatgaaatcagacccatgcaagtcactgactagtgctgagaaagtcggtgaacacattctcaaagagggcctaaccatctggaaccaaaagcaaggaaactcatgcaaggtggctaccaaagcatgcagcaataaagatgagaaaggagaaaagatcaatgcttccacagattcactggccaaggacctgattgtctctgcccttaagctgatccagtaccatctgacccagcagactaagggcaaagatacatgtgaagaagactgtcctggttccaccatgggctatatggctcagagtactcaatatgaaaagtgtggaggtggccaaagtgccaaagcactttcagtgaaacaactagaatctcacagagcccctggaccatccacctgtcaaaaggagaaccaacacctggactcccagaaaatggatatgtcaaacatcgttctaatgctgattcagaaactgcttaatgagaaccccttcaaatgtgaggatccatgcgaaggtgagaacaagtgttctgagcccagggcaagcaaagcagcttccatgtccaacagatctgacaaagcggaagaacaatgccaggagcatcaagaacttgactgtaccagtgggatgaagcaagcgaacgggcaatttatagataaactagtagaatctgtgatgaagctctgccttatcatggctaagtatagcaacgatggggcagcccttgctgagttggaagaacaagcagcctcggcaaataagcccaatttcaggggcaccagatgcattcacagtggtgcaatgccacagaactatcaagactctcttggacatgaagtaattgtcaataatcagtgctctacaaatagcttgcagaagcagctccaggctgtcctgcagtggattgcagcctcccagtttaacgtgcccatgctctacttcatgggagataaggatggacaactggaaaagcttcctcaggtttcagctaaagcagcagagaaggggtacagtgtaggaggtcttcttcaagaggtcatgaagtttgccaaggaacggcaaccagatgaagctgtgggaaaggtggccaggaaacagttgctggactggctgctcgctaacctggcacgtagtataatcaactttgaaaaactgagtcatcatcatcatcatcattaataa

SEQ ID NO: 134 - DNA, 1770 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaaggcacgtagtataatcaactttgaaaaactgagtcatcatcatcatcatcattaataa

SEQ ID NO: 135 - DNA, 2409 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 136 - DNA, 2541 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 137 - DNA, 2976 bp, Artificial Sequence
  Synthetic
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 138 - DNA, 3063 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgagttcctgcaacttcacacatgccacctttgtgcttattggtatcccaggattagagaaagcccatttctgggttggcttccctctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagttgtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaaggcacgtagtataatcaactttgaaaaactgagtcatcatcatcatcatcattaataa

SEQ ID NO: 139 - DNA, 2658 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaaggcacgtagtataatcaactttgaaaaactgagtcatcatcatcatcatcattaataa

SEQ ID NO: 140 - DNA, 3297 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 141 - DNA, 4665 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgatggcgtactctgatactacaatgatgtctgatgatattgactggttacgcagccacaggggtgtgtgcaaggtagatctctacaacccagaaggacagcaagatcaggaccggaaagtgatatgctttgtcgatgtgtccaccctgaatgtagaagataaagattacaaggatgctgctagttccagctcagaaggcaacttaaacctgggaagtctggaagaaaaagagattatcgtgatcaaggacactgagaagaaagaccagtctaagacagagggatctgtatgccttttcaaacaagctccctctgatcctgtaagtgtcctcaactggcttctcagtgatctccagaagtatgccttgggtttccaacatgcactgagcccctcaacctctacctgtaaacataaagtaggagacacagagggcgaatatcacagagcatcctctgagaactgctacagtgtctatgccgatcaagtgaacatagattatttgatgaacagacctcaaaacctacgtctagaaatgacagcagctaaaaacaccaacaataatcaaagtccttcagctcctccagccaaacctcctagcactcagagagcagtcatttcccctgatggagaatgttctatagatgacctttccttctacgtcaaccgactatcttctctggtaatccagatggcccataaggaaatcaaggagaagttggaaggtaaaagcaaatgccttcatcattcaatctgtccatcccctgggaacaaagagagaatcagtccccgaactcctgcgagcaagattgcttctgaaatggcctatgaagctgtggaactgacagctgcagaaatgcgtggcactggagaggagtccagggaaggtggccagaaaagctttctatatagcgaattatccaacaagagcaaaagtggagacaaacagatgtcccagagagagagcaaagaatttgcagattccatcagcaaggggctcatggtttatgcaaatcaggtggcatctgacatgatggtctctctcatgaagaccttgaaagtgcacagctctgggaagccaattccagcatctgtggtcctgaagagggtgttgctaaggcacaccaaggagattgtgtccgatttgattgattcttgcatgaagaacctgcataatattactggggtcctgatgactgactcagactttgtctcagctgtcaagagaaatctgttcaaccagtggaaacaaaatgctacagacatcatggaggccatgctgaagcgcttggtcagtgcccttataggtgaggagaaggagactaagtctcagagtctgtcatatgcatctttaaaagctgggtcccatgatcccaaatgcaggaatcagagtcttgaattctccaccatgaaagctgaaatgaaagagagggacaaaggcaaaatgaaatcagacccatgcaagtcactgactagtgctgagaaagtcggtgaacacattctcaaagagggcctaaccatctggaaccaaaagcaaggaaactcatgcaaggtggctaccaaagcatgcagcaataaagatgagaaaggagaaaagatcaatgcttccacagattcactggccaaggacctgattgtctctgcccttaagctgatccagtaccatctgacccagcagactaagggcaaagatacatgtgaagaagactgtcctggttccaccatgggctatatggctcagagtactcaatatgaaaagtgtggaggtggccaaagtgccaaagcactttcagtgaaacaactagaatctcacagagcccctggaccatccacctgtcaaaaggagaaccaacacctggactcccagaaaatggatatgtcaaacatcgttctaatgctgattcagaaactgcttaatgagaaccccttcaaatgtgaggatccatgcgaaggtgagaacaagtgttctgagcccagggcaagcaaagcagcttccatgtccaacagatctgacaaagcggaagaacaatgccaggagcatcaagaacttgactgtaccagtgggatgaagcaagcgaacgggcaatttatagataaactagtagaatctgtgatgaagctctgccttatcatggctaagtatagcaacgatggggcagcccttgctgagttggaagaacaagcagcctcggcaaataagcccaatttcaggggcaccagatgcattcacagtggtgcaatgccacagaactatcaagactctcttggacatgaagtaattgtcaataatcagtgctctacaaatagcttgcagaagcagctccaggctgtcctgcagtggattgcagcctcccagtttaacgtgcccatgctctacttcatgggagataaggatggacaactggaaaagcttcctcaggtttcagctaaagcagcagagaaggggtacagtgtaggaggtcttcttcaagaggtcatgaagtttgccaaggaacggcaaccagatgaagctgtgggaaaggtggccaggaaacagttgctggactggctgctcgctaacctggcacgtagtataatcaactttgaaaaactgagtcatcatcatcatcatcattaataa

SEQ ID NO: 142 - DNA, 3093 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaaggcacgtagtataatcaactttgaaaaactgagtcatcatcatcatcatcattaataa

SEQ ID NO: 143 - DNA, 3732 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 144 - DNA, 3864 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 145 - DNA, 4299 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataa

SEQ ID NO: 146 - DNA, 16 bp, Artificial Sequence
  Synthetic
catcgatcactctgga

SEQ ID NO: 147 - DNA, 19 bp, Artificial Sequence
  Synthetic
ctaactccaatgttacttg

SEQ ID NO: 148 - DNA, 20 bp, Artificial Sequence
  Synthetic
gcagcattgaaccagaggag

SEQ ID NO: 149 - DNA, 20 bp, Artificial Sequence
  Synthetic
cctggcagccctttctcaag

SEQ ID NO: 150 - DNA, 23 bp, Artificial Sequence
  Synthetic
cgagagattagctttgaggcctg

SEQ ID NO: 151 - DNA, 18 bp, Artificial Sequence
  Synthetic
gaggccgtttcttggccg

SEQ ID NO: 152 - DNA, 27 bp, Artificial Sequence
  Synthetic
ccagtctaagacagagggatctgtatg

SEQ ID NO: 153 - DNA, 22 bp, Artificial Sequence
  Synthetic
ggcctatgaagctgtggaactg

SEQ ID NO: 154 - DNA, 24 bp, Artificial Sequence
  Synthetic
ctagtgctgagaaagtcggtgaac

SEQ ID NO: 155 - DNA, 23 bp, Artificial Sequence
  Synthetic
ctgtaccagtgggatgaagcaag

SEQ ID NO: 156 - DNA, 41 bp, Artificial Sequence
  Synthetic
gaagtaaattatgatctcgagattgtgggaggctgggagtg

SEQ ID NO: 157 - DNA, 71 bp, Artificial Sequence
  Synthetic
gattaaatccactactagcgttgagttagtggcccgggttattaatgatgatgatgatgatgactcagttt

SEQ ID NO: 158 - protein, 441 aa, Artificial Sequence; identical to SEQ ID NO: 7
  Synthetic

SEQ ID NO: 159 - protein, 237 aa, Artificial Sequence; identical to SEQ ID NO: 46
  Synthetic

SEQ ID NO: 160 - protein, 141 aa, Artificial Sequence; identical to SEQ ID NO: 109
  Synthetic

SEQ ID NO: 161 - protein, 320 aa, Artificial Sequence
  Synthetic
MSSCNFTHATFVLIGIPGLEKAHFWVGFPLLSMYVVAMFGNCIVVFIVRTERSLHAPMYLFLCMLAAIDLALSTSTMPKILALFWFDSREISFEACLTQMFFIHALSAIESTILLAMAFDRYVAICHPLRHAAVLNNTVTAQIGIVAVVRGSLFFFPLPLLIKRLAFCHSNVLSHSYCVHQDVMKLAYADTLPNVVYGLTAILLVMGVDVMFISLSYFLIIRTVLQLPSKSERAKAFGTCVSHIGVVLAFYVPLIGLSVVHRFGNSLHPIVRVVMGDIYLLLPPVINPIIYGAKTKQIRTRVLAMFKISCDKDLQAVGGK

SEQ ID NO: 162 - protein, 185 aa, Artificial Sequence
  Synthetic
MSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGK

SEQ ID NO: 163 - protein, 417 aa, Artificial Sequence
  Synthetic
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 164 - protein, 398 aa, Artificial Sequence
  Synthetic
MAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 165 - protein, 854 aa, Artificial Sequence
  Synthetic
MMAYSDTTMMSDDIDWLRSHRGVCKVDLYNPEGQQDQDRKVICFVDVSTLNVEDKDYKDAASSSSEGNLNLGSLEEKEIIVIKDTEKKDQSKTEGSVCLFKQAPSDPVSVLNWLLSDLQKYALGFQHALSPSTSTCKHKVGDTEGEYHRASSENCYSVYADQVNIDYLMNRPQNLRLEMTAAKNTNNNQSPSAPPAKPPSTQRAVISPDGECSIDDLSFYVNRLSSLVIQMAHKEIKEKLEGKSKCLHHSICPSPGNKERISPRTPASKIASEMAYEAVELTAAEMRGTGEESREGGQKSFLYSELSNKSKSGDKQMSQRESKEFADSISKGLMVYANQVASDMMVSLMKTLKVHSSGKPIPASVVLKRVLLRHTKEIVSDLIDSCMKNLHNITGVLMTDSDFVSAVKRNLFNQWKQNATDIMEAMLKRLVSALIGEEKETKSQSLSYASLKAGSHDPKCRNQSLEFSTMKAEMKERDKGKMKSDPCKSLTSAEKVGEHILKEGLTIWNQKQGNSCKVATKACSNKDEKGEKINASTDSLAKDLIVSALKLIQYHLTQQTKGKDTCEEDCPGSTMGYMAQSTQYEKCGGGQSAKALSVKQLESHRAPGPSTCQKENQHLDSQKMDMSNIVLMLIQKLLNENPFKCEDPCEGENKCSEPRASKAASMSNRSDKAEEQCQEHQELDCTSGMKQANGQFIDKLVESVMKLCLIMAKYSNDGAALAELEEQAASANKPNFRGTRCIHSGAMPQNYQDSLGHEVIVNNQCSTNSLQKQLQAVLQWIAASQFNVPMLYFMGDKDGQLEKLPQVSAKAAEKGYSVGGLLQEVMKFAKERQPDEAVGKVARKQLLDWLLANL

SEQ ID NO: 166 - protein, 4 aa, Artificial Sequence
  Synthetic
GGGG

SEQ ID NO: 167 - protein, 17 aa, Artificial Sequence
  Synthetic
ARSIINFEKLSHHHHHH

SEQ ID NO: 168 - protein, 578 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMSSCNFTHATFVLIGIPGLEKAHFWVGFPLLSMYVVAMFGNCIVVFIVRTERSLHAPMYLFLCMLAAIDLALSTSTMPKILALFWFDSREISFEACLTQMFFIHALSAIESTILLAMAFDRYVAICHPLRHAAVLNNTVTAQIGIVAVVRGSLFFFPLPLLIKRLAFCHSNVLSHSYCVHQDVMKLAYADTLPNVVYGLTAILLVMGVDVMFISLSYFLIIRTVLQLPSKSERAKAFGTCVSHIGVVLAFYVPLIGLSVVHRFGNSLHPIVRVVMGDIYLLLPPVINPIIYGAKTKQIRTRVLAMFKISCDKDLQAVGGKARSIINFEKLSHHHHHH

SEQ ID NO: 169 - protein, 443 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKARSIINFEKLSHHHHHH

SEQ ID NO: 170 - protein, 656 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 171 - protein, 1112 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMMAYSDTTMMSDDIDWLRSHRGVCKVDLYNPEGQQDQDRKVICFVDVSTLNVEDKDYKDAASSSSEGNLNLGSLEEKEIIVIKDTEKKDQSKTEGSVCLFKQAPSDPVSVLNWLLSDLQKYALGFQHALSPSTSTCKHKVGDTEGEYHRASSENCYSVYADQVNIDYLMNRPQNLRLEMTAAKNTNNNQSPSAPPAKPPSTQRAVISPDGECSIDDLSFYVNRLSSLVIQMAHKEIKEKLEGKSKCLHHSICPSPGNKERISPRTPASKIASEMAYEAVELTAAEMRGTGEESREGGQKSFLYSELSNKSKSGDKQMSQRESKEFADSISKGLMVYANQVASDMMVSLMKTLKVHSSGKPIPASVVLKRVLLRHTKEIVSDLIDSCMKNLHNITGVLMTDSDFVSAVKRNLFNQWKQNATDIMEAMLKRLVSALIGEEKETKSQSLSYASLKAGSHDPKCRNQSLEFSTMKAEMKERDKGKMKSDPCKSLTSAEKVGEHILKEGLTIWNQKQGNSCKVATKACSNKDEKGEKINASTDSLAKDLIVSALKLIQYHLTQQTKGKDTCEEDCPGSTMGYMAQSTQYEKCGGGQSAKALSVKQLESHRAPGPSTCQKENQHLDSQKMDMSNIVLMLIQKLLNENPFKCEDPCEGENKCSEPRASKAASMSNRSDKAEEQCQEHQELDCTSGMKQANGQFIDKLVESVMKLCLIMAKYSNDGAALAELEEQAASANKPNFRGTRCIHSGAMPQNYQDSLGHEVIVNNQCSTNSLQKQLQAVLQWIAASQFNVPMLYFMGDKDGQLEKLPQVSAKAAEKGYSVGGLLQEVMKFAKERQPDEAVGKVARKQLLDWLLANLARSIINFEKLSHHHHHH

SEQ ID NO: 172 - protein, 588 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKARSIINFEKLSHHHHHH

SEQ ID NO: 173 - protein, 801 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 174 - protein, 845 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 175 - protein, 990 aa, Artificial Sequence
  Synthetic
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 176 - protein, 1019 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMSSCNFTHATFVLIGIPGLEKAHFWVGFPLLSMYVVAMFGNCIVVFIVRTERSLHAPMYLFLCMLAAIDLALSTSTMPKILALFWFDSREISFEACLTQMFFIHALSAIESTILLAMAFDRYVAICHPLRHAAVLNNTVTAQIGIVAVVRGSLFFFPLPLLIKRLAFCHSNVLSHSYCVHQDVMKLAYADTLPNVVYGLTAILLVMGVDVMFISLSYFLIIRTVLQLPSKSERAKAFGTCVSHIGVVLAFYVPLIGLSVVHRFGNSLHPIVRVVMGDIYLLLPPVINPIIYGAKTKQIRTRVLAMFKISCDKDLQAVGGKARSIINFEKLSHHHHHH

SEQ ID NO: 177 - protein, 884 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKARSIINFEKLSHHHHHH

SEQ ID NO: 178 - protein, 1097 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 179 - protein, 1553 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMMAYSDTTMMSDDIDWLRSHRGVCKVDLYNPEGQQDQDRKVICFVDVSTLNVEDKDYKDAASSSSEGNLNLGSLEEKEIIVIKDTEKKDQSKTEGSVCLFKQAPSDPVSVLNWLLSDLQKYALGFQHALSPSTSTCKHKVGDTEGEYHRASSENCYSVYADQVNIDYLMNRPQNLRLEMTAAKNTNNNQSPSAPPAKPPSTQRAVISPDGECSIDDLSFYVNRLSSLVIQMAHKEIKEKLEGKSKCLHHSICPSPGNKERISPRTPASKIASEMAYEAVELTAAEMRGTGEESREGGQKSFLYSELSNKSKSGDKQMSQRESKEFADSISKGLMVYANQVASDMMVSLMKTLKVHSSGKPIPASVVLKRVLLRHTKEIVSDLIDSCMKNLHNITGVLMTDSDFVSAVKRNLFNQWKQNATDIMEAMLKRLVSALIGEEKETKSQSLSYASLKAGSHDPKCRNQSLEFSTMKAEMKERDKGKMKSDPCKSLTSAEKVGEHILKEGLTIWNQKQGNSCKVATKACSNKDEKGEKINASTDSLAKDLIVSALKLIQYHLTQQTKGKDTCEEDCPGSTMGYMAQSTQYEKCGGGQSAKALSVKQLESHRAPGPSTCQKENQHLDSQKMDMSNIVLMLIQKLLNENPFKCEDPCEGENKCSEPRASKAASMSNRSDKAEEQCQEHQELDCTSGMKQANGQFIDKLVESVMKLCLIMAKYSNDGAALAELEEQAASANKPNFRGTRCIHSGAMPQNYQDSLGHEVIVNNQCSTNSLQKQLQAVLQWIAASQFNVPMLYFMGDKDGQLEKLPQVSAKAAEKGYSVGGLLQEVMKFAKERQPDEAVGKVARKQLLDWLLANLARSIINFEKLSHHHHHH

SEQ ID NO: 180 - protein, 1029 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKARSIINFEKLSHHHHHH

SEQ ID NO: 181 - protein, 1242 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 182 - protein, 1286 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 183 - protein, 1431 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANPGGGGGAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDGGGGMSSCNFTHATFVLIGIPGLEKAHFWVGFPRTERSLHAPMYLILALFWFDSREISFEACLTQMDRYVAICHPLRHAAVLNNTVTAQIGRLAFCHSNVLSHSYCVHQDVMKLAYADTLPNVVYGLTRTVLQLPSKSERAKAFGTCVHRFGNSLHPIVRGAKTKQIRTRVLAMFKISCDKDLQAVGGKGGGGMAQKEGGRTVPCCSRPKVAALTAGTRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLARSIINFEKLSHHHHHH

SEQ ID NO: 184 - DNA, 62 bp, Artificial Sequence
  Synthetic
gatcctcgaggagctcctgcagtctagagtcgacactagtggatccagatctcccggggatc

SEQ ID NO: 185 - DNA, 62 bp, Artificial Sequence
  Synthetic
gatccccgggagatctggatccactagtgtcgactctagactgcaggagctcctcgaggatc

SEQ ID NO: 186 - DNA, 16 bp, Artificial Sequence
  Synthetic
catcgatcactctgga

SEQ ID NO: 187 - DNA, 19 bp, Artificial Sequence
  Synthetic
ctaactccaatgttacttg

SEQ ID NO: 188 - DNA, 20 bp, Artificial Sequence
  Synthetic
cctggcagccctttctcaag

SEQ ID NO: 189 - DNA, 20 bp, Artificial Sequence
  Synthetic
gcagcattgaaccagaggag

SEQ ID NO: 190 - DNA, 23 bp, Artificial Sequence
  Synthetic
cgagagattagctttgaggcctg

SEQ ID NO: 191 - DNA, 18 bp, Artificial Sequence
  Synthetic
gaggccgtttcttggccg

SEQ ID NO: 192 - DNA, 5851 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgaggagctcctgcagtctagagtcgacactagtggatccagatctcccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 193 - DNA, 711 bp, Artificial Sequence; identical to SEQ ID NO: 83
  Synthetic

SEQ ID NO: 194 - DNA, 423 bp, Artificial Sequence; identical to SEQ ID NO: 84
  Synthetic

SEQ ID NO: 195 - DNA, 963 bp, Artificial Sequence
  Synthetic
atgagttcctgcaacttcacacatgccacctttgtgcttattggtatcccaggattagagaaagcccatttctgggttggcttccctctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagttgtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtga

SEQ ID NO: 196 - DNA, 555 bp, Artificial Sequence; identical to SEQ ID NO: 124
  Synthetic

SEQ ID NO: 197 - DNA, 1254 bp, Artificial Sequence
  Synthetic
atggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctga

SEQ ID NO: 198 - DNA, 1194 bp, Artificial Sequence; identical to SEQ ID NO: 126
  Synthetic

SEQ ID NO: 199 - DNA, 57 bp, Artificial Sequence; identical to SEQ ID NO: 88
  Synthetic

SEQ ID NO: 200 - DNA, 2988 bp, Artificial Sequence
  Synthetic
tctagaattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataacccggg

SEQ ID NO: 201 - DNA, 5635 bp, Artificial Sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatatctctagaattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataacccggggatatcccaatggcgcgccgagcttggctcgagcctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 202 - DNA, 8798 bp, Artificial Sequence
  Synthetic
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgaggagctcctgcagtctagaattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaaccccggtggtggaggtggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatggtggtggaggtatgagttcctgcaacttcacacatgccacctttgtgcttattggtatcccaggattagagaaagcccatttctgggttggcttccctaggacggaacgcagcctgcacgctccgatgtacctcatccttgcccttttctggtttgattcccgagagattagctttgaggcctgtcttacccagatggaccgttatgtggccatctgccacccactgcgccatgctgcagtgctcaacaatacagtaacagcccagattggccggctggccttctgccactccaatgtcctctcgcactcctattgtgtccaccaggatgtaatgaagttggcctatgcagacactttgcccaatgtggtatatggtcttactcgaacggttctgcaactgccttccaagtcagagcgggccaaggcctttggaacctgtgtacaccgctttggaaacagccttcatcccattgtgcgtggtgccaaaaccaaacagatcagaacacgggtgctggctatgttcaagatcagctgtgacaaggacttgcaggctgtgggaggcaagggtggtggaggtatggcgcagaaggagggtggccggactgtgccatgctgctccagacccaaggtggcagctctcactgcggggaccaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgcacgtagtataatcaactttgaaaaactgagtcatcatcatcatcatcattaataacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccacccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgtta